(19) United States
(12) Patent Application Publication
(10) Pub. No.: US 2024/0368571 A1
(43) Pub. Date: Nov. 7, 2024

(54) EUBACTERIUM RECTALE CAS12A MUTANTS

(71) Applicant: INTEGRATED DNA TECHNOLOGIES, INC., Coralville, IA (US)

(72) Inventors: Christopher Anthony VAKULSKAS, North Liberty, IA (US); Adam BRAINARD, Cedar Rapids, IA (US); Liyang ZHANG, North Liberty, IA (US)

(21) Appl. No.: 18/647,516

(22) Filed: Apr. 26, 2024

Related U.S. Application Data
(60) Provisional application No. 63/462,772, filed on Apr. 28, 2023.

(51) Int. Cl. C12N 9/22; C12N 15/11; C12N 15/70

(52) U.S. Cl. CPC C12N 9/22; C12N 15/111; C12N 15/70; C12N 2310/20

(57) ABSTRACT

Described herein is a series of recombinantly engineered Cas12a proteins from Eubacterium rectale. In one aspect, various mutations to the wild type Eubacterium rectale Cas12a polypeptide (ERCAS12A) are provided that have enhanced activity.

### CROSS-REFERENCE TO RELATED APPLICATIONS

This application claims priority to U.S. Provisional Patent Application No. 63/462,772, filed on Apr. 28, 2023, which is incorporated by reference herein in its entirety.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “013670-0028-WOO1_sequence_listing_xml_25 Apr. 2024.xml,” was created on Apr. 25, 2024, contains 1667 sequences, has a file size of 1.94 Mbytes (2,043,904 bytes), and is incorporated by reference in its entirety into the specification.

### BACKGROUND

Eubacterium rectale Cas12a (ERCAS12A) is an engineered RNA-guided endonuclease from Eubacterium rectale (now called Agathobacter rectalis) Clustered Regularly Interspaced Short Palindromic Repeat (CRISPR) adaptive immune system. Zetsche et al., Cell 163: 759-771 (2015), which is incorporated by reference herein for such teachings. Cas12a is guided to a ˜21-24-nt DNA target sequence, or commonly referred as protospacer, by a target site-specific ˜21-24-nt complementary RNA. The Cas12a-gRNA ribonucleoprotein (RNP) complex mediates double-stranded DNA breaks (DSBs), which are then repaired by either the non-homologous end joining (NHEJ) which typically introduces mutations or indels at the cut site, or the homology directed repair (HDR) system for precise editing if a suitable template nucleic acid is present.

Critical to the recognition of correct DNA target for ERCAS12A variants include both crRNA and the canonical “TTTV” protospacer adjacent motif (PAM), which is a 4-bp sequence immediately upstream of the protospacer. Compared to the 2-bp NGG PAM of Cas9 from Streptococcus pyogenes, Cas12a expanded the targetable loci in genome editing, particularly over the T-rich sites that are inaccessible to the Cas9 system. Jinek et al., Science 337: 816-821 (2012), which is incorporated by reference herein for such teachings. Although the occurrence of targetable sites of Cas12a on the genome is less frequent than Cas9, ERCAS12A is an alternative enzyme for use in genome engineering applications. Improving the cleavage activity and associated genome editing efficiency of ERCAS12A would greatly facilitate the development of human cell therapies with enhanced properties.

Previous studies improved the utility of Cas12a from Lachnospiraceae bacterium ND2006 (LbCas12a) and Acidaminococcus sp. (AsCas12a). See, e.g., Gao et al., Nat. Biotechnol. 35(8): 789-792 (2017); Kleinstiver et al., Nat. Biotechnol. 37(3): 276-282 (2019); Zhang et al., Nat. Communications 12: 3908 (2021); U.S. Pat. App. Pub. Nos. US 2021/0348144 A1 and US 2023/0040148 A1, each of which are incorporated by reference herein for such teachings. However, transferring beneficial mutations from AsCas12a and LbCas12a to ERCAS12A was not simple or predictable. Despite significant structural similarity between these homologous proteins, most point mutations that improved the activity of AsCas12a and LbCas12a were detrimental to ERCAS12A.

What is needed are mutations that enhance the activity of ERCAS12A.

### SUMMARY

One embodiment described herein is an isolated polypeptide comprising an Eubacterium rectale Cas12a (ERCAS12A) mutant having one or more amino acid substitutions introduced into a wild-type ERCAS12A polypeptide sequence of SEQ ID NO: 2, where the ERCAS12A mutant polypeptide provides an improvement in nuclease activity as compared to the wild-type ERCAS12A polypeptide. In one aspect, the ERCAS12A mutant has 98% to 99% identity to a polypeptide sequence selected from the even numbered sequences of SEQ ID NO: 4-210. In another aspect, the ERCAS12A mutant has a polypeptide sequence selected from the even numbered sequences of SEQ ID NO: 4-210. In another aspect, the ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353, 444, 465, 469, 470, 498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2. In another aspect, the ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2. In another aspect, the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210). In another aspect, the ERCAS12A mutant is selected from D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).

Another embodiment described herein is an isolated polynucleotide sequence encoding an Eubacterium rectale Cas12a (ERCAS12A) mutant having one or more amino acid substitutions introduced into a wild-type ERCAS12A polypeptide sequence of SEQ ID NO: 2, where the ERCAS12A mutant provides improved gene editing as compared to the wild-type ERCAS12A polypeptide. In one aspect, the encoded ERCAS12A mutant has 98% to 99% identity to a polypeptide sequence selected from the even numbered sequences of SEQ ID NO: 4-210. In another aspect, the encoded ERCAS12A mutant has a polypeptide sequence selected from the even numbered sequences of SEQ ID NO: 4-210. In another aspect, the ERCAS12A mutant polynucleotide sequence has 95% to 99% identity to a nucleotide sequence selected from the odd numbered sequences of SEQ ID NO: 3-209. In another aspect, the ERCAS12A mutant polynucleotide has a nucleotide sequence selected from the odd numbered sequences of SEQ ID NO: 3-209. In another aspect, the encoded ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353, 444, 465, 469, 470, 498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2. In another aspect, the encoded ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2. In another aspect, encoded ERCAS12A mutant polynucleotide is selected from is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210). In another aspect, the ERCAS12A mutant poly nucleotide is selected from Q18V (SEQ ID NO: 3); 135L (SEQ ID NO: 5); G39Q (SEQ ID NO: 7); G39K (SEQ ID NO: 9); 140Q (SEQ ID NO: 11); D56R (SEQ ID NO: 13); D56P (SEQ ID NO: 15); D59R (SEQ ID NO: 17); D59A (SEQ ID NO: 19); S158G (SEQ ID NO: 21); K165Y (SEQ ID NO: 23); S181V (SEQ ID NO: 25); A290K (SEQ ID NO: 27); D291N (SEQ ID NO: 29); V296R (SEQ ID NO: 31); P297V (SEQ ID NO: 33); P297R (SEQ ID NO: 35); P297F (SEQ ID NO: 37); D303R (SEQ ID NO: 39); D303C (SEQ ID NO: 41); E305K (SEQ ID NO: 43); Y307K (SEQ ID NO: 45); Q353R (SEQ ID NO: 47); L444W (SEQ ID NO: 49); F465R (SEQ ID NO: 51); S469K (SEQ ID NO: 53); V470K (SEQ ID NO: 55); 1498K (SEQ ID NO: 57); Y507W (SEQ ID NO: 59); Y507S (SEQ ID NO: 61); Y507R (SEQ ID NO: 63); Q510Y (SEQ ID NO: 65); Q510V (SEQ ID NO: 67); Q51OK (SEQ ID NO: 69); P512V (SEQ ID NO: 71); P512G (SEQ ID NO: 73); Y513S (SEQ ID NO: 75); Y513R (SEQ ID NO: 77); Y513L (SEQ ID NO: 79); Y513G (SEQ ID NO: 81); S514R (SEQ ID NO: 83); S514P (SEQ ID NO: 85); T515P (SEQ ID NO: 87); K516R (SEQ ID NO: 89); K516P (SEQ ID NO: 91); K517P (SEQ ID NO: 93); 1518P (SEQ ID NO: 95); L520M (SEQ ID NO: 97); F522A (SEQ ID NO: 99); A541V (SEQ ID NO: 101); G553M (SEQ ID NO: 103); N556R (SEQ ID NO: 105); N556E (SEQ ID NO: 107); D562V (SEQ ID NO: 109); D562K (SEQ ID NO: 111); D5621 (SEQ ID NO: 113); N583V (SEQ ID NO: 115); L585K (SEQ ID NO: 117); F653R (SEQ ID NO: 119); K712R (SEQ ID NO: 121); N716R (SEQ ID NO: 123); E731P (SEQ ID NO: 125); N732T (SEQ ID NO: 127); A744T (SEQ ID NO: 129); A744P (SEQ ID NO: 131); A744N (SEQ ID NO: 132); F748A (SEQ ID NO: 135); S751A (SEQ ID NO: 137); K754G (SEQ ID NO: 139); 1757W (SEQ ID NO: 141); 1757K (SEQ ID NO: 143); E771R (SEQ ID NO: 145); N787A (SEQ ID NO: 147); A825R (SEQ ID NO: 149); T826K (SEQ ID NO: 151); T826F (SEQ ID NO: 153); Y832R (SEQ ID NO: 155); Y836Q (SEQ ID NO: 157); Y836K (SEQ ID NO: 159); Y839L (SEQ ID NO: 161); F840L (SEQ ID NO: 163); Y907V (SEQ ID NO: 165); Y9071 (SEQ ID NO: 167); Q916Y (SEQ ID NO: 169); E935R (SEQ ID NO: 171); 1936R (SEQ ID NO: 173); K974G (SEQ ID NO: 175); K1019V (SEQ ID NO: 177); 135L/S469K (SEQ ID NO: 179); 135L/T826F (SEQ ID NO: 181); 140Q/S469K (SEQ ID NO: 183); 140Q/T826F (SEQ ID NO: 185); S469K/K516P (SEQ ID NO: 187); S469K/T826F (SEQ ID NO: 189); Y513G/Y832R (SEQ ID NO: 191); Y513G/F840L (SEQ ID NO: 193); 135L/S469K/T826F (SEQ ID NO: 195); 140Q/S469K/T826F (SEQ ID NO: 197); S469K/K516P/T826F (SEQ ID NO: 199); S469K/T826F/F840L (SEQ ID NO: 201); D303R/S469K/T826F/F840L (SEQ ID NO: 203); F465R/S469K/T826F/F840L (SEQ ID NO: 205); D303R/F465R/S469K/T826F/F840L (SEQ ID NO: 207); or D303R/S469K/K516P/T826F/F840L (SEQ ID NO: 209). In another aspect, the ERCAS12A mutant polynucleotide is selected from is selected from: D303R/S469K/T826F/F840L (SEQ ID NO: 203); F465R/S469K/T826F/F840L (SEQ ID NO: 205); D303R/F465R/S469K/T826F/F840L (SEQ ID NO: 207); or D303R/S469K/K516P/T826F/F840L (SEQ ID NO: 209).

Another embodiment described herein is a vector or plasmid comprising the polynucleotide sequences described herein.

Another embodiment described herein is a cell comprising a nucleic acid comprising one or more of the polynucleotide sequences described herein.

Another embodiment described herein is an isolated CRISPR ribonucleoprotein complex comprising a guide RNA and a ERCAS12A mutant polypeptide having one or more amino acid substitutions introduced into a wild-type ERCAS12A polypeptide sequence of SEQ ID NO: 2, where the ERCAS12A mutant provides improved gene editing as compared to the wild-type ERCAS12A polypeptide. In one aspect, the ERCAS12A mutant has a polypeptide sequence selected from the even numbered sequences of SEQ ID NO: 4-210. In another aspect, the ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353, 444, 465, 469, 470, 498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2. In another aspect, the ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2. In another aspect, the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210). In another aspect, the ERCAS12A mutant is selected from D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).

Another embodiment described herein is an isolated CRISPR/Cpf1 endonuclease system comprising a crRNA and a ERCAS12A mutant polypeptide having one or more amino acid substitutions introduced into a wild-type ERCAS12A polypeptide sequence of SEQ ID NO: 2 where the ERCAS12A mutant polypeptide provides improved genome editing as compared to the wild-type ERCAS12A polypeptide. In one aspect, the ERCAS12A mutant has a polypeptide sequence selected from the even numbered sequences of SEQ ID NO: 4-210. In another aspect, the ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353, 444, 465, 469, 470, 498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2. In another aspect, the ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2. In another aspect, the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210). In another aspect, the ERCAS12A mutant is selected from D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).

Another embodiment described herein is a method for performing gene editing comprising: contacting a candidate editing target site locus with a CRISPR ribonucleoprotein complex comprising a crRNA and an ERCAS12A mutant polypeptide comprising having one or more amino acid substitutions introduced into a wild-type ERCAS12A polypeptide sequence of SEQ ID NO: 2, where the ERCAS12A mutant provides an improvement in gene editing as compared to the wild-type ERCAS12A polypeptide. In one aspect, the ERCAS12A mutant has a polypeptide sequence selected from the even numbered sequences of SEQ ID NO: 4-210. In another aspect, the ERCAS12A mutant comprises ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353, 444, 465, 469, 470, 498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2. In another aspect, ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2. In another aspect, the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210). In another aspect, the ERCAS12A mutant is selected from D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).

Another embodiment described herein is a kit comprising a CRISPR ribonucleoprotein complex comprising a guide RNA and an ERCAS12A mutant polypeptide comprising one or more amino acid substitutions introduced into a wild-type ERCAS12A polypeptide sequence of SEQ ID NO: 2, where the ERCAS12A mutant polypeptide provides improved gene editing as compared to the wild-type ERCAS12A polypeptide. In one aspect, the ERCAS12A mutant has a polypeptide sequence selected from the even numbered sequences of SEQ ID NO: 4-210. In another aspect, the ERCAS12A mutant comprises wherein the ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q51OK, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2. In another aspect, the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210). In another aspect, the ERCAS12A mutant is selected from D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).

Another embodiment described herein is the use of a ERCAS12A mutant polypeptide for improving CRISPR/LbCas12a-associated nuclease activity at non-canonical TTTT PAM sites, wherein the ERCAS12A mutant one or more amino acid substitutions introduced into a wild-type ERCAS12A polypeptide sequence of SEQ ID NO: 2, where the ERCAS12A mutant provides improved gene editing as compared to the wild-type ERCAS12A polypeptide. In one aspect, the ERCAS12A mutant has a polypeptide sequence selected from the even numbered sequences of SEQ ID NO: 4-210. In another aspect, the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q51OK (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B show high-throughput characterization of ERCAS12A cleavage activity in E. coli. Three biological replicates of selection were performed and deep-sequenced to determine the enrichment of each ERCAS12A variant. FIG. 1A shows the phenotype scores of synonymous (n=834) and non-synonymous changes (n=22409) over the selection. Notably, most synonymous changes resulted in no apparent enrichment, serving as the baseline activity of wild-type ERCAS12A. FIG. 1B shows the enrichment scores for 23,243 ERCAS12A point mutations over two biological replicates. Results of the high-throughput screen are high reproducible (R²=0.66).

FIG. 2 shows DNA cleavage activity of ERCAS12A variants in E. coli. Each ERCAS12A mutant was cloned and evaluated in the context of E. coli. The survival rate of E. coli was determined by taking the ratio of colony counts with or without selection pressure. Most mutations resulted in higher survival rate than wild type (WT) ERCAS12A (˜10%). The raw data are shown in FIG. 3-5.

FIG. 3 shows examples of point mutations of ERCAS12A with enhanced DNA cleavage activity. Clearly, mutations increased the survival rate at TTTC and TTTT PAM sites, indicating an improved cleavage activity over the WT-ERCAS12A.

FIG. 4 shows examples of point mutations of ERCAS12A with enhanced DNA cleavage activity.

FIG. 5 shows examples of point mutations of ERCAS12A with enhanced DNA cleavage activity.

FIG. 6 shows cleavage activity of ERCAS12A variants in E. coli. Each ErCas12a mutant was cloned and evaluated in the context of E. coli. The survival rate of E. coli was determined by taking the ratio of colony counts with or without selection pressure. Most mutations resulted in higher survival rate than WT ERCAS12A (˜18%) FIG. 7A-D show the performance of ERCAS12A variants in human cells as RNP. ERCAS12A variant, and WT proteins were purified and assembled as RNPs using 6 different crRNAs. FIG. 7A-B show the editing efficiency of each variant measured by T7EI 48-hours post-delivery at 200 nM (FIG. 7A) or 50 nM RNP (FIG. 7B) in electroporation reaction. NC: negative control in the absence of ERCAS12A nuclease. FIG. 7C-D show normalized activity of each variant over WT ERCAS12A at 200 nM RNP (FIG. 7C) and 50 nM RNP (FIG. 7D) respectively. The dash line represents the baseline activity of WT protein.

FIG. 8A-C show the on-target editing efficiency of ERCAS12A point mutations in human cells. FIG. 8A shows the relative editing efficiency of ERCAS12A variants in human cells over two target sites. Plasmids encoding each variant were delivered by lipofection, and the editing efficiency was measured using a T7 endonuclease I assay (T7EI) 2 days post-delivery. FIG. 8B shows the editing efficiency of positive hits from lipofection experiment when delivered as RNP. FIG. 8C shows the normalized editing efficiency of ERCAS12A point mutations to WT.

FIG. 9A-B show the performance of stacked ERCAS12A variants with multiple point mutations in human cells as RNP. ERCAS12A variants, and WT proteins were purified and assembled as RNPs using 6 different crRNAs. FIG. 9A shows the editing efficiency of each variant measured by T7EI 48-hours post-delivery at 50 nM in electroporation reaction. NC: negative control in the absence of ERCAS12A nuclease. FIG. 9B shows the normalized activity of each variant over WT ERCAS12A at 50 nM RNP.

FIG. 10A-H shows the relative editing efficiency of ERCAS12A variants in human cells over 9 target sites. Plasmids encoding each variant were delivered by lipofection, and the editing efficiency was measured by NGS 3 days post-delivery. FIG. 10A shows AAVS1; FIG. 10B shows TGFBR2; FIG. 10C shows PD1; FIG. 10D shows TRAC1; FIG. 10D shows B2M-12; FIG. 10E shows CIITA; FIG. 10G shows CISH; and FIG. 10H shows TRAC2.

FIG. 11 shows the on-target editing efficiency of ERCAS12A Y513G in human cells. ERCAS12A Y513G, and WT proteins were purified and assembled as RNPs using 7 different crRNAs, and the editing efficiency was measured by NGS 2 days post-delivery.

FIG. 12A-D shows the on-target editing efficiency of ERCAS12A mutants in human cells. WT ERCAS12A, Y513G, S514P, Y513G/F840L and AsCas12a-Ultra (e.g., AsCas12a M537R/F870L) proteins were purified and assembled as RNPs using different crRNAs, and the editing efficiency was measured by NGS 2 days post-delivery. FIG. 12A shows AAVS1; FIG. 12B shows CISH; FIG. 12C showsTRAC1; FIG. 12D shows TRAC2.

FIG. 13 shows show the performance of stacked ERCAS12A variants with multiple point mutations in human cells as RNP. ERCAS12A variants, and WT proteins were purified and assembled as RNPs using different crRNAs. D303R/S469K/T826F/F840L; F465R/S469K/T826F/F840L; D303R/S469K/K516P/T826F/F840L) improved activity over ERCAS12A WT.

FIG. 14 shows a summary of the on-target editing efficiency of various ERCAS12A point mutations in human cells.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms “nucleic acid” and “polynucleotide” are interchangeable have the same meaning.

As used herein, the terms “amino acid sequence,” “polypeptide,” and “protein” are interchangeable have the same meaning.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the phrase “an effective amount” of a compound described herein refers to an amount of the compound described herein that will elicit the biological response, for example, reduction or inhibition of an enzyme or a protein activity, or ameliorate symptoms, alleviate conditions, slow or delay disease progression, or prevent a disease, etc.

As used herein, the terms “inhibit,” “inhibition,” or “inhibiting” refer to the reduction or suppression of a given condition, symptom, or disorder, or disease, or a significant decrease in the baseline activity of a biological activity or process.

As used herein, the terms “Eubacterium rectale Cas12a” or “ERCAS12A” are used interchangeably and refer to the Eubacterium rectale (Er) Cas12a protein (previously named Cpf1), a class 2/type V CRISPR RNA-guided endonuclease.

The terms “ERCAS12A,” “wild-type ERCAS12A,” or “WT ERCAS12A” refer to a protein having the amino acid sequence of the naturally occurring Eubacterium rectale Cas12a (e.g., SEQ ID NO: 2, encoded by the nucleotide sequence of SEQ ID NO: 1) and that has biochemical activity when combined with a suitable crRNA to form an active CRISPR/Cas12a endonuclease system.

The term “mutant ERCAS12A protein” or “variant ERCAS12A protein” are used interchangeably and refer to Eubacterium rectale Cas12a protein forms having a different amino acid sequence form the wild-type Eubacterium rectale Cas12a proteins and that have biochemical activity when combined with a suitable crRNA to form an active CRISPR-Cas12a endonuclease system. This includes orthologs and Cas12a variants having different amino acid sequences from the wild-type Eubacterium rectale Cas12a. The mutant ERCAS12A proteins described herein have at least one amino acid substitution to the wild-type ERCAS12A polypeptide sequence. In one aspect, the mutant ERCAS12A polypeptides have at least one amino acid substitution to the wild-type ERCAS12A polypeptide sequence and have an improvement in CRISPR/ERCAS12A-associated nuclease activity at non-canonical TTTT PAM sites as compared to the wild-type ERCAS12A enzyme.

As used herein, the term “substantially purified,” as applied to a composition, refers to a composition having at least 90% purity or greater, including 90% purity, 95% purity, 99% purity and greater than 99% purity.

As used herein, the term “isolated,” when used to describe a composition, such as a polynucleotide, a polypeptide or a ribonucleoprotein complex refers to a substantially purified composition, or in the case of a ribonucleoprotein complex, at least one component being a substantially purified component. In further respect to an isolated ribonucleoprotein complex, preferably all components are substantially purified.

As used herein, the term “affinity tag” refers to a ligand that permits detection and/or selection of an oligonucleotide sequence to which the ligand is attached. For the purposes of this disclosure, a bait may include an affinity tag. In particular, the affinity tag is positioned typically at either or both the N-terminus and/or C-terminus of a polypeptide through conventional chemical coupling technology or recombinant DNA technology. Exemplary affinity tags include biotin, digoxigenin, streptavidin, polyhistine, e.g., (His₆), glutathione-S-transferase (GST), HaloTag®, AviTag, Calmodulin-tag, polyglutamate tag, FLAG-tag, HA-tag, Myc-tag, S-tag, SBP-tag, Softag 3, V5 tag, Xpress tag, a hapten, among others.

As used herein, the term “eukaryotic cell,” includes those cells of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. Preferred human cells include cells derived from somatic cells and germ line cells. Exemplary somatic cells include cells from every major organ and tissue system, including the immune system and hematopoietic system.

As used herein, the phrase “the odd numbered sequences of SEQ ID NO: 3-209” refers to nucleotide sequences of SEQ ID NO: 3, 5, 7 . . . 207, or 209. See Table 14, where all odd numbered sequences are nucleotide sequences.

As used herein, the phrase “the even numbered sequences of SEQ ID NO: 4-210” refers to polypeptide sequences of SEQ ID NO: 4, 6, 8 . . . 206, or 210. See Table 14, where all even numbered sequences are polypeptide sequences.

Described herein are Eubacterium rectale Cas12a (ERCAS12A) variants and methods to enhance the utility of ERCAS12A and variants thereof.

A bacterial-based directed evolution of ERCAS12A was used to identify mutations with enhanced cleavage activity. First a deep-scanning mutagenesis library containing all possible point mutations on the amino acid level over the entire coding sequence of ERCAS12A was created. Most clones contained only one mutation. This type of library allowed us to directly evaluate the phenotype of each point mutation, by measuring their relative survival rates over the wild-type (WT) protein in the bacterial screen. Briefly, the screening strain harboring the toxin plasmid was transformed with ERCAS12A library and crRNA targeting the HPRT38346 site on the toxin plasmid. After recovery and IPTG induction, cells were plated on LB-Chloramphenicol media with arabinose and incubated at 37° C. overnight. ERCAS12A expression plasmids carried by the survived E. coli cells were extracted and purified. Both input and selected plasmid libraries were PCR amplified, randomly fragmented by Nextera library prep kit, and sequenced on Illumina NextSeq with ˜40 million reads per library. The frequencies of mutations at each position of ERCAS12A in both libraries were measured and normalized to the total coverage of each codon. The relative survival rate of each point mutation was calculated as the ratio of normalized frequency between selected and input library. Since the degree of cell survival under the arabinose selection is indicative of the cleavage activity of ERCAS12A variant at HPRT38346 protospacer, any variants that enriched during the selection over WT would be those with enhanced activity at TTTT PAM.

Large collection of putative mutations with enhanced activity in the context of E. coli is described. The phenotype of 22,049 point mutations of ERCAS12A in the bacterial screening that measuring the cleavage activity at non-canonical TTTT PAM. Two biological replicates were performed with phenotype measurements which enabled the ability to isolate a large collection of ERCAS12A variants with enhanced cleavage activity.

The genome editing efficiency of ERCAS12A variants with single or multiple point mutations in human cells were evaluated. ERCAS12A variants were column purified, and delivered to human HEK293 cells as ribonucleoprotein (RNP) to edit 6 genomic targets with canonical TTTV PAM. Editing efficiency of each mutant was quantified by T7EI assay and compared to WT ERCAS12A. This enabled rigorous verification of the activity of putative candidates in the context of human cells without protein overexpression. The editing efficiency was measured by T7 endonuclease I assay (T7E1) 72 hours post-delivery.

The genome editing efficiency of 10 ERCAS12A variants with single or multiple point mutations in human cells. ERCAS12A variants were column purified, and delivered to human HEK293 cells as ribonucleoprotein (RNP) to edit 6 genomic targets with canonical TTTV PAM. Editing efficiency of each mutant was quantified by T7EI assay and compared to WT ERCAS12A. This enabled rigorous verification of the activity of putative candidates in the context of human cells without protein overexpression. The editing efficiency was measured by T7 endonuclease I assay (T7EI) 72-hours post-delivery.

One embodiment described herein is a CRISPR-associated protein comprising a polypeptide encoding a variant of ERCAS12A. The isolated polypeptide comprising a ERCAS12A variant is selected from a polypeptide having one or more amino acid substitutions presented in Table 2 relative to wild type ERCAS12A (SEQ ID NO: 2).

Another embodiment described herein is an ErCas12a variant selected from a polypeptide a polypeptide having one or more amino acid substitutions presented in Table 2 relative to wild type ERCAS12A (SEQ ID NO: 2), wherein the selected ERCAS12A variant displays improved genome editing relative to wild type ERCAS12A (SEQ ID NO: 2), wherein the selected ERCAS12A variant displays improved genome editing relative to wild type ERCAS12A (SEQ ID NO: 2).

Another embodiment described herein is CRISPR ribonucleoprotein complex is provided. The CRISPR ribonucleoprotein including a polypeptide encoding a variant of ERCAS12A. The variant of ERCAS12A is selected from the ERCAS12A variant is selected from a polypeptide having one or more amino acid substitutions presented in Table 2 relative to wild type ERCAS12A (SEQ ID NO: 2).

Another embodiment described herein is method of increasing efficiency of gene editing at TTTN PAM sites in a cell with a CRISPR ribonucleoprotein complex is provided. The method includes the step of contacting a cell with the CRISPR ribonucleoprotein complex. The CRISPR ribonucleoprotein complex includes a guide RNA and a CRISPR-associated protein. The CRISPR-associated protein including a polypeptide encoding a variant of ERCAS12A. The variant of ERCAS12A is selected from the ERCAS12A variant is selected from a polypeptide having one or more amino acid substitutions presented in Table 2 relative to wild type ERCAS12A (SEQ ID NO: 2).

Another embodiment described herein is a kit comprising a guide RNA and a CRISPR-associated protein is provided. The CRISPR-associated protein includes a polypeptide encoding a variant of ERCAS12A.

Another embodiment described herein is a CRISPR-associated protein comprising a polypeptide encoding a variant of ERCAS12A, wherein comprises at least one variant amino acid selected from amino acid positions M1G, N2C, N31, T5D, T5F, T5V, N6P, N7A, N7D, N7E, N71, N7Q, N7Y, F8H, F8K, Q9D, N10A, N10E, F11E, F11Q, 112L, 112Q, 114E, 114H, 114L, 114N, 114V, S15K, S15M, S15T, S16H, S16M, S16N, Q181, Q18M, Q18N, Q18R, Q18T, Q18V, Q18W, A24E, 126P, P27C, P27F, P27G, P27H, P27K, P27L, P27M, P27R, P27Y, T28G, T30D, T30F, T30H, T30Q, T30V, T30W, T31A, T31C, T31D, T31G, T31H, T31L, T31M, T31N, T31P, T31S, Q32A, Q32C, Q32F, Q32G, Q321, Q32K, Q32L, Q32M, Q32N, Q32P, Q32S, Q32T, Q32V, Q32W, Q32Y, Q33F, Q331, Q33P, Q33V, Q33W, Q33Y, F34A, F34L, F34P, F34R, F34W, 135A, 135C, 135E, 135F, 135G, 135K, 135L, 135M, 135N, 135P, 135Q, 135S, 135T, 135V, 135W, 135Y, V36K, V36M, V36P, K37P, N38A, N38D, N38E, N38G, N38H, N381, N38L, N38M, N38P, N38Q, N38R, N38S, N38T, N38V, G39A, G39C, G39D, G39E, G39F, G39H, G391, G39K, G39L, G39M, G39N, G39P, G39Q, G39R, G39S, G39T, G39V, G39W, G39Y, 140C, 140D, 140E, 140K, 140L, 140N, 140P, 140Q, 140R, 140T, 140V, 140W, 140Y, E43C, E431, E43L, E43Q, E43S, E43T, E43V, E45F, E45R, L46C, L46D, L46E, L46G, L46H, L46K, L46N, L46S, L46T, E49C, E49F, E49H, E491, E49K, E49L, E49M, E49N, E49P, E49R, E49S, E49T, E49Y, N50Q, Q52C, Q52V, L54G, D56A, D56K, D56P, D56R, 157A, 157C, 157E, 157G, 157L, 157P, 157Q, 157T, 157V, D59A, D59G, D591, D59L, D59M, D59Q, D59R, D59S, D59T, D60A, D60F, D60G, D60K, D60L, D60M, D60N, D60P, D60R, D60T, D60V, D60Y, Y62A, Y62F, Y62N, Y62Q, Y62V, R63M, R63V, F65E, F65K, F65R, S67A, S67F, S67Q, S67V, S67W, S67Y, E68A, E68R, E68V, T69D, T69F, T69H, T69L, T69M, T69W, S71Q, 173E, D75R, 176F, 176G, 176Q, D77P, T79E, T79R, S80N, S80Q, S80V, L81E, L81H, L81W, L81Y, F82Q, K84F, M85A, M85E, M85F, M85G, M85N, M85Q, M85W, M85Y, E86N, 187A, 187G, 187H, 187W, Q88E, Q88N, Q88S, L89C, L89D, L89G, L89Q, L89S, K90P, N91V, G92D, G92F, D93L, D93M, D93N, D93P, D93R, D93T, N94P, K95E, K95F, K951, K95L, K95P, K95S, K95T, K95W, D96C, T97W, L98A, L98C, L98D, L98G, L98H, L98N, L98P, L98Q, L98S, L98T, 199W, 199Y, E101P, Q1021, Q102V, T103Y, E104P, Y105A, Y105G, Y105P, Y105R, Y105T, R1061, R106T, R106V, A108R, K111A, K111C, K111F, K111G, K111S, K111T, K111V, K111W, K112C, K112D, K112E, K112H, K112L, K112P, K112R, K112T, F113A, A114E, A114S, N115M, N115W, R118A, R118D, R118N, R118V, R118Y, F119D, F119H, K120A, K120C, K120P, K120T, K120V, N121F, M122W, F123E, S124N, L127Q, 1128Q, S129D, S129F, S129G, S1291, D1301, L132A, L132G, L132T, F135A, F135D, F135H, F135T, V136Y, 1137Q, H138A, H138M, H138S, H138V, N139A, N139C, N139F, N139G, N1391, N139L, N139P, N139Q, N139R, N140C, N140G, N140P, N140S, N140T, N141F, N141P, Y142E, Y142G, Y142K, Y142P, Y142R, Y142V, S143E, S143G, S1431, S143R, S143V, A144C, A144D, A1441, A144K, A144N, A144P, A144R, A144T, E1461, E146M, E146R, E146S, E146T, K147A, K147F, K147G, K147L, K147Q, E148C, E148P, E149L, E149R, K150A, K150C, K150G, K1501, K150L, K150V, K150W, K150Y, T151C, T151E, T151Q, T151V, V153K, 1154A, 1154E, 1154H, 1154P, 1154Q, L156C, S158D, S158G, S158K, S158N, F160H, S1631, F164Q, K165Y, Y1671, Y167L, Y167M, K169C, K169F, A172Y, C1741, C174T, C174V, A177M, A177W, D178R, D179P, D179W, S181H, S181N, S181V, S184C, S1841, S184L, S184M, S184V, 1188A, 1188C, 1188W, N190Q, N190R, N192C, N192G, A193F, A193H, A193L, A193N, A193Q, 1195W, F196T, F197E, F197H, F197K, F197Q, F197R, S198D, S198E, S198K, S198N, A200H, A2001, V202C, V202H, R205C, R205M, R205T, 1206D, 1206W, 1206Y, V207W, S209K, L210N, D214T, D214W, 1215A, 1215W, N216F, K217A, K217D, K217V, S219D, S219F, S219P, S219W, G220Y, D221C, D2211, D221L, D221N, D221P, D221R, D221S, M222C, M222P, K223C, K223F, K223G, K223P, D224H, S225A, S225W, S225Y, L226D, L226Y, E228F, E228P, E228T, E228W, M229S, S230E, S230G, L231D, L231K, L231R, E233S, 1234G, 1234W, Y235A, Y235L, Y235N, Y235Q, Y235T, Y235W, S236G, S236K, S236N, S236V, S236W, E238F, E238S, K239F, K239G, K239T, K239V, K239W, G241F, G241H, G241M, G241P, G241Q, G241W, F243H, F243R, 1244F, 1244G, 1244Q, 1244W, 1244Y, T245E, T245K, T245L, T245Q, T245V, T245Y, E247C, E2471, E247M, E247T, G248K, G248M, S250N, N253A, N253G, N253Q, N253W, D2541, D254M, D254Q, D254R, D254S, D254W, C256E, C256H, C256M, C256N, C256Q, K258L, V259N, V259Q, V259Y, S261F, S261H, F262G, F262K, F262P, F262R, M263C, M263E, M263S, L265K, L265W, Y266E, Y266P, Y266R, C267E, C267K, C267M, C267P, C267T, Q268C, Q268G, Q268V, K269Y, N270E, N270G, K271C, K271H, K2711, K271W, E272G, E272K, E272P, E272S, E272Y, N2731, N273R, N273S, N275G, L276K, L276N, Y277L, Y277M, Y277P, K278A, K278F, K278L, K278P, K278S, K278W, Q280D, Q280M, K281D, K281F, K281G, L282K, L282S, H283M, Q285M, Q285T, 1286C, C288G, C288K, 1289W, A2901, A290K, A290N, A290R, D291F, D291L, D291M, D291N, D291P, D291T, D291W, T292H, S293H, S293K, S293W, Y294L, Y294M, Y294V, E295C, E295M, E295R, E295T, V296A, V296D, V296G, V296H, V296K, V296M, V296N, V296P, V296Q, V296R, V296T, V296W, V296Y, P297C, P297F, P297H, P2971, P297K, P297M, P297N, P297R, P297V, P297W, P297Y, K299A, K299E, K299G, K299P, K299V, K299W, F300D, F300E, F300K, F300M, F300Q, E301A, E301C, E301F, E301G, E301K, E301M, E301P, E301Q, E301R, E301T, E301W, S302A, S302C, S302D, S302E, S302G, S302H, S3021, S302M, S302N, S302V, S302W, D303C, D303F, D303G, D303H, D3031, D303K, D303M, D303N, D303P, D303Q, D303R, D303S, D303V, D303W, D303Y, E304T, E305A, E305C, E305F, E305G, E305H, E3051, E305K, E305L, E305M, E305N, E305P, E305R, E305S, E305V, E305W, E305Y, V306C, V306F, V306W, V306Y, Y307A, Y307D, Y307E, Y307G, Y3071, Y307K, Y307L, Y307N, Y307P, Y307Q, Y307R, Y307S, Y307T, Y307V, Y307W, Q3081, Q308W, S309K, S309L, S309M, S309V, V310Y, N311F, N311L, G312A, G312E, G3121, G312K, G312Q, G312S, G312T, F313H, F313M, D3151, D315M, 1317W, S318D, S318K, S3191, S319K, S319N, K320F, H321F, H321V, 1322A, E324C, E324M, E324T, E324W, E324Y, R325K, R327K, R327M, R327V, D331C, D331M, Y333M, N334W, G335Q, Y336H, Y336M, Y336V, Y336W, N337G, D339M, D339Q, D339S, K340A, K340E, K340H, Y342M, Y342R, 1343V, V344E, K346C, F347G, F347R, Y348M, Y348V, E349K, E349N, E349Q, E349R, E349T, S350H, S350R, V351D, V351F, V351K, V351Y, S352M, Q353K, Q353M, Q353N, Q353R, T355M, Y356A, Y356C, Y356E, Y356G, Y3561, Y356M, Y356V, R357K, R357M, D358R, D358W, T361K, T364W, A365N, A365Y, E367M, E367P, 1368P, 1368Y, H369A, H369E, H369G, H3691, H369V, Y370A, Y370G, Y370N, Y370P, Y370R, N371C, N371G, N372A, N372F, N372W, 1373D, L374E, L374H, L374N, L374Y, P3751, P375Y, G376Q, N377L, N377P, N377V, G378N, G378W, K379A, K379F, K379G, K379R, K381L, K381P, K381W, D383M, D383P, K384D, K384G, K384P, V385C, V385Y, K386F, A388H, A3881, A388N, A388Y, V389E, K390C, N391W, N391Y, L393D, L393Y, Q394A, Q394C, Q394F, Q394G, Q3941, Q394M, Q394S, Q394T, Q394V, Q394W, K395F, K3951, K395V, 1397C, 1397Q, T398D, T398F, E399M, 1400E, E402H, S405K, S405M, S405R, N406A, N406V, Y4071, Y407P, Y407R, K408C, K408D, K4081, C410F, S411N, S411V, D413R, D413T, D413W, N414A, N414D, N414H, N414L, N414S, 1415C, 1415E, K416P, K416V, K416W, A417C, A417M, E418W, T419G, T419H, T419Y, Y420K, Y420Q, Y420R, Y420V, 1421 F, H422A, H422C, H422F, H422M, E423A, E423H, E423R, E423S, E423T, E423Y, S425E, S425K, H426G, H4261, 1427H, L428T, L428Y, N429A, N429P, F431A, F431R, E432C, E432L, E432R, E432T, Q434F, Q4341, Q434L, Q434P, Q434S, Q434T, Q434W, E435A, E435L, E435Y, L436A, L436D, L436E, L436G, L436K, L436P, L436R, L436S, L436T, L436W, K437C, K437H, K437P, K437W, Y438A, Y438E, Y438G, Y4381, Y438K, Y438L, Y438P, Y438R, Y438W, N439A, N439G, N439H, N439P, N439S, P440A, P440D, P440E, P440G, P440K, P440L, P440M, P440N, P440Q, P440R, P440S, P440T, E441F, E441G, E441H, E441K, E441N, E441R, 1442A, 1442G, 1442P, 1442Y, H443A, H443E, H443F, H443W, L444A, L444C, L444D, L444E, L444G, L444H, L4441, L444K, L444N, L444T, L444W, L444Y, V445C, V445E, V445K, V445P, V445Q, V445R, V445W, E446P, E446T, E446W, S447D, S447F, S4471, S447L, S447P, S447Q, S447W, S447Y, E448A, E448H, E4481, E448W, E448Y, L449D, K450C, K450G, K450S, K450V, A451C, A4511, A451M, S452G, S452P, S452V, E453F, E453W, L454R, L454S, L454Y, N456M, N456P, N456Q, N456R, V457E, V457T, V457Y, V460E, V460H, V4601, V460K, V460N, V460P, 1461C, 1461Y, N463A, F465A, F465E, F465G, F465K, F465N, F465Q, F465R, F465S, F465T, F465V, H466C, H466K, S469K, S469L, S469M, S469P, S469R, S469V, V470K, V470N, V470Y, M472G, T473F, T473G, T473R, E474P, L476G, L476H, L476K, L476N, L476R, L476T, V477D, V477E, V477M, V477Q, V477T, V477W, K479A, K479D, K479H, D480P, D480Q, N481A, N481D, N482A, N482D, N482H, N482Q, N482S, F483A, F483W, Y484G, Y484R, Y484V, A485H, A485K, A485L, A485Q, A485R, A485S, E486A, E486F, E486G, E486L, E486R, E486S, E486Y, E488Q, E488T, E489C, E489H, E489M, E489S, E489T, E489W, 1490A, 1490E, Y491E, Y491Q, D4921, D492K, D492L, E493C, E493R, Y495V, P496C, P496E, P4961, P496K, P496L, P496S, P496V, P496W, V497W, 1498G, 1498K, 1498P, 1498R, S499C, S499G, S499M, L500S, L503D, L503E, L503G, L503H, L5031, L503K, L503N, L503P, L503R, L503W, V504A, V504C, V504G, V504T, Y507A, Y507C, Y507E, Y507G, Y5071, Y507K, Y507L, Y507Q, Y507R, Y507S, Y507T, Y507V, Y507W, T509L, T509M, T509V, Q51OA, Q510C, Q510D, Q51OE, Q51OF, Q510G, Q510H, Q5101, Q510K, Q510M, Q510N, Q510P, Q510R, Q510S, Q510T, Q510V, Q51OW, Q510Y, K511F, K511G, K511S, K511V, K511Y, P512C, P512D, P512E, P512F, P512G, P512H, P5121, P512N, P512R, P512S, P512T, P512V, P512W, P512Y, Y513A, Y513C, Y513D, Y513E, Y513G, Y513H, Y5131, Y513K, Y513L, Y513M, Y513N, Y513P, Y513Q, Y513R, Y513S, Y513T, Y513V, S514A, S514D, S514F, S514G, S514H, S514K, S514L, S514N, S514P, S514R, S514W, T515A, T515C, T515G, T515L, T515M, T515P, T515S, T515Y, K516A, K516P, K516R, K516S, K516T, K517A, K517D, K517G, K517L, K517P, 1518E, 1518F, 1518L, 1518P, 1518Q, 1518S, 1518T, L520A, L520G, L520H, L520M, L520N, L520Q, L520S, N521E, N521F, N521L, N521P, N521R, F522A, F522C, F522G, F522M, G523A, G523Q, A5281, Y537F, S538C, S538N, A5411, A541L, A541M, A541N, A541Q, A541T, A541V, 1543A, 1543C, 1543F, 1543G, 1543H, 1543Q, 1543S, 1543T, 1543W, L544W, L544Y, M545E, M545Q, R546F, L549D, L552E, L552K, L552Q, G553A, G5531, G553L, G553M, G553S, G553T, G553V, 1554C, F555M, N556A, N556C, N556E, N556F, N556K, N556M, N556Q, N556R, N556V, N556W, A557K, A557S, K558E, K558F, K558H, K558M, K558S, K558T, N559A, N559E, N559G, D562C, D562F, D5621, D562K, D562L, D562M, D562Q, D562T, D562V, D562W, K564A, K564D, K564G, 1565F, 1565R, 1566A, 1566W, E567L, E567Q, E567W, N569P, N569W, T570D, T570F, S571M, S571Q, S571W, S571Y, E572M, K574F, K5741, K574V, G575D, G575W, Y577M, Y577P, K578A, K578C, K578F, M580Q, 1581H, N583A, N583G, N5831, N583S, N583T, N583V, L585K, P586F, G587C, G587H, G602A, E6041, E604T, T605C, T605G, K607C, S609C, S609E, S609K, A610H, A6101, A610N, A610Y, L613G, E6141, K617C, K617Q, K617V, Q618D, N619C, N619R, K620F, S624W, S625N, K6261, K626V, K626Y, H634K, L636H, D638C, D6381, D638S, Y639G, Y639H, Y639M, Y639N, Y639T, N642A, N642M, C643F, C643T, 1644A, 1644Y, A645M, A645N, A645Q, P648C, P6481, P648M, F653R, G654P, F655H, D656G, D656K, S658D, D659K, S661G, S661M, S661Q, E664F, E664R, E664S, S667R, G668M, G668Q, F669P, F669R, L675C, Q676S, Q676T, Q676V, Q676W, Y678F, Y678H, 1680A, 1680F, 1680H, 1680N, 1680Q, 1680S, 1680V, D6811, D681K, D681M, D681Q, D681R, D681T, D681W, W682A, W682D, W682E, W682G, W6821, W682K, W682M, W682N, W682Q, W682T, W682V, T683F, T683Y, 1685Y, S686R, S686T, E687N, K688D, K688V, D691R, L692M, L693A, L693D, L693E, L693G, L693M, L693Q, L693T, L693W, Q694C, Q694G, Q6941, Q694K, Q694R, Q694S, Q694W, Q694Y, E695S, G6971, G697N, G697P, Q698A, Q698F, L699C, L699F, L699Y, Y700A, Y700G, Y7001, Y700T, Y700V, L701P, L701W, F702A, F702C, F702G, F702H, F702L, F702M, F702N, F702Q, F702S, F702T, F702V, Q703C, K711L, K711P, K712A, K712C, K712F, K712G, K712H, K712Q, K712R, K712S, K712W, K712Y, S713R, T714N, T714W, T714Y, N716A, N716C, N7161, N716K, N716M, N716Q, N716R, N716T, N716V, D717A, D717P, L719K, K725Y, L727H, L7271, L727T, F728T, S729H, S729K, E730G, E7301, E730L, E730M, E730V, E731A, E731L, E731P, N732D, N732T, L7331, L733K, D735K, D735R, V737Q, L738E, N741C, N741G, E743N, A744C, A744N, A744P, A744S, A744T, E745P, F748A, F748C, F748D, F748E, F748G, F748H, F7481, F748M, F748N, F748Q, F748R, F748S, F748T, F748V, F748W, K750A, K750E, K750F, K750G, K750H, K750L, K750N, K750P, K750S, K750T, K750W, K750Y, S751A, S751C, S751D, S751E, S751F, S751G, S751H, S751L, S751M, S751N, S751P, S751Q, S751R, S751T, S751V, S752C, S752F, S752G, S752H, S7521, S752L, S752M, S752Q, S752V, S752W, S752Y, 1753E, K754A, K754C, K754D, K754E, K754F, K754G, K754H, K7541, K754L, K754N, K754P, K754Q, K754S, K754T, K754V, K754W, K754Y, P756H, P7561, P756K, P756L, P756M, P756N, P756S, P756T, 1757A, 1757C, 1757F, 1757G, 1757K, 1757L, 1757M, 1757P, 1757Q, 1757R, 1757S, 1757W, 1757Y, 1758A, 1758C, 1758E, 1758G, 1758K, 1758Q, 1758S, 1758T, 1758V, G762K, 1764Q, V766Y, N767R, R768K, T769L, E771A, E771C, E771F, E771H, E7711, E771K, E771M, E771Q, E771R, E771S, E771T, E771V, E771W, E771Y, A772W, E7731, E773P, E774H, E7741, E774K, E774L, E774M, E774Q, E774R, E774S, E774T, E774V, E774W, K775C, Q777R, Q777V, F778G, F778K, F778Q, F778R, 1783Q, V784F, V784H, V7841, V784L, V784R, V784W, V784Y, N787A, N787C, N787F, N787G, N787H, N787M, N787P, N787Q, N787R, N787S, N787W, 1788F, 1788Y, P789C, E790A, E790C, E790F, E790G, E790H, E7901, E790K, E790M, E790N, E790P, E790Q, E790R, E790S, E790T, E790V, E790W, E790Y, N791E, N791F, N791G, Y793Q, Y793R, Y793V, Y793W, Q794L, Q794P, Y797W, K798L, F800W, N801A, N801S, K803A, S804F, S8041, K806C, S809G, A813H, E823A, E823P, E823S, A825H, A825K, A825R, T826C, T826F, T826H, T8261, T826K, T826L, T826M, T826N, T826Q, T826R, T826W, T826Y, N827D, N827E, V829T, D831L, D831M, D831N, D831Q, D831R, Y832A, Y832E, Y832G, Y832K, Y832M, Y832N, Y832Q, Y832R, Y836A, Y836C, Y836D, Y836E, Y836G, Y836H, Y8361, Y836K, Y836L, Y836M, Y836N, Y836Q, Y836R, Y836S, Y836T, Y836V, D837P, Y839A, Y839C, Y839H, Y8391, Y839K, Y839L, Y839M, Y839N, Y839Q, Y839R, Y839S, Y839T, Y839V, L841C, L841G, L8411, L841V, L841Y, M843F, M843Q, P844C, 1845Q, T846G, T846L, T846Q, T846W, T846Y, N848D, F849R, A851N, A851T, 1857A, 1857N, D859P, R860E, R860K, 1861A, L862D, L862H, L862Y, A866D, A866Y, K869C, H872M, H872S, 1876L, V886A, V886G, V886S, T8911, C892D, C892H, C8921, C892T, G893K, N894V, E897Y, Q898G, F901G, F901L, F901M, F901Q, 1903V, Y9071, Y907M, Y907T, Y907V, Y909F, Y909W, Q91OA, Q91ON, Q910S, Q910Y, 1911A, 1911D, 1911E, 1911G, 1911H, 1911Q, 1911W, 1911Y, K912C, K912H, K912Y, K914A, K914C, K914F, K914M, K914Q, K914S, K914T, Q916A, Q916D, Q916L, Q916M, Q916N, Q916V, Q916W, Q916Y, G918D, G918E, G918F, G918H, G9181, G918K, G918L, G918M, G918Q, G918T, G918Y, A919E, A919F, A9191, A919L, A919M, A919Y, Q921A, Q921T, 1922A, 1922C, 1922H, 1922R, 1922W, 1922Y, K925C, E926N, E926S, E926T, K928W, E929C, E929G, E929N, E929P, E929S, G931H, G931Q, K934S, E935A, E935C, E935F, E935G, E935H, E935K, E935L, E935M, E935N, E935Q, E935R, E935S, E935T, E935W, E935Y, 1936A, 1936C, 1936F, 1936G, 1936K, 1936L, 1936Q, 1936R, 1936S, 1936T, 1936Y, L941C, L941N, S942A, L9431, V944C, 1945M, H946W, E947F, E947M, E947P, 1948L, S9491, S949K, S949M, S949Q, S949T, S949V, M951G, V952E, V952W, 1953A, 1953P, K954A, K954P, Y955C, Y955W, N956C, N956F, N956W, N956Y, A957F, A957P, A957T, 1958V, 1959F, D963C, F968W, K970C, K974A, K974C, K974E, K974G, K974M, K974P, K974Q, K974S, K974T, K974W, K974Y, V9751, R977H, K982S, K982Y, F983A, M986G, 1988E, 1988K, L991C, L991W, N992A, N992L, L994A, V995P, F996T, F996V, D998T, D998W, 1999H, 1999P, 11001W, T1002C, T1002L, T1002S, T1002V, E1003M, E1003S, N1004A, N1004G, N1004P, N1004Q, N1004R, L1007A, L1007C, L1007V, L1008F, Y10111, Y1011L, Y1011V, T1014C, Y1015A, Y1015E, Y1015G, Y1015M, Y1015Q, Y1015T, 11016A, 11016E, 11016N, 11016P, P1017D, P1017E, P1017G, P1017N, P1017Q, P1017W, K1019A, K1019C, K1019G, K1019S, K1019V, K1021A, K1021F, K1021V, K1021W, V1023C, V1023N, G1024Y, Q1026M, Q1026N, C1027N, C1029N, Y1032K, Y1032Q, Y1032R, Y1032W, A1036F, A1036H, K1040A, K10401, K1040T, 11050D, 11050H, 11050W, K1052C, K1052G, K10521, K1052W, F1053Y, K1054F, K10541, K1054L, K1054M, K1054V, K1054W, D1055C, D1055F, D10551, D1055L, D1055M, D1055P, D1055Q, D1055T, D1055V, L1056C, L1056E, L10561, T1057Y, V1058H, V1058W, A1060N, A1060Q, K1061C, K1061D, K1061E, K1061F, K10611, K1061L, K1061Q, K1061R, K1061S, K1061T, K1061W, R1062A, R1062D, R1062H, R1062M, R1062V, R1062Y, E1063F, E1063G, E1063W, 11065W, F1068N, D1069M, S1070E, R1072D, R1072E, D1074R, D1074S, S1075V, E1076T, K1077R, C1081A, C10811, T1083E, T1083K, T1083M, T1083Y, N1087A, N1087W, N1088A, N1088P, 11090G, T1091C, T1091W, Q1092A, Q1092C, Q1092F, Q10921, Q1092Y, N1093C, T10941, T1094P, T1094Y, V1095A, V1095D, V1095R, M1096C, S1097D, K1098C, K1098V, K1098W, S1099P, S1099Q, S1100D, S1100G, S1100H, S1100M, S1100Q, Y11061, K1111R, K1111V, R1113E, R11131, R1113K, R1113N, R1113T, R1118F, R1118M, R1118Q, F1119A, F1119N, F1119W, S1120D, D1124T, T1125D, T1125E, T1125H, T1125M, T1125W, 11126A, 11126C, 11126E, T1129H, T11291, T1129V, D1131L, D1131S, E1133T, K11341, T1135Q, T1135R, T1135V, E1137L, E1137N, E1137Q, M1138D, M1138G, T1139Q, T1139V, T1139Y, 11141C, 11141P, N1142A, W1143F, D1145F, G1146K, R1150N, Q1151C, D1155M, 11158A, V1159D, F1163C, F1163N, E1164P, 11165A, L1179D, L1179E, L1179N, R1182K, R1182N, R1182Q, R1182Y, D1183Y, Y1184W, R1186M, L1187M, L1192K, L1192Y, E1194C, E1194F, E1194S, N1195S, N1195Y, N1196A, 11197G, 11197W, A1204C, A1204D, A1204F, A1204M, A1204N, A1204Q, D1206C, D1206F, D12061, A1207Q, L1208A, L1208Q, D1211C, D1211S, D1211T, A1212C, A1212M, A1212S, A1212T, A1212V, N1215G, E1227T, 11228H, 11228Y, K1229T, Q1230C, Q1230G, Q1230T, E1233W, E1233Y, N1234A, N1234E, N1234F, N12341, W1235E, W1235Y, K1236F, K1236P, E1237A, E1237F, E1237G, E12371, G1239H, G1239K, G1239L, G1239Q, G1239T, K1240Y, F1241P, S1242E, S1242Q, D12441, K1245G, K1245V, S1249E, S1249Q, S1249W, N1250A, N1250G, N1250L, N1250P, D1252L, F1254T, D1255M, F1256R, F1256W, K1260F, K1260G, K1260H, R1261M, R1261N, R1261V, R1261W, R1261Y, Y1262M, L1263A, L1263E, L1263N, L1263Q, or combinations thereof, provided that the variant ERCAS12A provides an improvement in CRISPR/ERCAS12A associated nuclease activity.

In one aspect, the ERCAS12A variant comprises at least one variant amino acid selected from amino acid positions Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, 135L/S469K, 135L/T826F, 140Q/S469K, 140Q/T826F, S469K/T826F, S469K/K516P, Y513G/F840L, Y513G/Y832R, 135L/S469K/T826F, 140Q/S469K/T826F, S469K/K516P/T826F, S469K/T826F/F840L, D303R/S469K/T826F/F840L, F465R/S469K/T826F/F840L, D303R/F465R/S469K/T826F/F840L, D303R/S469K/K516P/T826F/F840L, or combinations thereof.

Another embodiment described herein is a CRISPR ribonucleoprotein complex is provided. The CRISPR ribonucleoprotein complex includes a guide RNA and a CRISPR-associated protein. The CRISPR-associated protein includes a polypeptide encoding a variant of ERCAS12A comprising at least one variant amino acid selected from amino acid positions Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, 135L/S469K, 135L/T826F, 140Q/S469K, 140Q/T826F, S469K/T826F, S469K/K516P, Y513G/F840L, Y513G/Y832R, 135L/S469K/T826F, 140Q/S469K/T826F, S469K/K516P/T826F, S469K/T826F/F840L, D303R/S469K/T826F/F840L, F465R/S469K/T826F/F840L, D303R/F465R/S469K/T826F/F840L, D303R/S469K/K516P/T826F/F840L, or combinations thereof, provided that the variant ERCAS12A provides an improvement in CRISPR/ERCAS12A-associated nuclease activity.

Another embodiment described herein is a method of increasing efficiency of gene editing in a cell with a CRISPR ribonucleoprotein complex is provided. The method includes a step of contacting a cell with the CRISPR ribonucleoprotein complex that includes a guide RNA and a CRISPR-associated protein. The CRISPR-associated protein includes a polypeptide encoding a variant of ERCAS12A comprising at least one variant amino acid selected from amino acid positions Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, 135L/S469K, 135L/T826F, 140Q/S469K, 140Q/T826F, S469K/T826F, S469K/K516P, Y513G/F840L, Y513G/Y832R, 135L/S469K/T826F, 140Q/S469K/T826F, S469K/K516P/T826F, S469K/T826F/F840L, D303R/S469K/T826F/F840L, F465R/S469K/T826F/F840L, D303R/F465R/S469K/T826F/F840L, D303R/S469K/K516P/T826F/F840L, or combinations thereof, provided that the variant ERCAS12A provides an improvement in CRISPR/ERCAS12A-associated nuclease activity.

Another embodiment described herein is a kit including a guide RNA and a CRISPR-associated protein comprising a polypeptide encoding a variant of ERCAS12A is provided. The variant of ERCAS12A comprising at least one variant amino acid selected from amino acid positions Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, 135L/S469K, 135L/T826F, 140Q/S469K, 140Q/T826F, S469K/T826F, S469K/K516P, Y513G/F840L, Y513G/Y832R, 135L/S469K/T826F, 140Q/S469K/T826F, S469K/K516P/T826F, S469K/T826F/F840L, D303R/S469K/T826F/F840L, F465R/S469K/T826F/F840L, D303R/F465R/S469K/T826F/F840L, D303R/S469K/K516P/T826F/F840L, or combinations thereof, provided that the variant ERCAS12A provides an improvement in CRISPR/ERCAS12A-associated nuclease activity.

Another embodiment described herein is a nucleic acid encoding a CRISPR-associated protein comprising a polypeptide encoding a variant of ERCAS12A is provided. The variant of ERCAS12A comprises at least one variant amino acid selected from amino acid positions Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, 135L/S469K, 135L/T826F, 140Q/S469K, 140Q/T826F, S469K/T826F, S469K/K516P, Y513G/F840L, Y513G/Y832R, 135L/S469K/T826F, 140Q/S469K/T826F, S469K/K516P/T826F, S469K/T826F/F840L, D303R/S469K/T826F/F840L, F465R/S469K/T826F/F840L, D303R/F465R/S469K/T826F/F840L, D303R/S469K/K516P/T826F/F840L, or combinations thereof, provided that the variant ERCAS12A provides an improvement in CRISPR/ERCAS12A-associated nuclease activity.

Another embodiment described herein is a polynucleotide sequence encoding a ERCAS12A variant. The polynucleotide sequence comprises a nucleotide sequence having at least 90-99% identity of any one of the odd numbered sequences of SEQ ID NO: 3-209 (e.g., SEQ ID NO: 3, 5, 7 . . . 207, or 209). In one aspect, polynucleotide sequence comprises any one of the odd numbered sequences of SEQ ID NO: 3-209 (e.g., SEQ ID NO: 3, 5, 7 . . . 207, or 209).

Another embodiment described herein is CAS endonuclease system comprising an expression cassette encoding a polynucleotide sequence encoding a ERCAS12A polypeptide is provided. The polynucleotide sequence comprises a nucleotide sequence having at least 90-99% identity of any one of the odd numbered sequences of SEQ ID NO: 3-209 (e.g., 3, 5, 7 . . . 207, or 209). In one aspect, polynucleotide sequence comprises any one of the odd numbered sequences of SEQ ID NO: 3-209 (e.g., 3, 5, 7 . . . 207, or 209).

Another embodiment described herein is a CAS endonuclease system comprising an amino acid sequence encoding a ERCAS12A polypeptide. The amino acid sequence includes a polypeptide comprising a ERCAS12A variant selected from a polypeptide having one or more amino acid substitutions presented in Table 1 relative to wild type ERCAS12A (SEQ ID NO: 2), wherein the ERCAS12A variant displays improved genome editing relative to wild type ERCAS12A (SEQ ID NO: 2).

Another embodiment described herein is a method of performing genome editing in eukaryotic cells. The method includes the step of introducing an CAS endonuclease system into the eukaryotic cell, said CAS endonuclease system comprising an expression cassette encoding a polynucleotide sequence encoding a ERCAS12A polypeptide, comprising a ERCAS12A variant selected from a polypeptide having one or more amino acid substitutions presented in Table 1 relative to wild type ERCAS12A (SEQ ID NO: 2), wherein the ERCAS12A variant displays improved genome editing relative to wild type ERCAS12A (SEQ ID NO: 2).

Another embodiment described herein is a method of performing genome editing in a eukaryotic cell. The method includes the step of introducing an CAS endonuclease system into the eukaryotic cell, said CAS endonuclease system comprising an amino acid sequence encoding a ERCAS12A polypeptide comprising a ERCAS12A variant selected from a polypeptide having one or more amino acid substitutions presented in Table 1 relative to wild type ERCAS12A (SEQ ID NO: 2), wherein the ERCAS12A variant displays improved genome editing relative to wild type ERCAS12A (SEQ ID NO: 2) Another embodiment described herein is an CRISPR-associated protein comprising a fusion polypeptide. The fusion polypeptide includes an ERCAS12A open reading frame, a nuclear localization signal, optionally an amino acid linker and optionally an affinity tag.

Another embodiment described herein is a method of performing genome editing in a eukaryotic cell. The method includes the step of introducing an CAS endonuclease system into the eukaryotic cell, said CAS endonuclease system comprising a CRISPR-associated protein.

Another embodiment is a polypeptide encoded by a nucleotide sequence described herein. In one aspect, the polypeptide has at least 85% to 99% identity to the even numbered sequences of SEQ ID NO: 4-210 (e.g., SEQ ID NO: 4, 6, 8 . . . 206, or 210). In another aspect, the polypeptide is selected from the even numbered sequences of SEQ ID NO: 4-210 (e.g., SEQ ID NO: 4, 6, 8 . . . 206, or 210).

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence or a polypeptide encoded by the nucleotide sequence produced by the methods or the means described herein.

Another embodiment described herein is the use of an effective amount of a polypeptide encoded by one or more of the odd numbered sequences of SEQ ID NO: 3-209 (e.g., 3, 5, 7 . . . 207, or 209).

Another embodiment described herein is a research tool comprising a polypeptide encoded by a nucleotide sequence described herein.

Another embodiment described herein is a reagent comprising a polypeptide encoded by a nucleotide sequence described herein.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences at least about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to (a) nucleotide sequences, or degenerate, homologous, or codon-optimized variants thereof, encoding polypeptides having the amino acid sequences in the even numbered sequences of SEQ ID NO: 4-210 (e.g., SEQ ID NO: 4, 6, 8 . . . 206, or 210); (b) nucleotide sequences, or degenerate, homologous, or codon-optimized variants thereof, encoding polypeptides having the amino acid sequences in the even numbered sequences of SEQ ID NO: 4-210 (e.g., SEQ ID NO: 4, 6, 8 . . . 206, or 210); and (c) nucleotide sequences capable of hybridizing to the complement of any of the nucleotide sequences in (a) or (b) above and capable of expressing functional polypeptides of amino acid sequences the even numbered sequences of SEQ ID NO: 4-210 (e.g., SEQ ID NO: 4, 6, 8 . . . 206, or 210).

By a polynucleotide having a nucleotide sequence at least, for example, 90-99% “identical” to a reference nucleotide sequence encoding the odd numbered sequences of SEQ ID NO: 3-209 (e.g., 3, 5, 7 . . . 207, or 209) is intended that the nucleotide sequence of the polynucleotide be identical to the reference sequence except that the polynucleotide sequence can include up to about 10 to 1 point mutations, additions, or deletions per each 100 nucleotides of the reference nucleotide sequence encoding the odd numbered sequences of SEQ ID NO: 3-209 (e.g., 3, 5, 7 . . . 207, or 209).

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences at least about 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2: 4 82-489 (1981). This algorithm can be extended to use with peptide sequences using the scoring matrix developed by Dayhoff, Atlas of Protein Sequences and Structure, M. O. Dayhoff ed., 5 suppl. 3: 353-358, National Biomedical Research Foundation, Washington, D.C., USA, and normalized by Gribskov, Nucl. Acids Res. 14(6): 6745-6763 (1986).

For example, due to the degeneracy of the genetic code, one having ordinary skill in the art will recognize that a large number of the nucleic acid molecules having a sequence at least about 50%, 55%, 60%, 65%, 70%, 75%, 80%, 85%, 90%, 91%, 92%, 93%, 94%, 95%, 96%, 97%, 98%, 99%, or 100% identical to the nucleic acid sequence shown in the odd numbered sequences of SEQ ID NO: 3-209 (e.g., 3, 5, 7 . . . 207, or 209), or degenerate, homologous, or codon-optimized variants thereof, will encode the even numbered sequences of SEQ ID NO: 4-210 (e.g., SEQ ID NO: 4, 6, 8 . . . 206, or 210).

The polynucleotides described herein include those encoding mutations, variations, substitutions, additions, deletions, and particular examples of the polypeptides described herein. For example, guidance concerning how to make phenotypically silent amino acid substitutions is provided in Bowie, J. U. et al., “Deciphering the Message in Protein Sequences: Tolerance to Amino Acid Substitutions,” Science 247: 1306-1310 (1990), wherein the authors indicate that proteins are surprisingly tolerant of amino acid substitutions.

Thus, fragments, derivatives, or analogs of the polypeptides of the even numbered sequences of SEQ ID NO: 4-210 (e.g., SEQ ID NO: 4, 6, 8 . . . 206, or 210) can be (i) ones in which one or more of the amino acid residues (e.g., 1, 2, 3, 4, 5, 7, 8, 9, 10, 11, 12, 13, 14, 15, 16, 17, 18, 19, 20, 25, 30, 35, 40, 45, or 50 residues, or even more) are substituted with a conserved or non-conserved amino acid residue (preferably a conserved amino acid residue). Such substituted amino acid residues may or may not be one encoded by the genetic code, or (ii) ones in which one or more of the amino acid residues includes a substituent group (e.g., 1, 2, 3, 4, 5, 7, 8, 9, 10, 15, 20, 25, 30, 35, 40, 45, or 50 residues or even more), or (iii) ones in which the mature polypeptide is fused with another polypeptide or compound, such as a compound to increase the half-life of the polypeptide (for example, polyethylene glycol), or (iv) ones in which the additional amino acids are fused to the mature polypeptide, such as an IgG Fc fusion region peptide or leader or secretory sequence or a sequence which is employed for purification of the mature polypeptide or a proprotein sequence. Such fragments, derivatives, and analogs are deemed to be within the scope of those skilled in the art from the teachings herein.

In addition, fragments, derivatives, or analogs of the polypeptides of the even numbered sequences of SEQ ID NO: 4-210 (e.g., SEQ ID NO: 4, 6, 8 . . . 206, or 210) can be substituted with one or more conserved or non-conserved amino acid residue (preferably a conserved amino acid residue). In some cases, these polypeptides, fragments, derivatives, or analogs thereof will have a polypeptide sequence at least about 50%, 55%, 60%, 65%, 70%, 75%, 80%, 85%, 90%, 91%, 92%, 93%, 94%, 95%, 96%, 97%, 98%, 99% or 100% identical to the polypeptide sequence shown in the even numbered sequences of SEQ ID NO: 4-210 (e.g., SEQ ID NO: 4, 6, 8 . . . 206, or 210) and will comprise functional or non-functional proteins or enzymes. Similarly, additions or deletions to the polypeptides can be made either at the N- or C-termini or within non-conserved regions of the polypeptide (which are assumed to be non-critical because they have not been photogenically conserved).

As described herein, in many cases the amino acid substitutions, mutations, additions, or deletions are preferably of a minor nature, such as conservative amino acid substitutions that do not significantly affect the folding or activity of the protein or additions or deletions to the N- or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1.

Another embodiment described herein is a polynucleotide vector comprising one or more nucleotide sequences described herein.

Another embodiment described herein is a cell comprising one or more nucleotide sequences described herein or a polynucleotide vector described herein.

It will be apparent to one of ordinary skill in the relevant art that suitable modifications and adaptations to the compositions, formulations, methods, processes, apparata, assemblies, and applications described herein can be made without departing from the scope of any embodiments or aspects thereof. The compositions, apparata, assemblies, and methods provided are exemplary and are not intended to limit the scope of any of the disclosed embodiments. All the various embodiments, aspects, and options disclosed herein can be combined in any variations or iterations. The scope of the compositions, formulations, methods, apparata, assemblies, and processes described herein include all actual or potential combinations of embodiments, aspects, options, examples, and preferences described herein. The compositions, formulations, apparata, assemblies, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. All patents and publications cited herein are incorporated by reference herein for the specific teachings thereof.

Various embodiments and aspects of the inventions described herein are summarized by the following clauses:
Clause 1. An isolated polypeptide comprising an Eubacterium rectale Cas12a (ERCAS12A) mutant having one or more amino acid substitutions introduced into a wild-type ERCAS12A polypeptide sequence of SEQ ID NO: 2, where the ERCAS12A mutant polypeptide provides an improvement in nuclease activity as compared to the wild-type ERCAS12A polypeptide.
Clause 2. The isolated polypeptide of clause 1, wherein the ERCAS12A mutant has 98% to 99% identity to a polypeptide sequence selected from the even numbered sequences of SEQ ID NO: 4-210.
Clause 3. The isolated polypeptide of clause 1 or 2, wherein the ERCAS12A mutant has a polypeptide sequence selected from the even numbered sequences of SEQ ID NO: 4-210.
Clause 4. The isolated polypeptide of any one of clauses 1-3, wherein the ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353, 444, 465, 469, 470, 498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2.
Clause 5. The isolated polypeptide of any one of clauses 1-4, wherein the ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2.
Clause 6. The isolated polypeptide of any one of clauses 1-5, wherein the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291 N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).
Clause 7. The isolated polypeptide of any one of clauses 1-6, wherein the ERCAS12A mutant is selected from D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).
Clause 8. An isolated polynucleotide sequence encoding an Eubacterium rectale Cas12a (ERCAS12A) mutant having one or more amino acid substitutions introduced into a wild-type ERCAS12A polypeptide sequence of SEQ ID NO: 2, where the ERCAS12A mutant provides improved gene editing as compared to the wild-type ERCAS12A polypeptide.
Clause 9. The isolated polynucleotide sequence of clause 8, wherein the encoded ERCAS12A mutant has 98% to 99% identity to a polypeptide sequence selected from the even numbered sequences of SEQ ID NO: 4-210.
Clause 10. The isolated polynucleotide sequence of clause 8 or 9, wherein the encoded ERCAS12A mutant has a polypeptide sequence selected from the even numbered sequences of SEQ ID NO: 4-210.
Clause 11. The isolated polynucleotide sequence of any one of clauses 8-10, wherein the ERCAS12A mutant polynucleotide sequence has 95% to 99% identity to a nucleotide sequence selected from the odd numbered sequences of SEQ ID NO: 3-209.
Clause 12. The isolated polynucleotide sequence of any one of clauses 8-11, wherein the ERCAS12A mutant polynucleotide has a nucleotide sequence selected from the odd numbered sequences of SEQ ID NO: 3-209.
Clause 13. The isolated polynucleotide sequence of any one of clauses 8-12, wherein the encoded ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353, 444, 465, 469, 470, 498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2.
Clause 14. The isolated polynucleotide sequence of any one of clauses 8-13, wherein the encoded ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2.
Clause 15. The isolated polynucleotide sequence of any one of clauses 8-14, wherein encoded ERCAS12A mutant polynucleotide is selected from is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).
Clause 16. The isolated polynucleotide sequence of any one of clauses 8-15, wherein the ERCAS12A mutant poly nucleotide is selected from Q18V (SEQ ID NO: 3); 135L (SEQ ID NO: 5); G39Q (SEQ ID NO: 7); G39K (SEQ ID NO: 9); 140Q (SEQ ID NO: 11); D56R (SEQ ID NO: 13); D56P (SEQ ID NO: 15); D59R (SEQ ID NO: 17); D59A (SEQ ID NO: 19); S158G (SEQ ID NO: 21); K165Y (SEQ ID NO: 23); S181V (SEQ ID NO: 25); A290K (SEQ ID NO: 27); D291N (SEQ ID NO: 29); V296R (SEQ ID NO: 31); P297V (SEQ ID NO: 33); P297R (SEQ ID NO: 35); P297F (SEQ ID NO: 37); D303R (SEQ ID NO: 39); D303C (SEQ ID NO: 41); E305K (SEQ ID NO: 43); Y307K (SEQ ID NO: 45); Q353R (SEQ ID NO: 47); L444W (SEQ ID NO: 49); F465R (SEQ ID NO: 51); S469K (SEQ ID NO: 53); V470K (SEQ ID NO: 55); 1498K (SEQ ID NO: 57); Y507W (SEQ ID NO: 59); Y507S (SEQ ID NO: 61); Y507R (SEQ ID NO: 63); Q510Y (SEQ ID NO: 65); Q510V (SEQ ID NO: 67); Q51OK (SEQ ID NO: 69); P512V (SEQ ID NO: 71); P512G (SEQ ID NO: 73); Y513S (SEQ ID NO: 75); Y513R (SEQ ID NO: 77); Y513L (SEQ ID NO: 79); Y513G (SEQ ID NO: 81); S514R (SEQ ID NO: 83); S514P (SEQ ID NO: 85); T515P (SEQ ID NO: 87); K516R (SEQ ID NO: 89); K516P (SEQ ID NO: 91); K517P (SEQ ID NO: 93); 1518P (SEQ ID NO: 95); L520M (SEQ ID NO: 97); F522A (SEQ ID NO: 99); A541V (SEQ ID NO: 101); G553M (SEQ ID NO: 103); N556R (SEQ ID NO: 105); N556E (SEQ ID NO: 107); D562V (SEQ ID NO: 109); D562K (SEQ ID NO: 111); D5621 (SEQ ID NO: 113); N583V (SEQ ID NO: 115); L585K (SEQ ID NO: 117); F653R (SEQ ID NO: 119); K712R (SEQ ID NO: 121); N716R (SEQ ID NO: 123); E731P (SEQ ID NO: 125); N732T (SEQ ID NO: 127); A744T (SEQ ID NO: 129); A744P (SEQ ID NO: 131); A744N (SEQ ID NO: 132); F748A (SEQ ID NO: 135); S751A (SEQ ID NO: 137); K754G (SEQ ID NO: 139); 1757W (SEQ ID NO: 141); 1757K (SEQ ID NO: 143); E771R (SEQ ID NO: 145); N787A (SEQ ID NO: 147); A825R (SEQ ID NO: 149); T826K (SEQ ID NO: 151); T826F (SEQ ID NO: 153); Y832R (SEQ ID NO: 155); Y836Q (SEQ ID NO: 157); Y836K (SEQ ID NO: 159); Y839L (SEQ ID NO: 161); F840L (SEQ ID NO: 163); Y907V (SEQ ID NO: 165); Y9071 (SEQ ID NO: 167); Q916Y (SEQ ID NO: 169); E935R (SEQ ID NO: 171); 1936R (SEQ ID NO: 173); K974G (SEQ ID NO: 175); K1019V (SEQ ID NO: 177); 135L/S469K (SEQ ID NO: 179); 135L/T826F (SEQ ID NO: 181); 140Q/S469K (SEQ ID NO: 183); 140Q/T826F (SEQ ID NO: 185); S469K/K516P (SEQ ID NO: 187); S469K/T826F (SEQ ID NO: 189); Y513G/Y832R (SEQ ID NO: 191); Y513G/F840L (SEQ ID NO: 193); 135L/S469K/T826F (SEQ ID NO: 195); 140Q/S469K/T826F (SEQ ID NO: 197); S469K/K516P/T826F (SEQ ID NO: 199); S469K/T826F/F840L (SEQ ID NO: 201); D303R/S469K/T826F/F840L (SEQ ID NO: 203); F465R/S469K/T826F/F840L (SEQ ID NO: 205); D303R/F465R/S469K/T826F/F840L (SEQ ID NO: 207); or D303R/S469K/K516P/T826F/F840L (SEQ ID NO: 209).
Clause 17. The isolated polynucleotide sequence of any one of clauses 8-16, wherein the ERCAS12A mutant polynucleotide is selected from is selected from: D303R/S469K/T826F/F840L (SEQ ID NO: 203); F465R/S469K/T826F/F840L (SEQ ID NO: 205); D303R/F465R/S469K/T826F/F840L (SEQ ID NO: 207); or D303R/S469K/K516P/T826F/F840L (SEQ ID NO: 209).
Clause 18. A vector or plasmid comprising the polynucleotide sequence of any one of clauses 8-17.
Clause 19. A cell comprising a nucleic acid comprising one or more of the polynucleotide sequences of any one of clauses 8-18.
Clause 20. An isolated CRISPR ribonucleoprotein complex comprising a guide RNA and a ERCAS12A mutant polypeptide having one or more amino acid substitutions introduced into a wild-type ERCAS12A polypeptide sequence of SEQ ID NO: 2, where the ERCAS12A mutant provides improved gene editing as compared to the wild-type ERCAS12A polypeptide.
Clause 21. The isolated CRISPR ribonucleoprotein complex of clause 20, wherein the ERCAS12A mutant has a polypeptide sequence selected from the even numbered sequences of SEQ ID NO: 4-210.
Clause 22. The isolated CRISPR ribonucleoprotein complex of clause 20 or 21, wherein the ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353, 444, 465, 469, 470, 498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2.
Clause 23. The isolated CRISPR ribonucleoprotein complex of any one of clauses 20-22, wherein the ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2.
Clause 24. The isolated CRISPR ribonucleoprotein complex of any one of clauses 20-23, wherein the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).
Clause 25. The isolated CRISPR ribonucleoprotein complex of any one of clauses 20-24, wherein the ERCAS12A mutant is selected from D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).
Clause 26. An isolated CRISPR/Cpf1 endonuclease system comprising a crRNA and a ERCAS12A mutant polypeptide having one or more amino acid substitutions introduced into a wild-type ERCAS12A polypeptide sequence of SEQ ID NO: 2, where the ERCAS12A mutant polypeptide provides improved genome editing as compared to the wild-type ERCAS12A polypeptide.
Clause 27. The isolated CRISPR/Cpf1 endonuclease system of clause 26, wherein the ERCAS12A mutant has a polypeptide sequence selected from the even numbered sequences of SEQ ID NO: 4-210.
Clause 28. The isolated CRISPR/Cpf1 endonuclease system of clause 26 or 27, wherein the ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353, 444, 465, 469, 470, 498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2.
Clause 29. The isolated CRISPR/Cpf1 endonuclease system of any one of clauses 26-28, wherein the ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2.
Clause 30. The isolated CRISPR/Cpf1 endonuclease system of any one of clauses 26-29, wherein the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).
Clause 31. The isolated CRISPR/Cpf1 endonuclease system of any one of clauses 26-30, wherein the ERCAS12A mutant is selected from D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).
Clause 32. A method for performing gene editing comprising:
   contacting a candidate editing target site locus with a CRISPR ribonucleoprotein complex comprising a crRNA and an ERCAS12A mutant polypeptide comprising having one or more amino acid substitutions introduced into a wild-type ERCAS12A polypeptide sequence of SEQ ID NO: 2, where the ERCAS12A mutant provides improved gene editing as compared to the wild-type ERCAS12A polypeptide.
Clause 33. The method of clause 32, wherein the ERCAS12A mutant has a polypeptide sequence selected from the even numbered sequences of SEQ ID NO: 4-210.
Clause 34. The method of clause 32 or 33, wherein the ERCAS12A mutant comprises ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353, 444, 465, 469, 470, 498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2.
Clause 35. The method of any one of clauses 32-34, wherein the ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2.
Clause 36. The method of any one of clauses 32-35, wherein the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).
Clause 37. The method of any one of clauses 32-36, wherein the ERCAS12A mutant is selected from D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).
Clause 38. A kit comprising a CRISPR ribonucleoprotein complex comprising a guide RNA and an ERCAS12A mutant polypeptide comprising one or more amino acid substitutions introduced into a wild-type ERCAS12A polypeptide sequence of SEQ ID NO: 2, where the ERCAS12A mutant provides improved gene editing as compared to the wild-type ERCAS12A polypeptide.
Clause 39. The kit of clause 38, wherein the ERCAS12A mutant has a polypeptide sequence selected from the even numbered sequences of SEQ ID NO: 4-210.
Clause 40. The kit of clause 38 or 39, wherein the ERCAS12A mutant comprises wherein the ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q510K, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2.
Clause 41. The kit of any one of clauses 38-40, wherein the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).
Clause 42. The kit of any one of clauses 38-41, wherein the ERCAS12A mutant is selected from D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).
Clause 43. Use of a ERCAS12A mutant polypeptide for improving gene editing, wherein the ERCAS12A mutant one or more amino acid substitutions introduced into a wild-type ERCAS12A polypeptide sequence of SEQ ID NO: 2, where the ERCAS12A mutant provides improved gene editing as compared to the wild-type ERCAS12A polypeptide.
Clause 44. The use of clause 43, wherein the ERCAS12A mutant has a polypeptide sequence selected from the even numbered sequences of SEQ ID NO: 4-210.
Clause 45. The use of clause 43 or 44, wherein the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291 N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).

### EXAMPLES

### Example 1

High-Throughput Measurement of DNA Cleavage Activity of ERCAS12A Variants at TTTT PAM Sites in E. coli.

The following Example demonstrates the ability of the invention to increase the efficiency of gene editing as compared to ERCAS12A (variant of E. rectalis Cas12a). The wild type nucleotide sequence of ERCAS12A (SEQ ID NO: 1) and amino acid sequence (SEQ ID NO: 2) are shown in Table 1.

TABLE 1
ERCAS12A Polynucleotide and Polypeptide
Sequences

Wild Type ERCAS12A Nucleotide Sequence
(SEQ ID NO: 1)
ATGAATAACGGCACCAACAACTTCCAGAACTTTATTGGTATTAGC
AGCCTGCAGAAAACCCTGCGTAATGCACTGATTCCGACCGAAACC
ACACAGCAGTTTATTGTGAAAAACGGCATCATCAAAGAAGATGAA
CTTCGCGGAGAAAATCGCCAGATTCTGAAAGATATCATGGATGAT
TATTATCGCGGTTTTATCAGCGAAACCCTGAGCAGCATTGATGAT
ATTGATTGGACCAGCCTGTTCGAGAAAATGGAAATCCAGCTGAAA
AATGGCGATAACAAAGACACCCTGATTAAAGAACAGGCCGAAAAA
CGTAAAGCCATCTACAAAAAATTCGCCGATGATGACCGCTTTAAA
AACATGTTCAGCGCAAAACTGATTAGCGATATTCTGCCGGAATTT
GTGATCCACAATAATAACTATAGCGCCAGCGAGAAAGAAGAGAAA
ACCCAGGTTATTAAACTGTTTAGCCGTTTTGCGACCAGCTTCAAA
GATTACTTCAAAAATCGTGCCAACTGCTTCAGCGCAGATGATATT
AGCAGTAGCAGCTGTCATCGTATTGTGAATGATAACGCCGAAATC
TTTTTTAGCAATGCCCTGGTTTATCGTCGCATCGTTAAAAATCTG
AGCAACGACGATATCAACAAAATCAGCGGTGATATGAAAGACAGC
CTGAAAGAAATGAGCCTGGAAAAAATCTACAGCTACGAAAAATAT
GGCGAGTTCATTACCCAAGAAGGCATCAGCTTTTATAACGATATT
TGCGGCAAAGTGAACAGCTTTATGAATCTGTATTGCCAGAAAAAC
AAAGAGAACAAAAACCTGTACAAACTGCGCAAACTGCATAAGCAG
ATTCTGTGTATTGCAGATACCAGCTATGAAGTGCCGTATAAATTC
GAAAGTGATGAAGAAGTGTACCAGAGCGTTAATGGTTTTCTGGAT
AACATCAGCAGCAAACACATTGTTGAACGTCTGCGTAAAATTGGC
GACAACTATAATGGCTATAACCTGGACAAAATTTACATCGTGAGC
AAGTTTTATGAACTGGTGAGCCAGAAAACATATCGTGATTGGGAA
ACAATTAATACCGCCTTGGAGATCCACTACAATAACATTCTGCCT
GGTAATGGTAAAAGCAAAGCCGACAAAGTTAAAAAGGCCGTGAAA
AATGATCTGCAGAAATCCATTACCGAAATCAACGAACTGGTCAGC
AACTATAAACTGTGTCCGGATGATAATATCAAGGCCGAAACCTAT
ATTCACGAAATCTCCCATATCCTGAACAACTTTGAAGCACAAGAG
CTGAAATATAACCCGGAAATTCATCTGGTTGAAAGCGAACTGAAA
GCCAGTGAACTGAAAAACGTTCTGGATGTGATTATGAACGCCTTT
CATTGGTGTAGCGTTTTTATGACCGAAGAACTGGTGGATAAGGAT
AACAACTTTTATGCCGAACTGGAAGAGATCTATGATGAAATTTAT
CCGGTGATCAGCCTGTATAATCTGGTGCGTAATTATGTGACCCAG
AAACCGTATAGCACCAAAAAAATCAAACTGAACTTTGGCATTCCG
ACACTGGCAGATGGTTGGAGCAAAAGTAAAGAATATAGCAACAAC
GCCATTATCCTGATGCGCGATAATCTGTACTATCTGGGTATCTTT
AACGCCAAAAACAAACCGGACAAAAAGATCATTGAAGGCAATACC
AGCGAAAACAAAGGCGACTATAAAAAGATGATCTACAATCTGCTG
CCTGGTCCGAATAAAATGATTCCGAAAGTTTTCCTGAGCAGTAAA
ACCGGTGTGGAAACATATAAACCGAGCGCATATATTCTGGAAGGC
TACAAACAGAACAAGCATCTGAAAAGCAGCAAAGACTTTGATATC
ACCTTTTGCCGTGATCTGATCGACTATTTCAAAAACTGCATTGCC
ATTCATCCGGAATGGAAAAACTTTGGCTTTGATTTTAGCGATACC
AGCACCTATGAAGATATCAGCGGTTTTTATCGTGAAGTTGAACTG
CAGGGCTATAAAATCGATTGGACCTATATTAGCGAGAAGGATATT
GATCTGCTGCAAGAAAAAGGTCAGCTGTACCTGTTTCAGATCTAC
AACAAAGACTTCAGCAAAAAAAGCACCGGCAATGATAATCTGCAT
ACCATGTATCTGAAGAACCTGTTTAGCGAAGAGAACCTGAAAGAC
ATTGTGCTGAAACTGAATGGTGAAGCCGAAATTTTCTTTCGTAAG
AGCAGCATCAAAAACCCGATCATCCATAAAAAAGGTAGCATTCTG
GTGAATCGTACGTATGAAGCAGAAGAAAAAGACCAGTTTGGCAAC
ATTCAGATTGTGCGTAAAACAATCCCGGAAAACATCTATCAAGAG
CTGTACAAATACTTTAACGATAAAAGCGATAAAGAACTGTCCGAT
GAAGCAGCCAAACTGAAGAATGTTGTTGGTCATCATGAAGCCGCA
ACCAACATTGTTAAAGATTATCGTTACACCTATGATAAATACTTT
CTGCACATGCCGATCACCATCAACTTTAAAGCAAATAAGACGAGC
TTCATCAACGATCGCATTCTGCAGTATATCGCCAAAGAAAAGGAT
CTGCATGTGATTGGTATTGATCGTGGTGAACGCAATCTGATTTAT
GTGAGCGTTATTGATACCTGCGGCAATATTGTGGAACAGAAAAGC
TTTAATATCGTGAACGGCTATGACTACCAGATCAAGCTGAAACAG
CAAGAAGGTGCACGTCAGATTGCACGTAAAGAATGGAAAGAAATC
GGCAAGATCAAAGAAATCAAAGAGGGTTATCTGAGCCTGGTGATT
CATGAAATTAGCAAAATGGTGATCAAATATAACGCCATCATTGCC
ATGGAAGATCTGAGTTATGGTTTCAAAAAAGGTCGCTTTAAGGTG
GAACGTCAGGTGTATCAGAAATTTGAAACGATGCTGATCAACAAA
CTGAATTATCTGGTGTTCAAGGACATCAGCATTACGGAAAATGGT
GGTCTGCTGAAAGGTTATCAGCTGACGTATATTCCGGAAAAGCTT
AAAAATGTGGGTCATCAGTGTGGCTGCATCTTTTATGTTCCGGCA
GCCTATACCAGCAAAATTGATCCGACCACCGGCTTTGTTAACATC
TTCAAATTCAAAGATCTGACCGTGGATGCCAAACGCGAATTTATC
AAAAAGTTTGATAGCATCCGCTACGACAGCGAGAAGAACTTATTT
TGTTTCACCTTCGACTACAACAACTTTATCACCCAGAATACGGTT
ATGAGCAAAAGCAGCTGGTCAGTTTATACCTATGGTGTGCGTATT
AAACGTCGCTTTGTGAATGGTCGCTTTTCAAATGAAAGCGACACC
ATCGATATCACCAAAGACATGGAAAAAACCCTGGAAATGACCGAT
ATTAACTGGCGTGATGGTCATGATCTGCGTCAGGATATTATCGAT
TATGAAATCGTGCAGCACATCTTTGAAATTTTTCGTCTGACCGTT
CAGATGCGTAATAGCCTGAGCGAGCTGGAAGATCGCGATTATGAT
CGTCTGATTAGTCCGGTTCTGAACGAAAACAACATCTTCTATGAT
AGCGCAAAAGCGGGTGATGCACTGCCGAAAGATGCCGATGCAAAT
GGTGCATATTGTATTGCACTGAAAGGCCTGTATGAAATCAAGCAG
ATTACCGAGAATTGGAAAGAGGATGGTAAATTCAGCCGTGATAAA
CTGAAAATCTCCAACAAGGATTGGTTCGATTTCATCCAGAATAAA
CGCTACCTG
Wild Type ERCAS12A Polypeptide Sequence
(SEQ ID NO: 2)
MNNGTNNFQNFIGISSLQKTLRNALIPTETTQQFIVKNGIIKEDE
LRGENRQILKDIMDDYYRGFISETLSSIDDIDWTSLFEKMEIQLK
NGDNKDTLIKEQTEYRKAIHKKFANDDRFKNMFSAKLISDILPEF
VIHNNNYSASEKEEKTQVIKLESRFATSFKDYFKNRANCESADDI
SSSSCHRIVNDNAEIFFSNALVYRRIVKSLSNDDINKISGDMKDS
LKEMSLEEIYSYEKYGEFITQEGISFYNDICGKVNSFMNLYCQKN
KENKNLYKLQKLHKQILCIADTSYEVPYKFESDEEVYQSVNGELD
NISSKHIVERLRKIGDNYNGYNLDKIYIVSKFYESVSQKTYRDWE
TINTALEIHYNNILPGNGKSKADKVKKAVKNDLQKSITEINELVS
NYKLCSDDNIKAETYIHEISHILNNFEAQELKYNPEIHLVESELK
ASELKNVLDVIMNAFHWCSVFMTEELVDKDNNFYAELEEIYDEIY
PVISLYNLVRNYVTQKPYSTKKIKLNFGIPTLADGWSKSKEYSNN
AIILMRDNLYYLGIFNAKNKPDKKIIEGNTSENKGDYKKMIYNLL
PGPNKMIPKVFLSSKTGVETYKPSAYILEGYKQNKHIKSSKDFDI
TFCHDLIDYFKNCIAIHPEWKNFGFDFSDTSTYEDISGFYREVEL
QGYKIDWTYISEKDIDLLQEKGQLYLFQIYNKDFSKKSTGNDNLH
TMYLKNLFSEENLKDIVLKLNGEAEIFFRKSSIKNPIIHKKGSIL
VNRTYEAEEKDQFGNIQIVRKNIPENIYQELYKYFNDKSDKELSD
EAAKLKNVVGHHEAATNIVKDYRYTYDKYFLHMPITINFKANKTG
FINDRILQYIAKEKDLHVIGIDRGERNLIYVSVIDTCGNIVEQKS
FNIVNGYDYQIKLKQQEGARQIARKEWKEIGKIKEIKEGYLSLVI
HEISKMVIKYNAIIAMEDLSYGFKKGRFKVERQVYQKFETMLINK
LNYLVFKDISITENGGLLKGYQLTYIPDKLKNVGHQCGCIFYVPA
AYTSKIDPTTGFVNIFKFKDLTVDAKREFIKKFDSIRYDSEKNLF
CFTFDYNNFITQNTVMSKSSWSVYTYGVRIKRRFVNGRESNESDT
IDITKDMEKTLEMTDINWRDGHDLRQDIIDYEIVQHIFEIFRLTV
QMRNSLSELEDRDYDRLISPVLNENNIFYDSAKAGDALPKDADAN
GAYCIALKGLYEIKQITENWKEDGKFSRDKLKISNKDWFDFIQNK
RYL

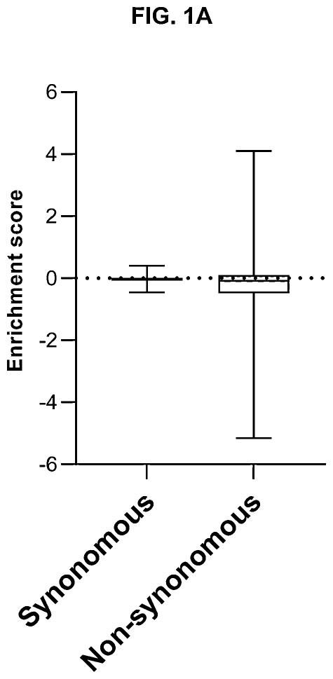

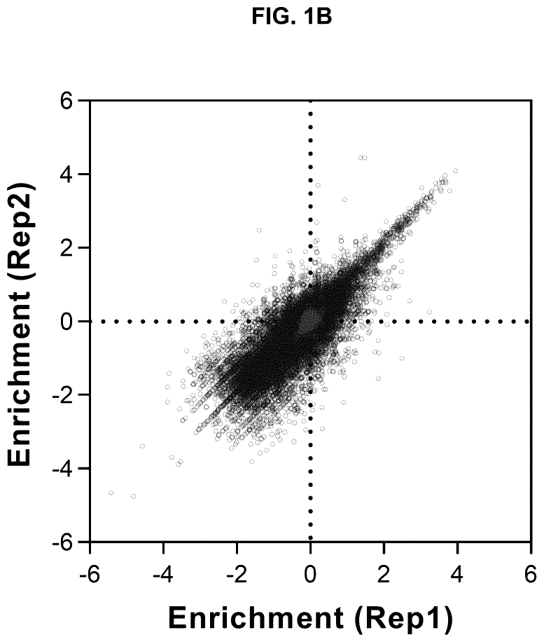

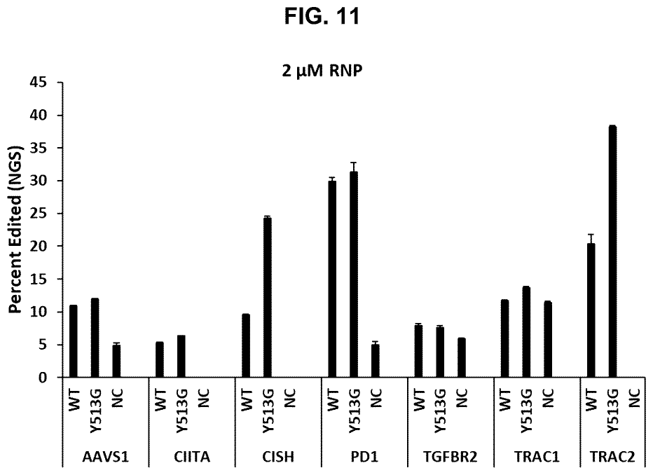

The phenotype scores of 22,409 ERCAS12A variants with single point mutation are listed in Table 2. Scores for synonymous changes (n=849, 69% of all positions) tightly clustered around 0, and thus serving as the baseline activity of ERCAS12A (SEQ ID NO: 2) (FIG. 1A). The enrichment measure for each ERCAS12A point mutation was highly consistent across biological replicates (FIG. 11B). Overall, 2,564 of 22,409 (˜11.4%) point mutations exhibited some benefits with score greater than the maximum score (0.41) of all synonymous changes (Table 2).

Specific mutated residues that conferred some benefit are shown in the sequence of the wild type ERCAS12A Polypeptide Sequence (SEQ ID NO: 2). Mutated residues are bolded; non-bold residues were not mutated. There were 2564 individual mutations at 763 specific residues of the 1263 amino acid residues (˜60%).

MNNGTNNFQNFIGISSLQKTLRNALIPTETTQQFIVKNGIIKEDE
LRGENRQILKDIMDDYYRGFISETLSSIDDIDWTSLFEKMEIQLK
NGDNKDTLIKEQAEKRKAIYKKFADDDRFKNMESAKLISDILPEF
VIHNNNYSASEKEEKTQVIKLFSRFATSFKDYFKNRANCFSADDI
SSSSCHRIVNDNAEIFFSNALVYRRIVKNLSNDDINKISGDMKDS
LKEMSLEKIYSYEKYGEFITQEGISFYNDICGKVNSFMNLYCQKN
KENKNLYKLRKLHKQILCIADTSYEVPYKFESDEEVYQSVNGELD
NISSKHIVERLRKIGDNYNGYNLDKIYIVSKFYELVSQKTYRDWE
TINTALEIHYNNILPGNGKSKADKVKKAVKNDLQKSITEINELVS
NYKLCPDDNIKAETYIHEISHILNNFEAQELKYNPEIHLVESELK
ASELKNVLDVIMNAFHWCSVFMTEELVDKDNNFYAELEEIYDEIY
PVISLYNLVRNYVTQKPYSTKKIKLNFGIPTLADGWSKSKEYSNN
AIILMRDNLYYLGIFNAKNKPDKKIIEGNTSENKGDYKKMIYNLL
PGPNKMIPKVFLSSKTGVETYKPSAYILEGYKQNKHLKSSKDFDI
TFCRDLIDYFKNCIAIHPEWKNFGFDFSDTSTYEDISGFYREVEL
QGYKIDWTYISEKDIDLLQEKGQLYLFQIYNKDFSKKSTGNDNLH
TMYLKNLFSEENLKDIVLKLNGEAEIFFRKSSIKNPIIHKKGSIL
VNRTYEAEEKDQFGNIQIVRKTIPENIYQELYKYENDKSDKELSD
EAAKLKNVVGHHEAATNIVKDYRYTYDKYFLHMPITINFKANKTS
FINDRILQYIAKEKDLHVIGIDRGERNLIYVSVIDTCGNIVEQKS
ENIVNGYDYQIKLKQQEGARQIARKEWKEIGKIKEIKEGYLSLVI
HEISKMVIKYNAIIAMEDLSYGEKKGRFKVERQVYQKFETMLINK
LNYLVFKDISITENGGLLKGYQLTYIPEKLKNVGHQCGCIFYVPA
AYTSKIDPTTGFVNIFKFKDLTVDAKREFIKKFDSIRYDSEKNLF
CFTFDYNNFITQNTVMSKSSWSVYTYGVRIKRRFVNGRESNESDT
IDITKDMEKTLEMTDINWRDGHDLRQDIIDYEIVQHIFEIFRLTV
QMRNSLSELEDRDYDRLISPVLNENNIFYDSAKAGDALPKDADAN
GAYCIALKGLYEIKQITENWKEDGKFSRDKLKISNKDWEDFIQNK
RYL

Table 2 shows ERCAS12A mutations with improved DNA cleavage activity in E. coli. The enrichment scores over 3 biological replicates were measured by deep sequencing. Mutants with average enrichment score greater than the maximal score of all synonymous variants (0.41) were considered as positive hits.

TABLE 2
ERCAS12A Mutations with Improved DNA
Cleavage Activity in E. coli

| | | | | | | | Enrich. |
|---|---|---|---|---|---|---|---|
| | | WT | Enrich. | Enrich. | Enrich. | Enrich. | (Std |
| No. | Mutant | Posit. | (REP1) | (REP 2) | (REP3) | (Avg) | Dev) |
| 1 | M1G | 1 | 0.23 | 1.37 | 0.7 | 0.77 | 0.47 |
| 2 | N2C | 2 | 0.23 | 0.28 | 2.21 | 0.9 | 0.92 |
| 3 | N3I | 3 | 0.63 | 0.68 | 0.01 | 0.44 | 0.31 |
| 4 | T5D | 5 | 2.31 | 0.98 | 0.7 | 1.33 | 0.7 |
| 5 | T5V | 5 | 0.74 | 0.8 | 0.7 | 0.75 | 0.04 |
| 6 | T5F | 5 | 0.52 | 0.69 | 0.42 | 0.54 | 0.11 |
| 7 | N6P | 6 | 0.7 | 0.62 | 0.8 | 0.71 | 0.07 |
| 8 | N7A | 7 | 1.53 | 1.26 | 0.99 | 1.26 | 0.22 |
| 9 | N7Q | 7 | 1.01 | 1.14 | 0.83 | 1 | 0.13 |
| 10 | N7E | 7 | 0.64 | 0.4 | 1.07 | 0.7 | 0.28 |
| 11 | N7I | 7 | 0.87 | 0.73 | 0.45 | 0.68 | 0.17 |
| 12 | N7D | 7 | 0.64 | 0.69 | 0.7 | 0.68 | 0.03 |
| 13 | N7Y | 7 | 0.49 | 0.55 | 0.65 | 0.56 | 0.07 |
| 14 | F8K | 8 | 1.34 | 2.08 | 1.96 | 1.79 | 0.33 |
| 15 | F8H | 8 | 0.64 | 0.51 | 0.24 | 0.47 | 0.17 |
| 16 | Q9D | 9 | 0.24 | 1.39 | 0.71 | 0.78 | 0.47 |
| 17 | N10A | 10 | 1.09 | 0.98 | 0.7 | 0.92 | 0.16 |
| 18 | NI0E | 10 | 0.24 | 1.67 | 0.01 | 0.64 | 0.74 |
| 19 | F11Q | 11 | 0.24 | 0.98 | 0.86 | 0.69 | 0.33 |
| 20 | F11E | 11 | 0.93 | 0.29 | 0.41 | 0.54 | 0.28 |
| 21 | I12Q | 12 | 0.64 | 1.13 | 0.52 | 0.76 | 0.27 |
| 22 | I12L | 12 | 0.53 | 0.28 | 0.45 | 0.42 | 0.1 |
| 23 | I14H | 14 | 1.06 | 0.78 | 1.08 | 0.97 | 0.14 |
| 24 | I14E | 14 | 1.2 | 0.9 | 0.68 | 0.93 | 0.21 |
| 25 | I14N | 14 | 0.93 | 0.86 | 0.87 | 0.89 | 0.03 |
| 26 | I14L | 14 | 0.74 | 0.46 | 0.47 | 0.56 | 0.13 |
| 27 | I14V | 14 | 0.39 | 0.48 | 0.79 | 0.55 | 0.17 |
| 28 | S15M | 15 | 1.6 | 1.36 | 1.89 | 1.62 | 0.22 |
| 29 | S15K | 15 | 1.75 | 1.11 | 0.49 | 1.12 | 0.51 |
| 30 | S15T | 15 | 0.72 | 0.67 | 0.93 | 0.77 | 0.11 |
| 31 | S16H | 16 | 1.82 | 1.77 | 1.08 | 1.56 | 0.34 |
| 32 | S16M | 16 | 1.02 | 1.14 | 1.01 | 1.06 | 0.06 |
| 33 | S16N | 16 | 0.72 | 1.11 | −0.42 | 0.47 | 0.65 |
| 34 | Q18I | 18 | 1.83 | 1.96 | 2.11 | 1.97 | 0.12 |
| 35 | Q18V | 18 | 1.05 | 1.62 | 1.83 | 1.5 | 0.33 |
| 36 | Q18M | 18 | 0.82 | 0.84 | 1.04 | 0.9 | 0.1 |
| 37 | Q18T | 18 | 1.22 | 0.73 | 0.38 | 0.77 | 0.34 |
| 38 | Q18N | 18 | 0.41 | 0.55 | 0.69 | 0.55 | 0.12 |
| 39 | Q18W | 18 | 1.05 | 0.95 | −0.43 | 0.52 | 0.68 |
| 40 | Q18R | 18 | 0.42 | 0.55 | 0.3 | 0.42 | 0.11 |
| 41 | A24E | 24 | 0.64 | 0.62 | 0.35 | 0.54 | 0.13 |
| 42 | I26P | 26 | 1.53 | 1.14 | 1.26 | 1.31 | 0.16 |
| 43 | P27F | 27 | 2.41 | 2.51 | 2.51 | 2.48 | 0.05 |
| 44 | P27M | 27 | 1.93 | 1.89 | 1.94 | 1.92 | 0.02 |
| 45 | P27Y | 27 | 1.79 | 1.29 | 1.7 | 1.6 | 0.22 |
| 46 | P27G | 27 | 1.41 | 1.38 | 1.64 | 1.48 | 0.12 |
| 47 | P27H | 27 | 1.47 | 1.17 | 1.32 | 1.32 | 0.12 |
| 48 | P27L | 27 | 0.96 | 0.94 | 0.99 | 0.97 | 0.02 |
| 49 | P27C | 27 | 0.66 | 0.62 | 0.5 | 0.59 | 0.07 |
| 50 | P27K | 27 | 0.87 | 0.29 | 0.37 | 0.51 | 0.26 |
| 51 | P27R | 27 | 0.49 | 0.42 | 0.36 | 0.42 | 0.05 |
| 52 | T28G | 28 | 0.57 | 0.61 | 0.43 | 0.53 | 0.07 |
| 53 | T30F | 30 | 2.47 | 2.16 | 2.14 | 2.26 | 0.15 |
| 54 | T30W | 30 | 1.88 | 1.66 | 1.5 | 1.68 | 0.16 |
| 55 | T30H | 30 | 1.91 | 1.89 | 0.79 | 1.53 | 0.52 |
| 56 | T30D | 30 | 1.42 | 0.62 | 0.86 | 0.97 | 0.34 |
| 57 | T30Q | 30 | 0.62 | 0.35 | 0.7 | 0.56 | 0.15 |
| 58 | T30V | 30 | 0.29 | 0.53 | 0.42 | 0.42 | 0.1 |
| 59 | T31G | 31 | 2.57 | 2.65 | 2.71 | 2.65 | 0.06 |
| 60 | T31C | 31 | 2.38 | 2.73 | 2.62 | 2.58 | 0.15 |
| 61 | T31N | 31 | 2.13 | 2.06 | 2.3 | 2.16 | 0.1 |
| 62 | T31A | 31 | 1.9 | 2.01 | 1.92 | 1.94 | 0.05 |
| 63 | T31P | 31 | 1.04 | 1.03 | 1.02 | 1.03 | 0.01 |
| 64 | T31M | 31 | 0.5 | 0.63 | 0.71 | 0.62 | 0.09 |
| 65 | T31L | 31 | 0.35 | 0.5 | 0.66 | 0.51 | 0.13 |
| 66 | T31H | 31 | 0.63 | 0.58 | 0.31 | 0.5 | 0.14 |
| 67 | T31S | 31 | 0.44 | 0.58 | 0.41 | 0.48 | 0.07 |
| 68 | T31D | 31 | 0.53 | 0.15 | 0.71 | 0.46 | 0.23 |
| 69 | Q32W | 32 | 2.47 | 2.62 | 2.72 | 2.61 | 0.1 |
| 70 | Q32M | 32 | 1.89 | 2.07 | 2 | 1.99 | 0.07 |
| 71 | Q32V | 32 | 1.78 | 1.92 | 1.87 | 1.86 | 0.06 |
| 72 | Q32F | 32 | 1.78 | 1.81 | 1.92 | 1.84 | 0.06 |
| 73 | Q32I | 32 | 1.7 | 1.87 | 1.77 | 1.78 | 0.07 |
| 74 | Q32C | 32 | 1.65 | 1.68 | 1.83 | 1.72 | 0.08 |
| 75 | Q32G | 32 | 1.52 | 1.61 | 1.76 | 1.63 | 0.1 |
| 76 | Q32T | 32 | 1.38 | 1.31 | 1.43 | 1.38 | 0.05 |
| 77 | Q32A | 32 | 1.12 | 1.4 | 1.34 | 1.29 | 0.12 |
| 78 | Q32L | 32 | 1.21 | 1.32 | 1.29 | 1.27 | 0.05 |
| 79 | Q32S | 32 | 1.22 | 1.27 | 1.28 | 1.25 | 0.03 |
| 80 | Q32Y | 32 | 1.05 | 0.78 | 1.05 | 0.96 | 0.13 |
| 81 | Q32N | 32 | 0.91 | 0.72 | 0.79 | 0.81 | 0.08 |
| 82 | Q32K | 32 | 0.67 | 0.77 | 0.71 | 0.71 | 0.04 |
| 83 | Q32P | 32 | 0.51 | 0.52 | 0.52 | 0.52 | 0.01 |
| 84 | Q33P | 33 | 1.24 | 1.44 | 1.41 | 1.37 | 0.09 |
| 85 | Q33F | 33 | 1.12 | 1.13 | 1.34 | 1.2 | 0.1 |
| 86 | Q33W | 33 | 0.94 | 1.5 | 0.57 | 1.01 | 0.38 |
| 87 | Q33V | 33 | 0.86 | 0.89 | 1.04 | 0.93 | 0.08 |
| 88 | Q33Y | 33 | 0.42 | 0.89 | 0.94 | 0.75 | 0.23 |
| 89 | Q33I | 33 | 0.62 | 0.47 | 0.96 | 0.68 | 0.2 |
| 90 | F34P | 34 | 2.4 | 2.47 | 2.57 | 2.48 | 0.07 |
| 91 | F34W | 34 | 0.84 | 0.82 | 0.87 | 0.84 | 0.02 |
| 92 | F34L | 34 | 0.54 | 0.73 | 0.7 | 0.65 | 0.08 |
| 93 | F34R | 34 | 0.57 | 0.69 | 0.38 | 0.55 | 0.13 |
| 94 | F34A | 34 | 0.66 | 0.39 | 0.58 | 0.54 | 0.11 |
| 95 | I35L | 35 | 3.18 | 3.38 | 3.39 | 3.32 | 0.1 |
| 96 | I35C | 35 | 3.05 | 3.16 | 3.31 | 3.18 | 0.11 |
| 97 | I35Q | 35 | 2.63 | 2.63 | 2.9 | 2.72 | 0.13 |
| 98 | I35A | 35 | 2.48 | 2.56 | 2.69 | 2.58 | 0.09 |
| 99 | I35S | 35 | 2.48 | 2.49 | 2.54 | 2.5 | 0.03 |
| 100 | I35E | 35 | 2.56 | 2.37 | 2.51 | 2.48 | 0.08 |
| 101 | I35M | 35 | 1.91 | 2.14 | 2.1 | 2.05 | 0.1 |
| 102 | I35N | 35 | 1.81 | 1.79 | 1.84 | 1.81 | 0.02 |
| 103 | I35F | 35 | 1.68 | 1.54 | 1.84 | 1.69 | 0.12 |
| 104 | I35T | 35 | 1.36 | 1.4 | 1.46 | 1.41 | 0.04 |
| 105 | I35Y | 35 | 1.26 | 1.38 | 1.52 | 1.38 | 0.11 |
| 106 | I35W | 35 | 1.06 | 1.27 | 1 | 1.11 | 0.12 |
| 107 | I35K | 35 | 1.13 | 0.97 | 1.22 | 1.11 | 0.1 |
| 108 | I35V | 35 | 0.59 | 0.7 | 0.9 | 0.73 | 0.13 |
| 109 | I35P | 35 | 0.38 | 0.51 | 0.66 | 0.52 | 0.11 |
| 110 | I35G | 35 | 0.55 | 0.16 | 0.72 | 0.48 | 0.23 |
| 111 | V36P | 36 | 1.7 | 1.88 | 1.96 | 1.85 | 0.11 |
| 112 | V36K | 36 | 1.65 | 1.58 | 1.84 | 1.69 | 0.11 |
| 113 | V36M | 36 | 0.47 | 0.67 | 0.59 | 0.58 | 0.08 |
| 114 | K37P | 37 | 1.86 | 2.01 | 1.99 | 1.95 | 0.06 |
| 115 | N38L | 38 | 1.96 | 2.04 | 2 | 2 | 0.03 |
| 116 | N38R | 38 | 1.58 | 1.8 | 1.75 | 1.71 | 0.1 |
| 117 | N38G | 38 | 1.52 | 1.73 | 1.83 | 1.69 | 0.13 |
| 118 | N38D | 38 | 1.5 | 1.64 | 1.93 | 1.69 | 0.18 |
| 119 | N38P | 38 | 1.37 | 1.54 | 1.65 | 1.52 | 0.11 |
| 120 | N38A | 38 | 1.27 | 1.39 | 1.38 | 1.35 | 0.06 |
| 121 | N38T | 38 | 1.19 | 1.18 | 1.03 | 1.13 | 0.07 |
| 122 | N38H | 38 | 0.84 | 1.06 | 0.95 | 0.95 | 0.09 |
| 123 | N38M | 38 | 0.9 | 0.8 | 0.82 | 0.84 | 0.04 |
| 124 | N38S | 38 | 0.77 | 0.92 | 0.76 | 0.81 | 0.08 |
| 125 | N38Q | 38 | 0.72 | 0.8 | 0.42 | 0.65 | 0.16 |
| 126 | N38I | 38 | 0.51 | 0.64 | 0.61 | 0.59 | 0.05 |
| 127 | N38E | 38 | 0.62 | 0.36 | 0.48 | 0.49 | 0.1 |
| 128 | N38V | 38 | 0.47 | 0.45 | 0.52 | 0.48 | 0.03 |
| 129 | G39Q | 39 | 3.42 | 3.64 | 3.58 | 3.55 | 0.09 |
| 130 | G39K | 39 | 3.24 | 3.42 | 3.48 | 3.38 | 0.1 |
| 131 | G39M | 39 | 2.99 | 3.15 | 3.13 | 3.09 | 0.07 |
| 132 | G39A | 39 | 2.97 | 3.12 | 3.15 | 3.08 | 0.08 |
| 133 | G39V | 39 | 2.9 | 3.13 | 3.18 | 3.07 | 0.12 |
| 134 | G39H | 39 | 2.87 | 2.99 | 2.88 | 2.91 | 0.05 |
| 135 | G39E | 39 | 2.75 | 2.95 | 2.96 | 2.89 | 0.1 |
| 136 | G39F | 39 | 2.56 | 2.93 | 2.87 | 2.79 | 0.16 |
| 137 | G39N | 39 | 2.65 | 2.81 | 2.85 | 2.77 | 0.09 |
| 138 | G39I | 39 | 2.66 | 2.79 | 2.82 | 2.76 | 0.07 |
| 139 | G39L | 39 | 2.59 | 2.76 | 2.81 | 2.72 | 0.09 |
| 140 | G39T | 39 | 2.41 | 2.53 | 2.53 | 2.49 | 0.06 |
| 141 | G39W | 39 | 2.2 | 2.41 | 2.31 | 2.31 | 0.09 |
| 142 | G39C | 39 | 2.12 | 2.12 | 2.14 | 2.13 | 0.01 |
| 143 | G39S | 39 | 2.05 | 2.06 | 2.19 | 2.1 | 0.06 |
| 144 | G39Y | 39 | 1.83 | 1.98 | 1.85 | 1.89 | 0.06 |
| 145 | G39P | 39 | 0.49 | 1.05 | 1.13 | 0.89 | 0.28 |
| 146 | G39D | 39 | 0.52 | 0.74 | 0.92 | 0.73 | 0.17 |
| 147 | G39R | 39 | 0.61 | 0.75 | 0.73 | 0.7 | 0.06 |
| 148 | I40Q | 40 | 2.91 | 3 | 3.08 | 3 | 0.07 |
| 149 | I40N | 40 | 2.6 | 2.81 | 2.77 | 2.73 | 0.09 |
| 150 | I40Y | 40 | 2.49 | 2.62 | 2.78 | 2.63 | 0.12 |
| 151 | I40R | 40 | 2.55 | 2.62 | 2.71 | 2.62 | 0.06 |
| 152 | I40E | 40 | 2.38 | 2.55 | 2.66 | 2.53 | 0.12 |
| 153 | I40C | 40 | 2.38 | 2.51 | 2.48 | 2.46 | 0.06 |
| 154 | I40V | 40 | 2.29 | 2.52 | 2.5 | 2.44 | 0.11 |
| 155 | I40T | 40 | 2.39 | 2.45 | 2.44 | 2.42 | 0.03 |
| 156 | I40K | 40 | 1.59 | 1.76 | 1.75 | 1.7 | 0.08 |
| 157 | I40D | 40 | 1.42 | 1.61 | 1.6 | 1.55 | 0.09 |
| 158 | I40W | 40 | 1.38 | 1.43 | 1.63 | 1.48 | 0.11 |
| 159 | I40L | 40 | 1.13 | 1.25 | 1.24 | 1.2 | 0.05 |
| 160 | I40P | 40 | 0.62 | 0.83 | 0.81 | 0.75 | 0.1 |
| 161 | E43T | 43 | 1.14 | 1.32 | 1.16 | 1.21 | 0.08 |
| 162 | E43Q | 43 | 0.92 | 0.98 | 1.19 | 1.03 | 0.11 |
| 163 | E43S | 43 | 0.73 | 0.98 | 0.81 | 0.84 | 0.11 |
| 164 | E43L | 43 | 0.85 | 0.71 | 0.71 | 0.75 | 0.07 |
| 165 | E43I | 43 | 0.77 | 0.76 | 0.69 | 0.74 | 0.04 |
| 166 | E43V | 43 | 0.65 | 0.68 | 0.62 | 0.65 | 0.02 |
| 167 | E43C | 43 | 0.38 | 0.73 | 0.65 | 0.59 | 0.15 |
| 168 | E45R | 45 | 0.96 | 1.13 | 0.66 | 0.92 | 0.19 |
| 169 | E45F | 45 | 0.52 | 0.57 | 0.74 | 0.61 | 0.09 |
| 170 | L46N | 46 | 1.25 | 1.4 | 1.6 | 1.42 | 0.15 |
| 171 | L46S | 46 | 1.29 | 1.55 | 1.41 | 1.41 | 0.11 |
| 172 | L46D | 46 | 1.12 | 1.35 | 1.31 | 1.26 | 0.1 |
| 173 | L46K | 46 | 0.93 | 0.87 | 1.23 | 1.01 | 0.16 |
| 174 | L46G | 46 | 0.79 | 0.76 | 1.05 | 0.87 | 0.13 |
| 175 | L46C | 46 | 0.79 | 0.85 | 0.64 | 0.76 | 0.09 |
| 176 | L46H | 46 | 0.75 | 0.48 | 0.91 | 0.71 | 0.17 |
| 177 | L46T | 46 | 0.6 | 0.7 | 0.79 | 0.7 | 0.08 |
| 178 | L46E | 46 | 0.57 | 0.56 | 0.55 | 0.56 | 0.01 |
| 179 | E49Y | 49 | 1.83 | 1.88 | 1.77 | 1.82 | 0.04 |
| 180 | E49N | 49 | 1.47 | 1.52 | 1.86 | 1.61 | 0.18 |
| 181 | E49P | 49 | 1.57 | 1.64 | 1.33 | 1.51 | 0.14 |
| 182 | E49F | 49 | 1.21 | 1.18 | 1.31 | 1.23 | 0.05 |
| 183 | E49R | 49 | 1.17 | 1.26 | 1.26 | 1.23 | 0.04 |
| 184 | E49M | 49 | 1.29 | 1.06 | 0.82 | 1.06 | 0.19 |
| 185 | E49T | 49 | 0.83 | 1.34 | 0.86 | 1.01 | 0.23 |
| 186 | E49H | 49 | 1.01 | 1.16 | 0.63 | 0.93 | 0.22 |
| 187 | E49L | 49 | 0.86 | 0.82 | 0.85 | 0.84 | 0.02 |
| 188 | E49C | 49 | 0.46 | 0.91 | 0.83 | 0.73 | 0.2 |
| 189 | E49I | 49 | 0.92 | 0.44 | 0.69 | 0.69 | 0.2 |
| 190 | E49S | 49 | 0.63 | 0.53 | 0.65 | 0.6 | 0.05 |
| 191 | E49K | 49 | 0.43 | 0.4 | 0.42 | 0.42 | 0.01 |
| 192 | N50Q | 50 | 0.51 | 0.11 | 0.91 | 0.51 | 0.33 |
| 193 | Q52V | 52 | 0.61 | 0.65 | 0.69 | 0.65 | 0.03 |
| 194 | Q52C | 52 | 0.62 | 0.58 | 0.42 | 0.54 | 0.09 |
| 195 | L54G | 54 | 0.59 | 0.55 | 0.51 | 0.55 | 0.03 |
| 196 | D56P | 56 | 1.15 | 1.41 | 1.52 | 1.36 | 0.15 |
| 197 | D56R | 56 | 0.98 | 0.95 | 0.95 | 0.96 | 0.01 |
| 198 | D56K | 56 | 0.61 | 1.03 | 0.58 | 0.74 | 0.21 |
| 199 | D56A | 56 | 0.57 | 0.29 | 0.68 | 0.51 | 0.16 |
| 200 | I57E | 57 | 1.3 | 1.28 | 1.3 | 1.3 | 0.01 |
| 201 | I57C | 57 | 1.48 | 1.27 | 0.89 | 1.21 | 0.25 |
| 202 | I57P | 57 | 0.95 | 0.93 | 0.84 | 0.91 | 0.05 |
| 203 | I57G | 57 | 0.74 | 0.65 | 0.98 | 0.79 | 0.14 |
| 204 | I57A | 57 | 0.91 | 0.59 | 0.86 | 0.79 | 0.14 |
| 205 | I57T | 57 | 0.47 | 0.66 | 0.55 | 0.56 | 0.08 |
| 206 | I57Q | 57 | 0.94 | 0.36 | 0.37 | 0.56 | 0.27 |
| 207 | I57V | 57 | 0.53 | 0.58 | 0.38 | 0.5 | 0.09 |
| 208 | I57L | 57 | 0.32 | 0.56 | 0.37 | 0.42 | 0.1 |
| 209 | D59A | 59 | 0.94 | 1.09 | 1.11 | 1.05 | 0.08 |
| 210 | D59I | 59 | 0.95 | 1.07 | 0.65 | 0.89 | 0.17 |
| 211 | D59S | 59 | 0.76 | 0.92 | 0.93 | 0.87 | 0.08 |
| 212 | D59T | 59 | 0.78 | 0.98 | 0.66 | 0.81 | 0.13 |
| 213 | D59R | 59 | 0.93 | 0.65 | 0.62 | 0.73 | 0.14 |
| 214 | D59G | 59 | 0.86 | 0.54 | 0.62 | 0.67 | 0.14 |
| 215 | D59M | 59 | 0.49 | 0.7 | 0.4 | 0.53 | 0.13 |
| 216 | D59L | 59 | 0.42 | 0.52 | 0.56 | 0.5 | 0.06 |
| 217 | D59Q | 59 | 0.67 | 0.29 | 0.38 | 0.45 | 0.16 |
| 218 | D60R | 60 | 0.92 | 1.26 | 1.38 | 1.18 | 0.19 |
| 219 | D60N | 60 | 1.23 | 0.68 | 1.22 | 1.04 | 0.26 |
| 220 | D60M | 60 | 0.76 | 0.82 | 0.77 | 0.78 | 0.02 |
| 221 | D60T | 60 | 0.48 | 0.89 | 0.83 | 0.73 | 0.18 |
| 222 | D60G | 60 | 0.6 | 0.73 | 0.85 | 0.73 | 0.1 |
| 223 | D60V | 60 | 0.92 | 0.74 | 0.39 | 0.69 | 0.22 |
| 224 | D60K | 60 | 0.9 | 0.4 | 0.61 | 0.64 | 0.2 |
| 225 | D60Y | 60 | 0.34 | 0.72 | 0.73 | 0.6 | 0.18 |
| 226 | D60P | 60 | 0.65 | 0.76 | 0.36 | 0.59 | 0.17 |
| 227 | D60F | 60 | 0.87 | −0.02 | 0.75 | 0.53 | 0.4 |
| 228 | D60L | 60 | 0.48 | 0.75 | 0.25 | 0.49 | 0.2 |
| 229 | D60A | 60 | 0.51 | 0.32 | 0.62 | 0.49 | 0.12 |
| 230 | Y62Q | 62 | 1.41 | 1.96 | 1.06 | 1.48 | 0.37 |
| 231 | Y62A | 62 | 1.37 | 0.78 | 0.85 | 1 | 0.26 |
| 232 | Y62F | 62 | 1.2 | 0.67 | 0.98 | 0.95 | 0.22 |
| 233 | Y62V | 62 | 0.63 | 1.04 | 0.79 | 0.82 | 0.17 |
| 234 | Y62N | 62 | 0.29 | 0.79 | 0.28 | 0.45 | 0.24 |
| 235 | R63M | 63 | 0.49 | 0.69 | 0.41 | 0.53 | 0.12 |
| 236 | R63V | 63 | 0.4 | 0.56 | 0.34 | 0.43 | 0.09 |
| 237 | F65E | 65 | 1.27 | 1.08 | 0.8 | 1.05 | 0.19 |
| 238 | F65K | 65 | 0.79 | 0.4 | 0.75 | 0.65 | 0.17 |
| 239 | F65R | 65 | 0.45 | 0.47 | 0.44 | 0.46 | 0.01 |
| 240 | S67V | 67 | 1.04 | 0.68 | 0.92 | 0.88 | 0.15 |
| 241 | S67Y | 67 | 0.77 | 0.77 | 0.68 | 0.74 | 0.04 |
| 242 | S67F | 67 | 0.76 | 0.84 | 0.56 | 0.72 | 0.12 |
| 243 | S67Q | 67 | 0.59 | 0.45 | 0.57 | 0.53 | 0.06 |
| 244 | S67A | 67 | 0.55 | 0.4 | 0.51 | 0.49 | 0.06 |
| 245 | S67W | 67 | 0.49 | 0.48 | 0.31 | 0.43 | 0.08 |
| 246 | E68V | 68 | 0.99 | 1.13 | 1.51 | 1.21 | 0.22 |
| 247 | E68A | 68 | 1.18 | 0.41 | 0.38 | 0.66 | 0.37 |
| 248 | E68R | 68 | 0.75 | 0.64 | 0.36 | 0.58 | 0.16 |
| 249 | T69F | 69 | 1.81 | 2.06 | 1.93 | 1.93 | 0.1 |
| 250 | T69M | 69 | 0.49 | 1.24 | 0.68 | 0.8 | 0.32 |
| 251 | T69D | 69 | 1.3 | 0.26 | 0.68 | 0.75 | 0.43 |
| 252 | T69H | 69 | 0.2 | 0.96 | 0.68 | 0.61 | 0.31 |
| 253 | T69W | 69 | 0.2 | 0.77 | 0.5 | 0.49 | 0.23 |
| 254 | T69L | 69 | 0.44 | 0.39 | 0.5 | 0.44 | 0.04 |
| 255 | S71Q | 71 | 0.61 | 1.28 | 0.39 | 0.76 | 0.38 |
| 256 | I73E | 73 | 0.38 | 0.5 | 0.56 | 0.48 | 0.07 |
| 257 | D75R | 75 | 0.81 | 0.27 | 0.84 | 0.64 | 0.26 |
| 258 | I76Q | 76 | 0.61 | 0.83 | 0.86 | 0.77 | 0.11 |
| 259 | I76G | 76 | 0.79 | 0.74 | 0.59 | 0.71 | 0.09 |
| 260 | I76F | 76 | 1.57 | −0.12 | 0.5 | 0.65 | 0.7 |
| 261 | D77P | 77 | 0.75 | 0.64 | 0.66 | 0.68 | 0.05 |
| 262 | T79E | 79 | 0.52 | 0.39 | 0.56 | 0.49 | 0.07 |
| 263 | T79R | 79 | 0.45 | 0.53 | 0.39 | 0.46 | 0.05 |
| 264 | S80N | 80 | 0.62 | 0.5 | 0.31 | 0.48 | 0.13 |
| 265 | S80Q | 80 | 0.44 | 0.62 | 0.32 | 0.46 | 0.12 |
| 266 | S80V | 80 | 0.36 | 0.42 | 0.48 | 0.42 | 0.05 |
| 267 | L81Y | 81 | 0.71 | 0.73 | 0.6 | 0.68 | 0.05 |
| 268 | L81H | 81 | 0.64 | 0.68 | 0.51 | 0.61 | 0.07 |
| 269 | L81W | 81 | 0.39 | 0.59 | 0.73 | 0.57 | 0.14 |
| 270 | L81E | 81 | 0.94 | 0.28 | 0.39 | 0.54 | 0.29 |
| 271 | F82Q | 82 | 0.38 | 0.42 | 0.66 | 0.49 | 0.12 |
| 272 | K84F | 84 | 0.4 | 0.66 | 0.49 | 0.52 | 0.11 |
| 273 | M85G | 85 | 1.06 | 1.18 | 1.18 | 1.14 | 0.06 |
| 274 | M85Q | 85 | 0.79 | 0.8 | 1.34 | 0.98 | 0.26 |
| 275 | M85Y | 85 | 1.11 | 0.78 | 0.94 | 0.95 | 0.14 |
| 276 | M85A | 85 | 0.85 | 0.79 | 1.09 | 0.91 | 0.13 |
| 277 | M85N | 85 | 0.67 | 0.98 | 0.82 | 0.82 | 0.13 |
| 278 | M85W | 85 | 0.56 | 0.44 | 1.11 | 0.7 | 0.29 |
| 279 | M85E | 85 | 0.7 | 0.51 | 0.64 | 0.62 | 0.08 |
| 280 | M85F | 85 | 0.42 | 0.58 | 0.42 | 0.47 | 0.08 |
| 281 | E86N | 86 | 0.44 | 1.02 | 0.66 | 0.71 | 0.24 |
| 282 | I87G | 87 | 0.7 | 0.79 | 0.46 | 0.65 | 0.14 |
| 283 | I87A | 87 | 0.68 | 0.55 | 0.29 | 0.51 | 0.16 |
| 284 | I87H | 87 | 0.25 | 0.65 | 0.62 | 0.5 | 0.18 |
| 285 | I87W | 87 | 0.38 | 0.46 | 0.43 | 0.42 | 0.03 |
| 286 | Q88N | 88 | 1.07 | 0.64 | 0.84 | 0.85 | 0.18 |
| 287 | Q88E | 88 | 0.28 | 1.1 | 0.97 | 0.78 | 0.36 |
| 288 | Q88S | 88 | 0.41 | 0.47 | 0.5 | 0.46 | 0.04 |
| 289 | L89Q | 89 | 0.97 | 0.96 | 1.21 | 1.05 | 0.12 |
| 290 | L89S | 89 | 0.47 | 0.54 | 0.6 | 0.54 | 0.05 |
| 291 | L89C | 89 | 0.6 | 0.61 | 0.37 | 0.52 | 0.11 |
| 292 | L89D | 89 | 0.63 | 0.32 | 0.47 | 0.48 | 0.13 |
| 293 | L89G | 89 | 0.21 | 0.8 | 0.33 | 0.45 | 0.25 |
| 294 | K90P | 90 | 1.13 | 1.38 | 1.11 | 1.21 | 0.12 |
| 295 | N91V | 91 | 0.84 | 0.37 | 0.47 | 0.56 | 0.2 |
| 296 | G92D | 92 | 0.21 | 1.04 | 0.37 | 0.54 | 0.36 |
| 297 | G92F | 92 | −0.07 | 0 | 1.51 | 0.48 | 0.73 |
| 298 | D93L | 93 | 0.83 | 0.76 | 1.16 | 0.92 | 0.17 |
| 299 | D93P | 93 | 0.68 | 0.87 | 0.7 | 0.75 | 0.09 |
| 300 | D93T | 93 | 0.46 | 0.45 | 0.96 | 0.62 | 0.24 |
| 301 | D93N | 93 | −0.04 | 0.65 | 0.81 | 0.47 | 0.37 |
| 302 | D93R | 93 | 0.36 | 0.36 | 0.66 | 0.46 | 0.14 |
| 303 | D93M | 93 | 0.68 | 0.66 | 0.01 | 0.45 | 0.31 |
| 304 | N94P | 94 | 1.28 | 0.92 | 0.59 | 0.93 | 0.28 |
| 305 | K95T | 95 | 0.8 | 1 | 1.03 | 0.94 | 0.1 |
| 306 | K95F | 95 | 0.5 | 0.8 | 0.95 | 0.75 | 0.19 |
| 307 | K95L | 95 | 0.9 | 0.62 | 0.48 | 0.67 | 0.17 |
| 308 | K95P | 95 | 0.82 | 0.69 | 0.44 | 0.65 | 0.16 |
| 309 | K95E | 95 | 0.97 | 0.23 | 0.64 | 0.61 | 0.3 |
| 310 | K95W | 95 | 0.43 | 0.46 | 0.61 | 0.5 | 0.08 |
| 311 | K95I | 95 | 0.64 | 0.41 | 0.4 | 0.48 | 0.11 |
| 312 | K95S | 95 | 0.4 | 0.55 | 0.37 | 0.44 | 0.08 |
| 313 | D96C | 96 | 0.91 | 0.51 | 0.57 | 0.66 | 0.18 |
| 314 | T97W | 97 | 0.95 | 0.29 | 0.42 | 0.55 | 0.28 |
| 315 | L98Q | 98 | 1.8 | 1.34 | 1.71 | 1.62 | 0.2 |
| 316 | L98A | 98 | 1.07 | 1.3 | 0.84 | 1.07 | 0.19 |
| 317 | L98H | 98 | 0.92 | 0.92 | 1.12 | 0.99 | 0.09 |
| 318 | L98D | 98 | 1.27 | 0.83 | 0.85 | 0.98 | 0.2 |
| 319 | L98N | 98 | 0.92 | 0.99 | 0.74 | 0.88 | 0.1 |
| 320 | L98G | 98 | 0.76 | 0.81 | 0.82 | 0.8 | 0.03 |
| 321 | L98S | 98 | 0.82 | 0.84 | 0.62 | 0.76 | 0.1 |
| 322 | L98T | 98 | 0.88 | 0.77 | 0.41 | 0.69 | 0.2 |
| 323 | L98C | 98 | 0.84 | 0.3 | 0.76 | 0.63 | 0.24 |
| 324 | L98P | 98 | 0.53 | 0.41 | 0.36 | 0.43 | 0.07 |
| 325 | I99W | 99 | 0.96 | 1.23 | 0.8 | 1 | 0.18 |
| 326 | I99Y | 99 | 0.83 | 0.79 | 0.69 | 0.77 | 0.06 |
| 327 | E101P | 101 | 0.39 | 0.6 | 0.6 | 0.53 | 0.1 |
| 328 | Q102I | 102 | 0.92 | 0.3 | 2.73 | 1.31 | 1.03 |
| 329 | Q102V | 102 | 0.63 | 0.9 | 1 | 0.84 | 0.16 |
| 330 | T103Y | 103 | 0.63 | 0.7 | 0.42 | 0.58 | 0.12 |
| 331 | E104P | 104 | 0.6 | 0.82 | 0.86 | 0.76 | 0.11 |
| 332 | Y105R | 105 | 1.49 | 1.87 | 1.86 | 1.74 | 0.18 |
| 333 | Y105P | 105 | 1.04 | 0.52 | 1.89 | 1.15 | 0.57 |
| 334 | Y105A | 105 | 0.63 | 0.29 | 1.52 | 0.82 | 0.52 |
| 335 | Y105T | 105 | 1 | 0.11 | 0.87 | 0.66 | 0.39 |
| 336 | Y105G | 105 | 0.22 | 0.52 | 0.94 | 0.56 | 0.29 |
| 337 | R106T | 106 | 0.79 | 0.3 | 0.71 | 0.6 | 0.21 |
| 338 | R106V | 106 | 0.29 | 0.65 | 0.52 | 0.49 | 0.15 |
| 339 | R106I | 106 | 0.23 | 0.7 | 0.38 | 0.44 | 0.2 |
| 340 | A108R | 108 | 0.73 | 0.43 | 0.41 | 0.52 | 0.15 |
| 341 | K111T | 111 | 0.75 | 0.63 | 0.78 | 0.72 | 0.07 |
| 342 | K111W | 111 | 0.62 | 0.71 | 0.59 | 0.64 | 0.05 |
| 343 | K111G | 111 | 0.6 | 0.61 | 0.69 | 0.63 | 0.04 |
| 344 | K111A | 111 | 0.56 | 0.69 | 0.56 | 0.6 | 0.06 |
| 345 | K111F | 111 | 0.44 | 0.59 | 0.7 | 0.57 | 0.11 |
| 346 | K111V | 111 | 0.69 | 0.51 | 0.43 | 0.54 | 0.11 |
| 347 | K111S | 111 | 0.48 | 0.62 | 0.52 | 0.54 | 0.06 |
| 348 | K111C | 111 | 0.32 | 0.39 | 0.56 | 0.42 | 0.1 |
| 349 | K112D | 112 | 1.73 | 0.3 | 2.17 | 1.4 | 0.8 |
| 350 | K112H | 112 | 0.92 | 1.76 | 0.72 | 1.14 | 0.45 |
| 351 | K112E | 112 | 0.99 | 1.12 | 1.03 | 1.05 | 0.06 |
| 352 | K112L | 112 | 0.79 | 1.4 | 0.43 | 0.87 | 0.4 |
| 353 | K112T | 112 | 0.52 | 0.81 | 0.87 | 0.73 | 0.16 |
| 354 | K112C | 112 | 0.63 | 0.3 | 0.72 | 0.55 | 0.18 |
| 355 | K112R | 112 | 0.55 | 0.13 | 0.96 | 0.55 | 0.34 |
| 356 | K112P | 112 | 0.41 | 0.63 | 0.5 | 0.51 | 0.09 |
| 357 | F113A | 113 | 0.23 | 0.42 | 0.72 | 0.45 | 0.2 |
| 358 | A114S | 114 | 1.21 | 1.28 | 0.72 | 1.07 | 0.25 |
| 359 | A114E | 114 | 1.07 | −0.11 | 0.87 | 0.61 | 0.52 |
| 360 | N115W | 115 | 0.7 | 0.99 | 0.02 | 0.57 | 0.4 |
| 361 | N115M | 115 | 0.42 | 0.25 | 0.78 | 0.48 | 0.22 |
| 362 | R118D | 118 | 2.53 | 2.24 | 1.81 | 2.19 | 0.29 |
| 363 | R118A | 118 | 0.92 | 0.99 | 0.93 | 0.95 | 0.03 |
| 364 | R118Y | 118 | 1.11 | −0.04 | 0.47 | 0.51 | 0.47 |
| 365 | R118N | 118 | 0.92 | 1.21 | −0.68 | 0.48 | 0.83 |
| 366 | R118V | 118 | 0.85 | −0.17 | 0.65 | 0.44 | 0.44 |
| 367 | F119H | 119 | 1.48 | 1.21 | 1.4 | 1.36 | 0.11 |
| 368 | F119D | 119 | 0.45 | 0.29 | 0.71 | 0.48 | 0.17 |
| 369 | K120A | 120 | 0.95 | 0.88 | 0.57 | 0.8 | 0.17 |
| 370 | K120C | 120 | 0.36 | 0.29 | 0.67 | 0.44 | 0.16 |
| 371 | K120V | 120 | 0.19 | 0.45 | 0.66 | 0.43 | 0.19 |
| 372 | K120T | 120 | 0.34 | 0.32 | 0.63 | 0.43 | 0.14 |
| 373 | K120P | 120 | 0.28 | 0.55 | 0.44 | 0.42 | 0.11 |
| 374 | N121F | 121 | 0.48 | 0.32 | 0.53 | 0.45 | 0.09 |
| 375 | M122W | 122 | 0.51 | 0.47 | 0.45 | 0.48 | 0.02 |
| 376 | F123E | 123 | 0.44 | 0.43 | 0.48 | 0.45 | 0.02 |
| 377 | S124N | 124 | 1.32 | 0.38 | 0.11 | 0.6 | 0.52 |
| 378 | L127Q | 127 | 0.4 | 0.46 | 1.34 | 0.73 | 0.43 |
| 379 | I128Q | 128 | 2.16 | 1.89 | 1.39 | 1.81 | 0.32 |
| 380 | S129G | 129 | 0.91 | 1.38 | 1.1 | 1.13 | 0.19 |
| 381 | S129F | 129 | 0.21 | 1.38 | 1.1 | 0.9 | 0.5 |
| 382 | S129D | 129 | −0.48 | 0.97 | 0.92 | 0.47 | 0.67 |
| 383 | S129I | 129 | 0.91 | 0.41 | 0.01 | 0.44 | 0.37 |
| 384 | D130I | 130 | 0.47 | 0.67 | 0.22 | 0.45 | 0.18 |
| 385 | L132T | 132 | 0.91 | 1.2 | 1.11 | 1.07 | 0.12 |
| 386 | L132G | 132 | 0.73 | 0.95 | 0.75 | 0.81 | 0.1 |
| 387 | L132A | 132 | 0.64 | 0.8 | 0.76 | 0.73 | 0.07 |
| 388 | F135D | 135 | 1.04 | 0.82 | 0.63 | 0.83 | 0.17 |
| 389 | F135A | 135 | 0.6 | 0.67 | 0.53 | 0.6 | 0.06 |
| 390 | F135H | 135 | 0.55 | 0.46 | 0.54 | 0.52 | 0.04 |
| 391 | F135T | 135 | 0.35 | 0.6 | 0.33 | 0.43 | 0.12 |
| 392 | V136Y | 136 | 0.59 | 0.65 | 0.32 | 0.52 | 0.15 |
| 393 | I137Q | 137 | 0.49 | 0.73 | 0.49 | 0.57 | 0.11 |
| 394 | H138M | 138 | 0.9 | 1.58 | 0.52 | 1 | 0.44 |
| 395 | H138A | 138 | 0.21 | 1.07 | 0.88 | 0.72 | 0.37 |
| 396 | H138S | 138 | 0.21 | 0.81 | 0.91 | 0.64 | 0.31 |
| 397 | H138V | 138 | 0.97 | 0.53 | 0.14 | 0.55 | 0.34 |
| 398 | N139Q | 139 | 1.72 | 2.15 | 2.61 | 2.16 | 0.36 |
| 399 | N139C | 139 | 1.42 | 1.13 | 1.39 | 1.31 | 0.13 |
| 400 | N139A | 139 | 0.72 | 0.68 | 1.43 | 0.95 | 0.34 |
| 401 | N139R | 139 | 0.93 | 1.05 | 0.36 | 0.78 | 0.3 |
| 402 | N139I | 139 | 0.79 | 0.28 | 0.9 | 0.65 | 0.27 |
| 403 | N139P | 139 | 0.21 | −0.13 | 1.68 | 0.59 | 0.78 |
| 404 | N139L | 139 | 0.29 | 0.53 | 0.52 | 0.45 | 0.11 |
| 405 | N139F | 139 | 0.72 | 0.28 | 0.29 | 0.43 | 0.21 |
| 406 | N139G | 139 | 0.76 | 0.18 | 0.31 | 0.42 | 0.25 |
| 407 | N140C | 140 | 1.92 | 1.89 | 2.02 | 1.94 | 0.06 |
| 408 | N140G | 140 | 0.71 | 0.9 | 0.66 | 0.76 | 0.1 |
| 409 | N140P | 140 | 0.81 | 0.83 | 0.6 | 0.75 | 0.1 |
| 410 | N140T | 140 | 0.68 | 0.84 | 0.51 | 0.68 | 0.14 |
| 411 | N140S | 140 | 0.58 | 0.54 | 0.62 | 0.58 | 0.03 |
| 412 | N141P | 141 | 0.51 | 0.06 | 1.04 | 0.54 | 0.4 |
| 413 | N141F | 141 | 0.1 | 0.53 | 0.75 | 0.46 | 0.27 |
| 414 | Y142G | 142 | 0.77 | 0.69 | 0.85 | 0.77 | 0.06 |
| 415 | Y142K | 142 | 0.77 | 0.89 | 0.52 | 0.73 | 0.15 |
| 416 | Y142E | 142 | 0.27 | 0.44 | 1.23 | 0.65 | 0.42 |
| 417 | Y142R | 142 | 0.74 | 0.86 | 0.33 | 0.64 | 0.22 |
| 418 | Y142P | 142 | 0.82 | 0.28 | 0.66 | 0.59 | 0.22 |
| 419 | Y142V | 142 | 0.54 | 0.61 | 0.19 | 0.45 | 0.19 |
| 420 | S143R | 143 | 0.36 | 0.47 | 1.68 | 0.84 | 0.6 |
| 421 | S143E | 143 | 0.7 | 0.69 | 0.42 | 0.6 | 0.13 |
| 422 | S143G | 143 | 0.91 | 0.38 | 0.35 | 0.55 | 0.26 |
| 423 | S143V | 143 | 0.77 | 0.65 | 0.17 | 0.53 | 0.26 |
| 424 | S143I | 143 | 0.41 | 0.83 | 0.12 | 0.45 | 0.29 |
| 425 | A144R | 144 | 0.83 | 1.42 | 2.34 | 1.53 | 0.62 |
| 426 | A144I | 144 | 1.31 | 0.29 | 1.96 | 1.19 | 0.69 |
| 427 | A144K | 144 | 1.21 | 0.54 | 1.06 | 0.94 | 0.29 |
| 428 | A144N | 144 | 0.21 | 0.8 | 0.86 | 0.62 | 0.29 |
| 429 | A144P | 144 | 0.37 | 0.29 | 0.93 | 0.53 | 0.28 |
| 430 | A144C | 144 | 0.42 | 0.58 | 0.59 | 0.53 | 0.08 |
| 431 | A144D | 144 | −0.7 | 0.98 | 1.23 | 0.5 | 0.86 |
| 432 | A144T | 144 | 0.83 | 0.21 | 0.2 | 0.42 | 0.29 |
| 433 | E146R | 146 | 1.36 | 0.54 | 3.04 | 1.65 | 1.04 |
| 434 | E146T | 146 | 1.75 | 1.38 | 1.11 | 1.42 | 0.26 |
| 435 | E146M | 146 | 0.77 | 1.1 | 0.42 | 0.76 | 0.28 |
| 436 | E146I | 146 | 0.62 | 0.51 | 1.11 | 0.75 | 0.26 |
| 437 | E146S | 146 | 0.55 | 0.47 | 0.67 | 0.56 | 0.08 |
| 438 | K147F | 147 | 0.68 | 0.82 | 1.11 | 0.87 | 0.18 |
| 439 | K147A | 147 | 0.4 | 1.24 | 0.48 | 0.71 | 0.38 |
| 440 | K147G | 147 | 0.68 | 0.62 | 0.8 | 0.7 | 0.07 |
| 441 | K147Q | 147 | 0.09 | 0.11 | 1.86 | 0.69 | 0.83 |
| 442 | K147L | 147 | 0.74 | 0.74 | 0.52 | 0.66 | 0.1 |
| 443 | E148C | 148 | 0.43 | 0.45 | 1.54 | 0.81 | 0.52 |
| 444 | E148P | 148 | 0.82 | 0.1 | 0.42 | 0.45 | 0.29 |
| 445 | E149L | 149 | 0.99 | 0.98 | 0.62 | 0.86 | 0.17 |
| 446 | E149R | 149 | 0.59 | 0.93 | 0.18 | 0.57 | 0.31 |
| 447 | K150G | 150 | 1.87 | 1.2 | 1.67 | 1.58 | 0.28 |
| 448 | K150C | 150 | 1.31 | 1.38 | 1.11 | 1.27 | 0.12 |
| 449 | K150A | 150 | 1.39 | 0.84 | 0.82 | 1.02 | 0.26 |
| 450 | K150I | 150 | −0.48 | 1.49 | 1.36 | 0.79 | 0.9 |
| 451 | K150W | 150 | 1.06 | 0.03 | 0.81 | 0.64 | 0.44 |
| 452 | K150V | 150 | 0.22 | 1.18 | 0.32 | 0.57 | 0.43 |
| 453 | K150L | 150 | 0.5 | 0.69 | 0.45 | 0.55 | 0.1 |
| 454 | K150Y | 150 | 0.29 | −0.08 | 1.34 | 0.52 | 0.6 |
| 455 | T151E | 151 | 0.67 | 0.75 | 0.75 | 0.72 | 0.03 |
| 456 | T151C | 151 | 0.5 | 0.9 | 0.36 | 0.59 | 0.23 |
| 457 | T151V | 151 | 0.55 | 0.67 | 0.45 | 0.56 | 0.09 |
| 458 | T151Q | 151 | 0.47 | 0.42 | 0.63 | 0.51 | 0.09 |
| 459 | V153K | 153 | 0.49 | 0.85 | 0.84 | 0.73 | 0.17 |
| 460 | I154P | 154 | 1.04 | 1.04 | 0.96 | 1.01 | 0.04 |
| 461 | I154E | 154 | 1.07 | 0.5 | 0.74 | 0.77 | 0.23 |
| 462 | I154A | 154 | 0.72 | 0.41 | 0.55 | 0.56 | 0.13 |
| 463 | I154Q | 154 | 0.75 | 0.33 | 0.27 | 0.45 | 0.22 |
| 464 | I154H | 154 | 0.33 | 0.4 | 0.55 | 0.43 | 0.09 |
| 465 | L156C | 156 | 0.59 | 0.25 | 0.43 | 0.43 | 0.14 |
| 466 | S158N | 158 | 1.14 | 0.84 | 0.92 | 0.97 | 0.13 |
| 467 | S158G | 158 | 0.75 | 0.58 | 0.83 | 0.72 | 0.1 |
| 468 | S158K | 158 | 0.46 | 0.55 | 0.49 | 0.5 | 0.04 |
| 469 | S158D | 158 | 0.37 | 0.48 | 0.55 | 0.47 | 0.08 |
| 470 | F160H | 160 | 0.91 | 0.29 | 0.71 | 0.64 | 0.26 |
| 471 | S163I | 163 | 0.71 | 0.83 | 0.72 | 0.75 | 0.06 |
| 472 | F164Q | 164 | 0.55 | 1.08 | 0.36 | 0.67 | 0.31 |
| 473 | K165Y | 165 | 0.32 | 0.72 | 0.5 | 0.52 | 0.16 |
| 474 | Y167M | 167 | 0.66 | 0.86 | 0.64 | 0.72 | 0.1 |
| 475 | Y167I | 167 | 0.62 | 0.51 | 0.91 | 0.68 | 0.17 |
| 476 | Y167L | 167 | 0.46 | 0.47 | 0.54 | 0.49 | 0.03 |
| 477 | K169C | 169 | 0.81 | 0.78 | 0.87 | 0.82 | 0.03 |
| 478 | K169F | 169 | 0.7 | 0.64 | 0.65 | 0.66 | 0.03 |
| 479 | A172Y | 172 | 0.22 | 1 | 1.42 | 0.88 | 0.5 |
| 480 | C174V | 174 | 0.47 | 0.7 | 0.8 | 0.66 | 0.14 |
| 481 | C174T | 174 | 0.78 | 0.44 | 0.67 | 0.63 | 0.14 |
| 482 | C174I | 174 | 0.37 | 0.46 | 0.51 | 0.45 | 0.06 |
| 483 | A177W | 177 | 1 | 0.31 | 0.63 | 0.65 | 0.28 |
| 484 | A177M | 177 | 0.42 | 0.42 | 0.44 | 0.43 | 0.01 |
| 485 | D178R | 178 | 0.73 | 0.57 | 0.59 | 0.63 | 0.07 |
| 486 | D179W | 179 | 0.71 | 0.78 | 0.81 | 0.76 | 0.04 |
| 487 | D179P | 179 | 0.34 | 0.23 | 0.7 | 0.42 | 0.2 |
| 488 | S181N | 181 | 0.38 | 0.39 | 0.99 | 0.58 | 0.29 |
| 489 | S181H | 181 | 0.84 | 0.76 | −0.04 | 0.52 | 0.4 |
| 490 | S181V | 181 | 0.42 | 0.38 | 0.54 | 0.45 | 0.07 |
| 491 | S184I | 184 | 1.57 | 1.58 | 1.65 | 1.6 | 0.03 |
| 492 | S184M | 184 | 1.34 | 1 | 1.36 | 1.23 | 0.16 |
| 493 | S184V | 184 | 0.59 | 0.64 | 0.75 | 0.66 | 0.07 |
| 494 | S184L | 184 | 0.34 | 0.52 | 0.5 | 0.45 | 0.08 |
| 495 | S184C | 184 | 0.57 | 0.52 | 0.2 | 0.43 | 0.16 |
| 496 | I188W | 188 | 1.14 | 0.72 | 0.72 | 0.86 | 0.2 |
| 497 | I188A | 188 | 0.86 | 0.59 | 0.88 | 0.78 | 0.13 |
| 498 | I188C | 188 | 0.6 | 0.52 | 0.59 | 0.57 | 0.04 |
| 499 | N190Q | 190 | 1.14 | 0.31 | 0.91 | 0.79 | 0.35 |
| 500 | N190R | 190 | 0.36 | 1.51 | 0.25 | 0.71 | 0.57 |
| 501 | N192G | 192 | 0.69 | 0.71 | 0.79 | 0.73 | 0.05 |
| 502 | N192C | 192 | 0.84 | 0.16 | 0.35 | 0.45 | 0.29 |
| 503 | A193H | 193 | 0.8 | 0.74 | 0.92 | 0.82 | 0.07 |
| 504 | A193F | 193 | 0.73 | 0.64 | 0.58 | 0.65 | 0.06 |
| 505 | A193L | 193 | 0.56 | 0.6 | 0.7 | 0.62 | 0.06 |
| 506 | A193N | 193 | 0.36 | 0.66 | 0.62 | 0.55 | 0.13 |
| 507 | A193Q | 193 | 0.42 | 0.65 | 0.55 | 0.54 | 0.09 |
| 508 | I195W | 195 | 0.64 | 0.57 | 0.46 | 0.56 | 0.07 |
| 509 | F196T | 196 | 0.57 | 0.27 | 0.52 | 0.45 | 0.13 |
| 510 | F197E | 197 | 0.63 | 0.73 | 0.66 | 0.67 | 0.05 |
| 511 | F197R | 197 | 0.61 | 0.58 | 0.5 | 0.56 | 0.05 |
| 512 | F197K | 197 | 0.58 | 0.48 | 0.62 | 0.56 | 0.06 |
| 513 | F197H | 197 | 0.38 | 0.59 | 0.42 | 0.46 | 0.09 |
| 514 | F197Q | 197 | 0.45 | 0.24 | 0.6 | 0.43 | 0.15 |
| 515 | S198D | 198 | 0.78 | 0.59 | 0.64 | 0.67 | 0.08 |
| 516 | S198E | 198 | 0.28 | 0.68 | 0.56 | 0.51 | 0.17 |
| 517 | S198K | 198 | 0.14 | 0.72 | 0.66 | 0.51 | 0.26 |
| 518 | S198N | 198 | −0.25 | 0.49 | 1.27 | 0.5 | 0.62 |
| 519 | A200H | 200 | 1.32 | 1.42 | 1.44 | 1.39 | 0.05 |
| 520 | A200I | 200 | 0.22 | 1.11 | 0.52 | 0.62 | 0.37 |
| 521 | V202H | 202 | 1.83 | 2.1 | 0.73 | 1.55 | 0.59 |
| 522 | V202C | 202 | 0.44 | 0.63 | 0.67 | 0.58 | 0.1 |
| 523 | R205M | 205 | 0.72 | 1.61 | 1.65 | 1.33 | 0.43 |
| 524 | R205T | 205 | 0.37 | 0.6 | 0.44 | 0.47 | 0.1 |
| 525 | R205C | 205 | 0.69 | 0.26 | 0.32 | 0.42 | 0.19 |
| 526 | I206Y | 206 | 0.72 | 0.75 | 1.08 | 0.85 | 0.16 |
| 527 | I206W | 206 | 1.1 | 0.1 | 0.35 | 0.52 | 0.43 |
| 528 | I206D | 206 | 0.99 | 0.13 | 0.44 | 0.52 | 0.36 |
| 529 | V207W | 207 | 0.42 | 0.4 | 0.54 | 0.46 | 0.06 |
| 530 | S209K | 209 | 0.57 | 0.51 | 0.63 | 0.57 | 0.05 |
| 531 | L210N | 210 | 0.57 | 0.23 | 0.57 | 0.46 | 0.16 |
| 532 | D214T | 214 | 0.36 | 0.62 | 0.76 | 0.58 | 0.17 |
| 533 | D214W | 214 | 0.49 | 0.5 | 0.56 | 0.51 | 0.03 |
| 534 | I215W | 215 | 1.13 | 0.78 | 0.37 | 0.76 | 0.31 |
| 535 | I215A | 215 | 0.26 | 0.31 | 0.73 | 0.43 | 0.21 |
| 536 | N216F | 216 | 0.62 | 0.25 | 0.67 | 0.51 | 0.19 |
| 537 | K217A | 217 | 0.4 | 0.49 | 1.2 | 0.69 | 0.36 |
| 538 | K217D | 217 | 0.4 | 0.64 | 0.73 | 0.59 | 0.14 |
| 539 | K217V | 217 | 0.52 | 0.55 | 0.58 | 0.55 | 0.02 |
| 540 | S219P | 219 | 0.21 | 1.16 | 0.88 | 0.75 | 0.4 |
| 541 | S219F | 219 | −0.07 | 0.71 | 0.85 | 0.5 | 0.41 |
| 542 | S219W | 219 | 1.24 | −0.2 | 0.37 | 0.47 | 0.59 |
| 543 | S219D | 219 | 0.12 | 0.62 | 0.63 | 0.46 | 0.24 |
| 544 | G220Y | 220 | 0.21 | 1 | 0.73 | 0.65 | 0.33 |
| 545 | D221N | 221 | 0.91 | 1 | 1.42 | 1.11 | 0.22 |
| 546 | D221I | 221 | 0.21 | 1 | 1.64 | 0.95 | 0.58 |
| 547 | D221C | 221 | 1.31 | 1.26 | 0.03 | 0.87 | 0.59 |
| 548 | D221R | 221 | 0.57 | 0.31 | 0.73 | 0.53 | 0.17 |
| 549 | D221P | 221 | 1 | 0.08 | 0.5 | 0.53 | 0.38 |
| 550 | D221L | 221 | 0.5 | 0.51 | 0.4 | 0.47 | 0.05 |
| 551 | D221S | 221 | −0.14 | 0.78 | 0.62 | 0.42 | 0.4 |
| 552 | M222P | 222 | 0.56 | 0.54 | 0.49 | 0.53 | 0.03 |
| 553 | M222C | 222 | 1.02 | 0.71 | −0.44 | 0.43 | 0.63 |
| 554 | K223F | 223 | 1.92 | 1.81 | 1.13 | 1.62 | 0.35 |
| 555 | K223P | 223 | 1.42 | 1 | 1.5 | 1.3 | 0.22 |
| 556 | K223C | 223 | 0.91 | 1.91 | 0.73 | 1.18 | 0.52 |
| 557 | K223G | 223 | 0.97 | 0.15 | 0.65 | 0.59 | 0.34 |
| 558 | D224H | 224 | −0.48 | 0.71 | 1.13 | 0.45 | 0.68 |
| 559 | S225A | 225 | 0.91 | 0.31 | 0.44 | 0.55 | 0.26 |
| 560 | S225Y | 225 | 0.91 | 1.41 | −0.66 | 0.55 | 0.88 |
| 561 | S225W | 225 | 0.8 | 0.31 | 0.5 | 0.54 | 0.2 |
| 562 | L226D | 226 | 1.06 | 1.51 | 0.73 | 1.1 | 0.32 |
| 563 | L226Y | 226 | 0.83 | 0.84 | 1.22 | 0.97 | 0.18 |
| 564 | E228P | 228 | 0.92 | 0.37 | 0.71 | 0.67 | 0.23 |
| 565 | E228F | 228 | 0.45 | 0.47 | 0.61 | 0.51 | 0.07 |
| 566 | E228W | 228 | 0.25 | 0.44 | 0.71 | 0.47 | 0.19 |
| 567 | E228T | 228 | 0.4 | 0.49 | 0.43 | 0.44 | 0.04 |
| 568 | M229S | 229 | 0.46 | 0.64 | 0.34 | 0.48 | 0.12 |
| 569 | S230G | 230 | 0.87 | 0.38 | 0.6 | 0.61 | 0.2 |
| 570 | S230E | 230 | 0.84 | 0.3 | 0.66 | 0.6 | 0.22 |
| 571 | L231K | 231 | 0.85 | 0.89 | 0.62 | 0.79 | 0.12 |
| 572 | L231D | 231 | 0.55 | 0.64 | 1.13 | 0.77 | 0.26 |
| 573 | L231R | 231 | 1.04 | 0.4 | 0.86 | 0.77 | 0.27 |
| 574 | E233S | 233 | 0.76 | −0.21 | 0.98 | 0.51 | 0.52 |
| 575 | I234W | 234 | 0.9 | 0.7 | 1.28 | 0.96 | 0.24 |
| 576 | I234G | 234 | 1.04 | 0.65 | −0.13 | 0.52 | 0.48 |
| 577 | Y235N | 235 | 0.21 | 1.22 | 1.21 | 0.88 | 0.47 |
| 578 | Y235L | 235 | 1.49 | 0.7 | 0.29 | 0.83 | 0.5 |
| 579 | Y235W | 235 | 1.17 | 0.3 | 0.62 | 0.69 | 0.36 |
| 580 | Y235A | 235 | 0.9 | −0.11 | 1.13 | 0.64 | 0.54 |
| 581 | Y235T | 235 | 0.72 | −0.11 | 0.88 | 0.5 | 0.43 |
| 582 | Y235Q | 235 | 0.21 | 1.22 | 0.03 | 0.48 | 0.52 |
| 583 | S236K | 236 | 1.09 | 0.48 | 1.19 | 0.92 | 0.31 |
| 584 | S236V | 236 | 0.9 | 0.99 | 0.72 | 0.87 | 0.11 |
| 585 | S236W | 236 | 0.56 | 0.86 | 0.59 | 0.67 | 0.14 |
| 586 | S236N | 236 | 1.24 | 0.99 | −0.48 | 0.58 | 0.76 |
| 587 | S236G | 236 | 0.55 | 0.52 | 0.29 | 0.45 | 0.12 |
| 588 | E238F | 238 | 0.37 | 0.51 | 1.15 | 0.68 | 0.34 |
| 589 | E238S | 238 | 0.46 | 0.75 | 0.57 | 0.59 | 0.12 |
| 590 | K239T | 239 | 1.27 | 0.7 | 0.47 | 0.81 | 0.33 |
| 591 | K239V | 239 | 0.62 | 0.39 | 0.61 | 0.54 | 0.1 |
| 592 | K239W | 239 | 0.57 | 0.55 | 0.48 | 0.53 | 0.04 |
| 593 | K239F | 239 | 0.33 | 0.16 | 0.89 | 0.46 | 0.31 |
| 594 | K239G | 239 | 0.5 | 0.12 | 0.72 | 0.44 | 0.25 |
| 595 | G241P | 241 | 2.15 | 0.66 | 0.38 | 1.06 | 0.77 |
| 596 | G241M | 241 | 1.07 | 1.2 | 0.76 | 1.01 | 0.18 |
| 597 | G241Q | 241 | 0.84 | 1.17 | 0.78 | 0.93 | 0.17 |
| 598 | G241W | 241 | 0.18 | 0.55 | 1.05 | 0.6 | 0.36 |
| 599 | G241H | 241 | 0.39 | 0.4 | 0.77 | 0.52 | 0.18 |
| 600 | G241F | 241 | 0.6 | 0.72 | 0.15 | 0.49 | 0.25 |
| 601 | F243R | 243 | 1.02 | 0.99 | 1.04 | 1.02 | 0.02 |
| 602 | F243H | 243 | 0.21 | 1.28 | 0.18 | 0.55 | 0.51 |
| 603 | I244Y | 244 | 1.22 | 1.31 | 0.84 | 1.12 | 0.2 |
| 604 | I244Q | 244 | 1.39 | 0.01 | 1.2 | 0.87 | 0.61 |
| 605 | I244G | 244 | 0.8 | 0.83 | 0.61 | 0.75 | 0.09 |
| 606 | I244F | 244 | 0.69 | 0.53 | 0.45 | 0.55 | 0.1 |
| 607 | I244W | 244 | 0.72 | 0.4 | 0.31 | 0.48 | 0.18 |
| 608 | T245Q | 245 | 1.41 | 1.14 | 1.41 | 1.32 | 0.13 |
| 609 | T245E | 245 | 0.9 | 0.99 | 1.12 | 1 | 0.09 |
| 610 | T245L | 245 | 1.15 | 0.61 | 1.2 | 0.99 | 0.27 |
| 611 | T245Y | 245 | 1.17 | 0.16 | 0.83 | 0.72 | 0.42 |
| 612 | T245V | 245 | 0.31 | 0.74 | 0.46 | 0.5 | 0.17 |
| 613 | T245K | 245 | 0.03 | 0.58 | 0.8 | 0.47 | 0.32 |
| 614 | E247T | 247 | 1.12 | 0.37 | 1.2 | 0.9 | 0.37 |
| 615 | E247M | 247 | 0.88 | 0.57 | 0.51 | 0.65 | 0.16 |
| 616 | E247C | 247 | 0.9 | 0.85 | −0.11 | 0.54 | 0.47 |
| 617 | E247I | 247 | 0.7 | 0.67 | −0.02 | 0.45 | 0.33 |
| 618 | G248M | 248 | 0.86 | 0.48 | 0.71 | 0.68 | 0.16 |
| 619 | G248K | 248 | 0.35 | 0.51 | 0.51 | 0.46 | 0.07 |
| 620 | S250N | 250 | 0.61 | 0.96 | 0.94 | 0.84 | 0.16 |
| 621 | N253Q | 253 | 1.71 | 1.21 | 1.41 | 1.44 | 0.21 |
| 622 | N253G | 253 | 1.12 | 1.54 | 0.72 | 1.13 | 0.34 |
| 623 | N253W | 253 | 1.12 | 0.7 | 0.58 | 0.8 | 0.23 |
| 624 | N253A | 253 | 1.06 | 0.29 | 1 | 0.78 | 0.35 |
| 625 | D254Q | 254 | 1.31 | 0.98 | 1.41 | 1.23 | 0.18 |
| 626 | D254S | 254 | 1.01 | 0.94 | 0.96 | 0.97 | 0.03 |
| 627 | D254W | 254 | 1.31 | 0.29 | 1.04 | 0.88 | 0.43 |
| 628 | D254I | 254 | 1.46 | 0.29 | 0.03 | 0.59 | 0.62 |
| 629 | D254R | 254 | 0.03 | 0.7 | 0.87 | 0.53 | 0.36 |
| 630 | D254M | 254 | 1.53 | 0.7 | −0.67 | 0.52 | 0.91 |
| 631 | C256Q | 256 | 0.43 | 0.74 | 0.62 | 0.6 | 0.13 |
| 632 | C256H | 256 | 0.56 | 0.3 | 0.77 | 0.54 | 0.19 |
| 633 | C256E | 256 | 0.4 | 0.61 | 0.46 | 0.49 | 0.09 |
| 634 | C256N | 256 | 0.6 | 0.3 | 0.51 | 0.47 | 0.13 |
| 635 | C256M | 256 | 0.49 | 0.54 | 0.38 | 0.47 | 0.07 |
| 636 | K258L | 258 | 0.69 | 0.52 | 0.3 | 0.51 | 0.16 |
| 637 | V259Y | 259 | 1.81 | 1.39 | 1.41 | 1.54 | 0.2 |
| 638 | V259Q | 259 | 0.21 | 0.29 | 1.63 | 0.71 | 0.65 |
| 639 | V259N | 259 | 0.9 | 0.29 | 0.72 | 0.63 | 0.26 |
| 640 | S261H | 261 | 0.9 | 0.29 | 0.87 | 0.68 | 0.28 |
| 641 | S261F | 261 | 0.36 | 1.06 | 0.18 | 0.53 | 0.38 |
| 642 | F262R | 262 | 1.67 | 1.83 | 1.22 | 1.57 | 0.26 |
| 643 | F262P | 262 | 1.59 | 0.28 | 1.12 | 1 | 0.54 |
| 644 | F262K | 262 | 1.45 | 0.51 | 0.58 | 0.85 | 0.43 |
| 645 | F262G | 262 | 0.42 | 0.69 | 1.03 | 0.71 | 0.25 |
| 646 | M263E | 263 | 0.87 | 0.41 | 0.64 | 0.64 | 0.19 |
| 647 | M263C | 263 | 0.64 | 0.63 | 0.53 | 0.6 | 0.05 |
| 648 | M263S | 263 | 0.53 | 0.45 | 0.27 | 0.42 | 0.11 |
| 649 | L265W | 265 | 1.59 | −0.3 | 0.89 | 0.73 | 0.78 |
| 650 | L265K | 265 | 0.61 | 0.59 | 0.43 | 0.54 | 0.08 |
| 651 | Y266R | 266 | 0.2 | 3.72 | 0.71 | 1.54 | 1.55 |
| 652 | Y266E | 266 | 0.81 | 0.79 | 0.31 | 0.64 | 0.23 |
| 653 | Y266P | 266 | 0.9 | −0.12 | 0.53 | 0.43 | 0.42 |
| 654 | C267K | 267 | 1.12 | 1.2 | 1.63 | 1.31 | 0.22 |
| 655 | C267M | 267 | 0.61 | 1.38 | 0.65 | 0.88 | 0.36 |
| 656 | C267E | 267 | 0.89 | 1.38 | 0.31 | 0.86 | 0.44 |
| 657 | C267T | 267 | 0.85 | 0.2 | 0.62 | 0.56 | 0.27 |
| 658 | C267P | 267 | 0.81 | 0.28 | 0.53 | 0.54 | 0.21 |
| 659 | Q268C | 268 | 1.08 | 1.45 | 1.05 | 1.19 | 0.18 |
| 660 | Q268G | 268 | 0.71 | 1.13 | 0.53 | 0.79 | 0.25 |
| 661 | Q268V | 268 | 1.01 | 1.2 | 0.02 | 0.74 | 0.52 |
| 662 | K269Y | 269 | 0.2 | 0.42 | 1.02 | 0.55 | 0.35 |
| 663 | N270G | 270 | 0.65 | 0.82 | 1.63 | 1.03 | 0.43 |
| 664 | N270E | 270 | 0.74 | 0.9 | 0.15 | 0.6 | 0.32 |
| 665 | K271W | 271 | 0.63 | 0.8 | 0.78 | 0.73 | 0.07 |
| 666 | K271H | 271 | 0.61 | 0.98 | 0.42 | 0.67 | 0.23 |
| 667 | K271C | 271 | −0.2 | 0.98 | 1 | 0.59 | 0.56 |
| 668 | K271I | 271 | 1.05 | 0.1 | 0.42 | 0.52 | 0.39 |
| 669 | E272S | 272 | 0.9 | 1.38 | 1.4 | 1.23 | 0.24 |
| 670 | E272P | 272 | 0.2 | 0.98 | 0.93 | 0.71 | 0.36 |
| 671 | E272K | 272 | 0.98 | 0.69 | 0.31 | 0.66 | 0.27 |
| 672 | E272G | 272 | 0.49 | −0.12 | 1.12 | 0.5 | 0.51 |
| 673 | E272Y | 272 | 0.71 | 0.57 | 0.02 | 0.43 | 0.3 |
| 674 | N273R | 273 | 0.65 | 0.56 | 0.47 | 0.56 | 0.08 |
| 675 | N273I | 273 | 0.49 | 1.13 | 0.02 | 0.55 | 0.46 |
| 676 | N273S | 273 | 0.46 | 0.25 | 0.91 | 0.54 | 0.28 |
| 677 | N275G | 275 | 0.36 | 0.61 | 0.75 | 0.57 | 0.16 |
| 678 | L276K | 276 | 1.12 | 1.2 | 1.97 | 1.43 | 0.38 |
| 679 | L276N | 276 | 1.45 | 0.98 | 0.42 | 0.95 | 0.42 |
| 680 | Y277P | 277 | 1.3 | 1.2 | 1.48 | 1.33 | 0.12 |
| 681 | Y277L | 277 | 0.83 | 0.59 | 0.2 | 0.54 | 0.26 |
| 682 | Y277M | 277 | 0.8 | 0.08 | 0.39 | 0.42 | 0.29 |
| 683 | K278W | 278 | 1.74 | 0.98 | 0.42 | 1.05 | 0.54 |
| 684 | K278A | 278 | 0.67 | 1.07 | 0.71 | 0.82 | 0.18 |
| 685 | K278P | 278 | 0.45 | 0.98 | 0.71 | 0.71 | 0.21 |
| 686 | K278S | 278 | 0.85 | 0.69 | 0.22 | 0.59 | 0.26 |
| 687 | K278F | 278 | −0.09 | 0.84 | 0.83 | 0.53 | 0.43 |
| 688 | K278L | 278 | 0.49 | 0.35 | 0.51 | 0.45 | 0.07 |
| 689 | Q280M | 280 | 0.89 | 0.5 | 0.58 | 0.66 | 0.17 |
| 690 | Q280D | 280 | 0.32 | 0.97 | 0.34 | 0.54 | 0.31 |
| 691 | K281G | 281 | 1.05 | 0.87 | 0.77 | 0.9 | 0.11 |
| 692 | K281F | 281 | 0.76 | 0.28 | 1.27 | 0.77 | 0.4 |
| 693 | K281D | 281 | −0.61 | 0.48 | 2.2 | 0.69 | 1.16 |
| 694 | L282S | 282 | −0.06 | 0.08 | 1.27 | 0.43 | 0.6 |
| 695 | L282K | 282 | 0.71 | 0.16 | 0.38 | 0.42 | 0.22 |
| 696 | H283M | 283 | 1.08 | 0.91 | 0.45 | 0.81 | 0.27 |
| 697 | Q285M | 285 | 1.44 | 1.52 | 2.1 | 1.68 | 0.29 |
| 698 | Q285T | 285 | −0.76 | 0.71 | 1.95 | 0.64 | 1.11 |
| 699 | I286C | 286 | 1.45 | 0.28 | 0.71 | 0.81 | 0.48 |
| 700 | C288G | 288 | 0.57 | 0.69 | 0.57 | 0.61 | 0.06 |
| 701 | C288K | 288 | 0.56 | 0.42 | 0.57 | 0.52 | 0.07 |
| 702 | I289W | 289 | 0.87 | 0.66 | 0.33 | 0.62 | 0.22 |
| 703 | A290K | 290 | 0.59 | 0.54 | 0.37 | 0.5 | 0.09 |
| 704 | A290I | 290 | 0.28 | 0.69 | 0.35 | 0.44 | 0.18 |
| 705 | A290N | 290 | 0.68 | 0.59 | 0.01 | 0.43 | 0.29 |
| 706 | A290R | 290 | 0.15 | 0.38 | 0.72 | 0.42 | 0.23 |
| 707 | D291T | 291 | 1.21 | 0.46 | 1.87 | 1.18 | 0.57 |
| 708 | D291M | 291 | 0.85 | 0.63 | 0.76 | 0.75 | 0.09 |
| 709 | D291N | 291 | 0.63 | 0.68 | 0.77 | 0.7 | 0.06 |
| 710 | D291F | 291 | 0.49 | 0.75 | 0.8 | 0.68 | 0.14 |
| 711 | D291P | 291 | 0.48 | 0.36 | 1.08 | 0.64 | 0.32 |
| 712 | D291L | 291 | 0.63 | 0.62 | 0.29 | 0.51 | 0.16 |
| 713 | D291W | 291 | 0.52 | 0.69 | 0.3 | 0.5 | 0.16 |
| 714 | T292H | 292 | 0.42 | 0.46 | 0.54 | 0.47 | 0.05 |
| 715 | S293K | 293 | 0.89 | 0.64 | 0.61 | 0.71 | 0.12 |
| 716 | S293W | 293 | 0.19 | 0.46 | 1.02 | 0.56 | 0.35 |
| 717 | S293H | 293 | 0.38 | 0.66 | 0.52 | 0.52 | 0.12 |
| 718 | Y294L | 294 | 0.25 | 0.34 | 0.88 | 0.49 | 0.28 |
| 719 | Y294M | 294 | 0.74 | 0.37 | 0.31 | 0.47 | 0.19 |
| 720 | Y294V | 294 | 0.55 | 0.37 | 0.46 | 0.46 | 0.07 |
| 721 | E295C | 295 | 0.19 | 0.24 | 2.85 | 1.09 | 1.24 |
| 722 | E295M | 295 | 1.33 | 0.37 | −0.01 | 0.56 | 0.56 |
| 723 | E295T | 295 | 0.64 | 0.58 | 0.36 | 0.52 | 0.12 |
| 724 | E295R | 295 | 0.49 | 0.55 | 0.46 | 0.5 | 0.04 |
| 725 | V296Q | 296 | 1.25 | 1.08 | 1.03 | 1.12 | 0.09 |
| 726 | V296R | 296 | 0.95 | 1.13 | 0.97 | 1.02 | 0.08 |
| 727 | V296N | 296 | 1.05 | 0.71 | 1.05 | 0.93 | 0.16 |
| 728 | V296P | 296 | 0.63 | 1.13 | 0.9 | 0.89 | 0.21 |
| 729 | V296H | 296 | 0.81 | 0.92 | 0.81 | 0.85 | 0.05 |
| 730 | V296W | 296 | 0.8 | 1.01 | 0.58 | 0.8 | 0.17 |
| 731 | V296K | 296 | 0.45 | 0.72 | 0.73 | 0.63 | 0.13 |
| 732 | V296D | 296 | 0.65 | 0.74 | 0.39 | 0.59 | 0.15 |
| 733 | V296T | 296 | 0.65 | 0.49 | 0.53 | 0.56 | 0.07 |
| 734 | V296Y | 296 | 0.7 | 0.54 | 0.35 | 0.53 | 0.14 |
| 735 | V296A | 296 | 0.47 | 0.44 | 0.64 | 0.52 | 0.09 |
| 736 | V296M | 296 | 0.76 | 0.45 | 0.31 | 0.5 | 0.19 |
| 737 | V296G | 296 | 0.38 | 0.43 | 0.48 | 0.43 | 0.04 |
| 738 | P297F | 297 | 1.34 | 1.36 | 1.49 | 1.4 | 0.07 |
| 739 | P297I | 297 | 1.46 | 0.88 | 0.93 | 1.09 | 0.26 |
| 740 | P297C | 297 | 1.26 | 1 | 0.7 | 0.99 | 0.23 |
| 741 | P297V | 297 | 0.85 | 0.76 | 1.06 | 0.89 | 0.12 |
| 742 | P297W | 297 | 0.83 | 0.91 | 0.91 | 0.88 | 0.04 |
| 743 | P297H | 297 | 0.76 | 0.73 | 0.52 | 0.67 | 0.11 |
| 744 | P297N | 297 | 0.77 | 0.64 | 0.53 | 0.64 | 0.1 |
| 745 | P297M | 297 | 0.69 | 0.5 | 0.6 | 0.59 | 0.08 |
| 746 | P297K | 297 | 0.54 | 0.61 | 0.56 | 0.57 | 0.03 |
| 747 | P297Y | 297 | 0.52 | 0.5 | 0.66 | 0.56 | 0.07 |
| 748 | P297R | 297 | 0.49 | 0.47 | 0.37 | 0.45 | 0.05 |
| 749 | K299P | 299 | 1.54 | 1.62 | 1.65 | 1.6 | 0.05 |
| 750 | K299E | 299 | 0.84 | 0.97 | 1.09 | 0.97 | 0.1 |
| 751 | K299G | 299 | 1.12 | 0.71 | 0.95 | 0.93 | 0.17 |
| 752 | K299A | 299 | 0.87 | 0.7 | 0.76 | 0.78 | 0.07 |
| 753 | K299W | 299 | 0.78 | 0.52 | 0.56 | 0.62 | 0.12 |
| 754 | K299V | 299 | 0.54 | 0.66 | 0.65 | 0.62 | 0.06 |
| 755 | F300E | 300 | 0.98 | 0.95 | 1.12 | 1.02 | 0.07 |
| 756 | F300K | 300 | 1.05 | 1.04 | 0.66 | 0.92 | 0.18 |
| 757 | F300M | 300 | 0.53 | 0.74 | 0.59 | 0.62 | 0.09 |
| 758 | F300Q | 300 | 0.8 | 0.71 | 0.22 | 0.58 | 0.26 |
| 759 | F300D | 300 | 0.71 | 0.34 | 0.37 | 0.47 | 0.17 |
| 760 | E301G | 301 | 1.94 | 1.88 | 2.02 | 1.95 | 0.06 |
| 761 | E301W | 301 | 1.42 | 1.71 | 1.45 | 1.53 | 0.13 |
| 762 | E301P | 301 | 1.58 | 1.48 | 0.99 | 1.35 | 0.26 |
| 763 | E301Q | 301 | 1.11 | 1.38 | 1.4 | 1.29 | 0.13 |
| 764 | E301M | 301 | 0.89 | 0.28 | 1.96 | 1.04 | 0.69 |
| 765 | E301A | 301 | 0.93 | 0.45 | 1.35 | 0.91 | 0.37 |
| 766 | E301K | 301 | 0.6 | 0.84 | 1.26 | 0.9 | 0.28 |
| 767 | E301R | 301 | 0.57 | 1.45 | 0.39 | 0.8 | 0.47 |
| 768 | E301F | 301 | 0.98 | 0.46 | 0.7 | 0.72 | 0.21 |
| 769 | E301C | 301 | 0.29 | 0.87 | 0.84 | 0.67 | 0.27 |
| 770 | E301T | 301 | 0.89 | 1.67 | −0.68 | 0.62 | 0.98 |
| 771 | S302I | 302 | 2.18 | 2.07 | 2.31 | 2.19 | 0.1 |
| 772 | S302D | 302 | 1.49 | 2.23 | 1.86 | 1.86 | 0.3 |
| 773 | S302V | 302 | 1.67 | 1.84 | 1.66 | 1.72 | 0.08 |
| 774 | S302G | 302 | 1.44 | 1.14 | 1.6 | 1.39 | 0.19 |
| 775 | S302E | 302 | 1.58 | 0.84 | 1.57 | 1.33 | 0.35 |
| 776 | S302M | 302 | 1.58 | 1.67 | 0.48 | 1.24 | 0.54 |
| 777 | S302A | 302 | 0.85 | 1.18 | 0.89 | 0.97 | 0.15 |
| 778 | S302W | 302 | 1.08 | 0.77 | 0.97 | 0.94 | 0.13 |
| 779 | S302N | 302 | 0.64 | 0.82 | 1.01 | 0.82 | 0.15 |
| 780 | S302C | 302 | 0.48 | 0.97 | 0.52 | 0.66 | 0.22 |
| 781 | S302H | 302 | 0.89 | 0.28 | 0.7 | 0.62 | 0.25 |
| 782 | D303C | 303 | 3.03 | 3.65 | 3.23 | 3.3 | 0.26 |
| 783 | D303M | 303 | 3.14 | 3.09 | 2.97 | 3.07 | 0.07 |
| 784 | D303F | 303 | 2.59 | 2.84 | 2.71 | 2.71 | 0.1 |
| 785 | D303Y | 303 | 2.02 | 2.05 | 1.96 | 2.01 | 0.04 |
| 786 | D303S | 303 | 1.93 | 1.97 | 1.96 | 1.95 | 0.02 |
| 787 | D303I | 303 | 1.47 | 1.72 | 1.58 | 1.59 | 0.1 |
| 788 | D303R | 303 | 1.54 | 1.66 | 1.34 | 1.51 | 0.13 |
| 789 | D303W | 303 | 1.23 | 1.6 | 1.48 | 1.44 | 0.15 |
| 790 | D303P | 303 | 1.46 | 1.6 | 1.22 | 1.43 | 0.15 |
| 791 | D303V | 303 | 1.55 | 1.2 | 1.26 | 1.34 | 0.15 |
| 792 | D303Q | 303 | 1.14 | 1.32 | 1.35 | 1.27 | 0.09 |
| 793 | D303G | 303 | 1 | 1.27 | 1.18 | 1.15 | 0.11 |
| 794 | D303H | 303 | 1.09 | 1.13 | 1.18 | 1.13 | 0.04 |
| 795 | D303K | 303 | 0.76 | 1.09 | 1.12 | 0.99 | 0.16 |
| 796 | D303N | 303 | 0.63 | 0.72 | 0.76 | 0.7 | 0.05 |
| 797 | E304T | 304 | 0.69 | 0.61 | 0.76 | 0.69 | 0.06 |
| 798 | E305W | 305 | 1.98 | 1.85 | 2.18 | 2.01 | 0.14 |
| 799 | E305C | 305 | 1.39 | 1.75 | 1.9 | 1.68 | 0.22 |
| 800 | E305F | 305 | 1.61 | 1.56 | 1.64 | 1.6 | 0.03 |
| 801 | E305S | 305 | 1.51 | 1.46 | 1.69 | 1.55 | 0.1 |
| 802 | E305Y | 305 | 1.58 | 1.47 | 1.48 | 1.51 | 0.05 |
| 803 | E305L | 305 | 1.46 | 1.44 | 1.4 | 1.44 | 0.03 |
| 804 | E305A | 305 | 1.26 | 1.41 | 1.5 | 1.39 | 0.1 |
| 805 | E305K | 305 | 1.42 | 1.27 | 1.28 | 1.33 | 0.07 |
| 806 | E3051 | 305 | 1.48 | 1.31 | 1.15 | 1.31 | 0.13 |
| 807 | E305V | 305 | 1.37 | 1.33 | 1.18 | 1.3 | 0.08 |
| 808 | E305G | 305 | 1.19 | 1.21 | 1.49 | 1.29 | 0.14 |
| 809 | E305M | 305 | 1.2 | 0.87 | 1.04 | 1.04 | 0.14 |
| 810 | E305R | 305 | 1.08 | 0.87 | 0.97 | 0.97 | 0.09 |
| 811 | E305N | 305 | 0.68 | 0.59 | 1.11 | 0.79 | 0.23 |
| 812 | E305P | 305 | 0.43 | 0.6 | 0.59 | 0.54 | 0.08 |
| 813 | E305H | 305 | −0.08 | 1.04 | 0.43 | 0.46 | 0.46 |
| 814 | V306W | 306 | 2.13 | 1.38 | 1.39 | 1.63 | 0.35 |
| 815 | V306Y | 306 | 1.69 | 0.68 | 0.7 | 1.02 | 0.47 |
| 816 | V306F | 306 | 1.17 | −0.13 | 0.38 | 0.47 | 0.53 |
| 817 | V306C | 306 | 0.23 | 0.72 | 0.32 | 0.43 | 0.21 |
| 818 | Y307Q | 307 | 1.88 | 2.18 | 2.02 | 2.03 | 0.12 |
| 819 | Y307K | 307 | 1.87 | 2.01 | 2.02 | 1.97 | 0.07 |
| 820 | Y307E | 307 | 1.83 | 2.04 | 1.94 | 1.94 | 0.09 |
| 821 | Y307R | 307 | 1.77 | 1.67 | 2.01 | 1.82 | 0.14 |
| 822 | Y307G | 307 | 1.81 | 1.72 | 1.86 | 1.8 | 0.06 |
| 823 | Y307A | 307 | 1.61 | 1.69 | 1.78 | 1.69 | 0.07 |
| 824 | Y307T | 307 | 1.62 | 1.72 | 1.68 | 1.68 | 0.04 |
| 825 | Y307I | 307 | 1.53 | 1.53 | 1.33 | 1.46 | 0.09 |
| 826 | Y307L | 307 | 1.26 | 1.1 | 1.25 | 1.2 | 0.08 |
| 827 | Y307W | 307 | 0.97 | 1.24 | 1.37 | 1.19 | 0.17 |
| 828 | Y307V | 307 | 1.08 | 1.17 | 1.15 | 1.13 | 0.04 |
| 829 | Y307S | 307 | 0.83 | 0.97 | 0.97 | 0.92 | 0.06 |
| 830 | Y307P | 307 | 0.92 | 0.52 | 0.71 | 0.72 | 0.16 |
| 831 | Y307D | 307 | 0.72 | 0.58 | 0.64 | 0.64 | 0.06 |
| 832 | Y307N | 307 | 0.4 | 0.54 | 0.48 | 0.47 | 0.06 |
| 833 | Q308I | 308 | 1.29 | 1.09 | 0.57 | 0.98 | 0.3 |
| 834 | Q308W | 308 | 0.53 | 0.54 | 0.51 | 0.53 | 0.01 |
| 835 | S309M | 309 | 0.57 | 0.95 | 0.81 | 0.78 | 0.15 |
| 836 | S309L | 309 | 0.44 | 0.68 | 0.49 | 0.54 | 0.1 |
| 837 | S309K | 309 | 0.52 | 0.36 | 0.61 | 0.5 | 0.1 |
| 838 | S309V | 309 | 0.5 | 0.22 | 0.59 | 0.43 | 0.16 |
| 839 | V310Y | 310 | 1.43 | 1.46 | 1.35 | 1.42 | 0.05 |
| 840 | N311F | 311 | 1.24 | 0.88 | 0.75 | 0.96 | 0.21 |
| 841 | N311L | 311 | 0.67 | 0.76 | 0.67 | 0.7 | 0.04 |
| 842 | G312I | 312 | 1.79 | 1.38 | 1.62 | 1.59 | 0.17 |
| 843 | G312T | 312 | 1.21 | 0.46 | 1.04 | 0.9 | 0.32 |
| 844 | G312E | 312 | 1.28 | 1.38 | 0.01 | 0.89 | 0.62 |
| 845 | G312S | 312 | 0.47 | 1.05 | 0.41 | 0.64 | 0.29 |
| 846 | G312Q | 312 | 0.87 | 0.97 | 0.01 | 0.62 | 0.43 |
| 847 | G312A | 312 | 0.6 | 0.53 | 0.52 | 0.55 | 0.03 |
| 848 | G312K | 312 | −0.22 | 0.79 | 0.7 | 0.42 | 0.46 |
| 849 | F313H | 313 | 0.76 | 0.16 | 0.9 | 0.6 | 0.32 |
| 850 | F313M | 313 | 0.29 | 0.43 | 0.76 | 0.49 | 0.19 |
| 851 | D315M | 315 | 0.52 | 1.3 | 0.59 | 0.8 | 0.36 |
| 852 | D315I | 315 | 1.05 | 0.05 | 0.79 | 0.63 | 0.42 |
| 853 | I317W | 317 | 0.5 | 0.64 | 0.49 | 0.54 | 0.07 |
| 854 | S318K | 318 | 0.8 | 0.58 | 0.54 | 0.64 | 0.11 |
| 855 | S318D | 318 | 0.54 | 0.69 | 0.3 | 0.51 | 0.16 |
| 856 | S319K | 319 | 0.55 | 0.84 | 0.35 | 0.58 | 0.2 |
| 857 | S319I | 319 | 0.87 | −0.13 | 0.79 | 0.51 | 0.46 |
| 858 | S319N | 319 | −0.02 | 0.84 | 0.6 | 0.48 | 0.36 |
| 859 | K320F | 320 | 0.82 | 0.74 | 0.26 | 0.61 | 0.25 |
| 860 | H321V | 321 | 0.38 | 0.71 | 0.94 | 0.68 | 0.23 |
| 861 | H321F | 321 | 0.72 | 0.18 | 0.94 | 0.61 | 0.32 |
| 862 | I322A | 322 | 0.78 | 0.77 | 0.74 | 0.76 | 0.02 |
| 863 | E324W | 324 | 1.97 | 2.35 | 1.79 | 2.04 | 0.23 |
| 864 | E324C | 324 | 0.18 | 2.7 | 0.77 | 1.22 | 1.08 |
| 865 | E324Y | 324 | 0.78 | 0.43 | 0.77 | 0.66 | 0.17 |
| 866 | E324M | 324 | 0.87 | 0.49 | 0.22 | 0.53 | 0.26 |
| 867 | E324T | 324 | 0.18 | 0.35 | 0.77 | 0.43 | 0.25 |
| 868 | R325K | 325 | 0.17 | 0.78 | 0.69 | 0.55 | 0.27 |
| 869 | R327V | 327 | 0.76 | 0.4 | 0.55 | 0.57 | 0.15 |
| 870 | R327M | 327 | 0.58 | 0.5 | 0.56 | 0.55 | 0.03 |
| 871 | R327K | 327 | 0.18 | 0.85 | 0.29 | 0.44 | 0.3 |
| 872 | D331M | 331 | 0.84 | 0.78 | 0.58 | 0.73 | 0.11 |
| 873 | D331C | 331 | 0.92 | 0.68 | 0.34 | 0.65 | 0.24 |
| 874 | Y333M | 333 | 0.61 | 0.55 | 0.18 | 0.45 | 0.19 |
| 875 | N334W | 334 | 0.78 | 0.97 | 0.78 | 0.84 | 0.09 |
| 876 | G335Q | 335 | 0.62 | 0.49 | 0.28 | 0.46 | 0.14 |
| 877 | Y336V | 336 | 1.79 | 1.88 | 0.01 | 1.23 | 0.86 |
| 878 | Y336H | 336 | 1.43 | 0.68 | 0.92 | 1.01 | 0.31 |
| 879 | Y336W | 336 | 0.87 | 1.28 | 0.41 | 0.86 | 0.36 |
| 880 | Y336M | 336 | 0.47 | 0.97 | 0.29 | 0.58 | 0.29 |
| 881 | N337G | 337 | 0.89 | 0.51 | 0.33 | 0.58 | 0.23 |
| 882 | D339Q | 339 | 1.28 | −0.42 | 0.7 | 0.52 | 0.7 |
| 883 | D339M | 339 | 0.87 | 0.68 | 0.01 | 0.52 | 0.37 |
| 884 | D339S | 339 | −0.13 | 1.91 | −0.4 | 0.46 | 1.03 |
| 885 | K340E | 340 | 0.69 | 1.12 | 1.47 | 1.09 | 0.32 |
| 886 | K340H | 340 | 1.28 | 0.97 | 0.01 | 0.75 | 0.54 |
| 887 | K340A | 340 | 0.87 | 0.27 | 1.1 | 0.75 | 0.35 |
| 888 | Y342R | 342 | 0.79 | 1.12 | 0.85 | 0.92 | 0.14 |
| 889 | Y342M | 342 | 0.99 | −0.01 | 0.41 | 0.46 | 0.41 |
| 890 | I343V | 343 | −0.51 | 0.97 | 0.85 | 0.44 | 0.67 |
| 891 | V344E | 344 | 1.28 | 0.97 | 1.1 | 1.12 | 0.13 |
| 892 | K346C | 346 | 0.18 | 1.88 | 0.7 | 0.92 | 0.71 |
| 893 | F347R | 347 | 0.87 | 1.66 | 1.61 | 1.38 | 0.36 |
| 894 | F347G | 347 | 0.18 | 0.27 | 0.92 | 0.46 | 0.33 |
| 895 | Y348M | 348 | 1.28 | 1.37 | 0 | 0.88 | 0.62 |
| 896 | Y348V | 348 | 0.18 | 0.27 | 1.39 | 0.61 | 0.55 |
| 897 | E349T | 349 | 2.26 | 0.96 | 1.1 | 1.44 | 0.58 |
| 898 | E349Q | 349 | 1.56 | 0.96 | 1.61 | 1.38 | 0.29 |
| 899 | E349R | 349 | 1.09 | 0.96 | 1.18 | 1.08 | 0.09 |
| 900 | E349N | 349 | 1.09 | 0.68 | 1.25 | 1.01 | 0.24 |
| 901 | E349K | 349 | 0.97 | 0.74 | 0.69 | 0.8 | 0.12 |
| 902 | S350R | 350 | 2.48 | 1.66 | 2.49 | 2.21 | 0.39 |
| 903 | S350H | 350 | 0.58 | 0.96 | 0 | 0.52 | 0.4 |
| 904 | V351K | 351 | 2.12 | 2.35 | 1.61 | 2.03 | 0.31 |
| 905 | V351Y | 351 | 1.28 | 1.19 | 1.71 | 1.39 | 0.23 |
| 906 | V351F | 351 | 1.43 | 0.27 | 1.1 | 0.93 | 0.49 |
| 907 | V351D | 351 | 1.79 | 0.56 | 0 | 0.78 | 0.75 |
| 908 | S352M | 352 | 0.18 | 1.19 | 1.39 | 0.92 | 0.53 |
| 909 | Q353K | 353 | 0.77 | 0.93 | 0.89 | 0.86 | 0.07 |
| 910 | Q353M | 353 | 0.58 | 0.43 | 1.38 | 0.8 | 0.42 |
| 911 | Q353R | 353 | 0.55 | 0.76 | 0.54 | 0.62 | 0.1 |
| 912 | Q353N | 353 | 0.17 | 0.54 | 0.57 | 0.43 | 0.18 |
| 913 | T355M | 355 | 0.83 | 0.47 | 0.4 | 0.57 | 0.19 |
| 914 | Y356M | 356 | 1.56 | 1.19 | 1.25 | 1.33 | 0.16 |
| 915 | Y356V | 356 | 0.96 | 0.86 | 0.79 | 0.87 | 0.07 |
| 916 | Y356G | 356 | −0.11 | 1.37 | 0.69 | 0.65 | 0.61 |
| 917 | Y356I | 356 | 0.06 | 0.85 | 1.02 | 0.64 | 0.42 |
| 918 | Y356C | 356 | 0.51 | 0.27 | 0.95 | 0.58 | 0.28 |
| 919 | Y356A | 356 | 1.16 | 0.02 | 0.51 | 0.56 | 0.46 |
| 920 | Y356E | 356 | −0.05 | 0.61 | 0.69 | 0.42 | 0.33 |
| 921 | R357M | 357 | 0.61 | 0.65 | 0.37 | 0.54 | 0.12 |
| 922 | R357K | 357 | 0.61 | 0.35 | 0.48 | 0.48 | 0.1 |
| 923 | D358R | 358 | 0.41 | 0.76 | 0.69 | 0.62 | 0.15 |
| 924 | D358W | 358 | 0.27 | 0.61 | 0.4 | 0.43 | 0.14 |
| 925 | T361K | 361 | 0.42 | 0.63 | 1.08 | 0.71 | 0.28 |
| 926 | T364W | 364 | 0.37 | 0.25 | 0.72 | 0.45 | 0.2 |
| 927 | A365Y | 365 | 1.04 | 0.8 | 0.14 | 0.66 | 0.38 |
| 928 | A365N | 365 | 0.62 | 0.4 | 0.64 | 0.56 | 0.11 |
| 929 | E367P | 367 | 0.76 | 0.68 | 0.52 | 0.65 | 0.1 |
| 930 | E367M | 367 | 0.58 | 0.41 | 0.29 | 0.43 | 0.12 |
| 931 | I368P | 368 | 1.4 | 0.72 | 0.91 | 1.01 | 0.29 |
| 932 | I368Y | 368 | 0.42 | 0.52 | 0.79 | 0.57 | 0.16 |
| 933 | H369A | 369 | 0.99 | 1.34 | 1.04 | 1.13 | 0.15 |
| 934 | H369E | 369 | 0.28 | 0.5 | 1.21 | 0.66 | 0.4 |
| 935 | H369G | 369 | 0.38 | 0.9 | 0.52 | 0.6 | 0.22 |
| 936 | H369V | 369 | 0.73 | 0.7 | 0.33 | 0.58 | 0.18 |
| 937 | H369I | 369 | 0.15 | 0.75 | 0.57 | 0.49 | 0.25 |
| 938 | Y370A | 370 | 0.71 | 0.99 | 0.53 | 0.74 | 0.19 |
| 939 | Y370N | 370 | 0.9 | 0.78 | 0.48 | 0.72 | 0.18 |
| 940 | Y370G | 370 | 0.78 | 0.39 | 0.81 | 0.66 | 0.19 |
| 941 | Y370R | 370 | 0.54 | 0.5 | 0.55 | 0.53 | 0.02 |
| 942 | Y370P | 370 | 0.47 | 0.49 | 0.58 | 0.51 | 0.05 |
| 943 | N371C | 371 | 1.1 | 1.2 | 0.76 | 1.02 | 0.19 |
| 944 | N371G | 371 | 0.61 | 0.77 | 0.07 | 0.49 | 0.3 |
| 945 | N372F | 372 | 1.16 | 0.83 | 0.85 | 0.94 | 0.15 |
| 946 | N372A | 372 | 0.52 | 0.72 | 0.47 | 0.57 | 0.11 |
| 947 | N372W | 372 | 0.52 | 0.62 | 0.19 | 0.44 | 0.18 |
| 948 | I373D | 373 | 0.6 | 0.8 | 0.39 | 0.59 | 0.17 |
| 949 | L374H | 374 | 0.89 | 0.68 | 1.1 | 0.89 | 0.17 |
| 950 | L374Y | 374 | 1.17 | 0.83 | −0.03 | 0.66 | 0.51 |
| 951 | L374E | 374 | 0.61 | 0.65 | 0.37 | 0.55 | 0.12 |
| 952 | L374N | 374 | 0.55 | 0.24 | 0.53 | 0.44 | 0.14 |
| 953 | P3751 | 375 | 0.45 | 0.58 | 0.45 | 0.49 | 0.06 |
| 954 | P375Y | 375 | 0.46 | 0.48 | 0.48 | 0.47 | 0.01 |
| 955 | G376Q | 376 | 0.63 | 1.05 | 1.12 | 0.93 | 0.22 |
| 956 | N377V | 377 | 0.94 | 0.89 | 0.45 | 0.76 | 0.22 |
| 957 | N377L | 377 | 0.55 | 0.42 | 0.46 | 0.48 | 0.05 |
| 958 | N377P | 377 | 0.81 | 0.28 | 0.15 | 0.42 | 0.28 |
| 959 | G378N | 378 | 0.41 | 0.9 | 0.49 | 0.6 | 0.21 |
| 960 | G378W | 378 | 0.4 | 0.45 | 0.59 | 0.48 | 0.08 |
| 961 | K379F | 379 | 0.65 | 0.75 | 0.59 | 0.66 | 0.07 |
| 962 | K379A | 379 | 0.52 | 0.53 | 0.48 | 0.51 | 0.02 |
| 963 | K379R | 379 | 0.17 | 0.65 | 0.66 | 0.49 | 0.23 |
| 964 | K379G | 379 | 0.58 | 0.53 | 0.21 | 0.44 | 0.16 |
| 965 | K381L | 381 | 0.56 | 0.63 | 0.51 | 0.57 | 0.05 |
| 966 | K381W | 381 | 0.57 | 0.82 | 0.23 | 0.54 | 0.24 |
| 967 | K381P | 381 | 0.45 | 0.24 | 0.56 | 0.42 | 0.13 |
| 968 | D383M | 383 | 0.61 | 0.56 | 0.5 | 0.56 | 0.05 |
| 969 | D383P | 383 | 0.5 | 0.51 | 0.33 | 0.45 | 0.08 |
| 970 | K384D | 384 | 0.15 | 0.86 | 0.78 | 0.59 | 0.32 |
| 971 | K384P | 384 | 0.58 | 0.41 | 0.56 | 0.52 | 0.08 |
| 972 | K384G | 384 | 0.62 | 0.61 | 0.25 | 0.49 | 0.17 |
| 973 | V385C | 385 | 0.41 | 0.61 | 0.5 | 0.51 | 0.08 |
| 974 | V385Y | 385 | 0.53 | 0.53 | 0.44 | 0.5 | 0.04 |
| 975 | K386F | 386 | 0.55 | 0.47 | 0.28 | 0.43 | 0.11 |
| 976 | A388I | 388 | 0.61 | 1.06 | 0.91 | 0.86 | 0.19 |
| 977 | A388N | 388 | 0.64 | 0.48 | 1.05 | 0.72 | 0.24 |
| 978 | A388H | 388 | 0.31 | 0.49 | 0.54 | 0.45 | 0.1 |
| 979 | A388Y | 388 | 0.57 | 0.65 | 0.08 | 0.43 | 0.25 |
| 980 | V389E | 389 | 0.82 | 0.22 | 0.41 | 0.48 | 0.25 |
| 981 | K390C | 390 | 0.19 | 0.6 | 0.54 | 0.44 | 0.18 |
| 982 | N391W | 391 | 0.7 | 0.71 | 0.93 | 0.78 | 0.11 |
| 983 | N391Y | 391 | 0.63 | 0.7 | 0.17 | 0.5 | 0.23 |
| 984 | L393D | 393 | 0.69 | 0.44 | 0.71 | 0.61 | 0.12 |
| 985 | L393Y | 393 | 0.41 | 0.54 | 0.5 | 0.48 | 0.05 |
| 986 | Q394C | 394 | 1.99 | 2.4 | 2.22 | 2.2 | 0.17 |
| 987 | Q394M | 394 | 0.79 | 0.78 | 0.8 | 0.79 | 0.01 |
| 988 | Q394G | 394 | 1.06 | 0.48 | 0.75 | 0.76 | 0.23 |
| 989 | Q394W | 394 | 0.8 | 0.29 | 0.96 | 0.68 | 0.29 |
| 990 | Q394S | 394 | 0.56 | 0.76 | 0.63 | 0.65 | 0.08 |
| 991 | Q394A | 394 | 0.59 | 0.68 | 0.61 | 0.63 | 0.04 |
| 992 | Q394F | 394 | 0.35 | 0.72 | 0.8 | 0.62 | 0.19 |
| 993 | Q394V | 394 | 0.58 | 0.52 | 0.7 | 0.6 | 0.07 |
| 994 | Q394T | 394 | 0.4 | 0.46 | 0.73 | 0.53 | 0.14 |
| 995 | Q394I | 394 | 0.52 | 0.32 | 0.41 | 0.42 | 0.08 |
| 996 | K395V | 395 | 0.41 | 0.57 | 0.6 | 0.53 | 0.08 |
| 997 | K395I | 395 | 0.3 | 0.52 | 0.62 | 0.48 | 0.13 |
| 998 | K395F | 395 | 0.5 | 0.44 | 0.33 | 0.42 | 0.07 |
| 999 | I397Q | 397 | 1.28 | 0.91 | 0.63 | 0.94 | 0.26 |
| 1000 | I397C | 397 | 0.6 | 0.58 | 0.55 | 0.58 | 0.02 |
| 1001 | T398D | 398 | 0.82 | 0.56 | 0.68 | 0.68 | 0.11 |
| 1002 | T398F | 398 | 0.35 | 0.68 | 0.67 | 0.57 | 0.15 |
| 1003 | E399M | 399 | 0.76 | 0.91 | 0.38 | 0.68 | 0.22 |
| 1004 | I400E | 400 | 1.1 | 0.21 | −0.06 | 0.42 | 0.5 |
| 1005 | E402H | 402 | 0.86 | 0.12 | 0.49 | 0.49 | 0.3 |
| 1006 | S405M | 405 | 0.99 | 0.63 | 1.01 | 0.87 | 0.17 |
| 1007 | S405K | 405 | 0.46 | 0.64 | 0.48 | 0.53 | 0.08 |
| 1008 | S405R | 405 | 0.34 | 0.69 | 0.38 | 0.47 | 0.16 |
| 1009 | N406A | 406 | 0.53 | 0.61 | 0.63 | 0.59 | 0.04 |
| 1010 | N406V | 406 | 0.34 | 0.61 | 0.34 | 0.43 | 0.13 |
| 1011 | Y407R | 407 | 0.81 | 1.3 | 1.23 | 1.12 | 0.22 |
| 1012 | Y407P | 407 | 1.1 | 0.49 | 0.44 | 0.68 | 0.3 |
| 1013 | Y407I | 407 | 0.56 | 0.49 | 0.62 | 0.56 | 0.05 |
| 1014 | K408I | 408 | 0.91 | 0.72 | 0.92 | 0.85 | 0.09 |
| 1015 | K408C | 408 | 0.88 | 0.45 | 0.29 | 0.54 | 0.25 |
| 1016 | K408D | 408 | 0.24 | 0.65 | 0.49 | 0.46 | 0.17 |
| 1017 | C410F | 410 | 0.33 | 0.57 | 0.36 | 0.42 | 0.11 |
| 1018 | S411N | 411 | 1.31 | 1.39 | 1.03 | 1.24 | 0.15 |
| 1019 | S411V | 411 | 0.7 | 0.46 | 0.76 | 0.64 | 0.13 |
| 1020 | D413W | 413 | 0.78 | 0.32 | 0.68 | 0.59 | 0.2 |
| 1021 | D413T | 413 | 0.73 | 0.67 | 0.29 | 0.56 | 0.19 |
| 1022 | D413R | 413 | 0.68 | 0.29 | 0.52 | 0.5 | 0.16 |
| 1023 | N414A | 414 | 1.39 | 0.21 | 1.19 | 0.93 | 0.51 |
| 1024 | N414D | 414 | 0.59 | 0.83 | 0.63 | 0.68 | 0.11 |
| 1025 | N414S | 414 | 0.4 | 1.09 | 0.34 | 0.61 | 0.34 |
| 1026 | N414L | 414 | 0.23 | 1.03 | 0.24 | 0.5 | 0.38 |
| 1027 | N414H | 414 | 0.83 | 0.4 | 0.12 | 0.45 | 0.29 |
| 1028 | I415E | 415 | 0.43 | 0.9 | −0.07 | 0.42 | 0.4 |
| 1029 | I415C | 415 | −0.27 | 0.5 | 1.03 | 0.42 | 0.53 |
| 1030 | K416P | 416 | 0.71 | 0.86 | 0.5 | 0.69 | 0.15 |
| 1031 | K416V | 416 | 0.6 | 0.56 | 0.34 | 0.5 | 0.11 |
| 1032 | K416W | 416 | 0.74 | 0.5 | 0.22 | 0.49 | 0.21 |
| 1033 | A417M | 417 | 1.18 | 1.31 | 1.28 | 1.25 | 0.06 |
| 1034 | A417C | 417 | 0.89 | 0.43 | 1.52 | 0.95 | 0.44 |
| 1035 | E418W | 418 | 0.45 | 0.84 | 0.38 | 0.56 | 0.2 |
| 1036 | T419G | 419 | 0.83 | 0.21 | 1.72 | 0.92 | 0.62 |
| 1037 | T419H | 419 | 0.83 | 0.9 | 1.03 | 0.92 | 0.08 |
| 1038 | T419Y | 419 | 0.83 | 1.31 | −0.76 | 0.46 | 0.88 |
| 1039 | Y420V | 420 | 0.83 | 1.12 | 1.32 | 1.09 | 0.2 |
| 1040 | Y420R | 420 | 0.39 | 0.82 | 1.16 | 0.79 | 0.32 |
| 1041 | Y420K | 420 | 0.62 | 0.48 | 0.99 | 0.7 | 0.22 |
| 1042 | Y420Q | 420 | 0.98 | 0.2 | 0.78 | 0.65 | 0.33 |
| 1043 | I421F | 421 | 1.23 | 2.15 | 1.72 | 1.7 | 0.38 |
| 1044 | H422M | 422 | 0.83 | 1.12 | 0.49 | 0.81 | 0.26 |
| 1045 | H422C | 422 | 0.13 | 1.59 | 0.33 | 0.68 | 0.64 |
| 1046 | H422A | 422 | 0.42 | 0.28 | 0.97 | 0.56 | 0.3 |
| 1047 | H422F | 422 | 0.64 | 0.49 | 0.44 | 0.52 | 0.09 |
| 1048 | E423R | 423 | 0.82 | 1.12 | 1.8 | 1.25 | 0.41 |
| 1049 | E423T | 423 | 0.54 | 1.3 | −0.07 | 0.59 | 0.56 |
| 1050 | E423S | 423 | 0.82 | 0.2 | 0.62 | 0.55 | 0.26 |
| 1051 | E423H | 423 | −0.09 | 0.79 | 0.88 | 0.53 | 0.44 |
| 1052 | E423A | 423 | 0.98 | 0.49 | −0.07 | 0.47 | 0.43 |
| 1053 | E423Y | 423 | 0.13 | 1.01 | 0.15 | 0.43 | 0.41 |
| 1054 | S425E | 425 | 0.72 | 0.79 | 0.52 | 0.67 | 0.12 |
| 1055 | S425K | 425 | 0.25 | 1.17 | 0.41 | 0.61 | 0.4 |
| 1056 | H426I | 426 | 2 | 2.15 | 1.94 | 2.03 | 0.09 |
| 1057 | H426G | 426 | 1.31 | 0.89 | 0.74 | 0.98 | 0.24 |
| 1058 | I427H | 427 | 0.64 | 0.81 | 0.66 | 0.7 | 0.07 |
| 1059 | L428T | 428 | 0.13 | 0.89 | 0.62 | 0.55 | 0.32 |
| 1060 | L428Y | 428 | 0.38 | 0.45 | 0.51 | 0.45 | 0.05 |
| 1061 | N429P | 429 | 1.14 | 0.42 | 1.18 | 0.91 | 0.35 |
| 1062 | N429A | 429 | 0.35 | 1.45 | 0.33 | 0.71 | 0.52 |
| 1063 | F431R | 431 | 1.04 | 1.45 | 0.33 | 0.94 | 0.46 |
| 1064 | F431A | 431 | 0.41 | 0.71 | 0.91 | 0.68 | 0.2 |
| 1065 | E432L | 432 | 1.04 | 1.01 | 0.84 | 0.96 | 0.09 |
| 1066 | E432R | 432 | 0.82 | 0.42 | 0.74 | 0.66 | 0.17 |
| 1067 | E432C | 432 | 0.64 | 0.71 | 0.21 | 0.52 | 0.22 |
| 1068 | E432T | 432 | 1.11 | 0.2 | 0.21 | 0.51 | 0.42 |
| 1069 | Q434F | 434 | 1.22 | 1.81 | 1.87 | 1.63 | 0.29 |
| 1070 | Q434W | 434 | 1.73 | 0.89 | 2.01 | 1.54 | 0.47 |
| 1071 | Q434S | 434 | 1.04 | 0.89 | 0.84 | 0.93 | 0.08 |
| 1072 | Q434I | 434 | 0.82 | 0.2 | 1.72 | 0.91 | 0.62 |
| 1073 | Q434L | 434 | 0.89 | 0.74 | 0.55 | 0.72 | 0.14 |
| 1074 | Q434P | 434 | −0.16 | 0.76 | 1.03 | 0.54 | 0.51 |
| 1075 | Q434T | 434 | 0.48 | 0.65 | 0.42 | 0.52 | 0.1 |
| 1076 | E435A | 435 | 0.97 | 0.89 | 0.62 | 0.83 | 0.15 |
| 1077 | E435L | 435 | 0.99 | 0.8 | 0.09 | 0.63 | 0.38 |
| 1078 | E435Y | 435 | 0.53 | 0.97 | 0.21 | 0.57 | 0.31 |
| 1079 | L436G | 436 | 2.18 | 2.4 | 2.21 | 2.26 | 0.1 |
| 1080 | L436E | 436 | 1.22 | 1.4 | 1.6 | 1.41 | 0.15 |
| 1081 | L436T | 436 | 0.82 | 0.99 | 0.8 | 0.87 | 0.08 |
| 1082 | L436D | 436 | 0.64 | 0.89 | 1.08 | 0.87 | 0.18 |
| 1083 | L436A | 436 | 1.35 | 0.2 | 1.03 | 0.86 | 0.48 |
| 1084 | L436S | 436 | 0.88 | 0.56 | 1.03 | 0.82 | 0.2 |
| 1085 | L436W | 436 | 0.82 | 0.47 | 0.96 | 0.75 | 0.21 |
| 1086 | L436K | 436 | 0.68 | 1.11 | 0.33 | 0.71 | 0.32 |
| 1087 | L436P | 436 | 1.29 | 0.38 | 0.4 | 0.69 | 0.42 |
| 1088 | L436R | 436 | 0.64 | 0.6 | 0.49 | 0.58 | 0.06 |
| 1089 | K437P | 437 | 0.64 | 1.3 | 1.03 | 0.99 | 0.27 |
| 1090 | K437C | 437 | 0.82 | 1.11 | 0.84 | 0.93 | 0.13 |
| 1091 | K437H | 437 | 1.3 | 0.2 | 0.74 | 0.75 | 0.45 |
| 1092 | K437W | 437 | −0.28 | 1.04 | 0.77 | 0.51 | 0.57 |
| 1093 | Y438R | 438 | 1.85 | 2.08 | 2.07 | 2 | 0.11 |
| 1094 | Y438A | 438 | 0.89 | 0.98 | 1.13 | 1 | 0.1 |
| 1095 | Y438W | 438 | 1.01 | 1.06 | 0.92 | 1 | 0.06 |
| 1096 | Y438P | 438 | 0.95 | 0.55 | 0.63 | 0.71 | 0.17 |
| 1097 | Y438K | 438 | 0.56 | 0.77 | 0.72 | 0.68 | 0.09 |
| 1098 | Y438I | 438 | 0.76 | 0.71 | 0.51 | 0.66 | 0.11 |
| 1099 | Y438G | 438 | 0.55 | 0.25 | 0.58 | 0.46 | 0.15 |
| 1100 | Y438L | 438 | 0.4 | 0.52 | 0.43 | 0.45 | 0.05 |
| 1101 | Y438E | 438 | 0.35 | 0.51 | 0.46 | 0.44 | 0.07 |
| 1102 | N439G | 439 | 2.07 | 1.81 | 1.72 | 1.86 | 0.15 |
| 1103 | N439P | 439 | 1.3 | 1.45 | 1.31 | 1.35 | 0.07 |
| 1104 | N439H | 439 | 1.38 | 0.89 | 0.84 | 1.04 | 0.24 |
| 1105 | N439A | 439 | 0.91 | −0.03 | 0.8 | 0.56 | 0.42 |
| 1106 | N439S | 439 | 0.65 | 0.2 | 0.39 | 0.42 | 0.19 |
| 1107 | P440K | 440 | 2.4 | 2.48 | 2.5 | 2.46 | 0.04 |
| 1108 | P440R | 440 | 1.82 | 1.89 | 1.94 | 1.89 | 0.05 |
| 1109 | P440N | 440 | 1.51 | 1.84 | 1.64 | 1.66 | 0.14 |
| 1110 | P440D | 440 | 1.42 | 2.2 | 1.22 | 1.61 | 0.42 |
| 1111 | P440T | 440 | 1.38 | 1.54 | 1.47 | 1.46 | 0.06 |
| 1112 | P440Q | 440 | 1.24 | 1.5 | 1.52 | 1.42 | 0.13 |
| 1113 | P440E | 440 | 1.26 | 1.42 | 1.46 | 1.38 | 0.08 |
| 1114 | P440G | 440 | 1.27 | 1.48 | 1.11 | 1.29 | 0.15 |
| 1115 | P440L | 440 | 0.9 | 1.28 | 1.21 | 1.13 | 0.16 |
| 1116 | P440S | 440 | 0.67 | 0.89 | 0.99 | 0.85 | 0.13 |
| 1117 | P440A | 440 | 0.77 | 0.52 | 0.68 | 0.66 | 0.1 |
| 1118 | P440M | 440 | 0.68 | 0.59 | 0.57 | 0.61 | 0.04 |
| 1119 | E441H | 441 | 1.42 | 1.81 | 1.39 | 1.54 | 0.19 |
| 1120 | E441R | 441 | 1.22 | 1.45 | 1.54 | 1.4 | 0.13 |
| 1121 | E441K | 441 | 1.12 | 1.03 | 1.31 | 1.15 | 0.12 |
| 1122 | E441N | 441 | 0.53 | 0.6 | 1.02 | 0.72 | 0.22 |
| 1123 | E441F | 441 | 0.63 | 0.71 | 0.62 | 0.65 | 0.04 |
| 1124 | E441G | 441 | 0.62 | 0.36 | 0.46 | 0.48 | 0.11 |
| 1125 | I442P | 442 | 1.33 | 1.93 | 1.47 | 1.57 | 0.26 |
| 1126 | I442Y | 442 | 0.81 | 0.84 | 1.23 | 0.96 | 0.19 |
| 1127 | I442A | 442 | 0.91 | 0.53 | 1.26 | 0.9 | 0.3 |
| 1128 | I442G | 442 | 1.14 | 0.08 | 0.03 | 0.42 | 0.51 |
| 1129 | H443E | 443 | 1.01 | 1.37 | 1.4 | 1.26 | 0.17 |
| 1130 | H443F | 443 | 1.51 | 0.6 | 1.32 | 1.14 | 0.39 |
| 1131 | H443W | 443 | 0.3 | 1.07 | 0.52 | 0.63 | 0.33 |
| 1132 | H443A | 443 | 0.41 | 1.18 | 0.22 | 0.6 | 0.42 |
| 1133 | L444W | 444 | 3.78 | 3.57 | 3.3 | 3.55 | 0.2 |
| 1134 | L444A | 444 | 2.07 | 2.42 | 2.4 | 2.29 | 0.16 |
| 1135 | L444E | 444 | 1.46 | 2.05 | 1.94 | 1.82 | 0.25 |
| 1136 | L444Y | 444 | 1.66 | 1.67 | 2.13 | 1.82 | 0.22 |
| 1137 | L444K | 444 | 1.73 | 1.81 | 1.63 | 1.73 | 0.07 |
| 1138 | L444N | 444 | 1.51 | 1.67 | 1.83 | 1.67 | 0.13 |
| 1139 | L444T | 444 | 1.45 | 1.46 | 1.57 | 1.49 | 0.05 |
| 1140 | L444C | 444 | 1.46 | 1.18 | 1.08 | 1.24 | 0.16 |
| 1141 | L444D | 444 | 0.53 | 1.5 | 1.6 | 1.21 | 0.49 |
| 1142 | L444H | 444 | 1.06 | 1.09 | 1.4 | 1.18 | 0.15 |
| 1143 | L444G | 444 | 1.04 | 0.71 | 1.27 | 1.01 | 0.23 |
| 1144 | L444I | 444 | 0.06 | 0.63 | 0.83 | 0.51 | 0.33 |
| 1145 | V445K | 445 | 2.65 | 2.76 | 2.89 | 2.77 | 0.1 |
| 1146 | V445Q | 445 | 2.02 | 1.05 | 1.82 | 1.63 | 0.42 |
| 1147 | V445P | 445 | 1.4 | 1.81 | 1.52 | 1.58 | 0.17 |
| 1148 | V445E | 445 | 1.4 | 1.33 | 1.35 | 1.36 | 0.03 |
| 1149 | V445R | 445 | 1.12 | 1.22 | 0.99 | 1.11 | 0.1 |
| 1150 | V445C | 445 | 0.57 | 0.89 | 0.29 | 0.58 | 0.25 |
| 1151 | V445W | 445 | 0.55 | 0.47 | 0.5 | 0.51 | 0.03 |
| 1152 | E446W | 446 | 0.82 | 1.3 | 1.88 | 1.33 | 0.43 |
| 1153 | E446P | 446 | 1.3 | 1.01 | 1.38 | 1.23 | 0.16 |
| 1154 | E446T | 446 | 0.53 | 0.61 | 0.34 | 0.49 | 0.11 |
| 1155 | S447L | 447 | 0.82 | 0.8 | 0.9 | 0.84 | 0.04 |
| 1156 | S447P | 447 | 0.8 | 0.9 | 0.75 | 0.82 | 0.06 |
| 1157 | S447W | 447 | 0.71 | 0.96 | 0.6 | 0.76 | 0.15 |
| 1158 | S447F | 447 | 0.4 | 0.66 | 0.63 | 0.56 | 0.11 |
| 1159 | S447Q | 447 | 0.67 | 0.54 | 0.4 | 0.54 | 0.11 |
| 1160 | S447D | 447 | 0.68 | 1.02 | −0.2 | 0.5 | 0.51 |
| 1161 | S447I | 447 | 0.51 | 0.36 | 0.46 | 0.45 | 0.06 |
| 1162 | S447Y | 447 | 0.18 | 0.75 | 0.38 | 0.44 | 0.23 |
| 1163 | E448W | 448 | 1.22 | 1.38 | 1.25 | 1.29 | 0.07 |
| 1164 | E448I | 448 | 1.3 | 1.12 | 1.07 | 1.16 | 0.1 |
| 1165 | E448Y | 448 | 0.27 | 0.81 | 1.13 | 0.74 | 0.36 |
| 1166 | E448H | 448 | 0.78 | 0.73 | 0.53 | 0.68 | 0.11 |
| 1167 | E448A | 448 | 0.4 | 0.2 | 0.64 | 0.42 | 0.18 |
| 1168 | L449D | 449 | 0.51 | 0.52 | 0.22 | 0.42 | 0.14 |
| 1169 | K450G | 450 | 1.38 | 0.54 | 0.96 | 0.96 | 0.34 |
| 1170 | K450S | 450 | 0.43 | 0.64 | 0.39 | 0.49 | 0.11 |
| 1171 | K450C | 450 | 0.26 | 0.53 | 0.6 | 0.46 | 0.15 |
| 1172 | K450V | 450 | 0.71 | 0.39 | 0.2 | 0.43 | 0.21 |
| 1173 | A451C | 451 | 0.82 | 0.82 | 0.52 | 0.72 | 0.14 |
| 1174 | A451M | 451 | 0.68 | 0.28 | 0.82 | 0.59 | 0.23 |
| 1175 | A451I | 451 | 0.26 | 0.55 | 0.68 | 0.5 | 0.18 |
| 1176 | S452P | 452 | 1.25 | 1.42 | 1.44 | 1.37 | 0.09 |
| 1177 | S452V | 452 | 0.53 | 0.73 | 0.41 | 0.56 | 0.13 |
| 1178 | S452G | 452 | 0.45 | 0.75 | 0.3 | 0.5 | 0.18 |
| 1179 | E453W | 453 | 1.35 | 1.25 | 1.43 | 1.34 | 0.07 |
| 1180 | E453F | 453 | 0.4 | 0.47 | 0.43 | 0.43 | 0.03 |
| 1181 | L454Y | 454 | 0.65 | 0.59 | 0.54 | 0.6 | 0.05 |
| 1182 | L454S | 454 | 0.62 | 0.39 | 0.53 | 0.51 | 0.1 |
| 1183 | L454R | 454 | 0.61 | 0.57 | 0.26 | 0.48 | 0.15 |
| 1184 | N456P | 456 | 1.3 | 1.07 | 1.6 | 1.32 | 0.21 |
| 1185 | N456M | 456 | 1.1 | 1.51 | 1.09 | 1.24 | 0.19 |
| 1186 | N456Q | 456 | 0.12 | 0.5 | 1.04 | 0.55 | 0.38 |
| 1187 | N456R | 456 | −0.28 | 0.56 | 1.12 | 0.46 | 0.58 |
| 1188 | V457T | 457 | 0.59 | 0.8 | 0.89 | 0.76 | 0.13 |
| 1189 | V457Y | 457 | 1.22 | 0.72 | 0.23 | 0.72 | 0.41 |
| 1190 | V457E | 457 | 0.82 | 0.5 | −0.06 | 0.42 | 0.36 |
| 1191 | V460N | 460 | 1.14 | 0.61 | 1.27 | 1.01 | 0.28 |
| 1192 | V460I | 460 | 1.29 | 0.68 | 0.64 | 0.87 | 0.3 |
| 1193 | V460K | 460 | 0.82 | 0.97 | 0.64 | 0.81 | 0.14 |
| 1194 | V460E | 460 | 0.67 | 1.05 | 0.57 | 0.76 | 0.21 |
| 1195 | V460P | 460 | 0.63 | 0.9 | 0.45 | 0.66 | 0.18 |
| 1196 | V460H | 460 | 0.41 | 0.9 | −0.06 | 0.42 | 0.39 |
| 1197 | I461C | 461 | 0.75 | 0.7 | 0.35 | 0.6 | 0.18 |
| 1198 | I461Y | 461 | 0.43 | 0.5 | 0.44 | 0.46 | 0.03 |
| 1199 | N463A | 463 | 0.4 | 0.34 | 0.59 | 0.44 | 0.11 |
| 1200 | F465R | 465 | 1.4 | 1.37 | 1.35 | 1.38 | 0.02 |
| 1201 | F465Q | 465 | 0.79 | 1.03 | 1.17 | 1 | 0.16 |
| 1202 | F465K | 465 | 0.83 | 1.05 | 1.06 | 0.98 | 0.1 |
| 1203 | F465E | 465 | 1.13 | 0.85 | 0.76 | 0.91 | 0.16 |
| 1204 | F465T | 465 | 0.89 | 0.73 | 0.81 | 0.81 | 0.07 |
| 1205 | F465A | 465 | 0.7 | 0.96 | 0.71 | 0.79 | 0.12 |
| 1206 | F465V | 465 | 0.73 | 0.94 | 0.51 | 0.73 | 0.18 |
| 1207 | F465N | 465 | 0.62 | 0.7 | 0.7 | 0.67 | 0.04 |
| 1208 | F465G | 465 | 0.55 | 0.44 | 0.62 | 0.54 | 0.07 |
| 1209 | F465S | 465 | 0.28 | 0.42 | 0.57 | 0.43 | 0.12 |
| 1210 | H466K | 466 | 0.65 | 0.87 | 0.58 | 0.7 | 0.13 |
| 1211 | H466C | 466 | 0.43 | 0.79 | 0.25 | 0.49 | 0.22 |
| 1212 | S469K | 469 | 0.98 | 1.19 | 1.14 | 1.1 | 0.09 |
| 1213 | S469P | 469 | 0.7 | 0.4 | 0.82 | 0.64 | 0.18 |
| 1214 | S469L | 469 | 0.6 | 0.57 | 0.66 | 0.61 | 0.03 |
| 1215 | S469M | 469 | 0.52 | 0.67 | 0.44 | 0.54 | 0.09 |
| 1216 | S469V | 469 | 0.44 | 0.45 | 0.6 | 0.5 | 0.07 |
| 1217 | S469R | 469 | 0.39 | 0.5 | 0.42 | 0.44 | 0.05 |
| 1218 | V470K | 470 | 1.04 | 1.06 | 1.11 | 1.07 | 0.03 |
| 1219 | V470N | 470 | 0.65 | 0.44 | 0.67 | 0.59 | 0.11 |
| 1220 | V470Y | 470 | 0.65 | 0.48 | 0.59 | 0.57 | 0.07 |
| 1221 | M472G | 472 | 0.35 | 0.88 | 0.57 | 0.6 | 0.22 |
| 1222 | T473R | 473 | 0.18 | 0.89 | 0.46 | 0.51 | 0.3 |
| 1223 | T473G | 473 | −0.23 | 1.62 | 0.05 | 0.48 | 0.82 |
| 1224 | T473F | 473 | 0.41 | 0.21 | 0.65 | 0.42 | 0.18 |
| 1225 | E474P | 474 | 0.78 | 1.04 | 0.87 | 0.9 | 0.11 |
| 1226 | L476R | 476 | 1.05 | 1.29 | 1.13 | 1.16 | 0.1 |
| 1227 | L476K | 476 | 0.61 | 0.49 | 0.75 | 0.62 | 0.11 |
| 1228 | L476H | 476 | 0.46 | 0.74 | 0.49 | 0.56 | 0.13 |
| 1229 | L476T | 476 | 0.39 | 0.58 | 0.49 | 0.48 | 0.08 |
| 1230 | L476G | 476 | 0.43 | 0.56 | 0.42 | 0.47 | 0.06 |
| 1231 | L476N | 476 | 0.47 | 0.5 | 0.36 | 0.44 | 0.06 |
| 1232 | V477Q | 477 | 1.14 | 1.43 | 1.34 | 1.3 | 0.12 |
| 1233 | V477T | 477 | 1.12 | 1.04 | 1.54 | 1.23 | 0.22 |
| 1234 | V477W | 477 | 1.3 | 1.87 | −0.73 | 0.81 | 1.12 |
| 1235 | V477E | 477 | 1.07 | 0.29 | 0.67 | 0.68 | 0.32 |
| 1236 | V477D | 477 | 0.62 | 0.75 | 0.15 | 0.51 | 0.26 |
| 1237 | V477M | 477 | 0.53 | 0.42 | 0.44 | 0.46 | 0.05 |
| 1238 | K479A | 479 | 0.13 | 1.2 | 1.16 | 0.83 | 0.5 |
| 1239 | K479H | 479 | 1.04 | −0.48 | 1.06 | 0.54 | 0.72 |
| 1240 | K479D | 479 | 0.44 | 0.59 | 0.45 | 0.49 | 0.07 |
| 1241 | D480Q | 480 | 0.69 | 0.33 | 0.88 | 0.63 | 0.23 |
| 1242 | D480P | 480 | 0.34 | 0.29 | 0.65 | 0.43 | 0.16 |
| 1243 | N481A | 481 | 2.07 | 0.91 | 1.06 | 1.35 | 0.52 |
| 1244 | N481D | 481 | 0.46 | 0.91 | 0.3 | 0.56 | 0.26 |
| 1245 | N482A | 482 | 1.79 | 1.66 | 2.07 | 1.84 | 0.17 |
| 1246 | N482H | 482 | 1.04 | 0.22 | 1.47 | 0.91 | 0.52 |
| 1247 | N482Q | 482 | 0.69 | 0.44 | 0.97 | 0.7 | 0.22 |
| 1248 | N482D | 482 | 0.13 | 0.91 | 0.25 | 0.43 | 0.34 |
| 1249 | N482S | 482 | 0.38 | 0.47 | 0.41 | 0.42 | 0.04 |
| 1250 | F483W | 483 | 0.64 | 0.77 | 0.35 | 0.58 | 0.18 |
| 1251 | F483A | 483 | 0.39 | 0.4 | 0.8 | 0.53 | 0.19 |
| 1252 | Y484G | 484 | 1.23 | 0.22 | 1.35 | 0.93 | 0.51 |
| 1253 | Y484V | 484 | 0.69 | 0.44 | 0.53 | 0.55 | 0.1 |
| 1254 | Y484R | 484 | 0.82 | 0.34 | 0.45 | 0.54 | 0.21 |
| 1255 | A485Q | 485 | 0.62 | 0.45 | 1.15 | 0.74 | 0.3 |
| 1256 | A485K | 485 | 0.63 | 1.08 | 0.37 | 0.69 | 0.29 |
| 1257 | A485R | 485 | 0.32 | 0.76 | 0.99 | 0.69 | 0.28 |
| 1258 | A485L | 485 | 0.73 | 0.67 | 0.45 | 0.62 | 0.12 |
| 1259 | A485H | 485 | 0.38 | 0.84 | 0.42 | 0.55 | 0.21 |
| 1260 | A485S | 485 | 0.7 | 0.4 | 0.46 | 0.52 | 0.13 |
| 1261 | E486L | 486 | 1.89 | 1.95 | 1.19 | 1.68 | 0.34 |
| 1262 | E486R | 486 | 1.23 | 1.61 | 1.22 | 1.35 | 0.18 |
| 1263 | E486S | 486 | 0.82 | 1.76 | 1.44 | 1.34 | 0.39 |
| 1264 | E486Y | 486 | 1.52 | 1.32 | 1.15 | 1.33 | 0.15 |
| 1265 | E486F | 486 | 0.69 | 1.14 | 0.53 | 0.79 | 0.26 |
| 1266 | E486A | 486 | 1.36 | 0.41 | 0.56 | 0.77 | 0.42 |
| 1267 | E486G | 486 | 0.54 | 0.52 | 0.19 | 0.42 | 0.16 |
| 1268 | E488Q | 488 | 0.42 | 1.43 | 0.82 | 0.89 | 0.41 |
| 1269 | E488T | 488 | 0.54 | 0.92 | 0.78 | 0.75 | 0.16 |
| 1270 | E489H | 489 | 2.08 | 1.14 | 1.23 | 1.48 | 0.42 |
| 1271 | E489C | 489 | 0.72 | 1.18 | 1.2 | 1.03 | 0.22 |
| 1272 | E489T | 489 | 0.86 | 1.06 | 0.53 | 0.82 | 0.22 |
| 1273 | E489M | 489 | 0.36 | 0.63 | 0.86 | 0.61 | 0.2 |
| 1274 | E489S | 489 | 0.33 | 0.64 | 0.53 | 0.5 | 0.13 |
| 1275 | E489W | 489 | 0.38 | 0.23 | 0.7 | 0.43 | 0.2 |
| 1276 | I490A | 490 | 0.47 | 0.56 | 1.07 | 0.7 | 0.27 |
| 1277 | I490E | 490 | 0.37 | 0.65 | 0.54 | 0.52 | 0.12 |
| 1278 | Y491E | 491 | 1.23 | 0.92 | 1.23 | 1.13 | 0.15 |
| 1279 | Y491Q | 491 | 0.83 | 0.22 | 0.89 | 0.65 | 0.3 |
| 1280 | D492I | 492 | 1.67 | 1.69 | 1.52 | 1.63 | 0.08 |
| 1281 | D492L | 492 | 0.4 | 0.76 | 0.56 | 0.57 | 0.15 |
| 1282 | D492K | 492 | 0.29 | 0.04 | 1.36 | 0.56 | 0.57 |
| 1283 | E493C | 493 | 0.65 | 0.59 | 0.08 | 0.44 | 0.25 |
| 1284 | E493R | 493 | 0.43 | 0.35 | 0.51 | 0.43 | 0.07 |
| 1285 | Y495V | 495 | 0.49 | 0.78 | 0.77 | 0.68 | 0.14 |
| 1286 | P496W | 496 | 0.14 | 1.62 | 2.28 | 1.35 | 0.9 |
| 1287 | P496E | 496 | 1.23 | 0.92 | 1.23 | 1.13 | 0.15 |
| 1288 | P496C | 496 | 0.98 | 1.08 | 0.96 | 1.01 | 0.05 |
| 1289 | P496V | 496 | 0.98 | 0.74 | 1.28 | 1 | 0.22 |
| 1290 | P496L | 496 | 0.7 | 0.96 | 0.65 | 0.77 | 0.14 |
| 1291 | P496K | 496 | 0.95 | 0.45 | 0.9 | 0.77 | 0.22 |
| 1292 | P496S | 496 | 0.57 | 0.34 | 0.4 | 0.43 | 0.1 |
| 1293 | P496I | 496 | 0.83 | −0.46 | 0.9 | 0.42 | 0.63 |
| 1294 | V497W | 497 | 0.17 | 0.88 | 1.05 | 0.7 | 0.38 |
| 1295 | I498R | 498 | 1.21 | 1.15 | 0.91 | 1.09 | 0.13 |
| 1296 | I498K | 498 | 0.86 | 0.75 | 0.64 | 0.75 | 0.09 |
| 1297 | I498P | 498 | 0.59 | 0.63 | 0.71 | 0.65 | 0.05 |
| 1298 | I498G | 498 | 0.28 | 0.66 | 0.63 | 0.52 | 0.17 |
| 1299 | S499M | 499 | 0.98 | 0.74 | 0.67 | 0.8 | 0.13 |
| 1300 | S499C | 499 | 1.23 | 1.04 | −0.31 | 0.65 | 0.68 |
| 1301 | S499G | 499 | 0.83 | 0.23 | 0.67 | 0.58 | 0.26 |
| 1302 | L500S | 500 | 0.27 | 0.95 | 0.17 | 0.47 | 0.34 |
| 1303 | L503N | 503 | 0.82 | 2.3 | 2.54 | 1.89 | 0.76 |
| 1304 | L503P | 503 | 1.23 | 1.14 | 0.99 | 1.12 | 0.1 |
| 1305 | L503H | 503 | 0.64 | 1.2 | 0.59 | 0.81 | 0.28 |
| 1306 | L503I | 503 | 1.31 | 0.45 | 0.67 | 0.81 | 0.37 |
| 1307 | L503G | 503 | 0.69 | 0.78 | 0.67 | 0.72 | 0.05 |
| 1308 | L503R | 503 | 0.31 | 0.69 | 0.94 | 0.65 | 0.26 |
| 1309 | L503D | 503 | 0.42 | 1.2 | 0.27 | 0.63 | 0.41 |
| 1310 | L503K | 503 | 0.31 | 0.69 | 0.86 | 0.62 | 0.23 |
| 1311 | L503E | 503 | 0.54 | 0.22 | 1.08 | 0.61 | 0.35 |
| 1312 | L503W | 503 | 0.42 | 1.07 | −0.02 | 0.49 | 0.45 |
| 1313 | V504A | 504 | 1.26 | 1.45 | 1.64 | 1.45 | 0.16 |
| 1314 | V504T | 504 | 0.96 | 1.09 | 1.21 | 1.08 | 0.1 |
| 1315 | V504G | 504 | 0.75 | 0.61 | 0.74 | 0.7 | 0.06 |
| 1316 | V504C | 504 | 0.21 | 0.67 | 0.55 | 0.48 | 0.19 |
| 1317 | Y507R | 507 | 2.44 | 2.56 | 2.77 | 2.59 | 0.13 |
| 1318 | Y507K | 507 | 2.45 | 2.47 | 2.36 | 2.43 | 0.05 |
| 1319 | Y507W | 507 | 1.96 | 2.04 | 2.26 | 2.09 | 0.13 |
| 1320 | Y507S | 507 | 1.92 | 1.99 | 2.05 | 1.99 | 0.05 |
| 1321 | Y507A | 507 | 1.79 | 1.86 | 1.78 | 1.81 | 0.04 |
| 1322 | Y507T | 507 | 1.5 | 1.73 | 1.79 | 1.67 | 0.13 |
| 1323 | Y507Q | 507 | 1.6 | 1.52 | 1.87 | 1.67 | 0.15 |
| 1324 | Y507C | 507 | 1.41 | 1.28 | 1.53 | 1.41 | 0.1 |
| 1325 | Y507V | 507 | 0.99 | 1.09 | 1.21 | 1.1 | 0.09 |
| 1326 | Y507E | 507 | 1.23 | 0.63 | 1.43 | 1.1 | 0.34 |
| 1327 | Y507G | 507 | 1.17 | 0.77 | 0.95 | 0.96 | 0.16 |
| 1328 | Y507L | 507 | 0.99 | 0.86 | 0.7 | 0.85 | 0.12 |
| 1329 | Y507I | 507 | 1 | 0.66 | 0.85 | 0.84 | 0.14 |
| 1330 | T509L | 509 | 0.62 | 1.01 | 1.1 | 0.91 | 0.21 |
| 1331 | T509V | 509 | 0.31 | 1.29 | 0.38 | 0.66 | 0.44 |
| 1332 | T509M | 509 | 1.01 | 0.81 | 0.07 | 0.63 | 0.4 |
| 1333 | Q510F | 510 | 2.46 | 2.65 | 2.77 | 2.63 | 0.13 |
| 1334 | Q510G | 510 | 2.19 | 2.39 | 2.52 | 2.37 | 0.14 |
| 1335 | Q510Y | 510 | 2.22 | 2.26 | 2.36 | 2.28 | 0.06 |
| 1336 | Q510S | 510 | 2.12 | 2.37 | 2.3 | 2.26 | 0.1 |
| 1337 | Q510C | 510 | 2.08 | 2.16 | 2.18 | 2.14 | 0.05 |
| 1338 | Q510V | 510 | 2.01 | 2.17 | 2.21 | 2.13 | 0.09 |
| 1339 | Q510W | 510 | 1.95 | 2.19 | 2.16 | 2.1 | 0.11 |
| 1340 | Q510N | 510 | 1.91 | 1.85 | 2.22 | 1.99 | 0.16 |
| 1341 | Q510A | 510 | 1.58 | 1.81 | 1.92 | 1.77 | 0.14 |
| 1342 | Q510D | 510 | 1.56 | 1.97 | 1.46 | 1.66 | 0.22 |
| 1343 | Q510I | 510 | 1.77 | 1.46 | 1.75 | 1.66 | 0.14 |
| 1344 | Q510H | 510 | 1.04 | 1.19 | 1.13 | 1.12 | 0.06 |
| 1345 | Q510K | 510 | 0.83 | 0.99 | 0.94 | 0.92 | 0.07 |
| 1346 | Q510M | 510 | 0.75 | 0.67 | 0.63 | 0.68 | 0.05 |
| 1347 | Q510E | 510 | 0.64 | 0.69 | 0.61 | 0.65 | 0.03 |
| 1348 | Q510P | 510 | 0.68 | 0.58 | 0.59 | 0.62 | 0.04 |
| 1349 | Q510T | 510 | 0.48 | 0.72 | 0.63 | 0.61 | 0.1 |
| 1350 | Q510R | 510 | 0.35 | 0.47 | 0.5 | 0.44 | 0.07 |
| 1351 | K511Y | 511 | 0.96 | 1.37 | 0.33 | 0.88 | 0.43 |
| 1352 | K511G | 511 | 1.45 | 0.85 | 0.29 | 0.87 | 0.47 |
| 1353 | K511S | 511 | 0.85 | 0.75 | 0.79 | 0.79 | 0.04 |
| 1354 | K511F | 511 | 0.99 | 0.78 | 0.38 | 0.72 | 0.25 |
| 1355 | K511V | 511 | 0.45 | 0.92 | 0.63 | 0.67 | 0.19 |
| 1356 | P512N | 512 | 2.51 | 2.77 | 2.8 | 2.69 | 0.13 |
| 1357 | P512F | 512 | 2.04 | 2.32 | 2.29 | 2.22 | 0.13 |
| 1358 | P512Y | 512 | 1.9 | 2.03 | 2.11 | 2.02 | 0.09 |
| 1359 | P512C | 512 | 1.96 | 1.98 | 2.03 | 1.99 | 0.03 |
| 1360 | P512G | 512 | 1.86 | 1.91 | 1.96 | 1.91 | 0.04 |
| 1361 | P512D | 512 | 1.74 | 1.88 | 1.85 | 1.82 | 0.06 |
| 1362 | P512I | 512 | 1.45 | 1.69 | 1.73 | 1.62 | 0.12 |
| 1363 | P512H | 512 | 1.53 | 1.61 | 1.58 | 1.57 | 0.03 |
| 1364 | P512V | 512 | 1.46 | 1.48 | 1.69 | 1.54 | 0.11 |
| 1365 | P512W | 512 | 1.08 | 1.29 | 1.4 | 1.26 | 0.13 |
| 1366 | P512S | 512 | 0.97 | 1.09 | 1.06 | 1.04 | 0.05 |
| 1367 | P512E | 512 | 0.88 | 0.74 | 0.81 | 0.81 | 0.06 |
| 1368 | P512T | 512 | 0.5 | 0.69 | 0.49 | 0.56 | 0.09 |
| 1369 | P512R | 512 | 0.47 | 0.48 | 0.43 | 0.46 | 0.02 |
| 1370 | Y513G | 513 | 3.41 | 3.77 | 3.79 | 3.66 | 0.18 |
| 1371 | Y513Q | 513 | 3.29 | 3.52 | 3.71 | 3.51 | 0.17 |
| 1372 | Y513R | 513 | 3.28 | 3.4 | 3.53 | 3.41 | 0.1 |
| 1373 | Y513K | 513 | 3.27 | 3.36 | 3.45 | 3.36 | 0.07 |
| 1374 | Y513N | 513 | 3.11 | 3.13 | 3.18 | 3.14 | 0.03 |
| 1375 | Y513I | 513 | 2.88 | 2.96 | 2.88 | 2.91 | 0.04 |
| 1376 | Y513A | 513 | 2.6 | 2.65 | 2.8 | 2.68 | 0.08 |
| 1377 | Y513S | 513 | 2.47 | 2.62 | 2.74 | 2.61 | 0.11 |
| 1378 | Y513E | 513 | 2.53 | 2.59 | 2.68 | 2.6 | 0.06 |
| 1379 | Y513V | 513 | 2.5 | 2.51 | 2.63 | 2.55 | 0.06 |
| 1380 | Y513L | 513 | 2.35 | 2.58 | 2.54 | 2.49 | 0.1 |
| 1381 | Y513D | 513 | 2.29 | 2.59 | 2.59 | 2.49 | 0.14 |
| 1382 | Y513C | 513 | 2.31 | 2.57 | 2.47 | 2.45 | 0.11 |
| 1383 | Y513M | 513 | 2.16 | 2.37 | 2.27 | 2.27 | 0.08 |
| 1384 | Y513T | 513 | 2.21 | 2.23 | 2.33 | 2.26 | 0.05 |
| 1385 | Y513H | 513 | 1.5 | 2.33 | 2.47 | 2.1 | 0.43 |
| 1386 | Y513P | 513 | 0.85 | 1.09 | 1.15 | 1.03 | 0.13 |
| 1387 | S514P | 514 | 3.68 | 3.78 | 3.95 | 3.8 | 0.11 |
| 1388 | S514W | 514 | 1.97 | 2.14 | 1.99 | 2.03 | 0.08 |
| 1389 | S514G | 514 | 0.96 | 1.29 | 1.4 | 1.22 | 0.19 |
| 1390 | S514H | 514 | 0.89 | 0.72 | 1 | 0.87 | 0.12 |
| 1391 | S514F | 514 | 1.16 | 0.79 | 0.66 | 0.87 | 0.21 |
| 1392 | S514D | 514 | 0.73 | 1.22 | 0.64 | 0.86 | 0.25 |
| 1393 | S514R | 514 | 0.97 | 0.56 | 0.89 | 0.81 | 0.18 |
| 1394 | S514A | 514 | 0.73 | 0.94 | 0.69 | 0.79 | 0.11 |
| 1395 | S514K | 514 | 0.69 | 0.76 | 0.61 | 0.68 | 0.06 |
| 1396 | S514N | 514 | 0.69 | 0.66 | 0.24 | 0.53 | 0.21 |
| 1397 | S514L | 514 | 0.6 | 0.47 | 0.46 | 0.51 | 0.06 |
| 1398 | T515G | 515 | 2.89 | 3.08 | 2.85 | 2.94 | 0.1 |
| 1399 | T515C | 515 | 1.25 | 1.53 | 1.85 | 1.54 | 0.24 |
| 1400 | T515P | 515 | 1.31 | 1.48 | 1.49 | 1.43 | 0.09 |
| 1401 | T515A | 515 | 0.83 | 0.95 | 1.01 | 0.93 | 0.08 |
| 1402 | T515L | 515 | 0.51 | 0.71 | 0.57 | 0.6 | 0.08 |
| 1403 | T515M | 515 | 0.66 | 0.38 | 0.66 | 0.57 | 0.13 |
| 1404 | T515S | 515 | 0.4 | 0.58 | 0.44 | 0.47 | 0.08 |
| 1405 | T515Y | 515 | 0.49 | 0.56 | 0.3 | 0.45 | 0.11 |
| 1406 | K516P | 516 | 2.25 | 2.32 | 2.58 | 2.38 | 0.14 |
| 1407 | K516S | 516 | 1.2 | 1.23 | 1.25 | 1.23 | 0.02 |
| 1408 | K516R | 516 | 0.94 | 0.89 | 0.93 | 0.92 | 0.02 |
| 1409 | K516A | 516 | 0.63 | 0.72 | 0.79 | 0.71 | 0.07 |
| 1410 | K516T | 516 | 0.43 | 0.54 | 0.55 | 0.51 | 0.05 |
| 1411 | K517P | 517 | 3.68 | 3.83 | 3.89 | 3.8 | 0.09 |
| 1412 | K517D | 517 | 1.33 | 1.61 | 1.08 | 1.34 | 0.22 |
| 1413 | K517L | 517 | 0.96 | 1.32 | 1.27 | 1.18 | 0.16 |
| 1414 | K517G | 517 | 1.12 | 0.61 | 1.13 | 0.95 | 0.24 |
| 1415 | K517A | 517 | 0.42 | 1.07 | 0.67 | 0.72 | 0.27 |
| 1416 | I518P | 518 | 3.56 | 3.55 | 3.85 | 3.65 | 0.14 |
| 1417 | I518T | 518 | 2.35 | 2.64 | 2.51 | 2.5 | 0.12 |
| 1418 | I518S | 518 | 1.38 | 1.39 | 1.52 | 1.43 | 0.07 |
| 1419 | I518Q | 518 | 1.23 | 1.48 | 1.43 | 1.38 | 0.11 |
| 1420 | I518E | 518 | 0.85 | 0.91 | 1.13 | 0.96 | 0.12 |
| 1421 | I518L | 518 | 0.54 | 0.5 | 0.82 | 0.62 | 0.14 |
| 1422 | I518F | 518 | 0.38 | 0.84 | 0.4 | 0.54 | 0.21 |
| 1423 | L520M | 520 | 3.57 | 3.87 | 4.02 | 3.82 | 0.19 |
| 1424 | L520N | 520 | 3.29 | 3.55 | 3.52 | 3.45 | 0.12 |
| 1425 | L520Q | 520 | 2.85 | 3.24 | 3.31 | 3.13 | 0.2 |
| 1426 | L520H | 520 | 2.54 | 2.25 | 2.58 | 2.46 | 0.14 |
| 1427 | L520S | 520 | 2.05 | 2.41 | 2.4 | 2.29 | 0.17 |
| 1428 | L520G | 520 | 1.86 | 1.7 | 1.72 | 1.76 | 0.07 |
| 1429 | L520A | 520 | 0.64 | 0.29 | 0.45 | 0.46 | 0.14 |
| 1430 | N521F | 521 | 1.74 | 1.07 | 0.82 | 1.21 | 0.39 |
| 1431 | N521P | 521 | 1.31 | 0.44 | 1.36 | 1.04 | 0.42 |
| 1432 | N521R | 521 | 0.82 | 0.56 | 0.93 | 0.77 | 0.16 |
| 1433 | N521L | 521 | −0.16 | 1.23 | 0.79 | 0.62 | 0.58 |
| 1434 | N521E | 521 | 0.47 | 0.4 | 0.76 | 0.54 | 0.16 |
| 1435 | F522A | 522 | 2.92 | 3.19 | 3.15 | 3.09 | 0.12 |
| 1436 | F522G | 522 | 2.43 | 2.47 | 2.5 | 2.46 | 0.03 |
| 1437 | F522M | 522 | 1.06 | 1.02 | 0.63 | 0.91 | 0.2 |
| 1438 | F522C | 522 | 0.85 | 0.79 | 0.84 | 0.83 | 0.02 |
| 1439 | G523Q | 523 | 1.92 | 0.92 | 0.67 | 1.17 | 0.54 |
| 1440 | G523A | 523 | 1.52 | 0.92 | 1.07 | 1.17 | 0.25 |
| 1441 | A528I | 528 | 0.53 | 0.92 | −0.03 | 0.47 | 0.39 |
| 1442 | Y537F | 537 | 0.17 | 0.79 | 0.67 | 0.54 | 0.27 |
| 1443 | S538C | 538 | 0.41 | 0.21 | 0.69 | 0.44 | 0.2 |
| 1444 | S538N | 538 | 0.46 | 0.41 | 0.4 | 0.42 | 0.03 |
| 1445 | A541N | 541 | 2.61 | 2.6 | 2.81 | 2.67 | 0.1 |
| 1446 | A541I | 541 | 2.27 | 2.51 | 2.54 | 2.44 | 0.12 |
| 1447 | A541V | 541 | 1.66 | 1.94 | 1.82 | 1.81 | 0.11 |
| 1448 | A541M | 541 | 1.68 | 0.62 | 1.46 | 1.25 | 0.46 |
| 1449 | A541L | 541 | 0.53 | 1.06 | 0.73 | 0.77 | 0.22 |
| 1450 | A541T | 541 | 0.65 | 0.78 | 0.86 | 0.77 | 0.09 |
| 1451 | A541Q | 541 | 0.41 | 0.91 | 0.87 | 0.73 | 0.23 |
| 1452 | I543H | 543 | 1.73 | 1.94 | 1.63 | 1.77 | 0.13 |
| 1453 | I543W | 543 | 2.2 | 1.12 | 1.2 | 1.51 | 0.49 |
| 1454 | I543F | 543 | 1.22 | 1.73 | 1.41 | 1.45 | 0.21 |
| 1455 | I543G | 543 | 1.6 | 0.9 | 0.54 | 1.01 | 0.44 |
| 1456 | I543T | 543 | 0.81 | 1.33 | 0.84 | 1 | 0.24 |
| 1457 | I543A | 543 | 0.71 | 0.39 | 0.83 | 0.64 | 0.18 |
| 1458 | I543C | 543 | 0.97 | 0.5 | −0.05 | 0.47 | 0.42 |
| 1459 | I543S | 543 | 0.41 | 0.47 | 0.45 | 0.45 | 0.02 |
| 1460 | I543Q | 543 | 0.81 | 0.21 | 0.24 | 0.42 | 0.28 |
| 1461 | L544Y | 544 | 1.02 | 1.42 | 1.36 | 1.27 | 0.17 |
| 1462 | L544W | 544 | 1.04 | 0.43 | 0.64 | 0.7 | 0.25 |
| 1463 | M545E | 545 | 0.52 | 0.21 | 1.45 | 0.73 | 0.53 |
| 1464 | M545Q | 545 | −0.22 | 1.21 | 0.3 | 0.43 | 0.59 |
| 1465 | R546F | 546 | 0.96 | 1.19 | 1.55 | 1.24 | 0.24 |
| 1466 | L549D | 549 | 1.03 | 0.61 | −0.06 | 0.53 | 0.45 |
| 1467 | L552E | 552 | 2.72 | 2.69 | 2.68 | 2.69 | 0.02 |
| 1468 | L552Q | 552 | 1.26 | 1.59 | 2.1 | 1.65 | 0.35 |
| 1469 | L552K | 552 | 0.81 | 0.61 | 0.92 | 0.78 | 0.13 |
| 1470 | G553M | 553 | 2 | 2.06 | 2 | 2.02 | 0.03 |
| 1471 | G553L | 553 | 1.77 | 1.79 | 1.83 | 1.8 | 0.03 |
| 1472 | G553T | 553 | 1.69 | 1.73 | 1.82 | 1.75 | 0.06 |
| 1473 | G553I | 553 | 1.1 | 1.39 | 1.28 | 1.26 | 0.12 |
| 1474 | G553V | 553 | 0.81 | 0.96 | 0.92 | 0.9 | 0.06 |
| 1475 | G553S | 553 | 0.66 | 0.73 | 0.76 | 0.72 | 0.04 |
| 1476 | G553A | 553 | 0.56 | 0.72 | 0.6 | 0.63 | 0.07 |
| 1477 | I554C | 554 | 0.69 | 0.94 | 1.23 | 0.95 | 0.22 |
| 1478 | F555M | 555 | 0.84 | 0.74 | 0.43 | 0.67 | 0.18 |
| 1479 | N556E | 556 | 3.02 | 3.2 | 2.94 | 3.05 | 0.11 |
| 1480 | N556Q | 556 | 1.85 | 1.99 | 2.42 | 2.09 | 0.24 |
| 1481 | N556C | 556 | 1.65 | 1.81 | 2.27 | 1.91 | 0.26 |
| 1482 | N556M | 556 | 1.62 | 1.73 | 1.85 | 1.74 | 0.09 |
| 1483 | N556R | 556 | 1.66 | 1.62 | 1.69 | 1.66 | 0.03 |
| 1484 | N556A | 556 | 1.36 | 1.66 | 1.82 | 1.61 | 0.19 |
| 1485 | N556V | 556 | 1.43 | 1.38 | 1.57 | 1.46 | 0.08 |
| 1486 | N556F | 556 | 0.93 | 1.07 | 1.14 | 1.05 | 0.09 |
| 1487 | N556W | 556 | 0.54 | 0.66 | 0.62 | 0.61 | 0.05 |
| 1488 | N556K | 556 | 0.4 | 0.44 | 0.45 | 0.43 | 0.02 |
| 1489 | A557S | 557 | 1.62 | 1.74 | 1.6 | 1.66 | 0.06 |
| 1490 | A557K | 557 | 1.21 | 1.23 | 0.96 | 1.13 | 0.12 |
| 1491 | K558H | 558 | 1.98 | 2.28 | 2.18 | 2.15 | 0.12 |
| 1492 | K558M | 558 | 2.06 | 1.99 | 0.62 | 1.56 | 0.66 |
| 1493 | K558S | 558 | 1.37 | 1.7 | 1.54 | 1.54 | 0.14 |
| 1494 | K558F | 558 | 1.41 | 0.71 | 1.66 | 1.26 | 0.4 |
| 1495 | K558E | 558 | 0.81 | 1.3 | 0.62 | 0.91 | 0.29 |
| 1496 | K558T | 558 | 0.81 | 1.12 | 0.74 | 0.89 | 0.16 |
| 1497 | N559G | 559 | 1.09 | 1.54 | 1.72 | 1.45 | 0.26 |
| 1498 | N559E | 559 | 1.21 | 1.58 | 1.18 | 1.33 | 0.18 |
| 1499 | N559A | 559 | 0.81 | 1.14 | 1.16 | 1.04 | 0.16 |
| 1500 | D562I | 562 | 1.59 | 1.52 | 1.75 | 1.62 | 0.09 |
| 1501 | D562L | 562 | 1.51 | 1.54 | 1.6 | 1.55 | 0.04 |
| 1502 | D562F | 562 | 1.43 | 1.32 | 1.34 | 1.36 | 0.05 |
| 1503 | D562T | 562 | 1.02 | 1.43 | 1.42 | 1.29 | 0.19 |
| 1504 | D562Q | 562 | 1.21 | 1.19 | 1.21 | 1.2 | 0.01 |
| 1505 | D562M | 562 | 1.01 | 1.13 | 1.18 | 1.11 | 0.08 |
| 1506 | D562W | 562 | 0.68 | 1 | 0.9 | 0.86 | 0.14 |
| 1507 | D562C | 562 | 0.59 | 0.93 | 1.05 | 0.86 | 0.19 |
| 1508 | D562V | 562 | 0.68 | 0.83 | 0.86 | 0.79 | 0.08 |
| 1509 | D562K | 562 | 0.31 | 0.57 | 0.66 | 0.51 | 0.15 |
| 1510 | K564A | 564 | 1.82 | 1.29 | 1.31 | 1.47 | 0.24 |
| 1511 | K564G | 564 | 0.88 | 1.45 | 0.08 | 0.8 | 0.56 |
| 1512 | K564D | 564 | 0.62 | 0.71 | 1.03 | 0.78 | 0.17 |
| 1513 | I565R | 565 | 0.29 | −0.32 | 1.93 | 0.63 | 0.95 |
| 1514 | I565F | 565 | 0.45 | 0.66 | 0.26 | 0.46 | 0.16 |
| 1515 | I566A | 566 | 0.8 | 1.15 | 0.71 | 0.89 | 0.19 |
| 1516 | I566W | 566 | 1.21 | −0.21 | 0.44 | 0.48 | 0.58 |
| 1517 | E567W | 567 | 1.09 | 1.49 | 1.23 | 1.27 | 0.17 |
| 1518 | E567Q | 567 | 0.7 | 0.98 | 1.09 | 0.92 | 0.17 |
| 1519 | E567L | 567 | 0.38 | 0.3 | 0.59 | 0.42 | 0.12 |
| 1520 | N569P | 569 | 0.84 | 0.95 | 0.8 | 0.86 | 0.06 |
| 1521 | N569W | 569 | 0.56 | 0.65 | 0.62 | 0.61 | 0.03 |
| 1522 | T570F | 570 | 1.72 | 1.8 | 1.31 | 1.61 | 0.21 |
| 1523 | T570D | 570 | 0.8 | 0.6 | 0.33 | 0.58 | 0.19 |
| 1524 | S571W | 571 | 1.21 | 1.8 | 1.72 | 1.58 | 0.26 |
| 1525 | S571Q | 571 | 1.9 | 1.29 | 1.31 | 1.5 | 0.28 |
| 1526 | S571Y | 571 | 1.03 | 1.8 | 1.43 | 1.42 | 0.32 |
| 1527 | S571M | 571 | −0.58 | 0.89 | 1.18 | 0.5 | 0.77 |
| 1528 | E572M | 572 | 1.5 | 0.2 | 0.62 | 0.77 | 0.54 |
| 1529 | K574F | 574 | 2.41 | 2.84 | 1.54 | 2.26 | 0.54 |
| 1530 | K574V | 574 | 1.41 | 0.71 | −0.48 | 0.55 | 0.78 |
| 1531 | K574I | 574 | 0.8 | 0.6 | −0.07 | 0.44 | 0.38 |
| 1532 | G575D | 575 | 0.8 | 1.3 | −0.07 | 0.68 | 0.57 |
| 1533 | G575W | 575 | 0.4 | 0.71 | 0.21 | 0.44 | 0.21 |
| 1534 | Y577P | 577 | 1.03 | 0.6 | 1.31 | 0.98 | 0.29 |
| 1535 | Y577M | 577 | −0.18 | 1.01 | 1.31 | 0.71 | 0.64 |
| 1536 | K578F | 578 | 1.29 | 0.42 | 1.02 | 0.91 | 0.36 |
| 1537 | K578C | 578 | 1.21 | 0.2 | 0.62 | 0.67 | 0.41 |
| 1538 | K578A | 578 | 0.68 | 0.4 | 0.82 | 0.63 | 0.18 |
| 1539 | M580Q | 580 | 0.44 | 1.15 | 0.88 | 0.82 | 0.29 |
| 1540 | I581H | 58 | 0.55 | 0.3 | 0.88 | 0.58 | 0.24 |
| 1541 | N583V | 583 | 0.67 | 1.13 | 1.07 | 0.96 | 0.2 |
| 1542 | N583T | 583 | 0.66 | 0.93 | 0.53 | 0.71 | 0.16 |
| 1543 | N583G | 583 | 0.42 | 0.89 | 0.55 | 0.62 | 0.2 |
| 1544 | N583A | 583 | 0.65 | 0.57 | 0.56 | 0.59 | 0.04 |
| 1545 | N583I | 583 | 0.69 | 0.62 | 0.39 | 0.57 | 0.13 |
| 1546 | N583S | 583 | 0.45 | 0.72 | 0.42 | 0.53 | 0.13 |
| 1547 | L585K | 585 | 0.51 | 0.5 | 0.87 | 0.63 | 0.17 |
| 1548 | P586F | 586 | 1.71 | 0.89 | 1.02 | 1.21 | 0.36 |
| 1549 | G587C | 587 | 1.2 | 0.89 | 1.31 | 1.13 | 0.18 |
| 1550 | G587H | 587 | 0.8 | 0.6 | 0.84 | 0.75 | 0.1 |
| 1551 | G602A | 602 | 0.8 | 1.48 | 1.21 | 1.16 | 0.28 |
| 1552 | E604I | 604 | 0.36 | 0.89 | 0.55 | 0.6 | 0.22 |
| 1553 | E604T | 604 | 0.51 | 0.82 | 0.41 | 0.58 | 0.17 |
| 1554 | T605C | 605 | 0.47 | 1.08 | 1.16 | 0.9 | 0.31 |
| 1555 | T605G | 605 | 0.8 | 0.89 | 0.74 | 0.81 | 0.06 |
| 1556 | K607C | 607 | 0.93 | 0.57 | 0.63 | 0.71 | 0.16 |
| 1557 | S609E | 609 | 0.45 | 1.03 | 0.73 | 0.74 | 0.24 |
| 1558 | S609C | 609 | 0.64 | 0.84 | 0.68 | 0.72 | 0.09 |
| 1559 | S609K | 609 | 0.67 | 0.35 | 0.85 | 0.63 | 0.21 |
| 1560 | A610N | 610 | 0.8 | 1.59 | 1.32 | 1.24 | 0.33 |
| 1561 | A610I | 610 | 0.9 | 0.67 | 1.04 | 0.87 | 0.15 |
| 1562 | A610H | 610 | 0.9 | 0.38 | 0.63 | 0.64 | 0.21 |
| 1563 | A610Y | 610 | −0.99 | 1.3 | 1.14 | 0.49 | 1.04 |
| 1564 | L613G | 613 | 0.99 | 1.08 | 1.17 | 1.08 | 0.07 |
| 1565 | E614I | 614 | 0.52 | 0.21 | 1.65 | 0.79 | 0.62 |
| 1566 | K617V | 617 | 0.39 | 0.45 | 0.54 | 0.46 | 0.06 |
| 1567 | K617C | 617 | 0.4 | 0.42 | 0.46 | 0.43 | 0.02 |
| 1568 | K617Q | 617 | 0.5 | 0.51 | 0.26 | 0.43 | 0.12 |
| 1569 | Q618D | 618 | 0.8 | 0.59 | 0.48 | 0.62 | 0.13 |
| 1570 | N619R | 619 | 0.26 | 0.59 | 0.88 | 0.58 | 0.26 |
| 1571 | N619C | 619 | 0.44 | 0.53 | 0.58 | 0.52 | 0.06 |
| 1572 | K620F | 620 | 0.55 | 0.21 | 0.52 | 0.43 | 0.15 |
| 1573 | S624W | 624 | 0.52 | 1.13 | 0.11 | 0.59 | 0.42 |
| 1574 | S625N | 625 | 0.44 | 0.48 | 0.44 | 0.46 | 0.02 |
| 1575 | K626I | 626 | 1.04 | 0.44 | 0.97 | 0.82 | 0.27 |
| 1576 | K626V | 626 | 0.81 | 0.12 | 0.82 | 0.58 | 0.33 |
| 1577 | K626Y | 626 | 0.52 | 0.37 | 0.73 | 0.54 | 0.15 |
| 1578 | H634K | 634 | 0.08 | 1.08 | 0.55 | 0.57 | 0.41 |
| 1579 | L636H | 636 | 0.82 | 0.22 | 0.66 | 0.57 | 0.25 |
| 1580 | D638I | 638 | 1.51 | 0.22 | −0.03 | 0.57 | 0.68 |
| 1581 | D638C | 638 | 0.85 | 0.24 | 0.38 | 0.49 | 0.26 |
| 1582 | D638S | 638 | 0.41 | 0.04 | 0.88 | 0.45 | 0.35 |
| 1583 | Y639N | 639 | 0.82 | 0.92 | 1.07 | 0.93 | 0.1 |
| 1584 | Y639M | 639 | 0.82 | 1.32 | −0.03 | 0.7 | 0.56 |
| 1585 | Y639G | 639 | 0.82 | 0.78 | 0.19 | 0.6 | 0.29 |
| 1586 | Y639T | 639 | 0.64 | 0.04 | 0.89 | 0.52 | 0.36 |
| 1587 | Y639H | 639 | 0.13 | 1.32 | −0.03 | 0.47 | 0.6 |
| 1588 | N642A | 642 | 0.28 | 0.83 | 1.12 | 0.74 | 0.35 |
| 1589 | N642M | 642 | 0.32 | 0.8 | 0.47 | 0.53 | 0.2 |
| 1590 | C643T | 643 | 0.13 | 1.24 | 0.66 | 0.68 | 0.45 |
| 1591 | C643F | 643 | 0.53 | 0.23 | 0.53 | 0.43 | 0.14 |
| 1592 | I644Y | 644 | 0.66 | 0.33 | 0.61 | 0.53 | 0.14 |
| 1593 | I644A | 644 | 0.44 | 0.34 | 0.72 | 0.5 | 0.16 |
| 1594 | A645M | 645 | 0.73 | 0.74 | 0.38 | 0.62 | 0.17 |
| 1595 | A645N | 645 | 0.47 | 0.52 | 0.58 | 0.52 | 0.04 |
| 1596 | A645Q | 645 | 0.57 | 0.45 | 0.42 | 0.48 | 0.07 |
| 1597 | P648C | 648 | 1.28 | 0.84 | 0.82 | 0.98 | 0.21 |
| 1598 | P648M | 648 | 0.13 | 1.02 | 0.76 | 0.64 | 0.38 |
| 1599 | P648I | 648 | 0.67 | 0.24 | 0.57 | 0.49 | 0.19 |
| 1600 | F653R | 653 | 0.95 | 0.56 | 1.02 | 0.85 | 0.2 |
| 1601 | G654P | 654 | 0.61 | 0.44 | 0.75 | 0.6 | 0.13 |
| 1602 | F655H | 655 | 0.45 | 0.72 | 0.99 | 0.72 | 0.22 |
| 1603 | D656K | 656 | 0.88 | 0.87 | 0.55 | 0.77 | 0.15 |
| 1604 | D656G | 656 | 0.5 | 0.78 | 0.73 | 0.67 | 0.12 |
| 1605 | S658D | 658 | 0.3 | 0.78 | 0.41 | 0.5 | 0.21 |
| 1606 | D659K | 659 | 0.45 | 0.35 | 0.61 | 0.47 | 0.1 |
| 1607 | S661G | 661 | 0.88 | 0.97 | 0.47 | 0.77 | 0.22 |
| 1608 | S661Q | 661 | 0.72 | 0.37 | 0.39 | 0.49 | 0.16 |
| 1609 | S661M | 661 | 0.06 | 0.93 | 0.44 | 0.48 | 0.36 |
| 1610 | E664R | 664 | 0.45 | 0.93 | 0.43 | 0.61 | 0.23 |
| 1611 | E664F | 664 | 0.61 | 0.71 | 0.46 | 0.59 | 0.1 |
| 1612 | E664S | 664 | 0.27 | 0.42 | 0.61 | 0.43 | 0.14 |
| 1613 | S667R | 667 | 1.52 | 0.53 | 0.97 | 1.01 | 0.41 |
| 1614 | G668Q | 668 | 2.7 | 0.56 | −0.3 | 0.99 | 1.26 |
| 1615 | G668M | 668 | 0.25 | 0.36 | 0.68 | 0.43 | 0.18 |
| 1616 | F669P | 669 | 1.46 | 1.42 | 1.43 | 1.44 | 0.02 |
| 1617 | F669R | 669 | 1.62 | −0.27 | 0.17 | 0.51 | 0.81 |
| 1618 | L675C | 675 | 1.34 | 1.23 | 1.78 | 1.45 | 0.24 |
| 1619 | Q676V | 676 | 1.02 | 1.2 | 1.06 | 1.1 | 0.08 |
| 1620 | Q676T | 676 | 0.88 | 1.2 | 0.66 | 0.91 | 0.22 |
| 1621 | Q676S | 676 | 0.6 | 1.13 | 0.89 | 0.87 | 0.22 |
| 1622 | Q676W | 676 | 0.14 | 0.5 | 0.93 | 0.52 | 0.32 |
| 1623 | Y678F | 678 | 1.28 | 1.81 | 1.71 | 1.6 | 0.23 |
| 1624 | Y678H | 678 | 0.67 | 1.05 | 1.32 | 1.01 | 0.27 |
| 1625 | I680A | 680 | 1.63 | 1.34 | 1.34 | 1.44 | 0.14 |
| 1626 | I680V | 680 | 1.2 | 1.4 | 1.26 | 1.29 | 0.08 |
| 1627 | I680Q | 680 | 1.29 | 1.1 | 1.36 | 1.25 | 0.11 |
| 1628 | I680S | 680 | 1.03 | 1.04 | 1.07 | 1.05 | 0.02 |
| 1629 | I680N | 680 | 1.06 | 1.07 | 0.79 | 0.97 | 0.13 |
| 1630 | I680H | 680 | 0.92 | 1.17 | 0.78 | 0.96 | 0.16 |
| 1631 | I680F | 680 | 0.52 | 0.58 | 0.38 | 0.49 | 0.09 |
| 1632 | D681I | 681 | 1.11 | 1.21 | 1.16 | 1.16 | 0.04 |
| 1633 | D681W | 681 | 1.11 | 0.9 | 1.01 | 1.01 | 0.08 |
| 1634 | D681M | 681 | 0.53 | 0.68 | 0.79 | 0.67 | 0.11 |
| 1635 | D681Q | 681 | 0.48 | 0.8 | 0.65 | 0.64 | 0.13 |
| 1636 | D681T | 681 | 0.59 | 0.62 | 0.7 | 0.64 | 0.05 |
| 1637 | D681R | 681 | 0.54 | 0.49 | 0.58 | 0.54 | 0.04 |
| 1638 | D681K | 681 | 0.45 | 0.42 | 0.47 | 0.45 | 0.02 |
| 1639 | W682D | 682 | 1.37 | 1.35 | 1.35 | 1.36 | 0.01 |
| 1640 | W682K | 682 | 0.98 | 1.08 | 1.1 | 1.05 | 0.05 |
| 1641 | W682I | 682 | 0.83 | 1.06 | 0.92 | 0.94 | 0.09 |
| 1642 | W682N | 682 | 0.91 | 0.82 | 1.02 | 0.92 | 0.08 |
| 1643 | W682T | 682 | 0.88 | 0.88 | 0.97 | 0.91 | 0.04 |
| 1644 | W682Q | 682 | 0.86 | 0.82 | 1 | 0.89 | 0.08 |
| 1645 | W682E | 682 | 0.87 | 0.85 | 0.83 | 0.85 | 0.02 |
| 1646 | W682A | 682 | 0.74 | 0.82 | 0.94 | 0.83 | 0.08 |
| 1647 | W682V | 682 | 0.7 | 0.86 | 0.77 | 0.78 | 0.06 |
| 1648 | W682M | 682 | 0.7 | 0.72 | 0.67 | 0.7 | 0.02 |
| 1649 | W682G | 682 | 0.58 | 0.58 | 0.55 | 0.57 | 0.01 |
| 1650 | T683F | 683 | 1.44 | 1.61 | 1.34 | 1.46 | 0.11 |
| 1651 | T683Y | 683 | 0.59 | 0.73 | 0.69 | 0.67 | 0.06 |
| 1652 | I685Y | 685 | 1.22 | 0.89 | 1.2 | 1.11 | 0.15 |
| 1653 | S686R | 686 | 0.57 | 0.99 | −0.22 | 0.45 | 0.5 |
| 1654 | S686T | 686 | 0.56 | 0.46 | 0.24 | 0.42 | 0.14 |
| 1655 | E687N | 687 | 0.54 | 0.58 | 0.39 | 0.51 | 0.08 |
| 1656 | K688D | 688 | 2.15 | 2.25 | 0.24 | 1.55 | 0.92 |
| 1657 | K688V | 688 | 0.42 | 0.44 | 0.47 | 0.44 | 0.02 |
| 1658 | D691R | 691 | 0.36 | 1.29 | 0.23 | 0.62 | 0.47 |
| 1659 | L692M | 692 | 0.5 | 0.61 | 0.62 | 0.58 | 0.05 |
| 1660 | L693Q | 693 | 1.87 | 1.34 | 1.83 | 1.68 | 0.24 |
| 1661 | L693W | 693 | 1.48 | 1.32 | 1.95 | 1.58 | 0.27 |
| 1662 | L693G | 693 | 0.98 | 1.55 | 1.45 | 1.33 | 0.25 |
| 1663 | L693E | 693 | 0.46 | 1 | 0.39 | 0.61 | 0.27 |
| 1664 | L693M | 693 | 0.48 | 0.53 | 0.54 | 0.52 | 0.03 |
| 1665 | L693A | 693 | 0.94 | 0.4 | 0.04 | 0.46 | 0.37 |
| 1666 | L693D | 693 | 0.54 | 0.53 | 0.27 | 0.45 | 0.13 |
| 1667 | L693T | 693 | 0.34 | 0.32 | 0.68 | 0.45 | 0.16 |
| 1668 | Q694G | 694 | 0.83 | 1.35 | 1.37 | 1.18 | 0.25 |
| 1669 | Q694W | 694 | 1.52 | 0.83 | 1.15 | 1.17 | 0.28 |
| 1670 | Q694C | 694 | 1.15 | 0.65 | 1.08 | 0.96 | 0.22 |
| 1671 | Q694R | 694 | 0.91 | 1.08 | 0.39 | 0.79 | 0.29 |
| 1672 | Q694S | 694 | 0.14 | 2.18 | −0.02 | 0.77 | 1 |
| 1673 | Q694Y | 694 | 0.29 | 0.79 | 0.64 | 0.57 | 0.21 |
| 1674 | Q694K | 694 | 0.46 | 0.47 | 0.68 | 0.53 | 0.1 |
| 1675 | Q694I | 694 | 0.29 | 0.65 | 0.39 | 0.44 | 0.15 |
| 1676 | E695S | 695 | 2.74 | 2.8 | 1.37 | 2.3 | 0.66 |
| 1677 | G697I | 697 | 1.32 | 1.7 | 0.9 | 1.3 | 0.33 |
| 1678 | G697N | 697 | 0.98 | 0.62 | 0.67 | 0.76 | 0.16 |
| 1679 | G697P | 697 | 0.46 | 0.47 | 0.61 | 0.51 | 0.07 |
| 1680 | Q698F | 698 | 0.14 | 1.26 | 1 | 0.8 | 0.48 |
| 1681 | Q698A | 698 | 0.14 | 0.81 | 0.39 | 0.45 | 0.28 |
| 1682 | L699Y | 699 | 0.43 | 1.29 | 0.68 | 0.8 | 0.36 |
| 1683 | L699F | 699 | 0.71 | 0.73 | 0.79 | 0.75 | 0.03 |
| 1684 | L699C | 699 | 0.36 | 0.25 | 1.31 | 0.64 | 0.47 |
| 1685 | Y700V | 700 | 1.28 | 1.46 | 1.08 | 1.27 | 0.15 |
| 1686 | Y700A | 700 | 0.94 | 1.21 | 1.2 | 1.12 | 0.12 |
| 1687 | Y700G | 700 | 0.7 | 1.26 | 1.12 | 1.03 | 0.24 |
| 1688 | Y700I | 700 | 0.95 | 0.65 | 0.21 | 0.6 | 0.3 |
| 1689 | Y700T | 700 | 0.54 | 0.25 | 0.68 | 0.49 | 0.18 |
| 1690 | L701P | 701 | 1.61 | 1.18 | 1.15 | 1.31 | 0.21 |
| 1691 | L701W | 701 | 0.94 | 0.87 | 1.27 | 1.02 | 0.17 |
| 1692 | F702G | 702 | 2.82 | 2.75 | 2.85 | 2.81 | 0.04 |
| 1693 | F702N | 702 | 2.71 | 2.69 | 2.89 | 2.77 | 0.09 |
| 1694 | F702A | 702 | 2.49 | 2.54 | 2.71 | 2.58 | 0.1 |
| 1695 | F702Q | 702 | 2.31 | 2.03 | 2.4 | 2.25 | 0.16 |
| 1696 | F702H | 702 | 1.96 | 2 | 1.85 | 1.94 | 0.06 |
| 1697 | F702T | 702 | 1.72 | 1.73 | 1.7 | 1.72 | 0.01 |
| 1698 | F702M | 702 | 1.52 | 1.73 | 1.7 | 1.65 | 0.09 |
| 1699 | F702S | 702 | 1.27 | 1.35 | 1.35 | 1.32 | 0.04 |
| 1700 | F702V | 702 | 0.95 | 0.94 | 1.03 | 0.97 | 0.04 |
| 1701 | F702C | 702 | 0.55 | 0.79 | 0.7 | 0.68 | 0.1 |
| 1702 | F702L | 702 | 0.46 | 0.52 | 0.51 | 0.5 | 0.03 |
| 1703 | Q703C | 703 | 1.24 | 1.63 | 2.06 | 1.64 | 0.34 |
| 1704 | K711P | 711 | 1.73 | 1.48 | 1.74 | 1.65 | 0.12 |
| 1705 | K711L | 711 | 0.31 | 0.46 | 0.54 | 0.44 | 0.1 |
| 1706 | K712W | 712 | 2.68 | 2.64 | 2.57 | 2.63 | 0.04 |
| 1707 | K712F | 712 | 2.35 | 2.71 | 2.74 | 2.6 | 0.18 |
| 1708 | K712Y | 712 | 2.42 | 2.5 | 2.39 | 2.44 | 0.04 |
| 1709 | K712G | 712 | 2.06 | 2.27 | 1.84 | 2.05 | 0.17 |
| 1710 | K712H | 712 | 1.88 | 2.19 | 2.08 | 2.05 | 0.13 |
| 1711 | K712S | 712 | 1.11 | 0.95 | 1.2 | 1.09 | 0.1 |
| 1712 | K712R | 712 | 0.85 | 1.37 | 0.88 | 1.03 | 0.24 |
| 1713 | K712C | 712 | 0.83 | 0.24 | 1.26 | 0.78 | 0.42 |
| 1714 | K712A | 712 | 0.86 | 0.24 | 0.98 | 0.69 | 0.32 |
| 1715 | K712Q | 712 | 0.13 | 0.93 | 0.58 | 0.55 | 0.33 |
| 1716 | S713R | 713 | 1.23 | 0.94 | −0.02 | 0.72 | 0.54 |
| 1717 | T714Y | 714 | 1.74 | 1.55 | 1.77 | 1.69 | 0.1 |
| 1718 | T714W | 714 | 0.65 | 0.65 | 0.75 | 0.68 | 0.05 |
| 1719 | T714N | 714 | 0.72 | 0.89 | 0.38 | 0.66 | 0.21 |
| 1720 | N716R | 716 | 1.71 | 1.88 | 1.96 | 1.85 | 0.11 |
| 1721 | N716M | 716 | 1.32 | 1.49 | 1.47 | 1.43 | 0.08 |
| 1722 | N716I | 716 | 1.18 | 1.25 | 1.29 | 1.24 | 0.04 |
| 1723 | N716V | 716 | 1.22 | 1.16 | 1.22 | 1.2 | 0.03 |
| 1724 | N716T | 716 | 1.04 | 1.22 | 1.12 | 1.12 | 0.07 |
| 1725 | N716C | 716 | 0.69 | 1.03 | 0.49 | 0.73 | 0.23 |
| 1726 | N716Q | 716 | 0.55 | 0.81 | 0.7 | 0.69 | 0.11 |
| 1727 | N716A | 716 | 0.53 | 0.49 | 0.45 | 0.49 | 0.04 |
| 1728 | N716K | 716 | 0.46 | 0.53 | 0.46 | 0.48 | 0.03 |
| 1729 | D717P | 717 | 2.05 | 2.25 | 2.06 | 2.12 | 0.09 |
| 1730 | D717A | 717 | 0.68 | 0.61 | 0.65 | 0.65 | 0.03 |
| 1731 | L719K | 719 | 0.42 | 0.58 | 0.63 | 0.54 | 0.09 |
| 1732 | K725Y | 725 | 0.81 | 0.68 | 0.46 | 0.65 | 0.14 |
| 1733 | L727H | 727 | 0.75 | 0.66 | 0.43 | 0.61 | 0.13 |
| 1734 | L727T | 727 | 0.14 | 0.64 | 0.89 | 0.56 | 0.31 |
| 1735 | L727I | 727 | 0.36 | −0.05 | 0.99 | 0.43 | 0.43 |
| 1736 | F728T | 728 | 1.39 | 0.93 | −0.02 | 0.76 | 0.59 |
| 1737 | S729H | 729 | 0.83 | 0.46 | 0.38 | 0.56 | 0.2 |
| 1738 | S729K | 729 | 1.06 | 0.23 | −0.02 | 0.42 | 0.46 |
| 1739 | E730L | 730 | 0.92 | 3.32 | 2.58 | 2.28 | 1 |
| 1740 | E730M | 730 | 1.75 | 1.33 | −0.02 | 1.02 | 0.76 |
| 1741 | E730I | 730 | 0.83 | 1.33 | 0.67 | 0.95 | 0.28 |
| 1742 | E730G | 730 | 1.24 | 0.64 | 0.54 | 0.8 | 0.31 |
| 1743 | E730V | 730 | 0.61 | 0.23 | 1.01 | 0.62 | 0.32 |
| 1744 | E731P | 731 | 1.47 | 4.46 | 3.64 | 3.19 | 1.26 |
| 1745 | E731A | 731 | 0.65 | 1.77 | −0.43 | 0.67 | 0.9 |
| 1746 | E731L | 731 | 0.65 | 0.23 | 0.83 | 0.57 | 0.25 |
| 1747 | N732T | 732 | 1.36 | 4.47 | 3.65 | 3.16 | 1.31 |
| 1748 | N732D | 732 | 1.24 | 1.22 | 0.49 | 0.98 | 0.35 |
| 1749 | L733I | 733 | 0.55 | 0.67 | 0.57 | 0.59 | 0.05 |
| 1750 | L733K | 733 | 2.09 | −0.08 | −0.59 | 0.47 | 1.16 |
| 1751 | D735R | 735 | 0.41 | 0.46 | 0.7 | 0.52 | 0.13 |
| 1752 | D735K | 735 | 0.83 | 0.32 | 0.31 | 0.49 | 0.25 |
| 1753 | V737Q | 737 | 0.82 | 1.14 | 0.37 | 0.77 | 0.32 |
| 1754 | L738E | 738 | 1.12 | 1.05 | 1.01 | 1.06 | 0.05 |
| 1755 | N741G | 741 | 1.15 | 1.17 | 1.44 | 1.26 | 0.13 |
| 1756 | N741C | 741 | 0.48 | 0.47 | 0.96 | 0.64 | 0.23 |
| 1757 | E743N | 743 | 0.72 | 0.89 | 0.91 | 0.84 | 0.09 |
| 1758 | A744N | 744 | 3.28 | 3.38 | 3.54 | 3.4 | 0.11 |
| 1759 | A744P | 744 | 2.39 | 2.69 | 2.65 | 2.58 | 0.13 |
| 1760 | A744T | 744 | 2.4 | 2.61 | 2.67 | 2.56 | 0.11 |
| 1761 | A744C | 744 | 1.68 | 1.77 | 1.8 | 1.75 | 0.05 |
| 1762 | A744S | 744 | 1.51 | 1.79 | 1.78 | 1.69 | 0.13 |
| 1763 | E745P | 745 | 0.71 | 1.04 | 0.67 | 0.8 | 0.17 |
| 1764 | F748G | 748 | 2.41 | 2.55 | 2.55 | 2.5 | 0.07 |
| 1765 | F748A | 748 | 2.38 | 2.37 | 2.59 | 2.44 | 0.1 |
| 1766 | F748S | 748 | 2.38 | 2.48 | 2.44 | 2.43 | 0.04 |
| 1767 | F748H | 748 | 2.2 | 2.28 | 2.47 | 2.32 | 0.11 |
| 1768 | F748W | 748 | 1.84 | 1.93 | 1.89 | 1.89 | 0.04 |
| 1769 | F748T | 748 | 1.75 | 1.74 | 1.95 | 1.81 | 0.1 |
| 1770 | F748R | 748 | 1.23 | 1.46 | 1.69 | 1.46 | 0.19 |
| 1771 | F748V | 748 | 1.24 | 1.25 | 1.2 | 1.23 | 0.02 |
| 1772 | F748E | 748 | 0.84 | 1.2 | 1.33 | 1.12 | 0.2 |
| 1773 | F748M | 748 | 0.73 | 1.28 | 1.36 | 1.12 | 0.28 |
| 1774 | F748N | 748 | 1.01 | 1.26 | 0.91 | 1.06 | 0.15 |
| 1775 | F748Q | 748 | 1.07 | 0.89 | 1.22 | 1.06 | 0.14 |
| 1776 | F748C | 748 | 1.02 | 0.78 | 1.06 | 0.95 | 0.12 |
| 1777 | F748D | 748 | 1.15 | 1 | 0.6 | 0.92 | 0.23 |
| 1778 | F748I | 748 | 0.24 | 0.63 | 0.41 | 0.43 | 0.16 |
| 1779 | K750P | 750 | 3.95 | 4.11 | 4.24 | 4.1 | 0.12 |
| 1780 | K750G | 750 | 3.13 | 3.25 | 3.33 | 3.24 | 0.08 |
| 1781 | K750A | 750 | 1.95 | 1.96 | 2.04 | 1.98 | 0.04 |
| 1782 | K750E | 750 | 1.74 | 1.89 | 1.8 | 1.81 | 0.06 |
| 1783 | K750W | 750 | 1.81 | 1.71 | 1.53 | 1.69 | 0.12 |
| 1784 | K750S | 750 | 1.59 | 1.63 | 1.72 | 1.65 | 0.06 |
| 1785 | K750F | 750 | 1.56 | 1.58 | 1.6 | 1.58 | 0.02 |
| 1786 | K750Y | 750 | 1.57 | 1.45 | 1.55 | 1.53 | 0.05 |
| 1787 | K750H | 750 | 1.29 | 1.37 | 1.41 | 1.36 | 0.05 |
| 1788 | K750N | 750 | 0.9 | 1.04 | 1 | 0.98 | 0.06 |
| 1789 | K750L | 750 | 0.76 | 0.55 | 0.68 | 0.66 | 0.09 |
| 1790 | K750T | 750 | 0.67 | 0.62 | 0.61 | 0.63 | 0.03 |
| 1791 | S751A | 751 | 3.59 | 3.78 | 3.93 | 3.77 | 0.14 |
| 1792 | S751G | 751 | 3.08 | 3.43 | 3.39 | 3.3 | 0.16 |
| 1793 | S751Q | 751 | 3.01 | 3.09 | 3.16 | 3.09 | 0.06 |
| 1794 | S751L | 751 | 2.97 | 3.09 | 3.19 | 3.08 | 0.09 |
| 1795 | S751H | 751 | 2.59 | 2.88 | 3.05 | 2.84 | 0.19 |
| 1796 | S751C | 751 | 2.51 | 2.83 | 2.79 | 2.71 | 0.15 |
| 1797 | S751P | 751 | 2.62 | 2.76 | 2.75 | 2.71 | 0.06 |
| 1798 | S751M | 751 | 2.35 | 2.54 | 2.57 | 2.48 | 0.1 |
| 1799 | S751N | 751 | 2.48 | 2.49 | 2.39 | 2.45 | 0.04 |
| 1800 | S751E | 751 | 2.32 | 2.4 | 2.38 | 2.37 | 0.03 |
| 1801 | S751R | 751 | 1.89 | 1.92 | 2 | 1.94 | 0.05 |
| 1802 | S751F | 751 | 1.44 | 1.37 | 1.59 | 1.47 | 0.09 |
| 1803 | S751V | 751 | 1.39 | 1.4 | 1.36 | 1.39 | 0.02 |
| 1804 | S751T | 751 | 1.23 | 1.28 | 1.29 | 1.27 | 0.03 |
| 1805 | S751D | 751 | 0.31 | 0.78 | 0.75 | 0.62 | 0.21 |
| 1806 | S752Y | 752 | 2.59 | 2.71 | 2.67 | 2.66 | 0.05 |
| 1807 | S752F | 752 | 2.27 | 2.46 | 2.5 | 2.41 | 0.1 |
| 1808 | S752H | 752 | 2.27 | 2.28 | 2.13 | 2.23 | 0.07 |
| 1809 | S752I | 752 | 1.9 | 2.14 | 2.01 | 2.02 | 0.1 |
| 1810 | S752C | 752 | 1.57 | 1.7 | 1.68 | 1.65 | 0.06 |
| 1811 | S752V | 752 | 1.39 | 1.56 | 1.46 | 1.47 | 0.07 |
| 1812 | S752M | 752 | 1.17 | 1.45 | 1.29 | 1.3 | 0.11 |
| 1813 | S752G | 752 | 1.27 | 1.22 | 1.16 | 1.22 | 0.04 |
| 1814 | S752W | 752 | 1.17 | 1.16 | 1.26 | 1.2 | 0.05 |
| 1815 | S752Q | 752 | 1.22 | 1.18 | 1.1 | 1.17 | 0.05 |
| 1816 | S752L | 752 | 0.41 | 0.48 | 0.49 | 0.46 | 0.04 |
| 1817 | I753E | 753 | 0.36 | 0.45 | 0.45 | 0.42 | 0.04 |
| 1818 | K754G | 754 | 3.52 | 3.77 | 3.8 | 3.69 | 0.13 |
| 1819 | K754H | 754 | 3.55 | 3.6 | 3.84 | 3.66 | 0.13 |
| 1820 | K754W | 754 | 3.22 | 3.06 | 3.2 | 3.16 | 0.07 |
| 1821 | K754S | 754 | 2.91 | 3.1 | 2.99 | 3 | 0.08 |
| 1822 | K754V | 754 | 2.66 | 2.78 | 2.85 | 2.76 | 0.08 |
| 1823 | K754Y | 754 | 2.63 | 2.79 | 2.85 | 2.76 | 0.09 |
| 1824 | K754I | 754 | 2.66 | 2.73 | 2.83 | 2.74 | 0.07 |
| 1825 | K754F | 754 | 2.67 | 2.65 | 2.72 | 2.68 | 0.03 |
| 1826 | K754D | 754 | 2.22 | 2.57 | 2.59 | 2.46 | 0.17 |
| 1827 | K754C | 754 | 2.06 | 2.73 | 2.33 | 2.37 | 0.27 |
| 1828 | K754A | 754 | 2.17 | 2.4 | 2.36 | 2.31 | 0.1 |
| 1829 | K754L | 754 | 2.09 | 2.28 | 2.35 | 2.24 | 0.11 |
| 1830 | K754P | 754 | 1.84 | 1.77 | 1.72 | 1.78 | 0.05 |
| 1831 | K754T | 754 | 1.42 | 1.56 | 1.55 | 1.51 | 0.07 |
| 1832 | K754N | 754 | 0.78 | 0.84 | 0.86 | 0.83 | 0.04 |
| 1833 | K754Q | 754 | 0.68 | 0.79 | 0.77 | 0.75 | 0.05 |
| 1834 | K754E | 754 | 0.65 | 0.68 | 0.73 | 0.68 | 0.03 |
| 1835 | P756M | 756 | 2.25 | 1.46 | 1.05 | 1.59 | 0.5 |
| 1836 | P756I | 756 | 1.62 | 1.46 | 1.65 | 1.58 | 0.08 |
| 1837 | P756K | 756 | 1.37 | 1.71 | 1.45 | 1.51 | 0.15 |
| 1838 | P756H | 756 | 1.03 | 1.31 | 1.61 | 1.32 | 0.24 |
| 1839 | P756N | 756 | 0.11 | 1.6 | 2.03 | 1.25 | 0.82 |
| 1840 | P756L | 756 | 0.97 | 1.4 | 0.84 | 1.07 | 0.24 |
| 1841 | P756S | 756 | 0.9 | 0.68 | 1.11 | 0.9 | 0.18 |
| 1842 | P756T | 756 | 0.44 | 0.82 | 0.91 | 0.72 | 0.21 |
| 1843 | I757W | 757 | 3.29 | 3.3 | 4.2 | 3.6 | 0.43 |
| 1844 | I757P | 757 | 2.89 | 3.01 | 2.85 | 2.91 | 0.07 |
| 1845 | I757K | 757 | 2.34 | 2.35 | 2.55 | 2.42 | 0.1 |
| 1846 | I757F | 757 | 1.95 | 2.16 | 2.3 | 2.14 | 0.14 |
| 1847 | I757A | 757 | 1.96 | 2.1 | 1.85 | 1.97 | 0.1 |
| 1848 | I757R | 757 | 1.45 | 1.91 | 1.45 | 1.6 | 0.22 |
| 1849 | I757Y | 757 | 1.72 | 1.75 | 1.25 | 1.57 | 0.23 |
| 1850 | I757L | 757 | 1.1 | 1.3 | 1.17 | 1.19 | 0.09 |
| 1851 | I757S | 757 | 0.81 | 1.06 | 0.87 | 0.91 | 0.11 |
| 1852 | I757Q | 757 | 0.85 | 0.1 | 1.11 | 0.69 | 0.43 |
| 1853 | I757C | 757 | 0.52 | −0.08 | 1.2 | 0.55 | 0.53 |
| 1854 | I757G | 757 | 0.35 | 0.58 | 0.5 | 0.48 | 0.09 |
| 1855 | I757M | 757 | 0.36 | 0.6 | 0.45 | 0.47 | 0.1 |
| 1856 | I758A | 758 | 1.77 | 1.91 | 1.77 | 1.82 | 0.07 |
| 1857 | I758E | 758 | 1.58 | 1.84 | 1.86 | 1.76 | 0.13 |
| 1858 | I758G | 758 | 1.39 | 1.66 | 1.48 | 1.51 | 0.11 |
| 1859 | I758Q | 758 | 1.66 | 1.29 | 1.39 | 1.45 | 0.16 |
| 1860 | I758S | 758 | 1.33 | 1.46 | 1.39 | 1.39 | 0.05 |
| 1861 | I758T | 758 | 0.97 | 1.07 | 1.44 | 1.16 | 0.2 |
| 1862 | I758C | 758 | 1.04 | 1.01 | 1.15 | 1.07 | 0.06 |
| 1863 | I758V | 758 | 0.95 | 1.06 | 1.03 | 1.01 | 0.05 |
| 1864 | I758K | 758 | 0.85 | 0.82 | 0.69 | 0.79 | 0.07 |
| 1865 | G762K | 762 | 0.46 | 0.05 | 0.89 | 0.47 | 0.34 |
| 1866 | I764Q | 764 | 0.7 | 0.75 | 0.49 | 0.65 | 0.11 |
| 1867 | V766Y | 766 | 0.51 | 0.89 | −0.06 | 0.45 | 0.39 |
| 1868 | N767R | 767 | 0.25 | 0.56 | 0.46 | 0.43 | 0.13 |
| 1869 | R768K | 768 | 1.4 | 0.89 | 1.48 | 1.26 | 0.26 |
| 1870 | T769L | 769 | 0.46 | 0.35 | 0.57 | 0.46 | 0.09 |
| 1871 | E771W | 771 | 1.53 | 1.44 | 1.65 | 1.54 | 0.09 |
| 1872 | E771A | 771 | 0.88 | 0.86 | 0.95 | 0.9 | 0.04 |
| 1873 | E771R | 771 | 0.77 | 0.75 | 0.58 | 0.7 | 0.09 |
| 1874 | E771C | 771 | 0.76 | 0.6 | 0.7 | 0.69 | 0.06 |
| 1875 | E771Y | 771 | 0.56 | 0.73 | 0.69 | 0.66 | 0.07 |
| 1876 | E771I | 771 | 0.7 | 0.68 | 0.36 | 0.58 | 0.16 |
| 1877 | E771T | 771 | 0.62 | 0.59 | 0.53 | 0.58 | 0.04 |
| 1878 | E771F | 771 | 0.52 | 0.75 | 0.43 | 0.57 | 0.14 |
| 1879 | E771K | 771 | 0.48 | 0.53 | 0.66 | 0.56 | 0.08 |
| 1880 | E771M | 771 | 0.5 | 0.43 | 0.69 | 0.54 | 0.11 |
| 1881 | E771S | 771 | 0.48 | 0.56 | 0.51 | 0.52 | 0.03 |
| 1882 | E771H | 771 | 0.47 | 0.38 | 0.69 | 0.51 | 0.13 |
| 1883 | E771Q | 771 | 0.37 | 0.64 | 0.49 | 0.5 | 0.11 |
| 1884 | E771V | 771 | 0.26 | 0.58 | 0.41 | 0.42 | 0.13 |
| 1885 | A772W | 772 | 0.79 | 0.19 | 0.89 | 0.62 | 0.31 |
| 1886 | E773I | 773 | 0.88 | 0.36 | 0.58 | 0.6 | 0.22 |
| 1887 | E773P | 773 | 0.68 | 0.48 | 0.25 | 0.47 | 0.17 |
| 1888 | E774W | 774 | 1.49 | 1.89 | 1.73 | 1.7 | 0.17 |
| 1889 | E774M | 774 | 1.75 | 1.58 | 1.27 | 1.53 | 0.2 |
| 1890 | E774S | 774 | 0.96 | 0.73 | 0.66 | 0.78 | 0.13 |
| 1891 | E774V | 774 | 0.51 | 0.88 | 0.93 | 0.77 | 0.19 |
| 1892 | E774T | 774 | 0.54 | 0.88 | 0.72 | 0.71 | 0.14 |
| 1893 | E774K | 774 | 0.54 | 0.74 | 0.74 | 0.67 | 0.09 |
| 1894 | E774L | 774 | 0.87 | 0.43 | 0.65 | 0.65 | 0.18 |
| 1895 | E774I | 774 | 0.8 | 0.78 | 0.12 | 0.56 | 0.32 |
| 1896 | E774R | 774 | 0.71 | 0.24 | 0.6 | 0.52 | 0.2 |
| 1897 | E774Q | 774 | 0.84 | 0.34 | 0.28 | 0.49 | 0.25 |
| 1898 | E774H | 774 | 0.8 | 0.39 | 0.22 | 0.47 | 0.24 |
| 1899 | K775C | 775 | 0.7 | 0.51 | 0.34 | 0.51 | 0.15 |
| 1900 | Q777R | 777 | 0.84 | 0.57 | 0.54 | 0.65 | 0.13 |
| 1901 | Q777V | 777 | 0.46 | 0.72 | 0.67 | 0.62 | 0.11 |
| 1902 | F778Q | 778 | 0.87 | 0.47 | 1.08 | 0.81 | 0.25 |
| 1903 | F778K | 778 | 0.92 | 0.68 | 0.48 | 0.69 | 0.18 |
| 1904 | F778R | 778 | 0.46 | 0.61 | 0.43 | 0.5 | 0.08 |
| 1905 | F778G | 778 | 0.57 | 0.28 | 0.4 | 0.42 | 0.12 |
| 1906 | I783Q | 783 | 0.61 | 1.03 | 0.91 | 0.85 | 0.18 |
| 1907 | V784F | 784 | 1.03 | 1.39 | 1.33 | 1.25 | 0.16 |
| 1908 | V784L | 784 | 1.06 | 0.92 | 1.09 | 1.03 | 0.07 |
| 1909 | V784Y | 784 | 0.81 | 1.23 | 0.9 | 0.98 | 0.18 |
| 1910 | V784R | 784 | 0.73 | 0.93 | 0.99 | 0.88 | 0.11 |
| 1911 | V784W | 784 | 0.47 | 0.74 | 1.04 | 0.75 | 0.23 |
| 1912 | V784H | 784 | −0.58 | 0.89 | 1.33 | 0.55 | 0.82 |
| 1913 | V784I | 784 | 0.6 | 0.42 | 0.43 | 0.48 | 0.08 |
| 1914 | N787A | 787 | 3.01 | 3.25 | 3.28 | 3.18 | 0.12 |
| 1915 | N787W | 787 | 2.32 | 2.6 | 2.15 | 2.36 | 0.19 |
| 1916 | N787G | 787 | 2.43 | 2.15 | 1.9 | 2.16 | 0.22 |
| 1917 | N787Q | 787 | 2.07 | 2.4 | 1.56 | 2.01 | 0.35 |
| 1918 | N787P | 787 | 1.92 | 1.98 | 1.98 | 1.96 | 0.03 |
| 1919 | N787R | 787 | 1.92 | 1.65 | 1.92 | 1.83 | 0.13 |
| 1920 | N787S | 787 | 1.73 | 1.74 | 1.95 | 1.81 | 0.1 |
| 1921 | N787C | 787 | 2.07 | 0.9 | 1.56 | 1.51 | 0.48 |
| 1922 | N787M | 787 | 1.28 | 1.36 | 1.53 | 1.39 | 0.1 |
| 1923 | N787H | 787 | 0.68 | 1.46 | 1.05 | 1.06 | 0.32 |
| 1924 | N787F | 787 | 0.76 | 0.84 | 0.97 | 0.86 | 0.09 |
| 1925 | I788Y | 788 | 0.53 | 0.79 | 1.15 | 0.82 | 0.25 |
| 1926 | I788F | 788 | 0.57 | 0.49 | 0.7 | 0.59 | 0.09 |
| 1927 | P789C | 789 | −0.97 | 1.51 | 0.8 | 0.44 | 1.04 |
| 1928 | E790I | 790 | 1.92 | 2.22 | 2.26 | 2.13 | 0.15 |
| 1929 | E790P | 790 | 1.59 | 1.82 | 2.22 | 1.88 | 0.26 |
| 1930 | E790F | 790 | 1.74 | 1.59 | 1.75 | 1.69 | 0.07 |
| 1931 | E790V | 790 | 1.82 | 1.64 | 1.45 | 1.64 | 0.15 |
| 1932 | E790R | 790 | 1.25 | 1.64 | 1.79 | 1.56 | 0.23 |
| 1933 | E790K | 790 | 1.47 | 1.59 | 1.51 | 1.52 | 0.05 |
| 1934 | E790A | 790 | 1.43 | 1.38 | 1.75 | 1.52 | 0.16 |
| 1935 | E790T | 790 | 1.71 | 0.9 | 1.82 | 1.47 | 0.41 |
| 1936 | E790N | 790 | 1.71 | 1.48 | 1.05 | 1.41 | 0.27 |
| 1937 | E790S | 790 | 1.64 | 1.11 | 1.05 | 1.27 | 0.26 |
| 1938 | E790Q | 790 | 0.96 | 1.57 | 1.08 | 1.2 | 0.26 |
| 1939 | E790W | 790 | 1.08 | 1.43 | 0.83 | 1.11 | 0.25 |
| 1940 | E790M | 790 | 1.38 | 1.02 | 0.92 | 1.11 | 0.2 |
| 1941 | E790C | 790 | 1.51 | 1.82 | −0.05 | 1.09 | 0.82 |
| 1942 | E790G | 790 | 1.07 | 0.97 | 0.9 | 0.98 | 0.07 |
| 1943 | E790Y | 790 | 1.13 | 0.79 | 0.68 | 0.87 | 0.19 |
| 1944 | E790H | 790 | 1.08 | 0.74 | 0.22 | 0.68 | 0.36 |
| 1945 | N791G | 791 | 2.07 | 0.2 | 0.65 | 0.97 | 0.8 |
| 1946 | N791E | 791 | 0.29 | 0.85 | 0.6 | 0.58 | 0.23 |
| 1947 | N791F | 791 | 0.57 | 0.65 | 0.4 | 0.54 | 0.1 |
| 1948 | Y793V | 793 | 1.31 | 0.7 | 1.27 | 1.09 | 0.28 |
| 1949 | Y793R | 793 | 0.7 | 0.95 | 0.59 | 0.75 | 0.15 |
| 1950 | Y793W | 793 | 0.31 | 0.79 | 0.83 | 0.64 | 0.24 |
| 1951 | Y793Q | 793 | 0.82 | 0.49 | 0.36 | 0.56 | 0.19 |
| 1952 | Q794P | 794 | 1 | 1.43 | 0.14 | 0.86 | 0.54 |
| 1953 | Q794L | 794 | 0.5 | 0.94 | 0.53 | 0.66 | 0.2 |
| 1954 | Y797W | 797 | 0.64 | 1.05 | −0.22 | 0.49 | 0.53 |
| 1955 | K798L | 798 | 1.17 | 0.46 | 0.78 | 0.8 | 0.29 |
| 1956 | F800W | 800 | 0.82 | 1.31 | 0.94 | 1.02 | 0.21 |
| 1957 | N801A | 801 | 0.82 | 0.9 | 1.35 | 1.02 | 0.23 |
| 1958 | N801S | 801 | 0.13 | −0.48 | 1.66 | 0.44 | 0.9 |
| 1959 | K803A | 803 | 1.23 | 1.6 | 0.65 | 1.16 | 0.39 |
| 1960 | S804F | 804 | 1.23 | 0.91 | 0.88 | 1 | 0.16 |
| 1961 | S804I | 804 | 0.42 | 1.51 | −0.04 | 0.63 | 0.65 |
| 1962 | K806C | 806 | 0.2 | 0.75 | 0.54 | 0.5 | 0.23 |
| 1963 | S809G | 809 | 0.59 | 0.27 | 0.62 | 0.49 | 0.16 |
| 1964 | A813H | 813 | 0.78 | 1.11 | 0.24 | 0.71 | 0.36 |
| 1965 | E823P | 823 | 1.14 | 0.88 | 0.84 | 0.96 | 0.13 |
| 1966 | E823A | 823 | 0.67 | 0.5 | 0.69 | 0.62 | 0.09 |
| 1967 | E823S | 823 | 0.19 | 0.88 | 0.19 | 0.42 | 0.33 |
| 1968 | A825H | 825 | 1.51 | 1.71 | 1.82 | 1.68 | 0.13 |
| 1969 | A825R | 825 | 1.11 | 1.34 | 1.4 | 1.28 | 0.12 |
| 1970 | A825K | 825 | 0.38 | 0.53 | 0.45 | 0.45 | 0.06 |
| 1971 | T826F | 826 | 3.15 | 3.35 | 3.35 | 3.28 | 0.1 |
| 1972 | T826Y | 826 | 3.03 | 3.3 | 3.28 | 3.2 | 0.12 |
| 1973 | T826W | 826 | 2.55 | 2.75 | 2.62 | 2.64 | 0.08 |
| 1974 | T826H | 826 | 1.94 | 2 | 2.23 | 2.06 | 0.12 |
| 1975 | T826C | 826 | 1.73 | 1.79 | 1.96 | 1.83 | 0.1 |
| 1976 | T826I | 826 | 1.56 | 1.77 | 1.89 | 1.74 | 0.13 |
| 1977 | T826M | 826 | 1.45 | 1.67 | 1.7 | 1.61 | 0.11 |
| 1978 | T826L | 826 | 1.1 | 1.3 | 1.4 | 1.27 | 0.12 |
| 1979 | T826K | 826 | 1.15 | 1.29 | 1.27 | 1.23 | 0.06 |
| 1980 | T826R | 826 | 1.07 | 1.13 | 1.24 | 1.15 | 0.07 |
| 1981 | T826Q | 826 | 0.89 | 0.72 | 0.8 | 0.8 | 0.07 |
| 1982 | T826N | 826 | 0.29 | 0.48 | 0.5 | 0.42 | 0.09 |
| 1983 | N827D | 827 | 0.95 | 0.69 | 0.72 | 0.79 | 0.12 |
| 1984 | N827E | 827 | 0.58 | 0.84 | 0.77 | 0.73 | 0.11 |
| 1985 | V829T | 829 | 1.87 | 1.96 | 1.9 | 1.91 | 0.04 |
| 1986 | D831L | 831 | 2.04 | 2.27 | 2.27 | 2.19 | 0.11 |
| 1987 | D831M | 831 | 0.83 | 0.75 | 0.97 | 0.85 | 0.09 |
| 1988 | D831Q | 831 | 1.12 | 0.84 | 0.39 | 0.78 | 0.3 |
| 1989 | D831R | 831 | 0.83 | 1.17 | −0.13 | 0.63 | 0.55 |
| 1990 | D831N | 831 | 0.83 | 0.7 | 0.1 | 0.54 | 0.32 |
| 1991 | Y832R | 832 | 3.17 | 3.35 | 3.45 | 3.32 | 0.12 |
| 1992 | Y832K | 832 | 2.7 | 2.97 | 3.04 | 2.9 | 0.15 |
| 1993 | Y832G | 832 | 2.08 | 2.22 | 2.29 | 2.2 | 0.09 |
| 1994 | Y832Q | 832 | 1.46 | 1.52 | 1.59 | 1.53 | 0.05 |
| 1995 | Y832A | 832 | 1.01 | 1.2 | 1.15 | 1.12 | 0.08 |
| 1996 | Y832N | 832 | 0.96 | 1.12 | 1.06 | 1.04 | 0.07 |
| 1997 | Y832M | 832 | 0.68 | 0.81 | 0.89 | 0.79 | 0.09 |
| 1998 | Y832E | 832 | 0.5 | 0.63 | 0.68 | 0.6 | 0.08 |
| 1999 | Y836K | 836 | 3.67 | 3.99 | 4.02 | 3.89 | 0.16 |
| 2000 | Y836Q | 836 | 3.61 | 3.78 | 3.87 | 3.75 | 0.1 |
| 2001 | Y836R | 836 | 3.11 | 3.28 | 3.31 | 3.23 | 0.09 |
| 2002 | Y836I | 836 | 3.21 | 2.99 | 3.08 | 3.09 | 0.09 |
| 2003 | Y836D | 836 | 2.87 | 2.91 | 3.25 | 3.01 | 0.17 |
| 2004 | Y836V | 836 | 2.83 | 3.07 | 3.08 | 3 | 0.12 |
| 2005 | Y836T | 836 | 2.81 | 3.05 | 2.89 | 2.92 | 0.1 |
| 2006 | Y836N | 836 | 2.71 | 2.95 | 3.04 | 2.9 | 0.14 |
| 2007 | Y836E | 836 | 2.67 | 3.07 | 2.76 | 2.84 | 0.17 |
| 2008 | Y836H | 836 | 2.89 | 2.92 | 2.68 | 2.83 | 0.1 |
| 2009 | Y836L | 836 | 2.48 | 2.69 | 2.81 | 2.66 | 0.14 |
| 2010 | Y836M | 836 | 2.61 | 2.72 | 2.65 | 2.66 | 0.05 |
| 2011 | Y836A | 836 | 2.7 | 2.43 | 2.57 | 2.57 | 0.11 |
| 2012 | Y836S | 836 | 2.09 | 2.37 | 2.37 | 2.28 | 0.13 |
| 2013 | Y836G | 836 | 1.93 | 1.69 | 1.94 | 1.85 | 0.12 |
| 2014 | Y836C | 836 | 0.84 | 0.93 | 0.83 | 0.86 | 0.04 |
| 2015 | D837P | 837 | 2.8 | 2.8 | 2.83 | 2.81 | 0.02 |
| 2016 | Y839L | 839 | 2.9 | 3.07 | 3.09 | 3.02 | 0.08 |
| 2017 | Y839H | 839 | 2.72 | 2.39 | 2.54 | 2.55 | 0.14 |
| 2018 | Y839I | 839 | 2.47 | 2.31 | 2.69 | 2.49 | 0.15 |
| 2019 | Y839T | 839 | 2.38 | 2.25 | 2.51 | 2.38 | 0.11 |
| 2020 | Y839Q | 839 | 2.34 | 2.25 | 2.46 | 2.35 | 0.09 |
| 2021 | Y839V | 839 | 2.1 | 2.35 | 2.31 | 2.25 | 0.11 |
| 2022 | Y839M | 839 | 2.11 | 2.12 | 2.31 | 2.18 | 0.09 |
| 2023 | Y839K | 839 | 2.16 | 2.04 | 2.23 | 2.15 | 0.08 |
| 2024 | Y839C | 839 | 1.75 | 2.16 | 2.02 | 1.98 | 0.17 |
| 2025 | Y839R | 839 | 2.1 | 1.79 | 1.93 | 1.94 | 0.13 |
| 2026 | Y839N | 839 | 1.81 | 1.83 | 2.07 | 1.91 | 0.12 |
| 2027 | Y839A | 839 | 0.73 | 0.93 | 1.42 | 1.03 | 0.29 |
| 2028 | Y839S | 839 | 0.58 | 0.82 | 0.72 | 0.71 | 0.1 |
| 2029 | L841I | 841 | 1.5 | 1.85 | 1.53 | 1.63 | 0.15 |
| 2030 | L841V | 841 | 0.91 | 0.89 | 1 | 0.94 | 0.05 |
| 2031 | L841C | 84 | 0.78 | 0.87 | 1.09 | 0.91 | 0.13 |
| 2032 | L841G | 841 | 0.5 | 1 | 0.75 | 0.75 | 0.2 |
| 2033 | L841Y | 841 | 0.47 | 0.53 | 0.62 | 0.54 | 0.06 |
| 2034 | M843F | 843 | 1.65 | 1.94 | 2.27 | 1.95 | 0.25 |
| 2035 | M843Q | 843 | 1.56 | 1.34 | 1.64 | 1.51 | 0.13 |
| 2036 | P844C | 844 | 0.14 | 0.93 | 1.37 | 0.82 | 0.51 |
| 2037 | I845Q | 845 | 1.75 | 0.24 | −0.01 | 0.66 | 0.78 |
| 2038 | T846G | 846 | 1.53 | 1.34 | 1.69 | 1.52 | 0.15 |
| 2039 | T846Q | 846 | 1.4 | 0.93 | 1.6 | 1.31 | 0.28 |
| 2040 | T846Y | 846 | 0.84 | 0.71 | 0.32 | 0.62 | 0.22 |
| 2041 | T846L | 846 | 0.59 | 0.63 | 0.44 | 0.55 | 0.08 |
| 2042 | T846W | 846 | 0.5 | 0.69 | 0.12 | 0.44 | 0.24 |
| 2043 | N848D | 848 | 1.06 | 0.93 | 0.91 | 0.97 | 0.07 |
| 2044 | F849R | 849 | 0.41 | 0.71 | 1.02 | 0.71 | 0.25 |
| 2045 | A851T | 851 | 1.06 | 0.5 | 0.68 | 0.75 | 0.23 |
| 2046 | A851N | 851 | 0.14 | 0.93 | 0.68 | 0.59 | 0.33 |
| 2047 | I857A | 857 | 0.66 | 0.24 | 0.5 | 0.46 | 0.17 |
| 2048 | I857N | 857 | 0.14 | 0.75 | 0.5 | 0.46 | 0.25 |
| 2049 | D859P | 859 | 0.43 | 1.22 | 1.38 | 1.01 | 0.41 |
| 2050 | R860E | 860 | 0.15 | 0.46 | 1.44 | 0.68 | 0.55 |
| 2051 | R860K | 860 | 0.84 | 0.73 | 0.21 | 0.59 | 0.27 |
| 2052 | I861A | 861 | 0.44 | 0.49 | 0.56 | 0.5 | 0.05 |
| 2053 | L862Y | 862 | 1.37 | 1.72 | 1.08 | 1.39 | 0.26 |
| 2054 | L862D | 862 | 0.7 | 0.46 | 0.9 | 0.69 | 0.18 |
| 2055 | L862H | 862 | 0.8 | 0.69 | 0.12 | 0.53 | 0.3 |
| 2056 | A866D | 866 | 1.44 | 0.93 | 1.37 | 1.25 | 0.23 |
| 2057 | A866Y | 866 | 0.6 | 0.39 | 0.63 | 0.54 | 0.11 |
| 2058 | K869C | 869 | 0.54 | 0.79 | 0.79 | 0.71 | 0.12 |
| 2059 | H872S | 872 | 1.05 | 0.3 | 0.2 | 0.51 | 0.38 |
| 2060 | H872M | 872 | 0.91 | 0.52 | −0.02 | 0.47 | 0.38 |
| 2061 | I876L | 876 | 0.71 | 0.67 | 0.63 | 0.67 | 0.03 |
| 2062 | V886S | 886 | 0.88 | 0.97 | 0.89 | 0.91 | 0.04 |
| 2063 | V886A | 886 | 0.57 | 0.58 | 0.56 | 0.57 | 0.01 |
| 2064 | V886G | 886 | 0.56 | 0.63 | 0.48 | 0.56 | 0.06 |
| 2065 | T891I | 891 | 0.31 | 0.82 | 0.4 | 0.51 | 0.22 |
| 2066 | C892T | 892 | 1.35 | 1.22 | 1.45 | 1.34 | 0.09 |
| 2067 | C892I | 892 | 1.06 | 0.93 | 0.54 | 0.85 | 0.22 |
| 2068 | C892D | 892 | 0.84 | 2.19 | −0.71 | 0.77 | 1.18 |
| 2069 | C892H | 892 | 0.15 | 1.09 | 0.83 | 0.69 | 0.4 |
| 2070 | G893K | 893 | 0.99 | 0.24 | 0.27 | 0.5 | 0.35 |
| 2071 | N894V | 894 | 0.42 | 1.06 | 0.91 | 0.8 | 0.28 |
| 2072 | E897Y | 897 | 0.53 | 0.48 | 1 | 0.67 | 0.23 |
| 2073 | Q898G | 898 | 0.84 | 0.93 | −0.02 | 0.58 | 0.43 |
| 2074 | F901M | 901 | 2.43 | 2.67 | 2.59 | 2.56 | 0.1 |
| 2075 | F901Q | 901 | 2.09 | 2.12 | 1.9 | 2.04 | 0.1 |
| 2076 | F901L | 901 | 0.61 | 0.71 | 0.75 | 0.69 | 0.06 |
| 2077 | F901G | 901 | 0.48 | 0.82 | 0.62 | 0.64 | 0.14 |
| 2078 | I903V | 903 | 0.36 | 0.43 | 0.47 | 0.42 | 0.05 |
| 2079 | Y907I | 907 | 3.11 | 3.15 | 3.13 | 3.13 | 0.02 |
| 2080 | Y907T | 907 | 1.74 | 1.42 | 1.12 | 1.43 | 0.25 |
| 2081 | Y907M | 907 | 0.84 | 1.1 | 1.8 | 1.25 | 0.41 |
| 2082 | Y907V | 907 | 0.9 | 0.8 | 1.15 | 0.95 | 0.15 |
| 2083 | Y909F | 909 | 1.11 | 1.01 | 1.31 | 1.14 | 0.12 |
| 2084 | Y909W | 909 | 1.02 | 1 | 1.13 | 1.05 | 0.06 |
| 2085 | Q910A | 910 | 0.83 | 0.46 | 1.35 | 0.88 | 0.37 |
| 2086 | Q910N | 910 | 1.24 | 0.57 | 0.56 | 0.79 | 0.32 |
| 2087 | Q910Y | 910 | 0.93 | 0.77 | 0.06 | 0.59 | 0.38 |
| 2088 | Q910S | 910 | 0.65 | 0.84 | 0.12 | 0.54 | 0.3 |
| 2089 | I911Q | 911 | 1.29 | 1.66 | 1.54 | 1.5 | 0.15 |
| 2090 | I911E | 911 | 1.41 | 1.31 | 1.59 | 1.44 | 0.12 |
| 2091 | I911A | 911 | 1.24 | 1.25 | 1.32 | 1.27 | 0.03 |
| 2092 | I911H | 911 | 0.95 | 0.95 | 0.83 | 0.91 | 0.06 |
| 2093 | I911D | 911 | 0.53 | 0.9 | 1 | 0.81 | 0.2 |
| 2094 | I911Y | 911 | 0.7 | 0.89 | 0.59 | 0.73 | 0.12 |
| 2095 | I911G | 911 | 0.65 | 0.49 | 0.54 | 0.56 | 0.07 |
| 2096 | I911W | 911 | 0.5 | 0.4 | 0.66 | 0.52 | 0.11 |
| 2097 | K912H | 912 | 1.08 | 0.93 | 0.69 | 0.9 | 0.16 |
| 2098 | K912Y | 912 | 0.79 | 0.95 | 0.51 | 0.75 | 0.18 |
| 2099 | K912C | 912 | 1.03 | −0.73 | 1.04 | 0.45 | 0.83 |
| 2100 | K914A | 914 | 1.64 | 1.3 | 1.54 | 1.49 | 0.14 |
| 2101 | K914S | 914 | 1.11 | 0.97 | 1.01 | 1.03 | 0.06 |
| 2102 | K914Q | 914 | 0.75 | 0.88 | 0.97 | 0.86 | 0.09 |
| 2103 | K914F | 914 | 0.89 | 0.73 | 0.87 | 0.83 | 0.07 |
| 2104 | K914T | 914 | 0.74 | 0.65 | 0.63 | 0.67 | 0.05 |
| 2105 | K914M | 914 | 0.7 | 0.54 | 0.68 | 0.64 | 0.07 |
| 2106 | K914C | 914 | 0.68 | 0.39 | 0.64 | 0.57 | 0.13 |
| 2107 | Q916Y | 916 | 1.04 | 1.29 | 1.02 | 1.12 | 0.12 |
| 2108 | Q916M | 916 | 0.77 | 1.08 | 1.16 | 1 | 0.17 |
| 2109 | Q916D | 916 | 1.06 | 0.5 | 1.13 | 0.89 | 0.28 |
| 2110 | Q916V | 916 | 0.82 | 0.62 | 1.05 | 0.83 | 0.18 |
| 2111 | Q916L | 916 | 0.62 | 0.79 | 0.7 | 0.7 | 0.07 |
| 2112 | Q916A | 916 | 0.65 | 0.85 | 0.6 | 0.7 | 0.11 |
| 2113 | Q916N | 916 | 0.87 | 0.15 | 0.73 | 0.59 | 0.31 |
| 2114 | Q916W | 916 | 0.45 | 0.76 | 0.51 | 0.58 | 0.14 |
| 2115 | G918E | 918 | 1.98 | 1.96 | 2.03 | 1.99 | 0.03 |
| 2116 | G918M | 918 | 1.82 | 1.68 | 1.66 | 1.72 | 0.07 |
| 2117 | G918Q | 918 | 1.29 | 1.44 | 1.38 | 1.37 | 0.06 |
| 2118 | G918L | 918 | 1.08 | 1.32 | 1.45 | 1.28 | 0.15 |
| 2119 | G918I | 918 | 1.16 | 1.37 | 1.3 | 1.28 | 0.09 |
| 2120 | G918T | 918 | 0.76 | 0.89 | 0.89 | 0.85 | 0.06 |
| 2121 | G918Y | 918 | 0.58 | 0.91 | 0.62 | 0.71 | 0.14 |
| 2122 | G918F | 918 | 0.68 | 0.63 | 0.61 | 0.64 | 0.03 |
| 2123 | G918H | 918 | 0.4 | 0.92 | 0.6 | 0.64 | 0.21 |
| 2124 | G918K | 918 | 0.62 | 0.52 | 0.72 | 0.62 | 0.08 |
| 2125 | G918D | 918 | 0.48 | 0.42 | 0.48 | 0.46 | 0.03 |
| 2126 | A919I | 919 | 1.99 | 2.11 | 1.95 | 2.02 | 0.07 |
| 2127 | A919E | 919 | 1.36 | 0.59 | 1.19 | 1.05 | 0.33 |
| 2128 | A919F | 919 | 0.83 | 0.89 | 0.92 | 0.88 | 0.04 |
| 2129 | A919L | 919 | 0.84 | 0.78 | 0.83 | 0.82 | 0.03 |
| 2130 | A919Y | 919 | 0.28 | 1.14 | 0.76 | 0.73 | 0.35 |
| 2131 | A919M | 919 | 0.51 | 0.57 | 0.33 | 0.47 | 0.1 |
| 2132 | Q921A | 921 | 0.83 | 0.55 | 0.47 | 0.62 | 0.15 |
| 2133 | Q921T | 921 | 0.74 | 0.23 | 0.65 | 0.54 | 0.22 |
| 2134 | I922A | 922 | 0.71 | 0.55 | 0.76 | 0.67 | 0.09 |
| 2135 | I922R | 922 | 0.55 | 0.73 | 0.58 | 0.62 | 0.08 |
| 2136 | I922Y | 922 | 0.46 | 0.63 | 0.57 | 0.55 | 0.07 |
| 2137 | I922W | 922 | 0.38 | 0.68 | 0.49 | 0.51 | 0.13 |
| 2138 | I922C | 922 | 0.34 | 0.64 | 0.37 | 0.45 | 0.14 |
| 2139 | I922H | 922 | 0.39 | 0.47 | 0.44 | 0.43 | 0.03 |
| 2140 | K925C | 925 | 0.82 | 0.39 | 0.65 | 0.62 | 0.18 |
| 2141 | E926S | 926 | 1.06 | 1.15 | 1.18 | 1.13 | 0.05 |
| 2142 | E926T | 926 | 0.97 | 0.99 | 0.98 | 0.98 | 0.01 |
| 2143 | E926N | 926 | 0.44 | 0.48 | 0.36 | 0.43 | 0.05 |
| 2144 | K928W | 928 | 0.78 | 0.7 | 0.67 | 0.72 | 0.05 |
| 2145 | E929C | 929 | 1.05 | 0.92 | 1.21 | 1.06 | 0.12 |
| 2146 | E929G | 929 | 0.74 | 0.67 | 0.8 | 0.74 | 0.05 |
| 2147 | E929S | 929 | 0.42 | 0.52 | 0.61 | 0.52 | 0.07 |
| 2148 | E929P | 929 | 0.41 | 0.5 | 0.62 | 0.51 | 0.09 |
| 2149 | E929N | 929 | 0.57 | 0.31 | 0.61 | 0.5 | 0.13 |
| 2150 | G931Q | 931 | 0.54 | 0.64 | 0.98 | 0.72 | 0.19 |
| 2151 | G931H | 931 | 0.25 | 0.62 | 0.43 | 0.43 | 0.15 |
| 2152 | K934S | 934 | 0.64 | 0.38 | 0.6 | 0.54 | 0.12 |
| 2153 | E935R | 935 | 2.72 | 2.95 | 3.05 | 2.91 | 0.14 |
| 2154 | E935M | 935 | 2.9 | 2.77 | 2.97 | 2.88 | 0.08 |
| 2155 | E935N | 935 | 2.75 | 2.91 | 2.8 | 2.82 | 0.07 |
| 2156 | E935A | 935 | 2.43 | 2.62 | 2.7 | 2.58 | 0.11 |
| 2157 | E935Q | 935 | 2.21 | 2.5 | 2.62 | 2.44 | 0.17 |
| 2158 | E935K | 935 | 2.25 | 2.53 | 2.53 | 2.43 | 0.13 |
| 2159 | E935L | 935 | 1.91 | 2.07 | 2.1 | 2.03 | 0.08 |
| 2160 | E935S | 935 | 1.75 | 2.08 | 1.96 | 1.93 | 0.14 |
| 2161 | E935Y | 935 | 1.93 | 1.85 | 1.75 | 1.84 | 0.07 |
| 2162 | E935H | 935 | 1.73 | 2.03 | 1.76 | 1.84 | 0.14 |
| 2163 | E935C | 935 | 1.39 | 1.55 | 1.56 | 1.5 | 0.08 |
| 2164 | E935G | 935 | 1.1 | 1.58 | 1.35 | 1.34 | 0.2 |
| 2165 | E935F | 935 | 1.18 | 1.5 | 1.34 | 1.34 | 0.13 |
| 2166 | E935T | 935 | 1.2 | 1.36 | 1.46 | 1.34 | 0.11 |
| 2167 | E935W | 935 | 0.78 | 1.29 | 1.5 | 1.19 | 0.3 |
| 2168 | I936T | 936 | 2.76 | 2.88 | 2.96 | 2.86 | 0.08 |
| 2169 | I936C | 936 | 2.11 | 1.85 | 1.91 | 1.96 | 0.11 |
| 2170 | I936A | 936 | 1.94 | 1.92 | 1.99 | 1.95 | 0.03 |
| 2171 | I936R | 936 | 1.72 | 1.75 | 1.81 | 1.76 | 0.04 |
| 2172 | I936K | 936 | 1.57 | 1.63 | 1.66 | 1.62 | 0.03 |
| 2173 | I936S | 936 | 1.38 | 1.37 | 1.44 | 1.4 | 0.03 |
| 2174 | I936Q | 936 | 1.16 | 1.48 | 1.29 | 1.31 | 0.13 |
| 2175 | I936Y | 936 | 1.17 | 1.18 | 1.25 | 1.2 | 0.03 |
| 2176 | I936F | 936 | 0.99 | 0.83 | 0.99 | 0.94 | 0.08 |
| 2177 | I936L | 936 | 0.99 | 0.85 | 0.92 | 0.92 | 0.06 |
| 2178 | I936G | 936 | 0.59 | 0.11 | 0.56 | 0.42 | 0.22 |
| 2179 | L941C | 941 | 1.77 | 1.64 | 1.64 | 1.68 | 0.06 |
| 2180 | L941N | 941 | 1.06 | 0.63 | 0.65 | 0.78 | 0.2 |
| 2181 | S942A | 942 | 1.93 | 2.14 | 1.99 | 2.02 | 0.09 |
| 2182 | L943I | 943 | 1.24 | 2.17 | 1.9 | 1.77 | 0.39 |
| 2183 | V944C | 944 | 0.63 | 0.58 | 0.6 | 0.6 | 0.02 |
| 2184 | I945M | 945 | 0.99 | 1.24 | 1.18 | 1.14 | 0.11 |
| 2185 | H946W | 946 | 1.36 | 1.51 | 1.36 | 1.41 | 0.07 |
| 2186 | E947P | 947 | 1.57 | 1.71 | 1.88 | 1.72 | 0.13 |
| 2187 | E947F | 947 | 0.49 | 0.47 | 0.74 | 0.56 | 0.13 |
| 2188 | E947M | 947 | 0.35 | 0.35 | 0.65 | 0.45 | 0.14 |
| 2189 | I948L | 948 | 1.03 | 0.84 | 0.77 | 0.88 | 0.11 |
| 2190 | S949M | 949 | 1.72 | 1.74 | 1.84 | 1.77 | 0.05 |
| 2191 | S949V | 949 | 1.54 | 1.69 | 1.58 | 1.6 | 0.06 |
| 2192 | S949T | 949 | 1.08 | 1.32 | 1.28 | 1.23 | 0.11 |
| 2193 | S949Q | 949 | 1.43 | 1.02 | 1.12 | 1.19 | 0.18 |
| 2194 | S949I | 949 | 0.9 | 0.66 | 1.02 | 0.86 | 0.15 |
| 2195 | S949K | 949 | 0.47 | 0.34 | 0.65 | 0.49 | 0.12 |
| 2196 | M951G | 951 | −0.15 | 0.79 | 0.77 | 0.47 | 0.44 |
| 2197 | V952E | 952 | 0.79 | 0.38 | 0.73 | 0.63 | 0.18 |
| 2198 | V952W | 952 | 0.78 | 0.41 | 0.6 | 0.6 | 0.15 |
| 2199 | I953A | 953 | 0.62 | 0.47 | 0.82 | 0.63 | 0.14 |
| 2200 | I953P | 953 | 0.68 | 0.51 | 0.52 | 0.57 | 0.08 |
| 2201 | K954A | 954 | 0.7 | 1.33 | 1.71 | 1.25 | 0.42 |
| 2202 | K954P | 954 | 1.13 | 0.84 | 0.66 | 0.88 | 0.2 |
| 2203 | Y955W | 955 | 0.93 | 1.26 | 1.65 | 1.28 | 0.29 |
| 2204 | Y955C | 955 | −0.55 | 1.15 | 1.22 | 0.61 | 0.82 |
| 2205 | N956C | 956 | 2.28 | 1.48 | 1.22 | 1.66 | 0.45 |
| 2206 | N956W | 956 | 1.46 | 1.04 | 1.47 | 1.32 | 0.2 |
| 2207 | N956F | 956 | 1.06 | 1.3 | 0.92 | 1.09 | 0.16 |
| 2208 | N956Y | 956 | 0.87 | 0.72 | 0.63 | 0.74 | 0.1 |
| 2209 | A957P | 957 | 1.02 | 1.17 | 1.14 | 1.11 | 0.06 |
| 2210 | A957F | 957 | 1.53 | 0.52 | 0.81 | 0.95 | 0.42 |
| 2211 | A957T | 957 | 0.32 | 0.93 | 0.45 | 0.56 | 0.26 |
| 2212 | I958V | 958 | 0.83 | 0.71 | 0.4 | 0.65 | 0.18 |
| 2213 | I959F | 959 | 1.49 | 1.01 | 0.95 | 1.15 | 0.24 |
| 2214 | D963C | 963 | 1.39 | −0.46 | 0.37 | 0.44 | 0.76 |
| 2215 | F968W | 968 | 0.29 | 0.56 | 0.63 | 0.49 | 0.15 |
| 2216 | K970C | 970 | 0.43 | 0.68 | 0.26 | 0.46 | 0.17 |
| 2217 | K974G | 974 | 3.37 | 3.23 | 3.31 | 3.31 | 0.06 |
| 2218 | K974P | 974 | 2.45 | 2.32 | 2.38 | 2.38 | 0.06 |
| 2219 | K974W | 974 | 1.25 | 2.18 | 1.77 | 1.74 | 0.38 |
| 2220 | K974A | 974 | 1.12 | 1.29 | 1.62 | 1.34 | 0.21 |
| 2221 | K974T | 974 | 1.29 | 1.44 | 0.92 | 1.22 | 0.22 |
| 2222 | K974Q | 974 | 0.71 | 1.22 | 1.08 | 1 | 0.21 |
| 2223 | K974Y | 974 | 0.99 | 0.56 | 1.44 | 1 | 0.36 |
| 2224 | K974C | 974 | 1.16 | 0.46 | 1.23 | 0.95 | 0.35 |
| 2225 | K974S | 974 | 0.95 | 0.69 | 0.67 | 0.77 | 0.12 |
| 2226 | K974M | 974 | 0.5 | 0.99 | −0.08 | 0.47 | 0.44 |
| 2227 | K974E | 974 | 0.64 | −0.23 | 0.95 | 0.45 | 0.5 |
| 2228 | V975I | 975 | 0.66 | 0.6 | 0.69 | 0.65 | 0.04 |
| 2229 | R977H | 977 | 0.59 | 0.54 | 0.28 | 0.47 | 0.14 |
| 2230 | K982Y | 982 | 0.78 | 1.03 | 0.37 | 0.73 | 0.27 |
| 2231 | K982S | 982 | 0.56 | 0.69 | 0.75 | 0.67 | 0.08 |
| 2232 | F983A | 983 | 0.76 | 0.4 | 0.71 | 0.62 | 0.16 |
| 2233 | M986G | 986 | 0.51 | 0.56 | 0.22 | 0.43 | 0.15 |
| 2234 | I988K | 988 | 1.54 | 1.35 | 1.94 | 1.61 | 0.25 |
| 2235 | I988E | 988 | 0.85 | 1.63 | 0.68 | 1.06 | 0.41 |
| 2236 | L991W | 991 | 1.25 | 1.34 | 1.93 | 1.51 | 0.3 |
| 2237 | L991C | 991 | 1.54 | 0.93 | 1.37 | 1.28 | 0.26 |
| 2238 | N992L | 992 | 1.41 | 0.93 | −0.02 | 0.78 | 0.59 |
| 2239 | N992A | 992 | 1.25 | 0.24 | −0.02 | 0.49 | 0.55 |
| 2240 | L994A | 994 | 1.77 | 1.63 | −0.01 | 1.13 | 0.81 |
| 2241 | V995P | 995 | 0.85 | 0.65 | 0.39 | 0.63 | 0.19 |
| 2242 | F996T | 996 | 0.85 | 0.24 | 0.68 | 0.59 | 0.26 |
| 2243 | F996V | 996 | 0.85 | 0.93 | −0.01 | 0.59 | 0.43 |
| 2244 | D998T | 998 | 1.91 | 1.74 | 1.6 | 1.75 | 0.13 |
| 2245 | D998W | 998 | 0.49 | 0.02 | 0.86 | 0.46 | 0.35 |
| 2246 | I999P | 999 | 0.85 | 2.19 | 1.59 | 1.54 | 0.55 |
| 2247 | I999H | 999 | 0.16 | 0.65 | 1.08 | 0.63 | 0.38 |
| 2248 | I1001W | 1001 | 0.85 | 0.24 | 0.68 | 0.59 | 0.26 |
| 2249 | T1002V | 1002 | 1.26 | 0.94 | −0.02 | 0.73 | 0.54 |
| 2250 | T1002C | 1002 | 1.07 | −0.45 | 0.9 | 0.51 | 0.68 |
| 2251 | T1002L | 1002 | 0.35 | 0.44 | 0.71 | 0.5 | 0.15 |
| 2252 | T1002S | 1002 | 0.74 | 0.25 | 0.39 | 0.46 | 0.21 |
| 2253 | E1003S | 1003 | 1.62 | 1.09 | 0.97 | 1.23 | 0.29 |
| 2254 | E1003M | 1003 | 0.16 | 0.58 | 1.21 | 0.65 | 0.43 |
| 2255 | N1004A | 1004 | 2.17 | 1.85 | −0.71 | 1.11 | 1.29 |
| 2256 | N1004G | 1004 | 0.85 | 0.65 | 1.08 | 0.86 | 0.18 |
| 2257 | N1004R | 1004 | 0.67 | 0.85 | 0.44 | 0.65 | 0.17 |
| 2258 | N1004P | 1004 | 0.63 | −0.27 | 1.21 | 0.52 | 0.61 |
| 2259 | N1004Q | 1004 | 0.34 | 0.94 | 0.17 | 0.48 | 0.33 |
| 2260 | L1007V | 1007 | 0.5 | 0.72 | 0.48 | 0.57 | 0.11 |
| 2261 | L1007C | 1007 | 0.38 | 0.69 | 0.5 | 0.53 | 0.13 |
| 2262 | L1007A | 1007 | 0.41 | 0.65 | 0.39 | 0.49 | 0.12 |
| 2263 | L1008F | 1008 | 0.86 | 0.47 | 1.08 | 0.81 | 0.25 |
| 2264 | Y1011V | 1011 | 0.64 | 1.12 | 1.21 | 0.99 | 0.25 |
| 2265 | Y1011L | 1011 | 0.84 | 0.94 | 0.63 | 0.8 | 0.13 |
| 2266 | Y1011I | 1011 | 0.3 | 1.01 | 0.81 | 0.71 | 0.3 |
| 2267 | T1014C | 1014 | 0.54 | 1.15 | 0.77 | 0.82 | 0.25 |
| 2268 | Y1015M | 1015 | 1.1 | 1.28 | 1.29 | 1.22 | 0.09 |
| 2269 | Y1015A | 1015 | 0.9 | 1.19 | 1.14 | 1.08 | 0.13 |
| 2270 | Y1015E | 1015 | 0.99 | 0.82 | 1.06 | 0.96 | 0.1 |
| 2271 | Y1015Q | 1015 | 0.63 | 0.97 | 0.98 | 0.86 | 0.16 |
| 2272 | Y1015G | 1015 | 0.7 | 0.66 | 0.56 | 0.64 | 0.06 |
| 2273 | Y1015T | 1015 | 0.45 | 0.5 | 0.45 | 0.47 | 0.02 |
| 2274 | I1016E | 1016 | 0.96 | 0.94 | 0.92 | 0.94 | 0.02 |
| 2275 | I1016N | 1016 | 1.09 | 0.8 | 0.71 | 0.87 | 0.16 |
| 2276 | I1016A | 1016 | 0.51 | 0.74 | 0.78 | 0.67 | 0.12 |
| 2277 | I1016P | 1016 | 0.43 | 0.56 | 0.5 | 0.5 | 0.05 |
| 2278 | P1017W | 1017 | 1.42 | 0.66 | 0.79 | 0.95 | 0.33 |
| 2279 | P1017G | 1017 | 0.92 | 1.35 | 0.46 | 0.91 | 0.36 |
| 2280 | P1017N | 1017 | 1.16 | 1.25 | 0.23 | 0.88 | 0.46 |
| 2281 | P1017E | 1017 | 1.37 | 0.4 | 0.27 | 0.68 | 0.49 |
| 2282 | P1017D | 1017 | 0.3 | 0.7 | 0.86 | 0.62 | 0.24 |
| 2283 | P1017Q | 1017 | 0.91 | 0.35 | 0.44 | 0.56 | 0.25 |
| 2284 | K1019A | 1019 | 0.86 | 0.92 | 1.06 | 0.95 | 0.09 |
| 2285 | K1019G | 1019 | 0.87 | 0.4 | 0.8 | 0.69 | 0.21 |
| 2286 | K1019V | 1019 | 0.56 | 0.62 | 0.5 | 0.56 | 0.05 |
| 2287 | K1019S | 1019 | 0.45 | 0.51 | 0.62 | 0.53 | 0.07 |
| 2288 | K1019C | 1019 | 0.17 | 0.86 | 0.23 | 0.42 | 0.31 |
| 2289 | K1021A | 1021 | 1.26 | 1.2 | 1.01 | 1.16 | 0.11 |
| 2290 | K1021V | 1021 | 0.86 | 1.11 | 0.67 | 0.88 | 0.18 |
| 2291 | K1021F | 1021 | 0.42 | 0.78 | 0.43 | 0.54 | 0.17 |
| 2292 | K1021W | 1021 | 0.68 | 0.06 | 0.75 | 0.5 | 0.31 |
| 2293 | V1023N | 1023 | 1.01 | 0.76 | 0.49 | 0.75 | 0.22 |
| 2294 | V1023C | 1023 | 0.33 | 0.8 | 0.57 | 0.57 | 0.19 |
| 2295 | G1024Y | 1024 | 0.2 | 0.7 | 0.67 | 0.52 | 0.23 |
| 2296 | Q1026M | 1026 | 1.26 | 0.9 | 1.19 | 1.12 | 0.16 |
| 2297 | Q1026N | 1026 | 0.86 | 0.65 | 0.81 | 0.77 | 0.09 |
| 2298 | C1027N | 1027 | 0.58 | 0.64 | 0.65 | 0.62 | 0.03 |
| 2299 | C1029N | 1029 | 0.4 | 0.54 | 0.6 | 0.51 | 0.09 |
| 2300 | Y1032Q | 1032 | 0.84 | 0.84 | 0.73 | 0.8 | 0.05 |
| 2301 | Y1032R | 1032 | 0.86 | 0.72 | 0.7 | 0.76 | 0.07 |
| 2302 | Y1032K | 1032 | 0.83 | 0.6 | 0.82 | 0.75 | 0.11 |
| 2303 | Y1032W | 1032 | 0.64 | 0.14 | 0.67 | 0.49 | 0.24 |
| 2304 | A1036H | 1036 | 0.93 | 0.65 | 0.55 | 0.71 | 0.16 |
| 2305 | A1036F | 1036 | 0.79 | 0.71 | 0.52 | 0.67 | 0.11 |
| 2306 | K1040A | 1040 | 2.24 | 0.93 | 2.37 | 1.85 | 0.65 |
| 2307 | K1040T | 1040 | 1.01 | 1.91 | −0.44 | 0.83 | 0.97 |
| 2308 | K1040I | 1040 | 1.26 | 0.23 | 0.66 | 0.72 | 0.42 |
| 2309 | I1050D | 1050 | 1.08 | 0.98 | 1.06 | 1.04 | 0.04 |
| 2310 | I1050W | 1050 | 0.65 | 0.69 | 0.54 | 0.63 | 0.06 |
| 2311 | I1050H | 1050 | 0.5 | 0.87 | 0.31 | 0.56 | 0.23 |
| 2312 | K1052W | 1052 | 0.76 | 1.25 | 0.94 | 0.98 | 0.2 |
| 2313 | K1052C | 1052 | 0.45 | 0.7 | 1.08 | 0.74 | 0.26 |
| 2314 | K1052G | 1052 | 0.44 | 0.27 | 0.56 | 0.42 | 0.12 |
| 2315 | K1052I | 1052 | 0.01 | 0.55 | 0.71 | 0.42 | 0.3 |
| 2316 | F1053Y | 1053 | −0.04 | 1.45 | −0.02 | 0.46 | 0.7 |
| 2317 | K1054L | 1054 | 1.1 | 1.21 | 1.17 | 1.16 | 0.05 |
| 2318 | K1054I | 1054 | 1.1 | 1.12 | 1 | 1.07 | 0.05 |
| 2319 | K1054W | 1054 | 0.73 | 1.15 | 0.76 | 0.88 | 0.19 |
| 2320 | K1054V | 1054 | 0.88 | 0.72 | 0.97 | 0.86 | 0.1 |
| 2321 | K1054F | 1054 | 0.94 | 0.52 | 0.73 | 0.73 | 0.17 |
| 2322 | K1054M | 1054 | 0.62 | 0.53 | 0.58 | 0.58 | 0.04 |
| 2323 | D1055I | 1055 | 1.78 | 1.49 | 1.85 | 1.71 | 0.15 |
| 2324 | D1055M | 1055 | 1.4 | 1.39 | 1.08 | 1.29 | 0.15 |
| 2325 | D1055F | 1055 | 0.93 | 1.63 | 1.21 | 1.26 | 0.29 |
| 2326 | D1055T | 1055 | 0.96 | 1.07 | 0.89 | 0.97 | 0.07 |
| 2327 | D1055P | 1055 | 0.57 | 1.22 | 0.99 | 0.93 | 0.27 |
| 2328 | D1055C | 1055 | 0.66 | 0.73 | 0.88 | 0.76 | 0.09 |
| 2329 | D1055L | 1055 | 0.3 | 0.86 | 0.98 | 0.71 | 0.3 |
| 2330 | D1055V | 1055 | 0.41 | 0.62 | 0.46 | 0.5 | 0.09 |
| 2331 | D1055Q | 1055 | 0.27 | 0.53 | 0.51 | 0.44 | 0.12 |
| 2332 | L1056E | 1056 | 0.86 | 0.94 | 0.9 | 0.9 | 0.03 |
| 2333 | L1056C | 1056 | 0.17 | 0.43 | 0.94 | 0.51 | 0.32 |
| 2334 | L1056I | 1056 | 0.24 | 0.5 | 0.78 | 0.5 | 0.22 |
| 2335 | T1057Y | 1057 | 0.57 | 0.65 | 0.67 | 0.63 | 0.04 |
| 2336 | V1058W | 1058 | 1.96 | 1.35 | 1.37 | 1.56 | 0.28 |
| 2337 | V1058H | 1058 | 0.62 | 0.25 | 0.92 | 0.6 | 0.28 |
| 2338 | A1060N | 1060 | 1.71 | 1.35 | 0.27 | 1.11 | 0.61 |
| 2339 | A1060Q | 1060 | 0.86 | 0.54 | 0.27 | 0.56 | 0.24 |
| 2340 | K1061W | 1061 | 1.96 | 2.04 | 2.28 | 2.1 | 0.14 |
| 2341 | K1061F | 1061 | 1.78 | 1.35 | 1.08 | 1.4 | 0.29 |
| 2342 | K1061D | 1061 | 0.86 | 0.76 | 1.08 | 0.9 | 0.13 |
| 2343 | K1061E | 1061 | 0.78 | 0.76 | 1.13 | 0.89 | 0.17 |
| 2344 | K1061C | 1061 | 1.09 | 1.17 | 0.39 | 0.88 | 0.35 |
| 2345 | K1061S | 1061 | 0.46 | 0.94 | 1.13 | 0.84 | 0.28 |
| 2346 | K1061L | 1061 | 0.57 | 0.61 | 0.52 | 0.57 | 0.04 |
| 2347 | K1061R | 1061 | 0.96 | 0.43 | 0.11 | 0.5 | 0.35 |
| 2348 | K1061I | 1061 | 0.54 | 0.69 | 0.18 | 0.47 | 0.21 |
| 2349 | K1061Q | 1061 | 0.86 | 0.54 | −0.02 | 0.46 | 0.36 |
| 2350 | K1061T | 1061 | 0.51 | 0.86 | −0.12 | 0.42 | 0.41 |
| 2351 | R1062D | 1062 | 2.47 | 1.35 | 1.08 | 1.64 | 0.6 |
| 2352 | R1062A | 1062 | 1.81 | 1.3 | 1.03 | 1.38 | 0.33 |
| 2353 | R1062M | 1062 | 1.02 | 1.64 | 0.27 | 0.98 | 0.56 |
| 2354 | R1062Y | 1062 | 1.47 | 0.77 | 0.68 | 0.97 | 0.36 |
| 2355 | R1062H | 1062 | 0.35 | 0.95 | 1.08 | 0.8 | 0.32 |
| 2356 | R1062V | 1062 | 0.87 | 1.48 | −0.24 | 0.7 | 0.71 |
| 2357 | E1063W | 1063 | 0.31 | 0.51 | 1.04 | 0.62 | 0.31 |
| 2358 | E1063F | 1063 | 0.79 | 0.09 | 0.42 | 0.43 | 0.29 |
| 2359 | E1063G | 1063 | 0.67 | −0.19 | 0.78 | 0.42 | 0.43 |
| 2360 | I1065W | 1065 | 0.26 | 0.63 | 0.45 | 0.45 | 0.15 |
| 2361 | F1068N | 1068 | 0.08 | 0.95 | 0.36 | 0.47 | 0.36 |
| 2362 | D1069M | 1069 | 0.4 | 0.66 | 1.38 | 0.81 | 0.41 |
| 2363 | S1070E | 1070 | 0.4 | 0.53 | 1.09 | 0.67 | 0.3 |
| 2364 | R1072E | 1072 | −0.51 | 1.51 | 0.4 | 0.47 | 0.83 |
| 2365 | R1072D | 1072 | −0.51 | 1.17 | 0.69 | 0.45 | 0.71 |
| 2366 | D1074S | 1074 | 1.25 | 1.02 | 0.9 | 1.06 | 0.15 |
| 2367 | D1074R | 1074 | 0.4 | 0.95 | 0.91 | 0.75 | 0.25 |
| 2368 | S1075V | 1075 | 0.52 | 0.36 | 0.53 | 0.47 | 0.08 |
| 2369 | E1076T | 1076 | 0.89 | 0.22 | 0.43 | 0.51 | 0.28 |
| 2370 | K1077R | 1077 | 0.72 | 0.76 | 0.79 | 0.76 | 0.03 |
| 2371 | C1081A | 1081 | 0.19 | 0.67 | 1.25 | 0.7 | 0.44 |
| 2372 | C1081I | 1081 | 0.7 | 0.77 | 0.29 | 0.59 | 0.21 |
| 2373 | T1083E | 1083 | 1.03 | 0.96 | 1.2 | 1.07 | 0.1 |
| 2374 | T1083M | 1083 | 0.47 | 1.25 | 1.3 | 1.01 | 0.38 |
| 2375 | T1083K | 1083 | 0.75 | 0.82 | 1.39 | 0.99 | 0.29 |
| 2376 | T1083Y | 1083 | 1.28 | 0.6 | 0.88 | 0.92 | 0.28 |
| 2377 | N1087A | 1087 | 1.01 | 1.13 | 0.6 | 0.91 | 0.23 |
| 2378 | N1087W | 1087 | 0.54 | 0.78 | 1.07 | 0.8 | 0.22 |
| 2379 | N1088P | 1088 | 2.39 | 0.96 | 0 | 1.12 | 0.98 |
| 2380 | N1088A | 1088 | 0.79 | 0.27 | 0.7 | 0.59 | 0.23 |
| 2381 | I1090G | 1090 | 0.19 | 1.88 | 1.95 | 1.34 | 0.81 |
| 2382 | T1091W | 1091 | 1.58 | 0.27 | 0.7 | 0.85 | 0.54 |
| 2383 | T1091C | 1091 | 0.37 | 0.74 | 0.59 | 0.57 | 0.15 |
| 2384 | Q1092C | 1092 | 1.8 | 0.96 | 1.8 | 1.52 | 0.39 |
| 2385 | Q1092I | 1092 | 0.88 | 0.27 | 1.1 | 0.75 | 0.35 |
| 2386 | Q1092A | 1092 | 1.17 | 0.56 | 0.29 | 0.67 | 0.37 |
| 2387 | Q1092F | 1092 | 0.59 | 1.04 | 0.29 | 0.64 | 0.31 |
| 2388 | Q1092Y | 1092 | 0.45 | 0.61 | 0.41 | 0.49 | 0.08 |
| 2389 | N1093C | 1093 | 0.05 | 0.67 | 0.76 | 0.49 | 0.31 |
| 2390 | T1094I | 1094 | 1.58 | 0.27 | 1.39 | 1.08 | 0.58 |
| 2391 | T1094Y | 1094 | 1.11 | 0.67 | 0.7 | 0.83 | 0.2 |
| 2392 | T1094P | 1094 | −0.5 | 0.96 | 1.51 | 0.66 | 0.85 |
| 2393 | V1095A | 1095 | 0.39 | 0.56 | 0.59 | 0.52 | 0.09 |
| 2394 | V1095D | 1095 | 0.41 | 0.59 | 0.49 | 0.49 | 0.07 |
| 2395 | V1095R | 1095 | 0.27 | 0.61 | 0.42 | 0.43 | 0.14 |
| 2396 | M1096C | 1096 | 1.1 | 0.96 | 0 | 0.69 | 0.49 |
| 2397 | S1097D | 1097 | 0.67 | 0.64 | 0.8 | 0.7 | 0.07 |
| 2398 | K1098W | 1098 | 0.88 | 0.96 | 0.51 | 0.79 | 0.2 |
| 2399 | K1098C | 1098 | 0.3 | 0.84 | 0.77 | 0.64 | 0.24 |
| 2400 | K1098V | 1098 | 0.58 | 0.66 | 0.51 | 0.58 | 0.06 |
| 2401 | S1099P | 1099 | 1.02 | 0.72 | 0.45 | 0.73 | 0.23 |
| 2402 | S1099Q | 1099 | 0.32 | 0.77 | 0.69 | 0.59 | 0.19 |
| 2403 | S1100D | 1100 | 1.29 | 0.96 | 1.1 | 1.12 | 0.13 |
| 2404 | S1100G | 1100 | 0.76 | 1.02 | 1.27 | 1.02 | 0.21 |
| 2405 | S1100Q | 1100 | 0.98 | 0.27 | 0.41 | 0.55 | 0.31 |
| 2406 | S1100H | 1100 | 0.59 | 0.18 | 0.77 | 0.52 | 0.25 |
| 2407 | S1100M | 1100 | 0.38 | 0.52 | 0.62 | 0.51 | 0.1 |
| 2408 | Y1106I | 1106 | 0.43 | 0.42 | 0.43 | 0.43 | 0.01 |
| 2409 | K1111R | 1111 | 0.63 | 0.59 | 0.5 | 0.57 | 0.06 |
| 2410 | K1111V | 1111 | 0.71 | 0.24 | 0.42 | 0.46 | 0.19 |
| 2411 | R1113N | 1113 | 0.94 | 0.9 | 1.05 | 0.97 | 0.06 |
| 2412 | R1113K | 1113 | 1.13 | 1.12 | 0.63 | 0.96 | 0.23 |
| 2413 | R1113I | 1113 | 0.67 | 0.65 | 0.69 | 0.67 | 0.02 |
| 2414 | R1113T | 1113 | 0.35 | 0.2 | 1.35 | 0.63 | 0.51 |
| 2415 | R1113E | 1113 | −0.1 | 0.92 | 0.51 | 0.44 | 0.42 |
| 2416 | R1118F | 1118 | 0.7 | 1.04 | 0.84 | 0.86 | 0.14 |
| 2417 | R1118Q | 1118 | −0.22 | 1.25 | 0.98 | 0.67 | 0.64 |
| 2418 | R1118M | 1118 | 0.08 | 1.01 | 0.64 | 0.58 | 0.38 |
| 2419 | F1119W | 1119 | 0.47 | 0.87 | 1.5 | 0.95 | 0.42 |
| 2420 | F1119A | 1119 | 0.3 | 0.93 | 0.31 | 0.52 | 0.29 |
| 2421 | F1119N | 1119 | 0.77 | 0.96 | −0.23 | 0.5 | 0.52 |
| 2422 | S1120D | 1120 | 0.59 | 0.49 | 0.65 | 0.58 | 0.06 |
| 2423 | D1124T | 1124 | 0.59 | 0.27 | 0.4 | 0.42 | 0.13 |
| 2424 | T1125W | 1125 | 2.27 | 2.41 | 1.1 | 1.92 | 0.59 |
| 2425 | T1125D | 1125 | 1.8 | 1.65 | 1.38 | 1.61 | 0.17 |
| 2426 | T1125M | 1125 | 0.64 | 0.72 | 1.63 | 1 | 0.45 |
| 2427 | T1125H | 1125 | 0.41 | 1.18 | 0.81 | 0.8 | 0.32 |
| 2428 | T1125E | 1125 | 0.75 | 0.27 | 0.69 | 0.57 | 0.21 |
| 2429 | I1126C | 1126 | 1.58 | 0.96 | 2.3 | 1.61 | 0.55 |
| 2430 | I1126E | 1126 | 1.58 | 1.37 | 1.61 | 1.52 | 0.11 |
| 2431 | I1126A | 1126 | 0.7 | 0.27 | 1.46 | 0.81 | 0.49 |
| 2432 | T1129V | 1129 | 1.8 | 1.66 | 1.79 | 1.75 | 0.06 |
| 2433 | T1129H | 1129 | 0.19 | 2.22 | 1.94 | 1.45 | 0.9 |
| 2434 | T1129I | 1129 | 0.88 | 0.78 | 0.69 | 0.78 | 0.08 |
| 2435 | D1131L | 1131 | 1 | 0.67 | 0.4 | 0.69 | 0.25 |
| 2436 | D1131S | 1131 | 0.51 | −0.2 | 1.28 | 0.53 | 0.61 |
| 2437 | E1133T | 1133 | 0.19 | 0.67 | 0.4 | 0.42 | 0.2 |
| 2438 | K1134I | 1134 | 0.19 | 0.67 | 1.13 | 0.66 | 0.39 |
| 2439 | T1135V | 1135 | 0.41 | 0.26 | 1.24 | 0.64 | 0.43 |
| 2440 | T1135Q | 1135 | 0.48 | 0.26 | 0.84 | 0.53 | 0.24 |
| 2441 | T1135R | 1135 | 0.96 | 0.55 | −0.19 | 0.44 | 0.48 |
| 2442 | E1137Q | 1137 | 0.51 | 0.26 | 0.62 | 0.46 | 0.15 |
| 2443 | E1137L | 1137 | 0.86 | 0.6 | −0.14 | 0.44 | 0.42 |
| 2444 | E1137N | 1137 | 0.45 | 0.53 | 0.33 | 0.44 | 0.08 |
| 2445 | M1138G | 1138 | 1.89 | 2.46 | 1.38 | 1.91 | 0.44 |
| 2446 | M1138D | 1138 | 3.23 | 0.26 | 0.68 | 1.39 | 1.31 |
| 2447 | T1139V | 1139 | 0.81 | 1.58 | 0.99 | 1.12 | 0.33 |
| 2448 | T1139Y | 1139 | 0.85 | 0.63 | 0.55 | 0.67 | 0.13 |
| 2449 | T1139Q | 1139 | 0.71 | 0.53 | 0.13 | 0.46 | 0.24 |
| 2450 | I1141P | 1141 | 0.57 | 0.96 | 0.95 | 0.82 | 0.18 |
| 2451 | I1141C | 1141 | 0.59 | 0.38 | 0.48 | 0.48 | 0.09 |
| 2452 | N1142A | 1142 | 0.33 | 0.58 | 0.46 | 0.46 | 0.1 |
| 2453 | W1143F | 1143 | 0.23 | 0.55 | 0.5 | 0.43 | 0.14 |
| 2454 | D1145F | 1145 | 0.74 | 0.31 | 0.3 | 0.45 | 0.2 |
| 2455 | G1146K | 1146 | 0.52 | 0.53 | 0.35 | 0.47 | 0.08 |
| 2456 | R1150N | 1150 | 1.1 | 1.18 | 1.08 | 1.12 | 0.04 |
| 2457 | Q1151C | 1151 | 0.82 | 0.43 | 0.3 | 0.52 | 0.22 |
| 2458 | D1155M | 1155 | 0.57 | 0.52 | 0.5 | 0.53 | 0.03 |
| 2459 | I1158A | 1158 | 0.79 | 0.33 | 0.54 | 0.55 | 0.19 |
| 2460 | V1159D | 1159 | 0.77 | 0.43 | 1 | 0.74 | 0.24 |
| 2461 | F1163C | 1163 | 0.44 | 0.94 | 0.59 | 0.66 | 0.21 |
| 2462 | F1163N | 1163 | 0.5 | 0.37 | 0.53 | 0.47 | 0.07 |
| 2463 | E1164P | 1164 | 1 | 0.25 | 0.2 | 0.48 | 0.36 |
| 2464 | I1165A | 1165 | 0.53 | 0.37 | 0.46 | 0.45 | 0.07 |
| 2465 | L1179D | 1179 | 1.06 | 1.38 | 1.22 | 1.22 | 0.13 |
| 2466 | L1179E | 1179 | 0.51 | 0.77 | 0.88 | 0.72 | 0.15 |
| 2467 | L1179N | 1179 | 0.62 | 0.55 | 0.44 | 0.53 | 0.07 |
| 2468 | R1182Y | 1182 | 1.57 | 0.25 | 1.58 | 1.13 | 0.62 |
| 2469 | R1182Q | 1182 | 1.06 | 1.04 | 1 | 1.03 | 0.02 |
| 2470 | R1182K | 1182 | 0.32 | 1.14 | 0.8 | 0.75 | 0.34 |
| 2471 | R1182N | 1182 | 0.59 | −0.04 | 0.78 | 0.44 | 0.35 |
| 2472 | D1183Y | 1183 | 0.89 | 1.03 | 0.91 | 0.94 | 0.06 |
| 2473 | Y1184W | 1184 | 0.68 | 0.5 | 0.35 | 0.51 | 0.13 |
| 2474 | R1186M | 1186 | 0.45 | 0.83 | 0.48 | 0.59 | 0.17 |
| 2475 | L1187M | 1187 | 1.65 | 0.01 | 0.46 | 0.71 | 0.69 |
| 2476 | L1192Y | 1192 | 2.13 | 1.34 | 1.35 | 1.61 | 0.37 |
| 2477 | L1192K | 1192 | 0.43 | 0.49 | 0.41 | 0.45 | 0.03 |
| 2478 | E1194C | 1194 | 0.67 | 0.8 | 1.02 | 0.83 | 0.14 |
| 2479 | E1194S | 1194 | 1.59 | −0.04 | −0.02 | 0.51 | 0.77 |
| 2480 | E1194F | 1194 | 0.52 | 0.62 | 0.2 | 0.45 | 0.18 |
| 2481 | N1195Y | 1195 | 1.9 | 0.51 | 0.11 | 0.84 | 0.76 |
| 2482 | N1195S | 1195 | 0.39 | 0.79 | 0.51 | 0.56 | 0.17 |
| 2483 | N1196A | 1196 | 0.74 | 0.61 | 0.52 | 0.62 | 0.09 |
| 2484 | I1197W | 1197 | 0.13 | 0.69 | 0.74 | 0.52 | 0.28 |
| 2485 | I1197G | 1197 | 0.4 | 0.84 | 0.11 | 0.45 | 0.3 |
| 2486 | A1204C | 1204 | 0.58 | 1.42 | 1.2 | 1.07 | 0.35 |
| 2487 | A1204M | 1204 | 0.76 | 0.67 | 1.13 | 0.86 | 0.2 |
| 2488 | A1204Q | 1204 | 1.16 | 0.53 | 0.39 | 0.69 | 0.33 |
| 2489 | A1204F | 1204 | 0.32 | 0.51 | 0.81 | 0.55 | 0.2 |
| 2490 | A1204D | 1204 | 0.66 | 0.56 | 0.36 | 0.53 | 0.13 |
| 2491 | A1204N | 1204 | 0.51 | 0.37 | 0.54 | 0.47 | 0.08 |
| 2492 | D1206C | 1206 | 1.22 | 1.02 | 1.05 | 1.1 | 0.09 |
| 2493 | D1206I | 1206 | 0.01 | 0.82 | 0.51 | 0.45 | 0.34 |
| 2494 | D1206F | 1206 | 0.38 | 0.3 | 0.65 | 0.44 | 0.15 |
| 2495 | A1207Q | 1207 | 0.97 | 0.58 | 0.54 | 0.7 | 0.19 |
| 2496 | L1208Q | 1208 | 0.78 | 0.03 | 0.51 | 0.44 | 0.31 |
| 2497 | L1208A | 1208 | 0.35 | 0.37 | 0.6 | 0.44 | 0.12 |
| 2498 | D1211C | 1211 | 0.9 | 0.61 | 0.74 | 0.75 | 0.12 |
| 2499 | D1211T | 1211 | 0.64 | 0.68 | 0.7 | 0.67 | 0.02 |
| 2500 | D1211S | 1211 | 0.63 | 0.51 | 0.69 | 0.61 | 0.07 |
| 2501 | A1212C | 1212 | 0.94 | 0.84 | 0.89 | 0.89 | 0.04 |
| 2502 | A1212T | 1212 | 0.8 | 0.74 | 0.7 | 0.75 | 0.04 |
| 2503 | A1212S | 1212 | 0.65 | 0.6 | 0.61 | 0.62 | 0.02 |
| 2504 | A1212V | 1212 | 0.51 | 0.52 | 0.68 | 0.57 | 0.08 |
| 2505 | A1212M | 1212 | 0.5 | 0.5 | 0.33 | 0.44 | 0.08 |
| 2506 | N1215G | 1215 | 0.62 | 0.65 | 0.91 | 0.73 | 0.13 |
| 2507 | E1227T | 1227 | 0.62 | 0.59 | 0.12 | 0.44 | 0.23 |
| 2508 | I1228H | 1228 | 1.39 | 0.87 | 0.4 | 0.89 | 0.4 |
| 2509 | I1228Y | 1228 | 0.89 | 0.18 | 0.65 | 0.57 | 0.29 |
| 2510 | K1229T | 1229 | 0.44 | 0.45 | 0.39 | 0.43 | 0.03 |
| 2511 | Q1230T | 1230 | 0.5 | 0.73 | 0.25 | 0.49 | 0.19 |
| 2512 | Q1230C | 1230 | −0.55 | 0.59 | 1.4 | 0.48 | 0.8 |
| 2513 | Q1230G | 1230 | 0.14 | 1.28 | −0.1 | 0.44 | 0.61 |
| 2514 | E1233W | 1233 | 0.7 | 1.1 | 1.69 | 1.17 | 0.41 |
| 2515 | E1233Y | 1233 | 0.45 | 0.68 | 0.4 | 0.51 | 0.13 |
| 2516 | N1234A | 1234 | 1.53 | 1.58 | 0.41 | 1.17 | 0.54 |
| 2517 | N1234I | 1234 | 0.43 | 1.04 | 1.29 | 0.92 | 0.36 |
| 2518 | N1234E | 1234 | 1.12 | 0.88 | −0.51 | 0.5 | 0.72 |
| 2519 | N1234F | 1234 | 0.83 | −0.22 | 0.75 | 0.45 | 0.48 |
| 2520 | W1235Y | 1235 | 1.64 | 1.69 | 1.15 | 1.5 | 0.24 |
| 2521 | W1235E | 1235 | 0.83 | 0.52 | 1 | 0.78 | 0.2 |
| 2522 | K1236F | 1236 | 0.54 | 1.03 | 0.74 | 0.77 | 0.2 |
| 2523 | K1236P | 1236 | 0.61 | 0.93 | 0.49 | 0.67 | 0.19 |
| 2524 | E1237I | 1237 | 0.63 | 0.62 | 0.72 | 0.66 | 0.04 |
| 2525 | E1237G | 1237 | 0.59 | 0.32 | 1.04 | 0.65 | 0.3 |
| 2526 | E1237A | 1237 | 0.51 | 0.68 | 0.54 | 0.58 | 0.07 |
| 2527 | E1237F | 1237 | 0 | 0.5 | 0.81 | 0.44 | 0.33 |
| 2528 | G1239H | 1239 | 1.24 | 2.06 | 1.15 | 1.48 | 0.41 |
| 2529 | G1239K | 1239 | 0.88 | 0.37 | 0.73 | 0.66 | 0.21 |
| 2530 | G1239T | 1239 | 0.68 | 0.35 | 0.59 | 0.54 | 0.14 |
| 2531 | G1239L | 1239 | 0.57 | 0.42 | 0.51 | 0.5 | 0.06 |
| 2532 | G1239Q | 1239 | 0.14 | 1.18 | 0.15 | 0.49 | 0.49 |
| 2533 | K1240Y | 1240 | 0.63 | 0.43 | 0.72 | 0.59 | 0.12 |
| 2534 | F1241P | 1241 | 0.97 | 0.98 | 0.49 | 0.81 | 0.23 |
| 2535 | S1242E | 1242 | 0.82 | 0.66 | 0.7 | 0.72 | 0.07 |
| 2536 | S1242Q | 1242 | 0.65 | 0.28 | 0.41 | 0.44 | 0.15 |
| 2537 | D1244I | 1244 | 0.5 | 0.85 | 0.85 | 0.73 | 0.16 |
| 2538 | K1245G | 1245 | 0.88 | 0.93 | 1.09 | 0.97 | 0.09 |
| 2539 | K1245V | 1245 | 0.53 | 0.43 | 0.54 | 0.5 | 0.05 |
| 2540 | S1249E | 1249 | 1.06 | 0.6 | 1.04 | 0.9 | 0.21 |
| 2541 | S1249Q | 1249 | 0.51 | 0.71 | 0.81 | 0.67 | 0.12 |
| 2542 | S1249W | 1249 | 0.4 | 0.65 | 0.27 | 0.44 | 0.16 |
| 2543 | N1250L | 1250 | 0.95 | 0.6 | 0.46 | 0.67 | 0.2 |
| 2544 | N1250P | 1250 | 0.58 | 0.63 | 0.46 | 0.56 | 0.07 |
| 2545 | N1250G | 1250 | 0.75 | 0.71 | 0.2 | 0.55 | 0.25 |
| 2546 | N1250A | 1250 | 0.79 | 0.53 | 0.17 | 0.5 | 0.25 |
| 2547 | D1252L | 1252 | 0.14 | 1.99 | 2.11 | 1.41 | 0.9 |
| 2548 | F1254T | 1254 | 0.83 | 0.19 | 0.3 | 0.44 | 0.28 |
| 2549 | D1255M | 1255 | 0.83 | 1.99 | 1.01 | 1.28 | 0.51 |
| 2550 | F1256R | 1256 | 0.83 | 0.19 | 0.6 | 0.54 | 0.26 |
| 2551 | F1256W | 1256 | 0.54 | 0.34 | 0.51 | 0.47 | 0.09 |
| 2552 | K1260G | 1260 | 1.06 | 1.71 | 1.78 | 1.51 | 0.33 |
| 2553 | K1260F | 1260 | 0.14 | 0.54 | 0.7 | 0.46 | 0.23 |
| 2554 | K1260H | 1260 | 0.78 | 0.31 | 0.24 | 0.44 | 0.24 |
| 2555 | R1261Y | 1261 | 1.06 | 0.9 | 1.42 | 1.12 | 0.22 |
| 2556 | R1261V | 1261 | 1.24 | 1.3 | 0.6 | 1.05 | 0.32 |
| 2557 | R1261W | 1261 | 1.59 | 0.76 | 0.32 | 0.89 | 0.53 |
| 2558 | R1261N | 1261 | 0.83 | 0.21 | 1.3 | 0.78 | 0.45 |
| 2559 | R1261M | 1261 | 1.24 | 0.21 | 0.6 | 0.68 | 0.43 |
| 2560 | Y1262M | 1262 | 0.14 | 0.75 | 0.53 | 0.47 | 0.25 |
| 2561 | L1263N | 1263 | 0.92 | 1.31 | 0.61 | 0.95 | 0.29 |
| 2562 | L1263Q | 1263 | 0.77 | 1.1 | 0.81 | 0.89 | 0.15 |
| 2563 | L1263A | 1263 | 0.81 | 0.87 | 0.51 | 0.73 | 0.16 |
| 2564 | L1263E | 1263 | 0.84 | 0.83 | 0.37 | 0.68 | 0.22 |

The polynucleotide and polypeptide sequences of the 2564 mutants shown in Table 2 are not provided. A person having ordinary skill in the art would understand that the mutations can be created from the wild type ERCAS12A nucleotide or polypeptide sequences by changing the codons for the wild type amino acid and changing them to a codon for the mutant amino acid. For Example, ERCAS12A mutant 1 shown in Table 2, “M1G” indicates that methionine 1 was changed to a glycine in the polypeptide sequence. Similarly, the wild type codon for methionine 1, “ATG,” can be changed to any one of the E. coli glycine codons “GGT,” GGC,” GGA,” or “GGG” to create the mutant polynucleotide coding sequence that would encode a glycine at position 1. The codons for the ERCAS12A amino acids can be optimized for a specific organism such as E. coli, Homo sapiens, or other organisms by changing the coding strand codons to codons which are most commonly used in the respective organsim.

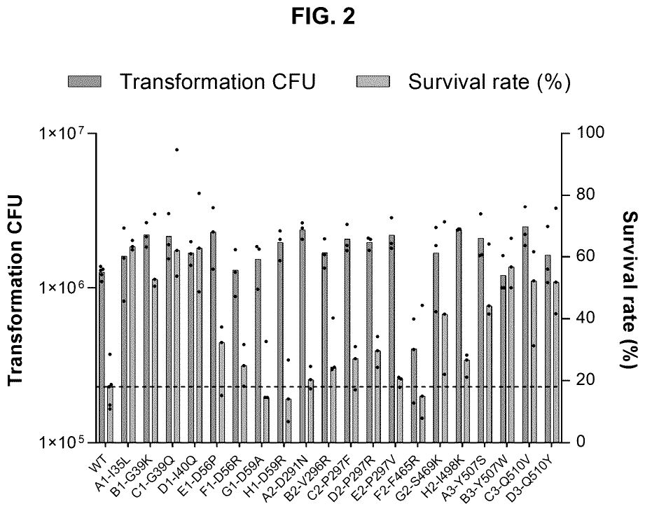

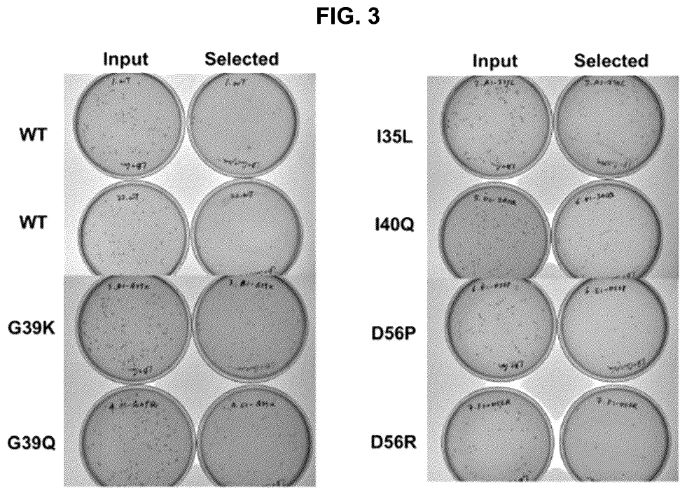

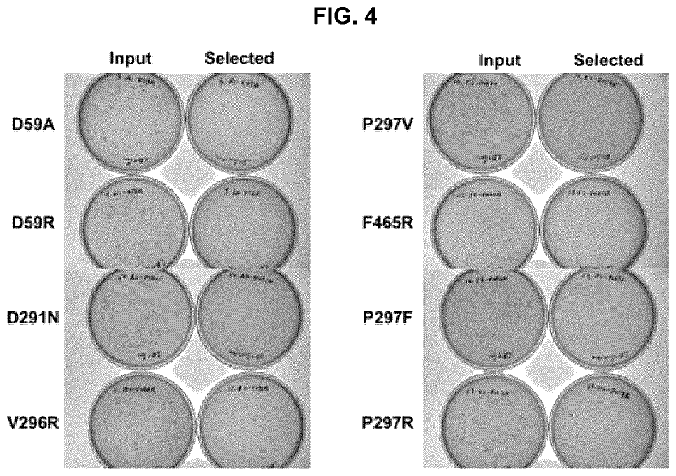

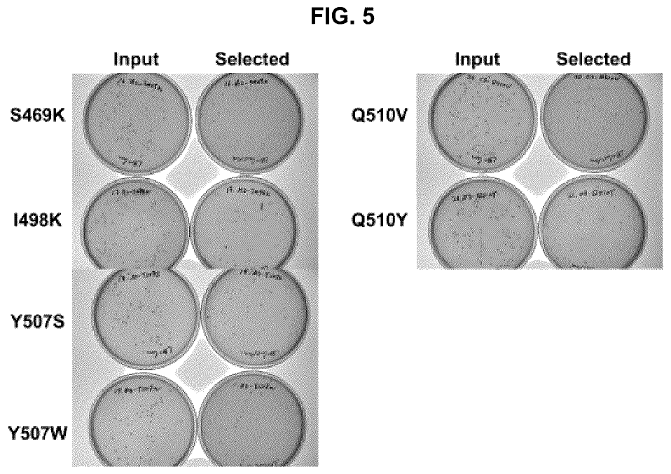

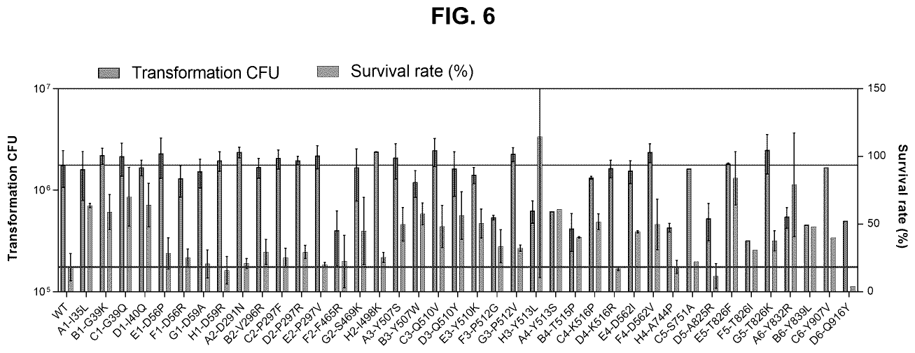

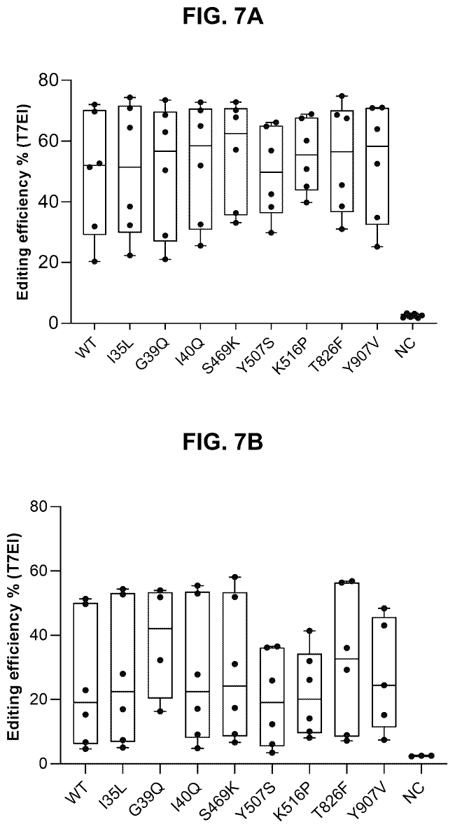

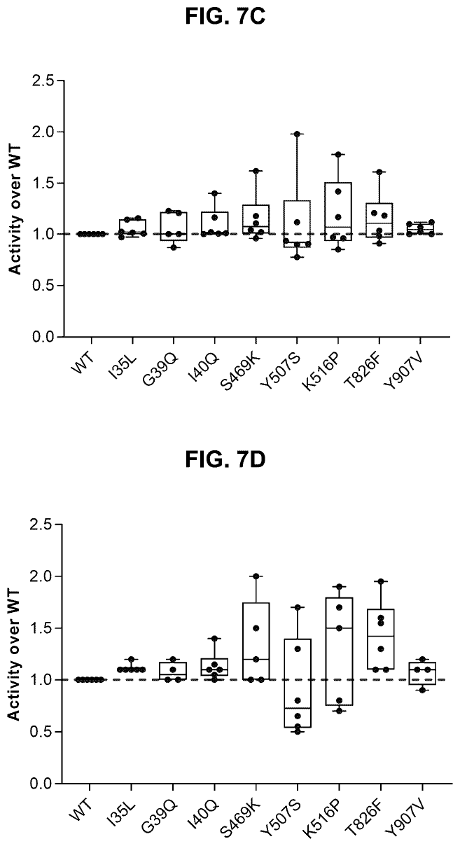

To demonstrate the utility of these results, a set of ERCAS12A variants were selectively cloned with point mutations (135L, G39K, G39Q, 140Q, D56P, D56R, D291N, V296R, P297F, P297V, F465R, S469K, 1498K, Y507S, Y507W, Q510V, and Q510Y), and tested their activities in the context of bacteria selection assay. As shown in FIG. 2-5 most of the characterized mutants improved the survival rate of bacteria over WT-ERCAS12A (SEQ ID NO: 2) upon selection, thus validating the result of high-throughput screening. Since the survival rate in the bacterial assay is linked to the intrinsic cleavage activity of ERCAS12A nuclease, these mutants hold great promise for human genome engineering with improved performance over WT enzyme.

Table 3 shows the primer sequences used to generate an ERCAS12A saturation mutagenesis library. Standard recombinant methods and techniques were used. The screening library was constructed using the method descried in Wrenbeck et al., Nat. Methods 13(11):928-930 (2016).

The screen works by transforming a screening strain harboring a toxin plasmid with the mutant ERCAS12A library and crRNA targeting the HPRT38346 site on the toxin plasmid. After recovery and IPTG induction, cells were plated on LB-chloramphenicol media with arabinose and incubated at 37° C. overnight. Functional ERCAS12A enzymes are capable of CRISPR activity and deactivate the toxin plasmid. If the mutant LbCas12a enzyme is not functional the transformed screening E. coli cells do not survive. ERCAS12A expression plasmids carried by the surviving E. coli cells were extracted and purified. Both input and selected plasmid libraries were amplified by PCR, randomly fragmented, and sequenced. The frequencies of mutations at each position of ERCAS12A in both libraries were measured and normalized to the total coverage of each codon. The relative survival rate of each point mutation was calculated as the ratio of normalized frequency between selected and input library. Since the degree of cell survival under the arabinose selection is indicative of the cleavage activity of ERCAS12A variant at HPRT38346 protospacer, any variants that enriched during the selection over WT are those with enhanced activity at the TTTT PAM.

TABLE 3
Primer Sequences for ERCAS12A Saturation Mutagenesis Library

| | SEQ ID | |
|---|---|---|
| Name | NO | DNA Sequence (5′→3′) |
| ERCAS12A- | 211 | /5Phos/taagtataagaaggagatatacatNNKAATAACGGTACCAATAACTTTCAA |
| P1 | | |
| ERCAS12A- | 212 | /5Phos/gtataagaaggagatatacatatgNNKAACGGTACCAATAACTTTCAAAAT |
| P2 | | |
| ERCAS12A- | 213 | /5Phos/taagaaggagatatacatatgAATNNKGGTACCAATAACTTTCAAAATTTT |
| P3 | | |
| ERCAS12A- | 214 | /5Phos/gaaggagatatacatatgAATAACNNKACCAATAACTTTCAAAATTTTATC |
| P4 | | |
| ERCAS12A- | 215 | /5Phos/ggagatatacatatgAATAACGGTNNKAATAACTTTCAAAATTTTATCGGG |
| P5 | | |
| ERCAS12A- | 216 | /5Phos/gatatacatatgAATAACGGTACCNNKAACTTTCAAAATTTTATCGGGATC |
| P6 | | |
| ERCAS12A- | 217 | /5Phos/atacatatgAATAACGGTACCAATNNKTTTCAAAATTTTATCGGGATCTCC |
| P7 | | |
| ERCAS12A- | 218 | /5Phos/catatgAATAACGGTACCAATAACNNKCAAAATTTTATCGGGATCTCCTCG |
| P8 | | |
| ERCAS12A- | 219 | /5Phos/atgAATAACGGTACCAATAACTTTNNKAATTTTATCGGGATCTCCTCGTTG |
| P9 | | |
| ERCAS12A- | 220 | /5Phos/AATAACGGTACCAATAACTTTCAANNKTTTATCGGGATCTCCTCGTTGCAA |
| P10 | | |
| ERCAS12A- | 221 | /5Phos/AACGGTACCAATAACTTTCAAAATNNKATCGGGATCTCCTCGTTGCAAAAA |
| P11 | | |
| ERCAS12A- | 222 | /5Phos/GGTACCAATAACTTTCAAAATTTTNNKGGGATCTCCTCGTTGCAAAAAACT |
| P12 | | |
| ERCAS12A- | 223 | /5Phos/ACCAATAACTTTCAAAATTTTATCNNKATCTCCTCGTTGCAAAAAACTCTT |
| P13 | | |
| ERCAS12A- | 224 | /5Phos/AATAACTTTCAAAATTTTATCGGGNNKTCCTCGTTGCAAAAAACTCTTCGT |
| P14 | | |
| ERCAS12A- | 225 | /5Phos/AACTTTCAAAATTTTATCGGGATCNNKTCGTTGCAAAAAACTCTTCGTAAT |
| P15 | | |
| ERCAS12A- | 226 | /5Phos/TTTCAAAATTTTATCGGGATCTCCNNKTTGCAAAAAACTCTTCGTAATGCT |
| P16 | | |
| ERCAS12A- | 227 | /5Phos/CAAAATTTTATCGGGATCTCCTCGNNKCAAAAAACTCTTCGTAATGCTTTA |
| P17 | | |
| ERCAS12A- | 228 | /5Phos/AATTTTATCGGGATCTCCTCGTTGNNKAAAACTCTTCGTAATGCTTTAATC |
| P18 | | |
| ERCAS12A- | 229 | /5Phos/TTTATCGGGATCTCCTCGTTGCAANNKACTCTTCGTAATGCTTTAATCCCG |
| P19 | | |
| ERCAS12A- | 230 | /5Phos/ATCGGGATCTCCTCGTTGCAAAAANNKCTTCGTAATGCTTTAATCCCGACT |
| P20 | | |
| ERCAS12A- | 231 | /5Phos/GGGATCTCCTCGTTGCAAAAAACTNNKCGTAATGCTTTAATCCCGACTGAG |
| P21 | | |
| ERCAS12A- | 232 | /5Phos/ATCTCCTCGTTGCAAAAAACTCTTNNKAATGCTTTAATCCCGACTGAGACA |
| 22 | | |
| ERCAS12A- | 233 | /5Phos/TCCTCGTTGCAAAAAACTCTTCGTNNKGCTTTAATCCCGACTGAGACAACA |
| P23 | | |
| ERCAS12A- | 234 | /5Phos/TCGTTGCAAAAAACTCTTCGTAATNNKTTAATCCCGACTGAGACAACACAG |
| P24 | | |
| ERCAS12A- | 235 | /5Phos/TTGCAAAAAACTCTTCGTAATGCTNNKATCCCGACTGAGACAACACAGCAG |
| P25 | | |
| ERCAS12A- | 236 | /5Phos/CAAAAAACTCTTCGTAATGCTTTANNKCCGACTGAGACAACACAGCAGTTC |
| P26 | | |
| ERCAS12A- | 237 | /5Phos/AAAACTCTTCGTAATGCTTTAATCNNKACTGAGACAACACAGCAGTTCATC |
| P27 | | |
| ERCAS12A- | 238 | /5Phos/ACTCTTCGTAATGCTTTAATCCCGNNKGAGACAACACAGCAGTTCATCGTT |
| P28 | | |
| ERCAS12A- | 239 | /5Phos/CTTCGTAATGCTTTAATCCCGACTNNKACAACACAGCAGTTCATCGTTAAA |
| P29 | | |
| ERCAS12A- | 240 | /5Phos/CGTAATGCTTTAATCCCGACTGAGNNKACACAGCAGTTCATCGTTAAAAAC |
| P30 | | |
| ERCAS12A- | 241 | /5Phos/AATGCTTTAATCCCGACTGAGACANNKCAGCAGTTCATCGTTAAAAACGGC |
| P31 | | |
| ERCAS12A- | 242 | /5Phos/GCTTTAATCCCGACTGAGACAACANNKCAGTTCATCGTTAAAAACGGCATC |
| P32 | | |
| ERCAS12A- | 243 | /5Phos/TTAATCCCGACTGAGACAACACAGNNKTTCATCGTTAAAAACGGCATCATT |
| P33 | | |
| ERCAS12A- | 244 | /5Phos/ATCCCGACTGAGACAACACAGCAGNNKATCGTTAAAAACGGCATCATTAAA |
| P34 | | |
| ERCAS12A- | 245 | /5Phos/CCGACTGAGACAACACAGCAGTTCNNKGTTAAAAACGGCATCATTAAAGAA |
| P35 | | |
| ERCAS12A- | 246 | /5Phos/ACTGAGACAACACAGCAGTTCATCNNKAAAAACGGCATCATTAAAGAAGAT |
| P36 | | |
| ERCAS12A- | 247 | /5Phos/GAGACAACACAGCAGTTCATCGTTNNKAACGGCATCATTAAAGAAGATGAG |
| P37 | | |
| ERCAS12A- | 248 | /5Phos/ACAACACAGCAGTTCATCGTTAAANNKGGCATCATTAAAGAAGATGAGCTG |
| P38 | | |
| ERCAS12A- | 249 | /5Phos/ACACAGCAGTTCATCGTTAAAAACNNKATCATTAAAGAAGATGAGCTGCGC |
| P39 | | |
| ERCAS12A- | 250 | /5Phos/CAGCAGTTCATCGTTAAAAACGGCNNKATTAAAGAAGATGAGCTGCGCGGC |
| P40 | | |
| ERCAS12A- | 251 | /5Phos/CAGTTCATCGTTAAAAACGGCATCNNKAAAGAAGATGAGCTGCGCGGCGAG |
| P41 | | |
| ERCAS12A- | 252 | /5Phos/TTCATCGTTAAAAACGGCATCATTNNKGAAGATGAGCTGCGCGGCGAGAAC |
| P42 | | |
| ERCAS12A- | 253 | /5Phos/ATCGTTAAAAACGGCATCATTAAANNKGATGAGCTGCGCGGCGAGAACCGT |
| P43 | | |
| ERCAS12A- | 254 | /5Phos/GTTAAAAACGGCATCATTAAAGAANNKGAGCTGCGCGGCGAGAACCGTCAG |
| P44 | | |
| ERCAS12A- | 255 | /5Phos/AAAAACGGCATCATTAAAGAAGATNNKCTGCGCGGCGAGAACCGTCAGATT |
| P45 | | |
| ERCAS12A- | 256 | /5Phos/AACGGCATCATTAAAGAAGATGAGNNKCGCGGCGAGAACCGTCAGATTTTA |
| P46 | | |
| ERCAS12A- | 257 | /5Phos/GGCATCATTAAAGAAGATGAGCTGNNKGGCGAGAACCGTCAGATTTTAAAG |
| P47 | | |
| ERCAS12A- | 258 | /5Phos/ATCATTAAAGAAGATGAGCTGCGCNNKGAGAACCGTCAGATTTTAAAGGAC |
| P48 | | |
| ERCAS12A | 259 | /5Phos/ATTAAAGAAGATGAGCTGCGCGGCNNKAACCGTCAGATTTTAAAGGACATC |
| P49 | | |
| ERCAS12A- | 260 | /5Phos/AAAGAAGATGAGCTGCGCGGCGAGNNKCGTCAGATTTTAAAGGACATCATG |
| P50 | | |
| ERCAS12A- | 261 | /5Phos/GAAGATGAGCTGCGCGGCGAGAACNNKCAGATTTTAAAGGACATCATGGAC |
| P51 | | |
| ERCAS12A | 262 | /5Phos/GATGAGCTGCGCGGCGAGAACCGTNNKATTTTAAAGGACATCATGGACGAC |
| P52 | | |
| ERCAS12A- | 263 | /5Phos/GAGCTGCGCGGCGAGAACCGTCAGNNKTTAAAGGACATCATGGACGACTAT |
| P53 | | |
| ERCAS12A- | 264 | /5Phos/CTGCGCGGCGAGAACCGTCAGATTNNKAAGGACATCATGGACGACTATTAC |
| P54 | | |
| ERCAS12A- | 265 | /5Phos/CGCGGCGAGAACCGTCAGATTTTANNKGACATCATGGACGACTATTACCGT |
| P55 | | |
| ERCAS12A- | 266 | /5Phos/GGCGAGAACCGTCAGATTTTAAAGNNKATCATGGACGACTATTACCGTGGG |
| P56 | | |
| ERCAS12A- | 267 | /5Phos/GAGAACCGTCAGATTTTAAAGGACNNKATGGACGACTATTACCGTGGGTTC |
| P57 | | |
| ERCAS12A- | 268 | /5Phos/AACCGTCAGATTTTAAAGGACATCNNKGACGACTATTACCGTGGGTTCATC |
| P58 | | |
| ERCAS12A- | 269 | /5Phos/CGTCAGATTTTAAAGGACATCATGNNKGACTATTACCGTGGGTTCATCTCA |
| P59 | | |
| ERCAS12A- | 270 | /5Phos/CAGATTTTAAAGGACATCATGGACNNKTATTACCGTGGGTTCATCTCAGAA |
| P60 | | |
| ERCAS12A- | 271 | /5Phos/ATTTTAAAGGACATCATGGACGACNNKTACCGTGGGTTCATCTCAGAAACA |
| P61 | | |
| ERCAS12A- | 272 | /5Phos/TTAAAGGACATCATGGACGACTATNNKCGTGGGTTCATCTCAGAAACATTA |
| P62 | | |
| ERCAS12A | 273 | /5Phos/AAGGACATCATGGACGACTATTACNNKGGGTTCATCTCAGAAACATTAAGC |
| P63 | | |
| ERCAS12A- | 274 | /5Phos/GACATCATGGACGACTATTACCGTNNKTTCATCTCAGAAACATTAAGCAGC |
| P64 | | |
| ERCAS12A- | 275 | /5Phos/ATCATGGACGACTATTACCGTGGGNNKATCTCAGAAACATTAAGCAGCATC |
| P65 | | |
| ERCAS12A- | 276 | /5Phos/ATGGACGACTATTACCGTGGGTTCNNKTCAGAAACATTAAGCAGCATCGAT |
| P66 | | |
| ERCAS12A- | 277 | /5Phos/GACGACTATTACCGTGGGTTCATCNNKGAAACATTAAGCAGCATCGATGAT |
| P67 | | |
| ERCAS12A- | 278 | /5Phos/GACTATTACCGTGGGTTCATCTCANNKACATTAAGCAGCATCGATGATATC |
| P68 | | |
| ERCAS12A- | 279 | /5Phos/TATTACCGTGGGTTCATCTCAGAANNKTTAAGCAGCATCGATGATATCGAC |
| P69 | | |
| ERCAS12A- | 280 | /5Phos/TACCGTGGGTTCATCTCAGAAACANNKAGCAGCATCGATGATATCGACTGG |
| P70 | | |
| ERCAS12A- | 281 | /5Phos/CGTGGGTTCATCTCAGAAACATTANNKAGCATCGATGATATCGACTGGACC |
| P71 | | |
| ERCAS12A- | 282 | /5Phos/GGGTTCATCTCAGAAACATTAAGCNNKATCGATGATATCGACTGGACCTCA |
| P72 | | |
| ERCAS12A- | 283 | /5Phos/TTCATCTCAGAAACATTAAGCAGCNNKGATGATATCGACTGGACCTCACTG |
| P73 | | |
| ERCAS12A- | 284 | /5Phos/ATCTCAGAAACATTAAGCAGCATCNNKGATATCGACTGGACCTCACTGTTT |
| P74 | | |
| ERCAS12A- | 285 | /5Phos/TCAGAAACATTAAGCAGCATCGATNNKATCGACTGGACCTCACTGTTTGAG |
| P75 | | |
| ERCAS12A- | 286 | /5Phos/GAAACATTAAGCAGCATCGATGATNNKGACTGGACCTCACTGTTTGAGAAG |
| P76 | | |
| ERCAS12A- | 287 | /5Phos/ACATTAAGCAGCATCGATGATATCNNKTGGACCTCACTGTTTGAGAAGATG |
| P77 | | |
| ERCAS12A- | 288 | /5Phos/TTAAGCAGCATCGATGATATCGACNNKACCTCACTGTTTGAGAAGATGGAG |
| P78 | | |
| ERCAS12A- | 289 | /5Phos/AGCAGCATCGATGATATCGACTGGNNKTCACTGTTTGAGAAGATGGAGATT |
| P79 | | |
| ERCAS12A- | 290 | /5Phos/AGCATCGATGATATCGACTGGACCNNKCTGTTTGAGAAGATGGAGATTCAA |
| P80 | | |
| ERCAS12A- | 291 | /5Phos/ATCGATGATATCGACTGGACCTCANNKTTTGAGAAGATGGAGATTCAACTT |
| P81 | | |
| ERCAS12A- | 292 | /5Phos/GATGATATCGACTGGACCTCACTGNNKGAGAAGATGGAGATTCAACTTAAG |
| P82 | | |
| ERCAS12A- | 293 | /5Phos/GATATCGACTGGACCTCACTGTTTNNKAAGATGGAGATTCAACTTAAGAAT |
| P83 | | |
| ERCAS12A- | 294 | /5Phos/ATCGACTGGACCTCACTGTTTGAGNNKATGGAGATTCAACTTAAGAATGGA |
| P84 | | |
| ERCAS12A- | 295 | /5Phos/GACTGGACCTCACTGTTTGAGAAGNNKGAGATTCAACTTAAGAATGGAGAC |
| P85 | | |
| ERCAS12A- | 296 | /5Phos/TGGACCTCACTGTTTGAGAAGATGNNKATTCAACTTAAGAATGGAGACAAC |
| P86 | | |
| ERCAS12A- | 297 | /5Phos/ACCTCACTGTTTGAGAAGATGGAGNNKCAACTTAAGAATGGAGACAACAAA |
| P87 | | |
| ERCAS12A- | 298 | /5Phos/TCACTGTTTGAGAAGATGGAGATTNNKCTTAAGAATGGAGACAACAAAGAC |
| P88 | | |
| ERCAS12A- | 299 | /5Phos/CTGTTTGAGAAGATGGAGATTCAANNKAAGAATGGAGACAACAAAGACACT |
| P89 | | |
| ERCAS12A- | 300 | /5Phos/TTTGAGAAGATGGAGATTCAACTTNNKAATGGAGACAACAAAGACACTTTA |
| P90 | | |
| ERCAS12A- | 301 | /5Phos/GAGAAGATGGAGATTCAACTTAAGNNKGGAGACAACAAAGACACTTTAATT |
| P91 | | |
| ERCAS12A- | 302 | /5Phos/AAGATGGAGATTCAACTTAAGAATNNKGACAACAAAGACACTTTAATTAAG |
| P92 | | |
| ERCAS12A- | 303 | /5Phos/ATGGAGATTCAACTTAAGAATGGANNKAACAAAGACACTTTAATTAAGGAG |
| P93 | | |
| ERCAS12A- | 304 | /5Phos/GAGATTCAACTTAAGAATGGAGACNNKAAAGACACTTTAATTAAGGAGCAG |
| P94 | | |
| ERCAS12A- | 305 | /5Phos/ATTCAACTTAAGAATGGAGACAACNNKGACACTTTAATTAAGGAGCAGACG |
| P95 | | |
| ERCAS12A- | 306 | /5Phos/CAACTTAAGAATGGAGACAACAAANNKACTTTAATTAAGGAGCAGACGGAA |
| P96 | | |
| ERCAS12A- | 307 | /5Phos/CTTAAGAATGGAGACAACAAAGACNNKTTAATTAAGGAGCAGACGGAATAT |
| P97 | | |
| ERCAS12A- | 308 | /5Phos/AAGAATGGAGACAACAAAGACACTNNKATTAAGGAGCAGACGGAATATCGT |
| P98 | | |
| ERCAS12A- | 309 | /5Phos/AATGGAGACAACAAAGACACTTTANNKAAGGAGCAGACGGAATATCGTAAA |
| P99 | | |
| ERCAS12A- | 310 | /5Phos/GGAGACAACAAAGACACTTTAATTNNKGAGCAGACGGAATATCGTAAAGCT |
| P100 | | |
| ERCAS12A- | 311 | /5Phos/GACAACAAAGACACTTTAATTAAGNNKCAGACGGAATATCGTAAAGCTATC |
| P101 | | |
| ERCAS12A- | 312 | /5Phos/AACAAAGACACTTTAATTAAGGAGNNKACGGAATATCGTAAAGCTATCCAT |
| P102 | | |
| ERCAS12A- | 313 | /5Phos/AAAGACACTTTAATTAAGGAGCAGNNKGAATATCGTAAAGCTATCCATAAA |
| P103 | | |
| ERCAS12A- | 314 | /5Phos/GACACTTTAATTAAGGAGCAGACGNNKTATCGTAAAGCTATCCATAAAAAA |
| P104 | | |
| ERCAS12A- | 315 | /5Phos/ACTTTAATTAAGGAGCAGACGGAANNKCGTAAAGCTATCCATAAAAAATTT |
| P105 | | |
| ERCAS12A- | 316 | /5Phos/TTAATTAAGGAGCAGACGGAATATNNKAAAGCTATCCATAAAAAATTTGCA |
| P106 | | |
| ERCAS12A- | 317 | /5Phos/ATTAAGGAGCAGACGGAATATCGTNNKGCTATCCATAAAAAATTTGCAAAC |
| P107 | | |
| ERCAS12A- | 318 | /5Phos/AAGGAGCAGACGGAATATCGTAAANNKATCCATAAAAAATTTGCAAACGAT |
| P108 | | |
| ERCAS12A- | 319 | /5Phos/GAGCAGACGGAATATCGTAAAGCTNNKCATAAAAAATTTGCAAACGATGAC |
| P109 | | |
| ERCAS12A- | 320 | /5Phos/CAGACGGAATATCGTAAAGCTATCNNKAAAAAATTTGCAAACGATGACCGT |
| P110 | | |
| ERCAS12A- | 321 | /5Phos/ACGGAATATCGTAAAGCTATCCATNNKAAATTTGCAAACGATGACCGTTTC |
| P111 | | |
| ERCAS12A- | 322 | /5Phos/GAATATCGTAAAGCTATCCATAAANNKTTTGCAAACGATGACCGTTTCAAA |
| P112 | | |
| ERCAS12A- | 323 | /5Phos/TATCGTAAAGCTATCCATAAAAAANNKGCAAACGATGACCGTTTCAAAAAT |
| P113 | | |
| ERCAS12A- | 324 | /5Phos/CGTAAAGCTATCCATAAAAAATTTNNKAACGATGACCGTTTCAAAAATATG |
| P114 | | |
| ERCAS12A- | 325 | /5Phos/AAAGCTATCCATAAAAAATTTGCANNKGATGACCGTTTCAAAAATATGTTT |
| P115 | | |
| ERCAS12A- | 326 | /5Phos/GCTATCCATAAAAAATTTGCAAACNNKGACCGTTTCAAAAATATGTTTTCC |
| P116 | | |
| ERCAS12A- | 327 | /5Phos/ATCCATAAAAAATTTGCAAACGATNNKCGTTTCAAAAATATGTTTTCCGCA |
| P117 | | |
| ERCAS12A- | 328 | /5Phos/CATAAAAAATTTGCAAACGATGACNNKTTCAAAAATATGTTTTCCGCAAAA |
| P118 | | |
| ERCAS12A- | 329 | /5Phos/AAAAAATTTGCAAACGATGACCGTNNKAAAAATATGTTTTCCGCAAAATTG |
| P119 | | |
| ERCAS12A- | 330 | /5Phos/AAATTTGCAAACGATGACCGTTTCNNKAATATGTTTTCCGCAAAATTGATT |
| P120 | | |
| ERCAS12A- | 331 | /5Phos/TTTGCAAACGATGACCGTTTCAAANNKATGTTTTCCGCAAAATTGATTAGC |
| P121 | | |
| ERCAS12A- | 332 | /5Phos/GCAAACGATGACCGTTTCAAAAATNNKTTTTCCGCAAAATTGATTAGCGAC |
| P122 | | |
| ERCAS12A- | 333 | /5Phos/AACGATGACCGTTTCAAAAATATGNNKTCCGCAAAATTGATTAGCGACATT |
| P123 | | |
| ERCAS12A- | 334 | /5Phos/GATGACCGTTTCAAAAATATGTTTNNKGCAAAATTGATTAGCGACATTTTA |
| P124 | | |
| ERCAS12A- | 335 | /5Phos/GACCGTTTCAAAAATATGTTTTCCNNKAAATTGATTAGCGACATTTTACCT |
| P125 | | |
| ERCAS12A- | 336 | /5Phos/CGTTTCAAAAATATGTTTTCCGCANNKTTGATTAGCGACATTTTACCTGAA |
| P126 | | |
| ERCAS12A- | 337 | /5Phos/TTCAAAAATATGTTTTCCGCAAAANNKATTAGCGACATTTTACCTGAATTC |
| P127 | | |
| ERCAS12A- | 338 | /5Phos/AAAAATATGTTTTCCGCAAAATTGNNKAGCGACATTTTACCTGAATTCGTA |
| P128 | | |
| ERCAS12A- | 339 | /5Phos/AATATGTTTTCCGCAAAATTGATTNNKGACATTTTACCTGAATTCGTAATC |
| P129 | | |
| ERCAS12A- | 340 | /5Phos/ATGTTTTCCGCAAAATTGATTAGCNNKATTTTACCTGAATTCGTAATCCAT |
| P130 | | |
| ERCAS12A- | 341 | /5Phos/TTTTCCGCAAAATTGATTAGCGACNNKTTACCTGAATTCGTAATCCATAAC |
| P131 | | |
| ERCAS12A- | 342 | /5Phos/TCCGCAAAATTGATTAGCGACATTNNKCCTGAATTCGTAATCCATAACAAC |
| P132 | | |
| ERCAS12A- | 343 | /5Phos/GCAAAATTGATTAGCGACATTTTANNKGAATTCGTAATCCATAACAACAAC |
| P133 | | |
| ERCAS12A- | 344 | /5Phos/AAATTGATTAGCGACATTTTACCTNNKTTCGTAATCCATAACAACAACTAT |
| P134 | | |
| ERCAS12A- | 345 | /5Phos/TTGATTAGCGACATTTTACCTGAANNKGTAATCCATAACAACAACTATTCC |
| P135 | | |
| ERCAS12A- | 346 | /5Phos/ATTAGCGACATTTTACCTGAATTCNNKATCCATAACAACAACTATTCCGCA |
| P136 | | |
| ERCAS12A- | 347 | /5Phos/AGCGACATTTTACCTGAATTCGTANNKCATAACAACAACTATTCCGCATCT |
| P137 | | |
| ERCAS12A- | 348 | /5Phos/GACATTTTACCTGAATTCGTAATCNNKAACAACAACTATTCCGCATCTGAA |
| P138 | | |
| ERCAS12A- | 349 | /5Phos/ATTTTACCTGAATTCGTAATCCATNNKAACAACTATTCCGCATCTGAAAAA |
| P139 | | |
| ERCAS12A- | 350 | /5Phos/TTACCTGAATTCGTAATCCATAACNNKAACTATTCCGCATCTGAAAAAGAG |
| P140 | | |
| ERCAS12A- | 351 | /5Phos/CCTGAATTCGTAATCCATAACAACNNKTATTCCGCATCTGAAAAAGAGGAG |
| P141 | | |
| ERCAS12A- | 352 | /5Phos/GAATTCGTAATCCATAACAACAACNNKTCCGCATCTGAAAAAGAGGAGAAG |
| P142 | | |
| ERCAS12A | 353 | /5Phos/TTCGTAATCCATAACAACAACTATNNKGCATCTGAAAAAGAGGAGAAGACC |
| P143 | | |
| ERCAS12A- | 354 | /5Phos/GTAATCCATAACAACAACTATTCCNNKTCTGAAAAAGAGGAGAAGACCCAA |
| P144 | | |
| ERCAS12A- | 355 | /5Phos/ATCCATAACAACAACTATTCCGCANNKGAAAAAGAGGAGAAGACCCAAGTT |
| P145 | | |
| ERCAS12A- | 356 | /5Phos/CATAACAACAACTATTCCGCATCTNNKAAAGAGGAGAAGACCCAAGTTATC |
| P146 | | |
| ERCAS12A- | 357 | /5Phos/AACAACAACTATTCCGCATCTGAANNKGAGGAGAAGACCCAAGTTATCAAA |
| P147 | | |
| ERCAS12A- | 358 | /5Phos/AACAACTATTCCGCATCTGAAAAANNKGAGAAGACCCAAGTTATCAAACTT |
| P148 | | |
| ERCAS12A- | 359 | /5Phos/AACTATTCCGCATCTGAAAAAGAGNNKAAGACCCAAGTTATCAAACTTTTT |
| P149 | | |
| ERCAS12A- | 360 | /5Phos/TATTCCGCATCTGAAAAAGAGGAGNNKACCCAAGTTATCAAACTTTTTTCA |
| P150 | | |
| ERCAS12A- | 361 | /5Phos/TCCGCATCTGAAAAAGAGGAGAAGNNKCAAGTTATCAAACTTTTTTCACGT |
| P151 | | |
| ERCAS12A- | 362 | /5Phos/GCATCTGAAAAAGAGGAGAAGACCNNKGTTATCAAACTTTTTTCACGTTTC |
| P152 | | |
| ERCAS12A- | 363 | /5Phos/TCTGAAAAAGAGGAGAAGACCCAANNKATCAAACTTTTTTCACGTTTCGCG |
| P153 | | |
| ERCAS12A- | 364 | /5Phos/GAAAAAGAGGAGAAGACCCAAGTTNNKAAACTTTTTTCACGTTTCGCGACA |
| P154 | | |
| ERCAS12A- | 365 | /5Phos/AAAGAGGAGAAGACCCAAGTTATCNNKCTTTTTTCACGTTTCGCGACATCT |
| P155 | | |
| ERCAS12A- | 366 | /5Phos/GAGGAGAAGACCCAAGTTATCAAANNKTTTTCACGTTTCGCGACATCTTTT |
| P156 | | |
| ERCAS12A- | 367 | /5Phos/GAGAAGACCCAAGTTATCAAACTTNNKTCACGTTTCGCGACATCTTTTAAA |
| P157 | | |
| ERCAS12A- | 368 | /5Phos/AAGACCCAAGTTATCAAACTTTTTNNKCGTTTCGCGACATCTTTTAAAGAC |
| P158 | | |
| ERCAS12A- | 369 | /5Phos/ACCCAAGTTATCAAACTTTTTTCANNKTTCGCGACATCTTTTAAAGACTAC |
| P159 | | |
| ERCAS12A- | 370 | /5Phos/CAAGTTATCAAACTTTTTTCACGTNNKGCGACATCTTTTAAAGACTACTTT |
| P160 | | |
| ERCAS12A- | 371 | /5Phos/GTTATCAAACTTTTTTCACGTTTCNNKACATCTTTTAAAGACTACTTTAAG |
| P161 | | |
| ERCAS12A- | 372 | /5Phos/ATCAAACTTTTTTCACGTTTCGCGNNKTCTTTTAAAGACTACTTTAAGAAT |
| P162 | | |
| ERCAS12A- | 373 | /5Phos/AAACTTTTTTCACGTTTCGCGACANNKTTTAAAGACTACTTTAAGAATCGT |
| P163 | | |
| ERCAS12A- | 374 | /5Phos/CTTTTTTCACGTTTCGCGACATCTNNKAAAGACTACTTTAAGAATCGTGCG |
| P164 | | |
| ERCAS12A- | 375 | /5Phos/TTTTCACGTTTCGCGACATCTTTTNNKGACTACTTTAAGAATCGTGCGAAT |
| P165 | | |
| ERCAS12A- | 376 | /5Phos/TCACGTTTCGCGACATCTTTTAAANNKTACTTTAAGAATCGTGCGAATTGT |
| P166 | | |
| ERCAS12A- | 377 | /5Phos/CGTTTCGCGACATCTTTTAAAGACNNKTTTAAGAATCGTGCGAATTGTTTC |
| P167 | | |
| ERCAS12A- | 378 | /5Phos/TTCGCGACATCTTTTAAAGACTACNNKAAGAATCGTGCGAATTGTTTCAGT |
| P168 | | |
| ERCAS12A- | 379 | /5Phos/GCGACATCTTTTAAAGACTACTTTNNKAATCGTGCGAATTGTTTCAGTGCA |
| P169 | | |
| ERCAS12A- | 380 | /5Phos/ACATCTTTTAAAGACTACTTTAAGNNKCGTGCGAATTGTTTCAGTGCAGAT |
| P170 | | |
| ERCAS12A- | 381 | /5Phos/TCTTTTAAAGACTACTTTAAGAATNNKGCGAATTGTTTCAGTGCAGATGAT |
| P171 | | |
| ERCAS12A- | 382 | /5Phos/TTTAAAGACTACTTTAAGAATCGTNNKAATTGTTTCAGTGCAGATGATATT |
| P172 | | |
| ERCAS12A- | 383 | /5Phos/AAAGACTACTTTAAGAATCGTGCGNNKTGTTTCAGTGCAGATGATATTTCG |
| P173 | | |
| ERCAS12A- | 384 | /5Phos/GACTACTTTAAGAATCGTGCGAATNNKTTCAGTGCAGATGATATTTCGTCT |
| P174 | | |
| ERCAS12A- | 385 | /5Phos/TACTTTAAGAATCGTGCGAATTGTNNKAGTGCAGATGATATTTCGTCTAGT |
| P175 | | |
| ERCAS12A- | 386 | /5Phos/TTTAAGAATCGTGCGAATTGTTTCNNKGCAGATGATATTTCGTCTAGTTCA |
| P176 | | |
| ERCAS12A- | 387 | /5Phos/AAGAATCGTGCGAATTGTTTCAGTNNKGATGATATTTCGTCTAGTTCATGT |
| P177 | | |
| ERCAS12A- | 388 | /5Phos/AATCGTGCGAATTGTTTCAGTGCANNKGATATTTCGTCTAGTTCATGTCAC |
| P178 | | |
| ERCAS12A- | 389 | /5Phos/CGTGCGAATTGTTTCAGTGCAGATNNKATTTCGTCTAGTTCATGTCACCGT |
| P179 | | |
| ERCAS12A- | 390 | /5Phos/GCGAATTGTTTCAGTGCAGATGATNNKTCGTCTAGTTCATGTCACCGTATC |
| P180 | | |
| ERCAS12A- | 391 | /5Phos/AATTGTTTCAGTGCAGATGATATTNNKTCTAGTTCATGTCACCGTATCGTG |
| P181 | | |
| ERCAS12A- | 392 | /5Phos/TGTTTCAGTGCAGATGATATTTCGNNKAGTTCATGTCACCGTATCGTGAAT |
| P182 | | |
| ERCAS12A- | 393 | /5Phos/TTCAGTGCAGATGATATTTCGTCTNNKTCATGTCACCGTATCGTGAATGAC |
| P183 | | |
| ERCAS12A- | 394 | /5Phos/AGTGCAGATGATATTTCGTCTAGTNNKTGTCACCGTATCGTGAATGACAAC |
| P184 | | |
| ERCAS12A- | 395 | /5Phos/GCAGATGATATTTCGTCTAGTTCANNKCACCGTATCGTGAATGACAACGCT |
| P185 | | |
| ERCAS12A- | 396 | /5Phos/GATGATATTTCGTCTAGTTCATGTNNKCGTATCGTGAATGACAACGCTGAG |
| P186 | | |
| ERCAS12A- | 397 | /5Phos/GATATTTCGTCTAGTTCATGTCACNNKATCGTGAATGACAACGCTGAGATT |
| P187 | | |
| ERCAS12A- | 398 | /5Phos/ATTTCGTCTAGTTCATGTCACCGTNNKGTGAATGACAACGCTGAGATTTTC |
| P188 | | |
| ERCAS12A- | 399 | /5Phos/TCGTCTAGTTCATGTCACCGTATCNNKAATGACAACGCTGAGATTTTCTTT |
| P189 | | |
| ERCAS12A- | 400 | /5Phos/TCTAGTTCATGTCACCGTATCGTGNNKGACAACGCTGAGATTTTCTTTTCT |
| P190 | | |
| ERCAS12A- | 401 | /5Phos/AGTTCATGTCACCGTATCGTGAATNNKAACGCTGAGATTTTCTTTTCTAAT |
| P191 | | |
| ERCAS12A- | 402 | /5Phos/TCATGTCACCGTATCGTGAATGACNNKGCTGAGATTTTCTTTTCTAATGCG |
| P192 | | |
| ERCAS12A- | 403 | /5Phos/TGTCACCGTATCGTGAATGACAACNNKGAGATTTTCTTTTCTAATGCGTTG |
| P193 | | |
| ERCAS12A- | 404 | /5Phos/CACCGTATCGTGAATGACAACGCTNNKATTTTCTTTTCTAATGCGTTGGTG |
| P194 | | |
| ERCAS12A- | 405 | /5Phos/CGTATCGTGAATGACAACGCTGAGNNKTTCTTTTCTAATGCGTTGGTGTAT |
| P195 | | |
| ERCAS12A- | 406 | /5Phos/ATCGTGAATGACAACGCTGAGATTNNKTTTTCTAATGCGTTGGTGTATCGC |
| P196 | | |
| ERCAS12A- | 407 | /5Phos/GTGAATGACAACGCTGAGATTTTCNNKTCTAATGCGTTGGTGTATCGCCGC |
| P197 | | |
| ERCAS12A- | 408 | /5Phos/AATGACAACGCTGAGATTTTCTTTNNKAATGCGTTGGTGTATCGCCGCATC |
| P198 | | |
| ERCAS12A- | 409 | /5Phos/GACAACGCTGAGATTTTCTTTTCTNNKGCGTTGGTGTATCGCCGCATCGTA |
| P199 | | |
| ERCAS12A- | 410 | /5Phos/AACGCTGAGATTTTCTTTTCTAATNNKTTGGTGTATCGCCGCATCGTAAAG |
| P200 | | |
| ERCAS12A- | 411 | /5Phos/GCTGAGATTTTCTTTTCTAATGCGNNKGTGTATCGCCGCATCGTAAAGTCC |
| P201 | | |
| ERCAS12A- | 412 | /5Phos/GAGATTTTCTTTTCTAATGCGTTGNNKTATCGCCGCATCGTAAAGTCCTTG |
| P202 | | |
| ERCAS12A- | 413 | /5Phos/ATTTTCTTTTCTAATGCGTTGGTGNNKCGCCGCATCGTAAAGTCCTTGTCG |
| P203 | | |
| ERCAS12A- | 414 | /5Phos/TTCTTTTCTAATGCGTTGGTGTATNNKCGCATCGTAAAGTCCTTGTCGAAT |
| P204 | | |
| ERCAS12A- | 415 | /5Phos/TTTTCTAATGCGTTGGTGTATCGCNNKATCGTAAAGTCCTTGTCGAATGAT |
| P205 | | |
| ERCAS12A- | 416 | /5Phos/TCTAATGCGTTGGTGTATCGCCGCNNKGTAAAGTCCTTGTCGAATGATGAT |
| P206 | | |
| ERCAS12A- | 417 | /5Phos/AATGCGTTGGTGTATCGCCGCATCNNKAAGTCCTTGTCGAATGATGATATC |
| P207 | | |
| ERCAS12A- | 418 | /5Phos/GCGTTGGTGTATCGCCGCATCGTANNKTCCTTGTCGAATGATGATATCAAC |
| P208 | | |
| ERCAS12A- | 419 | /5Phos/TTGGTGTATCGCCGCATCGTAAAGNNKTTGTCGAATGATGATATCAACAAA |
| P209 | | |
| ERCAS12A- | 420 | /5Phos/GTGTATCGCCGCATCGTAAAGTCCNNKTCGAATGATGATATCAACAAAATT |
| P210 | | |
| ERCAS12A- | 421 | /5Phos/TATCGCCGCATCGTAAAGTCCTTGNNKAATGATGATATCAACAAAATTAGT |
| P211 | | |
| ERCAS12A- | 422 | /5Phos/CGCCGCATCGTAAAGTCCTTGTCGNNKGATGATATCAACAAAATTAGTGGT |
| P212 | | |
| ERCAS12A- | 423 | /5Phos/CGCATCGTAAAGTCCTTGTCGAATNNKGATATCAACAAAATTAGTGGTGAC |
| P213 | | |
| ERCAS12A- | 424 | /5Phos/ATCGTAAAGTCCTTGTCGAATGATNNKATCAACAAAATTAGTGGTGACATG |
| P214 | | |
| ERCAS12A- | 425 | /5Phos/GTAAAGTCCTTGTCGAATGATGATNNKAACAAAATTAGTGGTGACATGAAA |
| P215 | | |
| ERCAS12A- | 426 | /5Phos/AAGTCCTTGTCGAATGATGATATCNNKAAAATTAGTGGTGACATGAAAGAC |
| P216 | | |
| ERCAS12A- | 427 | /5Phos/TCCTTGTCGAATGATGATATCAACNNKATTAGTGGTGACATGAAAGACTCC |
| P217 | | |
| ERCAS12A- | 428 | /5Phos/TTGTCGAATGATGATATCAACAAANNKAGTGGTGACATGAAAGACTCCCTG |
| P218 | | |
| ERCAS12A- | 429 | /5Phos/TCGAATGATGATATCAACAAAATTNNKGGTGACATGAAAGACTCCCTGAAA |
| P219 | | |
| ERCAS12A- | 430 | /5Phos/AATGATGATATCAACAAAATTAGTNNKGACATGAAAGACTCCCTGAAAGAA |
| P220 | | |
| ERCAS12A- | 431 | /5Phos/GATGATATCAACAAAATTAGTGGTNNKATGAAAGACTCCCTGAAAGAAATG |
| P221 | | |
| ERCAS12A- | 432 | /5Phos/GATATCAACAAAATTAGTGGTGACNNKAAAGACTCCCTGAAAGAAATGTCA |
| P222 | | |
| ERCAS12A- | 433 | /5Phos/ATCAACAAAATTAGTGGTGACATGNNKGACTCCCTGAAAGAAATGTCATTA |
| P223 | | |
| ERCAS12A- | 434 | /5Phos/AACAAAATTAGTGGTGACATGAAANNKTCCCTGAAAGAAATGTCATTAGAG |
| P224 | | |
| ERCAS12A- | 435 | /5Phos/AAAATTAGTGGTGACATGAAAGACNNKCTGAAAGAAATGTCATTAGAGGAG |
| P225 | | |
| ERCAS12A- | 436 | /5Phos/ATTAGTGGTGACATGAAAGACTCCNNKAAAGAAATGTCATTAGAGGAGATT |
| P226 | | |
| ERCAS12A- | 437 | /5Phos/AGTGGTGACATGAAAGACTCCCTGNNKGAAATGTCATTAGAGGAGATTTAC |
| P227 | | |
| ERCAS12A- | 438 | /5Phos/GGTGACATGAAAGACTCCCTGAAANNKATGTCATTAGAGGAGATTTACAGC |
| P228 | | |
| ERCAS12A- | 439 | /5Phos/GACATGAAAGACTCCCTGAAAGAANNKTCATTAGAGGAGATTTACAGCTAC |
| P229 | | |
| ERCAS12A- | 440 | /5Phos/ATGAAAGACTCCCTGAAAGAAATGNNKTTAGAGGAGATTTACAGCTACGAA |
| P230 | | |
| ERCAS12A- | 441 | /5Phos/AAAGACTCCCTGAAAGAAATGTCANNKGAGGAGATTTACAGCTACGAAAAA |
| P231 | | |
| ERCAS12A- | 442 | /5Phos/GACTCCCTGAAAGAAATGTCATTANNKGAGATTTACAGCTACGAAAAATAC |
| P232 | | |
| ERCAS12A- | 443 | /5Phos/TCCCTGAAAGAAATGTCATTAGAGNNKATTTACAGCTACGAAAAATACGGA |
| P233 | | |
| ERCAS12A- | 444 | /5Phos/CTGAAAGAAATGTCATTAGAGGAGNNKTACAGCTACGAAAAATACGGAGAG |
| P234 | | |
| ERCAS12A- | 445 | /5Phos/AAAGAAATGTCATTAGAGGAGATTNNKAGCTACGAAAAATACGGAGAGTTC |
| P235 | | |
| ERCAS12A- | 446 | /5Phos/GAAATGTCATTAGAGGAGATTTACNNKTACGAAAAATACGGAGAGTTCATC |
| P236 | | |
| ERCAS12A- | 447 | /5Phos/ATGTCATTAGAGGAGATTTACAGCNNKGAAAAATACGGAGAGTTCATCACT |
| P237 | | |
| ERCAS12A- | 448 | /5Phos/TCATTAGAGGAGATTTACAGCTACNNKAAATACGGAGAGTTCATCACTCAG |
| P238 | | |
| ERCAS12A- | 449 | /5Phos/TTAGAGGAGATTTACAGCTACGAANNKTACGGAGAGTTCATCACTCAGGAA |
| P239 | | |
| ERCAS12A- | 450 | /5Phos/GAGGAGATTTACAGCTACGAAAAANNKGGAGAGTTCATCACTCAGGAAGGT |
| P240 | | |
| ERCAS12A- | 451 | /5Phos/GAGATTTACAGCTACGAAAAATACNNKGAGTTCATCACTCAGGAAGGTATC |
| P241 | | |
| ERCAS12A- | 452 | /5Phos/ATTTACAGCTACGAAAAATACGGANNKTTCATCACTCAGGAAGGTATCTCC |
| P242 | | |
| ERCAS12A- | 453 | /5Phos/TACAGCTACGAAAAATACGGAGAGNNKATCACTCAGGAAGGTATCTCCTTT |
| P243 | | |
| ERCAS12A- | 454 | /5Phos/AGCTACGAAAAATACGGAGAGTTCNNKACTCAGGAAGGTATCTCCTTTTAC |
| P244 | | |
| ERCAS12A- | 455 | /5Phos/TACGAAAAATACGGAGAGTTCATCNNKCAGGAAGGTATCTCCTTTTACAAC |
| P245 | | |
| ERCAS12A- | 456 | /5Phos/GAAAAATACGGAGAGTTCATCACTNNKGAAGGTATCTCCTTTTACAACGAT |
| P246 | | |
| ERCAS12A- | 457 | /5Phos/AAATACGGAGAGTTCATCACTCAGNNKGGTATCTCCTTTTACAACGATATT |
| P247 | | |
| ERCAS12A- | 458 | /5Phos/TACGGAGAGTTCATCACTCAGGAANNKATCTCCTTTTACAACGATATTTGC |
| P248 | | |
| ERCAS12A- | 459 | /5Phos/GGAGAGTTCATCACTCAGGAAGGTNNKTCCTTTTACAACGATATTTGCGGC |
| P249 | | |
| ERCAS12A- | 460 | /5Phos/GAGTTCATCACTCAGGAAGGTATCNNKTTTTACAACGATATTTGCGGCAAA |
| P250 | | |
| ERCAS12A- | 461 | /5Phos/TTCATCACTCAGGAAGGTATCTCCNNKTACAACGATATTTGCGGCAAAGTT |
| P251 | | |
| ERCAS12A- | 462 | /5Phos/ATCACTCAGGAAGGTATCTCCTTTNNKAACGATATTTGCGGCAAAGTTAAT |
| P252 | | |
| ERCAS12A- | 463 | /5Phos/ACTCAGGAAGGTATCTCCTTTTACNNKGATATTTGCGGCAAAGTTAATAGT |
| P253 | | |
| ERCAS12A- | 464 | /5Phos/CAGGAAGGTATCTCCTTTTACAACNNKATTTGCGGCAAAGTTAATAGTTTC |
| P254 | | |
| ERCAS12A- | 465 | /5Phos/GAAGGTATCTCCTTTTACAACGATNNKTGCGGCAAAGTTAATAGTTTCATG |
| P255 | | |
| ERCAS12A- | 466 | /5Phos/GGTATCTCCTTTTACAACGATATTNNKGGCAAAGTTAATAGTTTCATGAAT |
| P256 | | |
| ERCAS12A- | 467 | /5Phos/ATCTCCTTTTACAACGATATTTGCNNKAAAGTTAATAGTTTCATGAATCTG |
| P257 | | |
| ERCAS12A- | 468 | /5Phos/TCCTTTTACAACGATATTTGCGGCNNKGTTAATAGTTTCATGAATCTGTAT |
| P258 | | |
| ERCAS12A- | 469 | /5Phos/TTTTACAACGATATTTGCGGCAAANNKAATAGTTTCATGAATCTGTATTGT |
| P259 | | |
| ERCAS12A- | 470 | /5Phos/TACAACGATATTTGCGGCAAAGTTNNKAGTTTCATGAATCTGTATTGTCAG |
| P260 | | |
| ERCAS12A- | 471 | /5Phos/AACGATATTTGCGGCAAAGTTAATNNKTTCATGAATCTGTATTGTCAGAAG |
| P261 | | |
| ERCAS12A- | 472 | /5Phos/GATATTTGCGGCAAAGTTAATAGTNNKATGAATCTGTATTGTCAGAAGAAT |
| P262 | | |
| ERCAS12A- | 473 | /5Phos/ATTTGCGGCAAAGTTAATAGTTTCNNKAATCTGTATTGTCAGAAGAATAAG |
| P263 | | |
| ERCAS12A- | 474 | /5Phos/TGCGGCAAAGTTAATAGTTTCATGNNKCTGTATTGTCAGAAGAATAAGGAA |
| P264 | | |
| ERCAS12A- | 475 | /5Phos/GGCAAAGTTAATAGTTTCATGAATNNKTATTGTCAGAAGAATAAGGAAAAC |
| P265 | | |
| ERCAS12A- | 476 | /5Phos/AAAGTTAATAGTTTCATGAATCTGNNKTGTCAGAAGAATAAGGAAAACAAG |
| P266 | | |
| ERCAS12A- | 477 | /5Phos/GTTAATAGTTTCATGAATCTGTATNNKCAGAAGAATAAGGAAAACAAGAAT |
| P267 | | |
| ERCAS12A- | 478 | /5Phos/AATAGTTTCATGAATCTGTATTGTNNKAAGAATAAGGAAAACAAGAATCTG |
| P268 | | |
| ERCAS12A- | 479 | /5Phos/AGTTTCATGAATCTGTATTGTCAGNNKAATAAGGAAAACAAGAATCTGTAC |
| P269 | | |
| ERCAS12A- | 480 | /5Phos/TTCATGAATCTGTATTGTCAGAAGNNKAAGGAAAACAAGAATCTGTACAAG |
| P270 | | |
| ERCAS12A- | 481 | /5Phos/ATGAATCTGTATTGTCAGAAGAATNNKGAAAACAAGAATCTGTACAAGTTA |
| P271 | | |
| ERCAS12A- | 482 | /5Phos/AATCTGTATTGTCAGAAGAATAAGNNKAACAAGAATCTGTACAAGTTACAA |
| P272 | | |
| ERCAS12A- | 483 | /5Phos/CTGTATTGTCAGAAGAATAAGGAANNKAAGAATCTGTACAAGTTACAAAAG |
| P273 | | |
| ERCAS12A- | 484 | /5Phos/TATTGTCAGAAGAATAAGGAAAACNNKAATCTGTACAAGTTACAAAAGCTT |
| P274 | | |
| ERCAS12A- | 485 | /5Phos/TGTCAGAAGAATAAGGAAAACAAGNNKCTGTACAAGTTACAAAAGCTTCAT |
| P275 | | |
| ERCAS12A- | 486 | /5Phos/CAGAAGAATAAGGAAAACAAGAATNNKTACAAGTTACAAAAGCTTCATAAA |
| P276 | | |
| ERCAS12A- | 487 | /5Phos/AAGAATAAGGAAAACAAGAATCTGNNKAAGTTACAAAAGCTTCATAAACAG |
| P277 | | |
| ERCAS12A- | 488 | /5Phos/AATAAGGAAAACAAGAATCTGTACNNKTTACAAAAGCTTCATAAACAGATC |
| P278 | | |
| ERCAS12A- | 489 | /5Phos/AAGGAAAACAAGAATCTGTACAAGNNKCAAAAGCTTCATAAACAGATCTTG |
| P279 | | |
| ERCAS12A- | 490 | /5Phos/GAAAACAAGAATCTGTACAAGTTANNKAAGCTTCATAAACAGATCTTGTGC |
| P280 | | |
| ERCAS12A- | 491 | /5Phos/AACAAGAATCTGTACAAGTTACAANNKCTTCATAAACAGATCTTGTGCATC |
| P281 | | |
| ERCAS12A- | 492 | /5Phos/AAGAATCTGTACAAGTTACAAAAGNNKCATAAACAGATCTTGTGCATCGCC |
| P282 | | |
| ERCAS12A- | 493 | /5Phos/AATCTGTACAAGTTACAAAAGCTTNNKAAACAGATCTTGTGCATCGCCGAC |
| P283 | | |
| ERCAS12A- | 494 | /5Phos/CTGTACAAGTTACAAAAGCTTCATNNKCAGATCTTGTGCATCGCCGACACG |
| P284 | | |
| ERCAS12A- | 495 | /5Phos/TACAAGTTACAAAAGCTTCATAAANNKATCTTGTGCATCGCCGACACGTCT |
| P285 | | |
| ERCAS12A- | 496 | /5Phos/AAGTTACAAAAGCTTCATAAACAGNNKTTGTGCATCGCCGACACGTCTTAT |
| P286 | | |
| ERCAS12A- | 497 | /5Phos/TTACAAAAGCTTCATAAACAGATCNNKTGCATCGCCGACACGTCTTATGAA |
| P287 | | |
| ERCAS12A- | 498 | /5Phos/CAAAAGCTTCATAAACAGATCTTGNNKATCGCCGACACGTCTTATGAAGTC |
| P288 | | |
| ERCAS12A- | 499 | /5Phos/AAGCTTCATAAACAGATCTTGTGCNNKGCCGACACGTCTTATGAAGTCCCG |
| P289 | | |
| ERCAS12A- | 500 | /5Phos/CTTCATAAACAGATCTTGTGCATCNNKGACACGTCTTATGAAGTCCCGTAT |
| P290 | | |
| ERCAS12A- | 501 | /5Phos/CATAAACAGATCTTGTGCATCGCCNNKACGTCTTATGAAGTCCCGTATAAA |
| P291 | | |
| ERCAS12A- | 502 | /5Phos/AAACAGATCTTGTGCATCGCCGACNNKTCTTATGAAGTCCCGTATAAATTT |
| P292 | | |
| ERCAS12A- | 503 | /5Phos/CAGATCTTGTGCATCGCCGACACGNNKTATGAAGTCCCGTATAAATTTGAA |
| P293 | | |
| ERCAS12A- | 504 | /5Phos/ATCTTGTGCATCGCCGACACGTCTNNKGAAGTCCCGTATAAATTTGAAAGC |
| P294 | | |
| ERCAS12A- | 505 | /5Phos/TTGTGCATCGCCGACACGTCTTATNNKGTCCCGTATAAATTTGAAAGCGAC |
| P295 | | |
| ERCAS12A- | 506 | /5Phos/TGCATCGCCGACACGTCTTATGAANNKCCGTATAAATTTGAAAGCGACGAA |
| P296 | | |
| ERCAS12A- | 507 | /5Phos/ATCGCCGACACGTCTTATGAAGTCNNKTATAAATTTGAAAGCGACGAAGAA |
| P297 | | |
| ERCAS12A- | 508 | /5Phos/GCCGACACGTCTTATGAAGTCCCGNNKAAATTTGAAAGCGACGAAGAAGTA |
| P298 | | |
| ERCAS12A- | 509 | /5Phos/GACACGTCTTATGAAGTCCCGTATNNKTTTGAAAGCGACGAAGAAGTATAT |
| P299 | | |
| ERCAS12A- | 510 | /5Phos/ACGTCTTATGAAGTCCCGTATAAANNKGAAAGCGACGAAGAAGTATATCAG |
| P300 | | |
| ERCAS12A- | 511 | /5Phos/TCTTATGAAGTCCCGTATAAATTTNNKAGCGACGAAGAAGTATATCAGAGT |
| P301 | | |
| ERCAS12A- | 512 | /5Phos/TATGAAGTCCCGTATAAATTTGAANNKGACGAAGAAGTATATCAGAGTGTG |
| P302 | | |
| ERCAS12A- | 513 | /5Phos/GAAGTCCCGTATAAATTTGAAAGCNNKGAAGAAGTATATCAGAGTGTGAAT |
| P303 | | |
| ERCAS12A- | 514 | /5Phos/GTCCCGTATAAATTTGAAAGCGACNNKGAAGTATATCAGAGTGTGAATGGA |
| P304 | | |
| ERCAS12A- | 515 | /5Phos/CCGTATAAATTTGAAAGCGACGAANNKGTATATCAGAGTGTGAATGGATTC |
| P305 | | |
| ERCAS12A- | 516 | /5Phos/TATAAATTTGAAAGCGACGAAGAANNKTATCAGAGTGTGAATGGATTCTTG |
| P306 | | |
| ERCAS12A- | 517 | /5Phos/AAATTTGAAAGCGACGAAGAAGTANNKCAGAGTGTGAATGGATTCTTGGAC |
| P307 | | |
| ERCAS12A- | 518 | /5Phos/TTTGAAAGCGACGAAGAAGTATATNNKAGTGTGAATGGATTCTTGGACAAT |
| P308 | | |
| ERCAS12A- | 519 | /5Phos/GAAAGCGACGAAGAAGTATATCAGNNKGTGAATGGATTCTTGGACAATATC |
| P309 | | |
| ERCAS12A- | 520 | /5Phos/AGCGACGAAGAAGTATATCAGAGTNNKAATGGATTCTTGGACAATATCTCA |
| P310 | | |
| ERCAS12A- | 521 | /5Phos/GACGAAGAAGTATATCAGAGTGTGNNKGGATTCTTGGACAATATCTCATCA |
| P311 | | |
| ERCAS12A- | 522 | /5Phos/GAAGAAGTATATCAGAGTGTGAATNNKTTCTTGGACAATATCTCATCAAAA |
| P312 | | |
| ERCAS12A- | 523 | /5Phos/GAAGTATATCAGAGTGTGAATGGANNKTTGGACAATATCTCATCAAAACAC |
| P313 | | |
| ERCAS12A- | 524 | /5Phos/GTATATCAGAGTGTGAATGGATTCNNKGACAATATCTCATCAAAACACATT |
| P314 | | |
| ERCAS12A- | 525 | /5Phos/TATCAGAGTGTGAATGGATTCTTGNNKAATATCTCATCAAAACACATTGTT |
| P315 | | |
| ERCAS12A- | 526 | /5Phos/CAGAGTGTGAATGGATTCTTGGACNNKATCTCATCAAAACACATTGTTGAA |
| P316 | | |
| ERCAS12A- | 527 | /5Phos/AGTGTGAATGGATTCTTGGACAATNNKTCATCAAAACACATTGTTGAACGT |
| P317 | | |
| ERCAS12A- | 528 | /5Phos/GTGAATGGATTCTTGGACAATATCNNKTCAAAACACATTGTTGAACGTTTG |
| P318 | | |
| ERCAS12A- | 529 | /5Phos/AATGGATTCTTGGACAATATCTCANNKAAACACATTGTTGAACGTTTGCGC |
| P319 | | |
| ERCAS12A- | 530 | /5Phos/GGATTCTTGGACAATATCTCATCANNKCACATTGTTGAACGTTTGCGCAAA |
| P320 | | |
| ERCAS12A- | 531 | /5Phos/TTCTTGGACAATATCTCATCAAAANNKATTGTTGAACGTTTGCGCAAAATC |
| P321 | | |
| ERCAS12A- | 532 | /5Phos/TTGGACAATATCTCATCAAAACACNNKGTTGAACGTTTGCGCAAAATCGGC |
| P322 | | |
| ERCAS12A- | 533 | /5Phos/GACAATATCTCATCAAAACACATTNNKGAACGTTTGCGCAAAATCGGCGAC |
| P323 | | |
| ERCAS12A- | 534 | /5Phos/AATATCTCATCAAAACACATTGTTNNKCGTTTGCGCAAAATCGGCGACAAC |
| P324 | | |
| ERCAS12A- | 535 | /5Phos/ATCTCATCAAAACACATTGTTGAANNKTTGCGCAAAATCGGCGACAACTAT |
| P325 | | |
| ERCAS12A- | 536 | /5Phos/TCATCAAAACACATTGTTGAACGTNNKCGCAAAATCGGCGACAACTATAAC |
| P326 | | |
| ERCAS12A- | 537 | /5Phos/TCAAAACACATTGTTGAACGTTTGNNKAAAATCGGCGACAACTATAACGGA |
| P327 | | |
| ERCAS12A- | 538 | /5Phos/AAACACATTGTTGAACGTTTGCGCNNKATCGGCGACAACTATAACGGATAC |
| P328 | | |
| ERCAS12A- | 539 | /5Phos/CACATTGTTGAACGTTTGCGCAAANNKGGCGACAACTATAACGGATACAAC |
| P329 | | |
| ERCAS12A- | 540 | /5Phos/ATTGTTGAACGTTTGCGCAAAATCNNKGACAACTATAACGGATACAACTTG |
| P330 | | |
| ERCAS12A- | 541 | /5Phos/GTTGAACGTTTGCGCAAAATCGGCNNKAACTATAACGGATACAACTTGGAT |
| P331 | | |
| ERCAS12A- | 542 | /5Phos/GAACGTTTGCGCAAAATCGGCGACNNKTATAACGGATACAACTTGGATAAA |
| P332 | | |
| ERCAS12A- | 543 | /5Phos/CGTTTGCGCAAAATCGGCGACAACNNKAACGGATACAACTTGGATAAAATT |
| P333 | | |
| ERCAS12A- | 544 | /5Phos/TTGCGCAAAATCGGCGACAACTATNNKGGATACAACTTGGATAAAATTTAT |
| P334 | | |
| ERCAS12A- | 545 | /5Phos/CGCAAAATCGGCGACAACTATAACNNKTACAACTTGGATAAAATTTATATC |
| P335 | | |
| ERCAS12A- | 546 | /5Phos/AAAATCGGCGACAACTATAACGGANNKAACTTGGATAAAATTTATATCGTA |
| P336 | | |
| ERCAS12A- | 547 | /5Phos/ATCGGCGACAACTATAACGGATACNNKTTGGATAAAATTTATATCGTATCA |
| P337 | | |
| ERCAS12A- | 548 | /5Phos/GGCGACAACTATAACGGATACAACNNKGATAAAATTTATATCGTATCAAAA |
| P338 | | |
| ERCAS12A- | 549 | /5Phos/GACAACTATAACGGATACAACTTGNNKAAAATTTATATCGTATCAAAATTT |
| P339 | | |
| ERCAS12A- | 550 | /5Phos/AACTATAACGGATACAACTTGGATNNKATTTATATCGTATCAAAATTTTAT |
| P340 | | |
| ERCAS12A- | 551 | /5Phos/TATAACGGATACAACTTGGATAAANNKTATATCGTATCAAAATTTTATGAA |
| P341 | | |
| ERCAS12A- | 552 | /5Phos/AACGGATACAACTTGGATAAAATTNNKATCGTATCAAAATTTTATGAATCA |
| P342 | | |
| ERCAS12A- | 553 | /5Phos/GGATACAACTTGGATAAAATTTATNNKGTATCAAAATTTTATGAATCAGTA |
| P343 | | |
| ERCAS12A- | 554 | /5Phos/TACAACTTGGATAAAATTTATATCNNKTCAAAATTTTATGAATCAGTATCC |
| P344 | | |
| ERCAS12A- | 555 | /5Phos/AACTTGGATAAAATTTATATCGTANNKAAATTTTATGAATCAGTATCCCAA |
| P345 | | |
| ERCAS12A- | 556 | /5Phos/TTGGATAAAATTTATATCGTATCANNKTTTTATGAATCAGTATCCCAAAAG |
| P346 | | |
| ERCAS12A- | 557 | /5Phos/GATAAAATTTATATCGTATCAAAANNKTATGAATCAGTATCCCAAAAGACT |
| P347 | | |
| ERCAS12A- | 558 | /5Phos/AAAATTTATATCGTATCAAAATTTNNKGAATCAGTATCCCAAAAGACTTAC |
| P348 | | |
| ERCAS12A- | 559 | /5Phos/ATTTATATCGTATCAAAATTTTATNNKTCAGTATCCCAAAAGACTTACCGT |
| P349 | | |
| ERCAS12A- | 560 | /5Phos/TATATCGTATCAAAATTTTATGAANNKGTATCCCAAAAGACTTACCGTGAT |
| P350 | | |
| ERCAS12A- | 561 | /5Phos/ATCGTATCAAAATTTTATGAATCANNKTCCCAAAAGACTTACCGTGATTGG |
| P351 | | |
| ERCAS12A- | 562 | /5Phos/GTATCAAAATTTTATGAATCAGTANNKCAAAAGACTTACCGTGATTGGGAG |
| P352 | | |
| ERCAS12A- | 563 | /5Phos/TCAAAATTTTATGAATCAGTATCCNNKAAGACTTACCGTGATTGGGAGACT |
| P353 | | |
| ERCAS12A- | 564 | /5Phos/AAATTTTATGAATCAGTATCCCAANNKACTTACCGTGATTGGGAGACTATT |
| P354 | | |
| ERCAS12A- | 565 | /5Phos/TTTTATGAATCAGTATCCCAAAAGNNKTACCGTGATTGGGAGACTATTAAT |
| P355 | | |
| ERCAS12A- | 566 | /5Phos/TATGAATCAGTATCCCAAAAGACTNNKCGTGATTGGGAGACTATTAATACG |
| P356 | | |
| ERCAS12A- | 567 | /5Phos/GAATCAGTATCCCAAAAGACTTACNNKGATTGGGAGACTATTAATACGGCG |
| P357 | | |
| ERCAS12A- | 568 | /5Phos/TCAGTATCCCAAAAGACTTACCGTNNKTGGGAGACTATTAATACGGCGTTA |
| P358 | | |
| ERCAS12A- | 569 | /5Phos/GTATCCCAAAAGACTTACCGTGATNNKGAGACTATTAATACGGCGTTAGAA |
| P359 | | |
| ERCAS12A- | 570 | /5Phos/TCCCAAAAGACTTACCGTGATTGGNNKACTATTAATACGGCGTTAGAAATC |
| P360 | | |
| ERCAS12A- | 571 | /5Phos/CAAAAGACTTACCGTGATTGGGAGNNKATTAATACGGCGTTAGAAATCCAT |
| P361 | | |
| ERCAS12A- | 572 | /5Phos/AAGACTTACCGTGATTGGGAGACTNNKAATACGGCGTTAGAAATCCATTAC |
| P362 | | |
| ERCAS12A- | 573 | /5Phos/ACTTACCGTGATTGGGAGACTATTNNKACGGCGTTAGAAATCCATTACAAC |
| P363 | | |
| ERCAS12A- | 574 | /5Phos/TACCGTGATTGGGAGACTATTAATNNKGCGTTAGAAATCCATTACAACAAC |
| P364 | | |
| ERCAS12A- | 575 | /5Phos/CGTGATTGGGAGACTATTAATACGNNKTTAGAAATCCATTACAACAACATT |
| P365 | | |
| ERCAS12A- | 576 | /5Phos/GATTGGGAGACTATTAATACGGCGNNKGAAATCCATTACAACAACATTCTG |
| P366 | | |
| ERCAS12A- | 577 | /5Phos/TGGGAGACTATTAATACGGCGTTANNKATCCATTACAACAACATTCTGCCG |
| P367 | | |
| ERCAS12A- | 578 | /5Phos/GAGACTATTAATACGGCGTTAGAANNKCATTACAACAACATTCTGCCGGGT |
| P368 | | |
| ERCAS12A- | 579 | /5Phos/ACTATTAATACGGCGTTAGAAATCNNKTACAACAACATTCTGCCGGGTAAC |
| P369 | | |
| ERCAS12A- | 580 | /5Phos/ATTAATACGGCGTTAGAAATCCATNNKAACAACATTCTGCCGGGTAACGGT |
| P370 | | |
| ERCAS12A- | 581 | /5Phos/AATACGGCGTTAGAAATCCATTACNNKAACATTCTGCCGGGTAACGGTAAA |
| P371 | | |
| ERCAS12A- | 582 | /5Phos/ACGGCGTTAGAAATCCATTACAACNNKATTCTGCCGGGTAACGGTAAATCG |
| P372 | | |
| ERCAS12A- | 583 | /5Phos/GCGTTAGAAATCCATTACAACAACNNKCTGCCGGGTAACGGTAAATCGAAA |
| P373 | | |
| ERCAS12A- | 584 | /5Phos/TTAGAAATCCATTACAACAACATTNNKCCGGGTAACGGTAAATCGAAAGCG |
| P374 | | |
| ERCAS12A- | 585 | /5Phos/GAAATCCATTACAACAACATTCTGNNKGGTAACGGTAAATCGAAAGCGGAC |
| P375 | | |
| ERCAS12A- | 586 | /5Phos/ATCCATTACAACAACATTCTGCCGNNKAACGGTAAATCGAAAGCGGACAAA |
| P376 | | |
| ERCAS12A- | 587 | /5Phos/CATTACAACAACATTCTGCCGGGTNNKGGTAAATCGAAAGCGGACAAAGTG |
| P377 | | |
| ERCAS12A- | 588 | /5Phos/TACAACAACATTCTGCCGGGTAACNNKAAATCGAAAGCGGACAAAGTGAAG |
| P378 | | |
| ERCAS12A- | 589 | /5Phos/AACAACATTCTGCCGGGTAACGGTNNKTCGAAAGCGGACAAAGTGAAGAAG |
| P379 | | |
| ERCAS12A- | 590 | /5Phos/AACATTCTGCCGGGTAACGGTAAANNKAAAGCGGACAAAGTGAAGAAGGCG |
| P380 | | |
| ERCAS12A- | 591 | /5Phos/ATTCTGCCGGGTAACGGTAAATCGNNKGCGGACAAAGTGAAGAAGGCGGTT |
| P381 | | |
| ERCAS12A- | 592 | /5Phos/CTGCCGGGTAACGGTAAATCGAAANNKGACAAAGTGAAGAAGGCGGTTAAG |
| P382 | | |
| ERCAS12A- | 593 | /5Phos/CCGGGTAACGGTAAATCGAAAGCGNNKAAAGTGAAGAAGGCGGTTAAGAAC |
| P383 | | |
| ERCAS12A- | 594 | /5Phos/GGTAACGGTAAATCGAAAGCGGACNNKGTGAAGAAGGCGGTTAAGAACGAT |
| P384 | | |
| ERCAS12A- | 595 | /5Phos/AACGGTAAATCGAAAGCGGACAAANNKAAGAAGGCGGTTAAGAACGATCTT |
| P385 | | |
| ERCAS12A- | 596 | /5Phos/GGTAAATCGAAAGCGGACAAAGTGNNKAAGGCGGTTAAGAACGATCTTCAG |
| P386 | | |
| ERCAS12A- | 597 | /5Phos/AAATCGAAAGCGGACAAAGTGAAGNNKGCGGTTAAGAACGATCTTCAGAAG |
| P387 | | |
| ERCAS12A- | 598 | /5Phos/TCGAAAGCGGACAAAGTGAAGAAGNNKGTTAAGAACGATCTTCAGAAGTCT |
| P388 | | |
| ERCAS12A- | 599 | /5Phos/AAAGCGGACAAAGTGAAGAAGGCGNNKAAGAACGATCTTCAGAAGTCTATC |
| P389 | | |
| ERCAS12A- | 600 | /5Phos/GCGGACAAAGTGAAGAAGGCGGTTNNKAACGATCTTCAGAAGTCTATCACG |
| P390 | | |
| ERCAS12A- | 601 | /5Phos/GACAAAGTGAAGAAGGCGGTTAAGNNKGATCTTCAGAAGTCTATCACGGAG |
| P391 | | |
| ERCAS12A- | 602 | /5Phos/AAAGTGAAGAAGGCGGTTAAGAACNNKCTTCAGAAGTCTATCACGGAGATT |
| P392 | | |
| ERCAS12A- | 603 | /5Phos/GTGAAGAAGGCGGTTAAGAACGATNNKCAGAAGTCTATCACGGAGATTAAT |
| P393 | | |
| ERCAS12A- | 604 | /5Phos/AAGAAGGCGGTTAAGAACGATCTTNNKAAGTCTATCACGGAGATTAATGAG |
| P394 | | |
| ERCAS12A- | 605 | /5Phos/AAGGCGGTTAAGAACGATCTTCAGNNKTCTATCACGGAGATTAATGAGCTG |
| P395 | | |
| ERCAS12A- | 606 | /5Phos/GCGGTTAAGAACGATCTTCAGAAGNNKATCACGGAGATTAATGAGCTGGTT |
| P396 | | |
| ERCAS12A- | 607 | /5Phos/GTTAAGAACGATCTTCAGAAGTCTNNKACGGAGATTAATGAGCTGGTTTCC |
| P397 | | |
| ERCAS12A- | 608 | /5Phos/AAGAACGATCTTCAGAAGTCTATCNNKGAGATTAATGAGCTGGTTTCCAAT |
| P398 | | |
| ERCAS12A- | 609 | /5Phos/AACGATCTTCAGAAGTCTATCACGNNKATTAATGAGCTGGTTTCCAATTAC |
| P399 | | |
| ERCAS12A- | 610 | /5Phos/GATCTTCAGAAGTCTATCACGGAGNNKAATGAGCTGGTTTCCAATTACAAG |
| P400 | | |
| ERCAS12A- | 611 | /5Phos/CTTCAGAAGTCTATCACGGAGATTNNKGAGCTGGTTTCCAATTACAAGTTG |
| P401 | | |
| ERCAS12A- | 612 | /5Phos/CAGAAGTCTATCACGGAGATTAATNNKCTGGTTTCCAATTACAAGTTGTGC |
| P402 | | |
| ERCAS12A- | 613 | /5Phos/AAGTCTATCACGGAGATTAATGAGNNKGTTTCCAATTACAAGTTGTGCTCG |
| P403 | | |
| ERCAS12A- | 614 | /5Phos/TCTATCACGGAGATTAATGAGCTGNNKTCCAATTACAAGTTGTGCTCGGAT |
| P404 | | |
| ERCAS12A- | 615 | /5Phos/ATCACGGAGATTAATGAGCTGGTTNNKAATTACAAGTTGTGCTCGGATGAT |
| P405 | | |
| ERCAS12A- | 616 | /5Phos/ACGGAGATTAATGAGCTGGTTTCCNNKTACAAGTTGTGCTCGGATGATAAC |
| P406 | | |
| ERCAS12A- | 617 | /5Phos/GAGATTAATGAGCTGGTTTCCAATNNKAAGTTGTGCTCGGATGATAACATC |
| P407 | | |
| ERCAS12A- | 618 | /5Phos/ATTAATGAGCTGGTTTCCAATTACNNKTTGTGCTCGGATGATAACATCAAA |
| P408 | | |
| ERCAS12A- | 619 | /5Phos/AATGAGCTGGTTTCCAATTACAAGNNKTGCTCGGATGATAACATCAAAGCT |
| P409 | | |
| ERCAS12A- | 620 | /5Phos/GAGCTGGTTTCCAATTACAAGTTGNNKTCGGATGATAACATCAAAGCTGAA |
| P410 | | |
| ERCAS12A- | 621 | /5Phos/CTGGTTTCCAATTACAAGTTGTGCNNKGATGATAACATCAAAGCTGAAACC |
| P411 | | |
| ERCAS12A- | 622 | /5Phos/GTTTCCAATTACAAGTTGTGCTCGNNKGATAACATCAAAGCTGAAACCTAT |
| P412 | | |
| ERCAS12A- | 623 | /5Phos/TCCAATTACAAGTTGTGCTCGGATNNKAACATCAAAGCTGAAACCTATATC |
| P413 | | |
| ERCAS12A- | 624 | /5Phos/AATTACAAGTTGTGCTCGGATGATNNKATCAAAGCTGAAACCTATATCCAT |
| P414 | | |
| ERCAS12A- | 625 | /5Phos/TACAAGTTGTGCTCGGATGATAACNNKAAAGCTGAAACCTATATCCATGAA |
| P415 | | |
| ERCAS12A- | 626 | /5Phos/AAGTTGTGCTCGGATGATAACATCNNKGCTGAAACCTATATCCATGAAATT |
| P416 | | |
| ERCAS12A- | 627 | /5Phos/TTGTGCTCGGATGATAACATCAAANNKGAAACCTATATCCATGAAATTTCT |
| P417 | | |
| ERCAS12A- | 628 | /5Phos/TGCTCGGATGATAACATCAAAGCTNNKACCTATATCCATGAAATTTCTCAT |
| P418 | | |
| ERCAS12A- | 629 | /5Phos/TCGGATGATAACATCAAAGCTGAANNKTATATCCATGAAATTTCTCATATT |
| P419 | | |
| ERCAS12A- | 630 | /5Phos/GATGATAACATCAAAGCTGAAACCNNKATCCATGAAATTTCTCATATTCTT |
| P420 | | |
| ERCAS12A- | 631 | /5Phos/GATAACATCAAAGCTGAAACCTATNNKCATGAAATTTCTCATATTCTTAAT |
| P421 | | |
| ERCAS12A- | 632 | /5Phos/AACATCAAAGCTGAAACCTATATCNNKGAAATTTCTCATATTCTTAATAAC |
| P422 | | |
| ERCAS12A- | 633 | /5Phos/ATCAAAGCTGAAACCTATATCCATNNKATTTCTCATATTCTTAATAACTTC |
| P423 | | |
| ERCAS12A- | 634 | /5Phos/AAAGCTGAAACCTATATCCATGAANNKTCTCATATTCTTAATAACTTCGAG |
| P424 | | |
| ERCAS12A- | 635 | /5Phos/GCTGAAACCTATATCCATGAAATTNNKCATATTCTTAATAACTTCGAGGCA |
| P425 | | |
| ERCAS12A- | 636 | /5Phos/GAAACCTATATCCATGAAATTTCTNNKATTCTTAATAACTTCGAGGCACAA |
| P426 | | |
| ERCAS12A- | 637 | /5Phos/ACCTATATCCATGAAATTTCTCATNNKCTTAATAACTTCGAGGCACAAGAA |
| P427 | | |
| ERCAS12A- | 638 | /5Phos/TATATCCATGAAATTTCTCATATTNNKAATAACTTCGAGGCACAAGAATTA |
| P428 | | |
| ERCAS12A- | 639 | /5Phos/ATCCATGAAATTTCTCATATTCTTNNKAACTTCGAGGCACAAGAATTAAAA |
| P429 | | |
| ERCAS12A- | 640 | /5Phos/CATGAAATTTCTCATATTCTTAATNNKTTCGAGGCACAAGAATTAAAATAT |
| P430 | | |
| ERCAS12A- | 641 | /5Phos/GAAATTTCTCATATTCTTAATAACNNKGAGGCACAAGAATTAAAATATAAC |
| P431 | | |
| ERCAS12A- | 642 | /5Phos/ATTTCTCATATTCTTAATAACTTCNNKGCACAAGAATTAAAATATAACCCA |
| P432 | | |
| ERCAS12A- | 643 | /5Phos/TCTCATATTCTTAATAACTTCGAGNNKCAAGAATTAAAATATAACCCAGAA |
| P433 | | |
| ERCAS12A- | 644 | /5Phos/CATATTCTTAATAACTTCGAGGCANNKGAATTAAAATATAACCCAGAAATT |
| P434 | | |
| ERCAS12A- | 645 | /5Phos/ATTCTTAATAACTTCGAGGCACAANNKTTAAAATATAACCCAGAAATTCAT |
| P435 | | |
| ERCAS12A- | 646 | /5Phos/CTTAATAACTTCGAGGCACAAGAANNKAAATATAACCCAGAAATTCATCTG |
| P436 | | |
| ERCAS12A- | 647 | /5Phos/AATAACTTCGAGGCACAAGAATTANNKTATAACCCAGAAATTCATCTGGTT |
| P437 | | |
| ERCAS12A- | 648 | /5Phos/AACTTCGAGGCACAAGAATTAAAANNKAACCCAGAAATTCATCTGGTTGAG |
| P438 | | |
| ERCAS12A- | 649 | /5Phos/TTCGAGGCACAAGAATTAAAATATNNKCCAGAAATTCATCTGGTTGAGAGC |
| P439 | | |
| ERCAS12A- | 650 | /5Phos/GAGGCACAAGAATTAAAATATAACNNKGAAATTCATCTGGTTGAGAGCGAA |
| P440 | | |
| ERCAS12A- | 651 | /5Phos/GCACAAGAATTAAAATATAACCCANNKATTCATCTGGTTGAGAGCGAACTT |
| P441 | | |
| ERCAS12A- | 652 | /5Phos/CAAGAATTAAAATATAACCCAGAANNKCATCTGGTTGAGAGCGAACTTAAA |
| P442 | | |
| ERCAS12A- | 653 | /5Phos/GAATTAAAATATAACCCAGAAATTNNKCTGGTTGAGAGCGAACTTAAAGCT |
| P443 | | |
| ERCAS12A- | 654 | /5Phos/TTAAAATATAACCCAGAAATTCATNNKGTTGAGAGCGAACTTAAAGCTTCA |
| P444 | | |
| ERCAS12A- | 655 | /5Phos/AAATATAACCCAGAAATTCATCTGNNKGAGAGCGAACTTAAAGCTTCAGAA |
| P445 | | |
| ERCAS12A- | 656 | /5Phos/TATAACCCAGAAATTCATCTGGTTNNKAGCGAACTTAAAGCTTCAGAATTA |
| P446 | | |
| ERCAS12A- | 657 | /5Phos/AACCCAGAAATTCATCTGGTTGAGNNKGAACTTAAAGCTTCAGAATTAAAA |
| P447 | | |
| ERCAS12A- | 658 | /5Phos/CCAGAAATTCATCTGGTTGAGAGCNNKCTTAAAGCTTCAGAATTAAAAAAT |
| P448 | | |
| ERCAS12A- | 659 | /5Phos/GAAATTCATCTGGTTGAGAGCGAANNKAAAGCTTCAGAATTAAAAAATGTT |
| P449 | | |
| ERCAS12A- | 660 | /5Phos/ATTCATCTGGTTGAGAGCGAACTTNNKGCTTCAGAATTAAAAAATGTTTTG |
| P450 | | |
| ERCAS12A- | 661 | /5Phos/CATCTGGTTGAGAGCGAACTTAAANNKTCAGAATTAAAAAATGTTTTGGAC |
| P451 | | |
| ERCAS12A- | 662 | /5Phos/CTGGTTGAGAGCGAACTTAAAGCTNNKGAATTAAAAAATGTTTTGGACGTA |
| P452 | | |
| ERCAS12A- | 663 | /5Phos/GTTGAGAGCGAACTTAAAGCTTCANNKTTAAAAAATGTTTTGGACGTAATT |
| P453 | | |
| ERCAS12A- | 664 | /5Phos/GAGAGCGAACTTAAAGCTTCAGAANNKAAAAATGTTTTGGACGTAATTATG |
| P454 | | |
| ERCAS12A- | 665 | /5Phos/AGCGAACTTAAAGCTTCAGAATTANNKAATGTTTTGGACGTAATTATGAAC |
| P455 | | |
| ERCAS12A- | 666 | /5Phos/GAACTTAAAGCTTCAGAATTAAAANNKGTTTTGGACGTAATTATGAACGCC |
| P456 | | |
| ERCAS12A- | 667 | /5Phos/CTTAAAGCTTCAGAATTAAAAAATNNKTTGGACGTAATTATGAACGCCTTC |
| P457 | | |
| ERCAS12A- | 668 | /5Phos/AAAGCTTCAGAATTAAAAAATGTINNKGACGTAATTATGAACGCCTTCCAT |
| P458 | | |
| ERCAS12A- | 669 | /5Phos/GCTTCAGAATTAAAAAATGTTTTGNNKGTAATTATGAACGCCTTCCATTGG |
| P459 | | |
| ERCAS12A- | 670 | /5Phos/TCAGAATTAAAAAATGTTTTGGACNNKATTATGAACGCCTTCCATTGGTGC |
| P460 | | |
| ERCAS12A- | 671 | /5Phos/GAATTAAAAAATGTTTTGGACGTANNKATGAACGCCTTCCATTGGTGCAGC |
| P461 | | |
| ERCAS12A- | 672 | /5Phos/TTAAAAAATGTTTTGGACGTAATTNNKAACGCCTTCCATTGGTGCAGCGTG |
| P462 | | |
| ERCAS12A- | 673 | /5Phos/AAAAATGTTTTGGACGTAATTATGNNKGCCTTCCATTGGTGCAGCGTGTTT |
| P463 | | |
| ERCAS12A- | 674 | /5Phos/AATGTTTTGGACGTAATTATGAACNNKTTCCATTGGTGCAGCGTGTTTATG |
| P464 | | |
| ERCAS12A- | 675 | /5Phos/GTTTTGGACGTAATTATGAACGCCNNKCATTGGTGCAGCGTGTTTATGACA |
| P465 | | |
| ERCAS12A- | 676 | /5Phos/TTGGACGTAATTATGAACGCCTTCNNKTGGTGCAGCGTGTTTATGACAGAG |
| P466 | | |
| ERCAS12A- | 677 | /5Phos/GACGTAATTATGAACGCCTTCCATNNKTGCAGCGTGTTTATGACAGAGGAG |
| P467 | | |
| ERCAS12A- | 678 | /5Phos/GTAATTATGAACGCCTTCCATTGGNNKAGCGTGTTTATGACAGAGGAGTTA |
| P468 | | |
| ERCAS12A- | 679 | /5Phos/ATTATGAACGCCTTCCATTGGTGCNNKGTGTTTATGACAGAGGAGTTAGTA |
| P469 | | |
| ERCAS12A- | 680 | /5Phos/ATGAACGCCTTCCATTGGTGCAGCNNKTTTATGACAGAGGAGTTAGTAGAT |
| P470 | | |
| ERCAS12A- | 681 | /5Phos/AACGCCTTCCATTGGTGCAGCGTGNNKATGACAGAGGAGTTAGTAGATAAG |
| P471 | | |
| ERCAS12A- | 682 | /5Phos/GCCTTCCATTGGTGCAGCGTGTTTNNKACAGAGGAGTTAGTAGATAAGGAT |
| P472 | | |
| ERCAS12A- | 683 | /5Phos/TTCCATTGGTGCAGCGTGTTTATGNNKGAGGAGTTAGTAGATAAGGATAAC |
| P473 | | |
| ERCAS12A- | 684 | /5Phos/CATTGGTGCAGCGTGTTTATGACANNKGAGTTAGTAGATAAGGATAACAAC |
| P474 | | |
| ERCAS12A- | 685 | /5Phos/TGGTGCAGCGTGTTTATGACAGAGNNKTTAGTAGATAAGGATAACAACTTT |
| P475 | | |
| ERCAS12A- | 686 | /5Phos/TGCAGCGTGTTTATGACAGAGGAGNNKGTAGATAAGGATAACAACTTTTAT |
| P476 | | |
| ERCAS12A- | 687 | /5Phos/AGCGTGTTTATGACAGAGGAGTTANNKGATAAGGATAACAACTTTTATGCA |
| P477 | | |
| ERCAS12A- | 688 | /5Phos/GTGTTTATGACAGAGGAGTTAGTANNKAAGGATAACAACTTTTATGCAGAA |
| P478 | | |
| ERCAS12A- | 689 | /5Phos/TTTATGACAGAGGAGTTAGTAGATNNKGATAACAACTTTTATGCAGAATTG |
| P479 | | |
| ERCAS12A- | 690 | /5Phos/ATGACAGAGGAGTTAGTAGATAAGNNKAACAACTTTTATGCAGAATTGGAA |
| P480 | | |
| ERCAS12A- | 691 | /5Phos/ACAGAGGAGTTAGTAGATAAGGATNNKAACTTTTATGCAGAATTGGAAGAG |
| P481 | | |
| ERCAS12A- | 692 | /5Phos/GAGGAGTTAGTAGATAAGGATAACNNKTTTTATGCAGAATTGGAAGAGATC |
| P482 | | |
| ERCAS12A- | 693 | /5Phos/GAGTTAGTAGATAAGGATAACAACNNKTATGCAGAATTGGAAGAGATCTAT |
| P483 | | |
| ERCAS12A- | 694 | /5Phos/TTAGTAGATAAGGATAACAACTTTNNKGCAGAATTGGAAGAGATCTATGAT |
| P484 | | |
| ERCAS12A- | 695 | /5Phos/GTAGATAAGGATAACAACTTTTATNNKGAATTGGAAGAGATCTATGATGAG |
| P485 | | |
| ERCAS12A- | 696 | /5Phos/GATAAGGATAACAACTTTTATGCANNKTTGGAAGAGATCTATGATGAGATC |
| P486 | | |
| ERCAS12A- | 697 | /5Phos/AAGGATAACAACTTTTATGCAGAANNKGAAGAGATCTATGATGAGATCTAT |
| P487 | | |
| ERCAS12A- | 698 | /5Phos/GATAACAACTTTTATGCAGAATTGNNKGAGATCTATGATGAGATCTATCCA |
| P488 | | |
| ERCAS12A- | 699 | /5Phos/AACAACTTTTATGCAGAATTGGAANNKATCTATGATGAGATCTATCCAGTG |
| P489 | | |
| ERCAS12A- | 700 | /5Phos/AACTTTTATGCAGAATTGGAAGAGNNKTATGATGAGATCTATCCAGTGATT |
| P490 | | |
| ERCAS12A- | 701 | /5Phos/TTTTATGCAGAATTGGAAGAGATCNNKGATGAGATCTATCCAGTGATTTCA |
| P491 | | |
| ERCAS12A- | 702 | /5Phos/TATGCAGAATTGGAAGAGATCTATNNKGAGATCTATCCAGTGATTTCACTG |
| P492 | | |
| ERCAS12A- | 703 | /5Phos/GCAGAATTGGAAGAGATCTATGATNNKATCTATCCAGTGATTTCACTGTAT |
| P493 | | |
| ERCAS12A- | 704 | /5Phos/GAATTGGAAGAGATCTATGATGAGNNKTATCCAGTGATTTCACTGTATAAC |
| P494 | | |
| ERCAS12A- | 705 | /5Phos/TTGGAAGAGATCTATGATGAGATCNNKCCAGTGATTTCACTGTATAACTTA |
| P495 | | |
| ERCAS12A- | 706 | /5Phos/GAAGAGATCTATGATGAGATCTATNNKGTGATTTCACTGTATAACTTAGTC |
| P496 | | |
| ERCAS12A- | 707 | /5Phos/GAGATCTATGATGAGATCTATCCANNKATTTCACTGTATAACTTAGTCCGT |
| P497 | | |
| ERCAS12A- | 708 | /5Phos/ATCTATGATGAGATCTATCCAGTGNNKTCACTGTATAACTTAGTCCGTAAT |
| P498 | | |
| ERCAS12A- | 709 | /5Phos/TATGATGAGATCTATCCAGTGATTNNKCTGTATAACTTAGTCCGTAATTAC |
| P499 | | |
| ERCAS12A- | 710 | /5Phos/GATGAGATCTATCCAGTGATTTCANNKTATAACTTAGTCCGTAATTACGTC |
| P500 | | |
| ERCAS12A- | 711 | /5Phos/GAGATCTATCCAGTGATTTCACTGNNKAACTTAGTCCGTAATTACGTCACT |
| P501 | | |
| ERCAS12A- | 712 | /5Phos/ATCTATCCAGTGATTTCACTGTATNNKTTAGTCCGTAATTACGTCACTCAG |
| P502 | | |
| ERCAS12A- | 713 | /5Phos/TATCCAGTGATTTCACTGTATAACNNKGTCCGTAATTACGTCACTCAGAAG |
| P503 | | |
| ERCAS12A- | 714 | /5Phos/CCAGTGATTTCACTGTATAACTTANNKCGTAATTACGTCACTCAGAAGCCG |
| P504 | | |
| ERCAS12A- | 715 | /5Phos/GTGATTTCACTGTATAACTTAGTCNNKAATTACGTCACTCAGAAGCCGTAC |
| P505 | | |
| ERCAS12A- | 716 | /5Phos/ATTTCACTGTATAACTTAGTCCGTNNKTACGTCACTCAGAAGCCGTACTCA |
| P506 | | |
| ERCAS12A- | 717 | /5Phos/TCACTGTATAACTTAGTCCGTAATNNKGTCACTCAGAAGCCGTACTCAACT |
| P507 | | |
| ERCAS12A- | 718 | /5Phos/CTGTATAACTTAGTCCGTAATTACNNKACTCAGAAGCCGTACTCAACTAAA |
| P508 | | |
| ERCAS12A- | 719 | /5Phos/TATAACTTAGTCCGTAATTACGTCNNKCAGAAGCCGTACTCAACTAAAAAG |
| P509 | | |
| ERCAS12A- | 720 | /5Phos/AACTTAGTCCGTAATTACGTCACTNNKAAGCCGTACTCAACTAAAAAGATC |
| P510 | | |
| ERCAS12A- | 721 | /5Phos/TTAGTCCGTAATTACGTCACTCAGNNKCCGTACTCAACTAAAAAGATCAAG |
| P511 | | |
| ERCAS12A- | 722 | /5Phos/GTCCGTAATTACGTCACTCAGAAGNNKTACTCAACTAAAAAGATCAAGTTA |
| P512 | | |
| ERCAS12A- | 723 | /5Phos/CGTAATTACGTCACTCAGAAGCCGNNKTCAACTAAAAAGATCAAGTTAAAT |
| P513 | | |
| ERCAS12A- | 724 | /5Phos/AATTACGTCACTCAGAAGCCGTACNNKACTAAAAAGATCAAGTTAAATTTT |
| P514 | | |
| ERCAS12A- | 725 | /5Phos/TACGTCACTCAGAAGCCGTACTCANNKAAAAAGATCAAGTTAAATTTTGGA |
| P515 | | |
| ERCAS12A- | 726 | /5Phos/GTCACTCAGAAGCCGTACTCAACTNNKAAGATCAAGTTAAATTTTGGAATT |
| P516 | | |
| ERCAS12A- | 727 | /5Phos/ACTCAGAAGCCGTACTCAACTAAANNKATCAAGTTAAATTTTGGAATTCCG |
| P517 | | |
| ERCAS12A- | 728 | /5Phos/CAGAAGCCGTACTCAACTAAAAAGNNKAAGTTAAATTTTGGAATTCCGACA |
| P518 | | |
| ERCAS12A- | 729 | /5Phos/AAGCCGTACTCAACTAAAAAGATCNNKTTAAATTTTGGAATTCCGACATTG |
| P519 | | |
| ERCAS12A- | 730 | /5Phos/CCGTACTCAACTAAAAAGATCAAGNNKAATTTTGGAATTCCGACATTGGCG |
| P520 | | |
| ERCAS12A- | 731 | /5Phos/TACTCAACTAAAAAGATCAAGTTANNKTTTGGAATTCCGACATTGGCGGAT |
| P521 | | |
| ERCAS12A- | 732 | /5Phos/TCAACTAAAAAGATCAAGTTAAATNNKGGAATTCCGACATTGGCGGATGGT |
| P522 | | |
| ERCAS12A- | 733 | /5Phos/ACTAAAAAGATCAAGTTAAATTTTNNKATTCCGACATTGGCGGATGGTTGG |
| P523 | | |
| ERCAS12A- | 734 | /5Phos/AAAAAGATCAAGTTAAATTTTGGANNKCCGACATTGGCGGATGGTTGGAGT |
| P524 | | |
| ERCAS12A- | 735 | /5Phos/AAGATCAAGTTAAATTTTGGAATTNNKACATTGGCGGATGGTTGGAGTAAG |
| P525 | | |
| ERCAS12A- | 736 | /5Phos/ATCAAGTTAAATTTTGGAATTCCGNNKTTGGCGGATGGTTGGAGTAAGTCT |
| P526 | | |
| ERCAS12A- | 737 | /5Phos/AAGTTAAATTTTGGAATTCCGACANNKGCGGATGGTTGGAGTAAGTCTAAG |
| P527 | | |
| ERCAS12A- | 738 | /5Phos/TTAAATTTTGGAATTCCGACATTGNNKGATGGTTGGAGTAAGTCTAAGGAA |
| P528 | | |
| ERCAS12A- | 739 | /5Phos/AATTTTGGAATTCCGACATTGGCGNNKGGTTGGAGTAAGTCTAAGGAATAC |
| P529 | | |
| ERCAS12A- | 740 | /5Phos/TTTGGAATTCCGACATTGGCGGATNNKTGGAGTAAGTCTAAGGAATACTCA |
| P530 | | |
| ERCAS12A- | 741 | /5Phos/GGAATTCCGACATTGGCGGATGGTNNKAGTAAGTCTAAGGAATACTCAAAT |
| P531 | | |
| ERCAS12A- | 742 | /5Phos/ATTCCGACATTGGCGGATGGTTGGNNKAAGTCTAAGGAATACTCAAATAAC |
| P532 | | |
| ERCAS12A- | 743 | /5Phos/CCGACATTGGCGGATGGTTGGAGTNNKTCTAAGGAATACTCAAATAACGCT |
| P533 | | |
| ERCAS12A- | 744 | /5Phos/ACATTGGCGGATGGTTGGAGTAAGNNKAAGGAATACTCAAATAACGCTATT |
| P534 | | |
| ERCAS12A- | 745 | /5Phos/TTGGCGGATGGTTGGAGTAAGTCTNNKGAATACTCAAATAACGCTATTATC |
| P535 | | |
| ERCAS12A- | 746 | /5Phos/GCGGATGGTTGGAGTAAGTCTAAGNNKTACTCAAATAACGCTATTATCTTA |
| P536 | | |
| ERCAS12A- | 747 | /5Phos/GATGGTTGGAGTAAGTCTAAGGAANNKTCAAATAACGCTATTATCTTAATG |
| P537 | | |
| ERCAS12A- | 748 | /5Phos/GGTTGGAGTAAGTCTAAGGAATACNNKAATAACGCTATTATCTTAATGCGT |
| P538 | | |
| ERCAS12A- | 749 | /5Phos/TGGAGTAAGTCTAAGGAATACTCANNKAACGCTATTATCTTAATGCGTGAC |
| P539 | | |
| ERCAS12A- | 750 | /5Phos/AGTAAGTCTAAGGAATACTCAAATNNKGCTATTATCTTAATGCGTGACAAC |
| P540 | | |
| ERCAS12A- | 751 | /5Phos/AAGTCTAAGGAATACTCAAATAACNNKATTATCTTAATGCGTGACAACCTG |
| P541 | | |
| ERCAS12A- | 752 | /5Phos/TCTAAGGAATACTCAAATAACGCTNNKATCTTAATGCGTGACAACCTGTAT |
| P542 | | |
| ERCAS12A- | 753 | /5Phos/AAGGAATACTCAAATAACGCTATTNNKTTAATGCGTGACAACCTGTATTAC |
| P543 | | |
| ERCAS12A- | 754 | /5Phos/GAATACTCAAATAACGCTATTATCNNKATGCGTGACAACCTGTATTACTTG |
| P544 | | |
| ERCAS12A- | 755 | /5Phos/TACTCAAATAACGCTATTATCTTANNKCGTGACAACCTGTATTACTTGGGT |
| P545 | | |
| ERCAS12A- | 756 | /5Phos/TCAAATAACGCTATTATCTTAATGNNKGACAACCTGTATTACTTGGGTATT |
| P546 | | |
| ERCAS12A- | 757 | /5Phos/AATAACGCTATTATCTTAATGCGTNNKAACCTGTATTACTTGGGTATTTTC |
| P547 | | |
| ERCAS12A- | 758 | /5Phos/AACGCTATTATCTTAATGCGTGACNNKCTGTATTACTTGGGTATTTTCAAT |
| P548 | | |
| ERCAS12A- | 759 | /5Phos/GCTATTATCTTAATGCGTGACAACNNKTATTACTTGGGTATTTTCAATGCC |
| P549 | | |
| ERCAS12A- | 760 | /5Phos/ATTATCTTAATGCGTGACAACCTGNNKTACTTGGGTATTTTCAATGCCAAA |
| P550 | | |
| ERCAS12A- | 761 | /5Phos/ATCTTAATGCGTGACAACCTGTATNNKTTGGGTATTTTCAATGCCAAAAAT |
| P551 | | |
| ERCAS12A- | 762 | /5Phos/TTAATGCGTGACAACCTGTATTACNNKGGTATTTTCAATGCCAAAAATAAG |
| P552 | | |
| ERCAS12A- | 763 | /5Phos/ATGCGTGACAACCTGTATTACTTGNNKATTTTCAATGCCAAAAATAAGCCC |
| P553 | | |
| ERCAS12A- | 764 | /5Phos/CGTGACAACCTGTATTACTTGGGTNNKTTCAATGCCAAAAATAAGCCCGAT |
| P554 | | |
| ERCAS12A- | 765 | /5Phos/GACAACCTGTATTACTTGGGTATTNNKAATGCCAAAAATAAGCCCGATAAA |
| P555 | | |
| ERCAS12A- | 766 | /5Phos/AACCTGTATTACTTGGGTATTTTCNNKGCCAAAAATAAGCCCGATAAAAAA |
| P556 | | |
| ERCAS12A- | 767 | /5Phos/CTGTATTACTTGGGTATTTTCAATNNKAAAAATAAGCCCGATAAAAAAATC |
| P557 | | |
| ERCAS12A- | 768 | /5Phos/TATTACTTGGGTATTTTCAATGCCNNKAATAAGCCCGATAAAAAAATCATT |
| P558 | | |
| ERCAS12A- | 769 | /5Phos/TACTTGGGTATTTTCAATGCCAAANNKAAGCCCGATAAAAAAATCATTGAA |
| P559 | | |
| ERCAS12A- | 770 | /5Phos/TTGGGTATTTTCAATGCCAAAAATNNKCCCGATAAAAAAATCATTGAAGGG |
| P560 | | |
| ERCAS12A- | 771 | /5Phos/GGTATTTTCAATGCCAAAAATAAGNNKGATAAAAAAATCATTGAAGGGAAT |
| P561 | | |
| ERCAS12A- | 772 | /5Phos/ATTTTCAATGCCAAAAATAAGCCCNNKAAAAAAATCATTGAAGGGAATACT |
| P562 | | |
| ERCAS12A- | 773 | /5Phos/TTCAATGCCAAAAATAAGCCCGATNNKAAAATCATTGAAGGGAATACTTCA |
| P563 | | |
| ERCAS12A- | 774 | /5Phos/AATGCCAAAAATAAGCCCGATAAANNKATCATTGAAGGGAATACTTCAGAA |
| P564 | | |
| ERCAS12A- | 775 | /5Phos/GCCAAAAATAAGCCCGATAAAAAANNKATTGAAGGGAATACTTCAGAAAAT |
| P565 | | |
| ERCAS12A- | 776 | /5Phos/AAAAATAAGCCCGATAAAAAAATCNNKGAAGGGAATACTTCAGAAAATAAG |
| P566 | | |
| ERCAS12A- | 777 | /5Phos/AATAAGCCCGATAAAAAAATCATTNNKGGGAATACTTCAGAAAATAAGGGA |
| P567 | | |
| ERCAS12A- | 778 | /5Phos/AAGCCCGATAAAAAAATCATTGAANNKAATACTTCAGAAAATAAGGGAGAT |
| P568 | | |
| ERCAS12A- | 779 | /5Phos/CCCGATAAAAAAATCATTGAAGGGNNKACTTCAGAAAATAAGGGAGATTAC |
| P569 | | |
| ERCAS12A- | 780 | /5Phos/GATAAAAAAATCATTGAAGGGAATNNKTCAGAAAATAAGGGAGATTACAAA |
| P570 | | |
| ERCAS12A- | 781 | /5Phos/AAAAAAATCATTGAAGGGAATACTNNKGAAAATAAGGGAGATTACAAAAAA |
| P571 | | |
| ERCAS12A | 782 | /5Phos/AAAATCATTGAAGGGAATACTTCANNKAATAAGGGAGATTACAAAAAAATG |
| P572 | | |
| ERCAS12A- | 783 | /5Phos/ATCATTGAAGGGAATACTTCAGAANNKAAGGGAGATTACAAAAAAATGATT |
| P573 | | |
| ERCAS12A- | 784 | /5Phos/ATTGAAGGGAATACTTCAGAAAATNNKGGAGATTACAAAAAAATGATTTAT |
| P574 | | |
| ERCAS12A- | 785 | /5Phos/GAAGGGAATACTTCAGAAAATAAGNNKGATTACAAAAAAATGATTTATAAC |
| P575 | | |
| ERCAS12A- | 786 | /5Phos/GGGAATACTTCAGAAAATAAGGGANNKTACAAAAAAATGATTTATAACCTT |
| P576 | | |
| ERCAS12A- | 787 | /5Phos/AATACTTCAGAAAATAAGGGAGATNNKAAAAAAATGATTTATAACCTTTTG |
| P577 | | |
| ERCAS12A- | 788 | /5Phos/ACTTCAGAAAATAAGGGAGATTACNNKAAAATGATTTATAACCTTTTGCCA |
| P578 | | |
| ERCAS12A- | 789 | /5Phos/TCAGAAAATAAGGGAGATTACAAANNKATGATTTATAACCTTTTGCCAGGC |
| P579 | | |
| ERCAS12A- | 790 | /5Phos/GAAAATAAGGGAGATTACAAAAAANNKATTTATAACCTTTTGCCAGGCCCA |
| P580 | | |
| ERCAS12A | 791 | /5Phos/AATAAGGGAGATTACAAAAAAATGNNKTATAACCTTTTGCCAGGCCCAAAC |
| P581 | | |
| ERCAS12A- | 792 | /5Phos/AAGGGAGATTACAAAAAAATGATTNNKAACCTTTTGCCAGGCCCAAACAAA |
| P582 | | |
| ERCAS12A- | 793 | /5Phos/GGAGATTACAAAAAAATGATTTATNNKCTTTTGCCAGGCCCAAACAAAATG |
| P583 | | |
| ERCAS12A- | 794 | /5Phos/GATTACAAAAAAATGATTTATAACNNKTTGCCAGGCCCAAACAAAATGATC |
| P584 | | |
| ERCAS12A- | 795 | /5Phos/TACAAAAAAATGATTTATAACCTTNNKCCAGGCCCAAACAAAATGATCCCC |
| P585 | | |
| ERCAS12A- | 796 | /5Phos/AAAAAAATGATTTATAACCTTTTGNNKGGCCCAAACAAAATGATCCCCAAG |
| P586 | | |
| ERCAS12A- | 797 | /5Phos/AAAATGATTTATAACCTTTTGCCANNKCCAAACAAAATGATCCCCAAGGTG |
| P587 | | |
| ERCAS12A- | 798 | /5Phos/ATGATTTATAACCTTTTGCCAGGCNNKAACAAAATGATCCCCAAGGTGTTT |
| P588 | | |
| ERCAS12A- | 799 | /5Phos/ATTTATAACCTTTTGCCAGGCCCANNKAAAATGATCCCCAAGGTGTTTTTG |
| P589 | | |
| ERCAS12A- | 800 | /5Phos/TATAACCTTTTGCCAGGCCCAAACNNKATGATCCCCAAGGTGTTTTTGAGT |
| P590 | | |
| ERCAS12A- | 801 | /5Phos/AACCTTTTGCCAGGCCCAAACAAANNKATCCCCAAGGTGTTTTTGAGTAGT |
| P591 | | |
| ERCAS12A- | 802 | /5Phos/CTTTTGCCAGGCCCAAACAAAATGNNKCCCAAGGTGTTTTTGAGTAGTAAG |
| P592 | | |
| ERCAS12A- | 803 | /5Phos/TTGCCAGGCCCAAACAAAATGATCNNKAAGGTGTTTTTGAGTAGTAAGACG |
| P593 | | |
| ERCAS12A- | 804 | /5Phos/CCAGGCCCAAACAAAATGATCCCCNNKGTGTTTTTGAGTAGTAAGACGGGA |
| P594 | | |
| ERCAS12A- | 805 | /5Phos/GGCCCAAACAAAATGATCCCCAAGNNKTTTTTGAGTAGTAAGACGGGAGTA |
| P595 | | |
| ERCAS12A- | 806 | /5Phos/CCAAACAAAATGATCCCCAAGGTGNNKTTGAGTAGTAAGACGGGAGTAGAA |
| P596 | | |
| ERCAS12A- | 807 | /5Phos/AACAAAATGATCCCCAAGGTGTTTNNKAGTAGTAAGACGGGAGTAGAAACA |
| P597 | | |
| ERCAS12A- | 808 | /5Phos/AAAATGATCCCCAAGGTGTTTTTGNNKAGTAAGACGGGAGTAGAAACATAT |
| P598 | | |
| ERCAS12A- | 809 | /5Phos/ATGATCCCCAAGGTGTTTTTGAGTNNKAAGACGGGAGTAGAAACATATAAG |
| P599 | | |
| ERCAS12A- | 810 | /5Phos/ATCCCCAAGGTGTTTTTGAGTAGTNNKACGGGAGTAGAAACATATAAGCCA |
| P600 | | |
| ERCAS12A- | 811 | /5Phos/CCCAAGGTGTTTTTGAGTAGTAAGNNKGGAGTAGAAACATATAAGCCATCA |
| P601 | | |
| ERCAS12A- | 812 | /5Phos/AAGGTGTTTTTGAGTAGTAAGACGNNKGTAGAAACATATAAGCCATCAGCG |
| P602 | | |
| ERCAS12A- | 813 | /5Phos/GTGTTTTTGAGTAGTAAGACGGGANNKGAAACATATAAGCCATCAGCGTAC |
| P603 | | |
| ERCAS12A- | 814 | /5Phos/TTTTTGAGTAGTAAGACGGGAGTANNKACATATAAGCCATCAGCGTACATT |
| P604 | | |
| ERCAS12A- | 815 | /5Phos/TTGAGTAGTAAGACGGGAGTAGAANNKTATAAGCCATCAGCGTACATTCTT |
| P605 | | |
| ERCAS12A- | 816 | /5Phos/AGTAGTAAGACGGGAGTAGAAACANNKAAGCCATCAGCGTACATTCTTGAA |
| P606 | | |
| ERCAS12A- | 817 | /5Phos/AGTAAGACGGGAGTAGAAACATATNNKCCATCAGCGTACATTCTTGAAGGG |
| P607 | | |
| ERCAS12A- | 818 | /5Phos/AAGACGGGAGTAGAAACATATAAGNNKTCAGCGTACATTCTTGAAGGGTAC |
| P608 | | |
| ERCAS12A- | 819 | /5Phos/ACGGGAGTAGAAACATATAAGCCANNKGCGTACATTCTTGAAGGGTACAAA |
| P609 | | |
| ERCAS12A- | 820 | /5Phos/GGAGTAGAAACATATAAGCCATCANNKTACATTCTTGAAGGGTACAAACAA |
| P610 | | |
| ERCAS12A- | 821 | /5Phos/GTAGAAACATATAAGCCATCAGCGNNKATTCTTGAAGGGTACAAACAAAAT |
| P611 | | |
| ERCAS12A- | 822 | /5Phos/GAAACATATAAGCCATCAGCGTACNNKCTTGAAGGGTACAAACAAAATAAA |
| P612 | | |
| ERCAS12A- | 823 | /5Phos/ACATATAAGCCATCAGCGTACATTNNKGAAGGGTACAAACAAAATAAACAC |
| P613 | | |
| ERCAS12A- | 824 | /5Phos/TATAAGCCATCAGCGTACATTCTTNNKGGGTACAAACAAAATAAACACATC |
| P614 | | |
| ERCAS12A- | 825 | /5Phos/AAGCCATCAGCGTACATTCTTGAANNKTACAAACAAAATAAACACATCAAG |
| P615 | | |
| ERCAS12A- | 826 | /5Phos/CCATCAGCGTACATTCTTGAAGGGNNKAAACAAAATAAACACATCAAGAGC |
| P616 | | |
| ERCAS12A- | 827 | /5Phos/TCAGCGTACATTCTTGAAGGGTACNNKCAAAATAAACACATCAAGAGCAGC |
| P617 | | |
| ERCAS12A- | 828 | /5Phos/GCGTACATTCTTGAAGGGTACAAANNKAATAAACACATCAAGAGCAGCAAG |
| P618 | | |
| ERCAS12A- | 829 | /5Phos/TACATTCTTGAAGGGTACAAACAANNKAAACACATCAAGAGCAGCAAGGAC |
| P619 | | |
| ERCAS12A- | 830 | /5Phos/ATTCTTGAAGGGTACAAACAAAATNNKCACATCAAGAGCAGCAAGGACTTT |
| P620 | | |
| ERCAS12A- | 831 | /5Phos/CTTGAAGGGTACAAACAAAATAAANNKATCAAGAGCAGCAAGGACTTTGAC |
| P621 | | |
| ERCAS12A- | 832 | /5Phos/GAAGGGTACAAACAAAATAAACACNNKAAGAGCAGCAAGGACTTTGACATT |
| P622 | | |
| ERCAS12A- | 833 | /5Phos/GGGTACAAACAAAATAAACACATCNNKAGCAGCAAGGACTTTGACATTACA |
| P623 | | |
| ERCAS12A- | 834 | /5Phos/TACAAACAAAATAAACACATCAAGNNKAGCAAGGACTTTGACATTACATTT |
| P624 | | |
| ERCAS12A- | 835 | /5Phos/AAACAAAATAAACACATCAAGAGCNNKAAGGACTTTGACATTACATTTTGT |
| P625 | | |
| ERCAS12A- | 836 | /5Phos/CAAAATAAACACATCAAGAGCAGCNNKGACTTTGACATTACATTTTGTCAT |
| P626 | | |
| ERCAS12A- | 837 | /5Phos/AATAAACACATCAAGAGCAGCAAGNNKTTTGACATTACATTTTGTCATGAC |
| P627 | | |
| ERCAS12A- | 838 | /5Phos/AAACACATCAAGAGCAGCAAGGACNNKGACATTACATTTTGTCATGACTTA |
| P628 | | |
| ERCAS12A- | 839 | /5Phos/CACATCAAGAGCAGCAAGGACTTTNNKATTACATTTTGTCATGACTTAATC |
| P629 | | |
| ERCAS12A- | 840 | /5Phos/ATCAAGAGCAGCAAGGACTTTGACNNKACATTTTGTCATGACTTAATCGAC |
| P630 | | |
| ERCAS12A- | 841 | /5Phos/AAGAGCAGCAAGGACTTTGACATTNNKTTTTGTCATGACTTAATCGACTAC |
| P631 | | |
| ERCAS12A- | 842 | /5Phos/AGCAGCAAGGACTTTGACATTACANNKTGTCATGACTTAATCGACTACTTT |
| P632 | | |
| ERCAS12A- | 843 | /5Phos/AGCAAGGACTTTGACATTACATTTNNKCATGACTTAATCGACTACTTTAAA |
| P633 | | |
| ERCAS12A- | 844 | /5Phos/AAGGACTTTGACATTACATTTTGTNNKGACTTAATCGACTACTTTAAAAAT |
| P634 | | |
| ERCAS12A- | 845 | /5Phos/GACTTTGACATTACATTTTGTCATNNKTTAATCGACTACTTTAAAAATTGC |
| P635 | | |
| ERCAS12A- | 846 | /5Phos/TTTGACATTACATTTTGTCATGACNNKATCGACTACTTTAAAAATTGCATT |
| P636 | | |
| ERCAS12A- | 847 | /5Phos/GACATTACATTTTGTCATGACTTANNKGACTACTTTAAAAATTGCATTGCC |
| P637 | | |
| ERCAS12A- | 848 | /5Phos/ATTACATTTTGTCATGACTTAATCNNKTACTTTAAAAATTGCATTGCCATT |
| P638 | | |
| ERCAS12A- | 849 | /5Phos/ACATTTTGTCATGACTTAATCGACNNKTTTAAAAATTGCATTGCCATTCAC |
| P639 | | |
| ERCAS12A- | 850 | /5Phos/TTTTGTCATGACTTAATCGACTACNNKAAAAATTGCATTGCCATTCACCCT |
| P640 | | |
| ERCAS12A- | 851 | /5Phos/TGTCATGACTTAATCGACTACTTTNNKAATTGCATTGCCATTCACCCTGAG |
| P641 | | |
| ERCAS12A- | 852 | /5Phos/CATGACTTAATCGACTACTTTAAANNKTGCATTGCCATTCACCCTGAGTGG |
| P642 | | |
| ERCAS12A- | 853 | /5Phos/GACTTAATCGACTACTTTAAAAATNNKATTGCCATTCACCCTGAGTGGAAA |
| P643 | | |
| ERCAS12A- | 854 | /5Phos/TTAATCGACTACTTTAAAAATTGCNNKGCCATTCACCCTGAGTGGAAAAAT |
| P644 | | |
| ERCAS12A- | 855 | /5Phos/ATCGACTACTTTAAAAATTGCATTNNKATTCACCCTGAGTGGAAAAATTTT |
| P645 | | |
| ERCAS12A- | 856 | /5Phos/GACTACTTTAAAAATTGCATTGCCNNKCACCCTGAGTGGAAAAATTTTGGT |
| P646 | | |
| ERCAS12A- | 857 | /5Phos/TACTTTAAAAATTGCATTGCCATTNNKCCTGAGTGGAAAAATTTTGGTTTC |
| P647 | | |
| ERCAS12A- | 858 | /5Phos/TTTAAAAATTGCATTGCCATTCACNNKGAGTGGAAAAATTTTGGTTTCGAC |
| P648 | | |
| ERCAS12A- | 859 | /5Phos/AAAAATTGCATTGCCATTCACCCTNNKTGGAAAAATTTTGGTTTCGACTTC |
| P649 | | |
| ERCAS12A- | 860 | /5Phos/AATTGCATTGCCATTCACCCTGAGNNKAAAAATTTTGGTTTCGACTTCAGT |
| P650 | | |
| ERCAS12A- | 861 | /5Phos/TGCATTGCCATTCACCCTGAGTGGNNKAATTTTGGTTTCGACTTCAGTGAT |
| P651 | | |
| ERCAS12A- | 862 | /5Phos/ATTGCCATTCACCCTGAGTGGAAANNKTTTGGTTTCGACTTCAGTGATACC |
| P652 | | |
| ERCAS12A- | 863 | /5Phos/GCCATTCACCCTGAGTGGAAAAATNNKGGTTTCGACTTCAGTGATACCTCT |
| P653 | | |
| ERCAS12A- | 864 | /5Phos/ATTCACCCTGAGTGGAAAAATTTTNNKTTCGACTTCAGTGATACCTCTACC |
| P654 | | |
| ERCAS12A- | 865 | /5Phos/CACCCTGAGTGGAAAAATTTTGGTNNKGACTTCAGTGATACCTCTACCTAT |
| P655 | | |
| ERCAS12A- | 866 | /5Phos/CCTGAGTGGAAAAATTTTGGTTTCNNKTTCAGTGATACCTCTACCTATGAA |
| P656 | | |
| ERCAS12A- | 867 | /5Phos/GAGTGGAAAAATTTTGGTTTCGACNNKAGTGATACCTCTACCTATGAAGAT |
| P657 | | |
| ERCAS12A- | 868 | /5Phos/TGGAAAAATTTTGGTTTCGACTTCNNKGATACCTCTACCTATGAAGATATC |
| P658 | | |
| ERCAS12A- | 869 | /5Phos/AAAAATTTTGGTTTCGACTTCAGTNNKACCTCTACCTATGAAGATATCTCT |
| P659 | | |
| ERCAS12A- | 870 | /5Phos/AATTTTGGTTTCGACTTCAGTGATNNKTCTACCTATGAAGATATCTCTGGT |
| P660 | | |
| ERCAS12A- | 871 | /5Phos/TTTGGTTTCGACTTCAGTGATACCNNKACCTATGAAGATATCTCTGGTTTT |
| P661 | | |
| ERCAS12A- | 872 | /5Phos/GGTTTCGACTTCAGTGATACCTCTNNKTATGAAGATATCTCTGGTTTTTAT |
| P662 | | |
| ERCAS12A- | 873 | /5Phos/TTCGACTTCAGTGATACCTCTACCNNKGAAGATATCTCTGGTTTTTATCGT |
| P663 | | |
| ERCAS12A- | 874 | /5Phos/GACTTCAGTGATACCTCTACCTATNNKGATATCTCTGGTTTTTATCGTGAG |
| P664 | | |
| ERCAS12A- | 875 | /5Phos/TTCAGTGATACCTCTACCTATGAANNKATCTCTGGTTTTTATCGTGAGGTA |
| P665 | | |
| ERCAS12A- | 876 | /5Phos/AGTGATACCTCTACCTATGAAGATNNKTCTGGTTTTTATCGTGAGGTAGAA |
| P666 | | |
| ERCAS12A- | 877 | /5Phos/GATACCTCTACCTATGAAGATATCNNKGGTTTTTATCGTGAGGTAGAACTT |
| P667 | | |
| ERCAS12A- | 878 | /5Phos/ACCTCTACCTATGAAGATATCTCTNNKTTTTATCGTGAGGTAGAACTTCAG |
| P668 | | |
| ERCAS12A- | 879 | /5Phos/TCTACCTATGAAGATATCTCTGGTNNKTATCGTGAGGTAGAACTTCAGGGT |
| P669 | | |
| ERCAS12A- | 880 | /5Phos/ACCTATGAAGATATCTCTGGTTTTNNKCGTGAGGTAGAACTTCAGGGTTAC |
| P670 | | |
| ERCAS12A- | 881 | /5Phos/TATGAAGATATCTCTGGTTTTTATNNKGAGGTAGAACTTCAGGGTTACAAG |
| P671 | | |
| ERCAS12A- | 882 | /5Phos/GAAGATATCTCTGGTTTTTATCGTNNKGTAGAACTTCAGGGTTACAAGATC |
| P672 | | |
| ERCAS12A- | 883 | /5Phos/GATATCTCTGGTTTTTATCGTGAGNNKGAACTTCAGGGTTACAAGATCGAT |
| P673 | | |
| ERCAS12A- | 884 | /5Phos/ATCTCTGGTTTTTATCGTGAGGTANNKCTTCAGGGTTACAAGATCGATTGG |
| P674 | | |
| ERCAS12A- | 885 | /5Phos/TCTGGTTTTTATCGTGAGGTAGAANNKCAGGGTTACAAGATCGATTGGACG |
| P675 | | |
| ERCAS12A- | 886 | /5Phos/GGTTTTTATCGTGAGGTAGAACTTNNKGGTTACAAGATCGATTGGACGTAC |
| P676 | | |
| ERCAS12A- | 887 | /5Phos/TTTTATCGTGAGGTAGAACTTCAGNNKTACAAGATCGATTGGACGTACATT |
| P677 | | |
| ERCAS12A- | 888 | /5Phos/TATCGTGAGGTAGAACTTCAGGGTNNKAAGATCGATTGGACGTACATTTCC |
| P678 | | |
| ERCAS12A- | 889 | /5Phos/CGTGAGGTAGAACTTCAGGGTTACNNKATCGATTGGACGTACATTTCCGAG |
| P679 | | |
| ERCAS12A- | 890 | /5Phos/GAGGTAGAACTTCAGGGTTACAAGNNKGATTGGACGTACATTTCCGAGAAA |
| P680 | | |
| ERCAS12A- | 891 | /5Phos/GTAGAACTTCAGGGTTACAAGATCNNKTGGACGTACATTTCCGAGAAAGAC |
| P681 | | |
| ERCAS12A- | 892 | /5Phos/GAACTTCAGGGTTACAAGATCGATNNKACGTACATTTCCGAGAAAGACATT |
| P682 | | |
| ERCAS12A- | 893 | /5Phos/CTTCAGGGTTACAAGATCGATTGGNNKTACATTTCCGAGAAAGACATTGAT |
| P683 | | |
| ERCAS12A- | 894 | /5Phos/CAGGGTTACAAGATCGATTGGACGNNKATTTCCGAGAAAGACATTGATCTT |
| P684 | | |
| ERCAS12A- | 895 | /5Phos/GGTTACAAGATCGATTGGACGTACNNKTCCGAGAAAGACATTGATCTTTTA |
| P685 | | |
| ERCAS12A- | 896 | /5Phos/TACAAGATCGATTGGACGTACATTNNKGAGAAAGACATTGATCTTTTACAA |
| P686 | | |
| ERCAS12A- | 897 | /5Phos/AAGATCGATTGGACGTACATTTCCNNKAAAGACATTGATCTTTTACAAGAG |
| P687 | | |
| ERCAS12A- | 898 | /5Phos/ATCGATTGGACGTACATTTCCGAGNNKGACATTGATCTTTTACAAGAGAAG |
| P688 | | |
| ERCAS12A- | 899 | /5Phos/GATTGGACGTACATTTCCGAGAAANNKATTGATCTTTTACAAGAGAAGGGT |
| P689 | | |
| ERCAS12A- | 900 | /5Phos/TGGACGTACATTTCCGAGAAAGACNNKGATCTTTTACAAGAGAAGGGTCAG |
| P690 | | |
| ERCAS12A- | 901 | /5Phos/ACGTACATTTCCGAGAAAGACATTNNKCTTTTACAAGAGAAGGGTCAGCTT |
| P691 | | |
| ERCAS12A- | 902 | /5Phos/TACATTTCCGAGAAAGACATTGATNNKTTACAAGAGAAGGGTCAGCTTTAC |
| P692 | | |
| ERCAS12A- | 903 | /5Phos/ATTTCCGAGAAAGACATTGATCTTNNKCAAGAGAAGGGTCAGCTTTACTTA |
| P693 | | |
| ERCAS12A- | 904 | /5Phos/TCCGAGAAAGACATTGATCTTTTANNKGAGAAGGGTCAGCTTTACTTATTT |
| P694 | | |
| ERCAS12A- | 905 | /5Phos/GAGAAAGACATTGATCTTTTACAANNKAAGGGTCAGCTTTACTTATTTCAG |
| P695 | | |
| ERCAS12A- | 906 | /5Phos/AAAGACATTGATCTTTTACAAGAGNNKGGTCAGCTTTACTTATTTCAGATC |
| P696 | | |
| ERCAS12A- | 907 | /5Phos/GACATTGATCTTTTACAAGAGAAGNNKCAGCTTTACTTATTTCAGATCTAC |
| P697 | | |
| ERCAS12A- | 908 | /5Phos/ATTGATCTTTTACAAGAGAAGGGTNNKCTTTACTTATTTCAGATCTACAAT |
| P698 | | |
| ERCAS12A- | 909 | /5Phos/GATCTTTTACAAGAGAAGGGTCAGNNKTACTTATTTCAGATCTACAATAAA |
| P699 | | |
| ERCAS12A- | 910 | /5Phos/CTTTTACAAGAGAAGGGTCAGCTTNNKTTATTTCAGATCTACAATAAAGAC |
| P700 | | |
| ERCAS12A- | 911 | /5Phos/TTACAAGAGAAGGGTCAGCTTTACNNKTTTCAGATCTACAATAAAGACTTT |
| P701 | | |
| ERCAS12A- | 912 | /5Phos/CAAGAGAAGGGTCAGCTTTACTTANNKCAGATCTACAATAAAGACTTTTCG |
| P702 | | |
| ERCAS12A- | 913 | /5Phos/GAGAAGGGTCAGCTTTACTTATTTNNKATCTACAATAAAGACTTTTCGAAA |
| P703 | | |
| ERCAS12A- | 914 | /5Phos/AAGGGTCAGCTTTACTTATTTCAGNNKTACAATAAAGACTTTTCGAAAAAA |
| P704 | | |
| ERCAS12A- | 915 | /5Phos/GGTCAGCTTTACTTATTTCAGATCNNKAATAAAGACTTTTCGAAAAAATCC |
| P705 | | |
| ERCAS12A- | 916 | /5Phos/CAGCTTTACTTATTTCAGATCTACNNKAAAGACTTTTCGAAAAAATCCACG |
| P706 | | |
| ERCAS12A- | 917 | /5Phos/CTTTACTTATTTCAGATCTACAATNNKGACTTTTCGAAAAAATCCACGGGC |
| P707 | | |
| ERCAS12A- | 918 | /5Phos/TACTTATTTCAGATCTACAATAAANNKTTTTCGAAAAAATCCACGGGCAAC |
| P708 | | |
| ERCAS12A- | 919 | /5Phos/TTATTTCAGATCTACAATAAAGACNNKTCGAAAAAATCCACGGGCAACGAC |
| P709 | | |
| ERCAS12A- | 920 | /5Phos/TTTCAGATCTACAATAAAGACTTTNNKAAAAAATCCACGGGCAACGACAAC |
| P710 | | |
| ERCAS12A- | 921 | /5Phos/CAGATCTACAATAAAGACTTTTCGNNKAAATCCACGGGCAACGACAACCTT |
| P711 | | |
| ERCAS12A- | 922 | /5Phos/ATCTACAATAAAGACTTTTCGAAANNKTCCACGGGCAACGACAACCTTCAT |
| P712 | | |
| ERCAS12A- | 923 | /5Phos/TACAATAAAGACTTTTCGAAAAAANNKACGGGCAACGACAACCTTCATACA |
| P713 | | |
| ERCAS12A- | 924 | /5Phos/AATAAAGACTTTTCGAAAAAATCCNNKGGCAACGACAACCTTCATACAATG |
| P714 | | |
| ERCAS12A- | 925 | /5Phos/AAAGACTTTTCGAAAAAATCCACGNNKAACGACAACCTTCATACAATGTAC |
| P715 | | |
| ERCAS12A- | 926 | /5Phos/GACTTTTCGAAAAAATCCACGGGCNNKGACAACCTTCATACAATGTACTTA |
| P716 | | |
| ERCAS12A- | 927 | /5Phos/TTTTCGAAAAAATCCACGGGCAACNNKAACCTTCATACAATGTACTTAAAG |
| P717 | | |
| ERCAS12A- | 928 | /5Phos/TCGAAAAAATCCACGGGCAACGACNNKCTTCATACAATGTACTTAAAGAAC |
| P718 | | |
| ERCAS12A- | 929 | /5Phos/AAAAAATCCACGGGCAACGACAACNNKCATACAATGTACTTAAAGAACCTG |
| P719 | | |
| ERCAS12A- | 930 | /5Phos/AAATCCACGGGCAACGACAACCTTNNKACAATGTACTTAAAGAACCTGTTC |
| P720 | | |
| ERCAS12A- | 931 | /5Phos/TCCACGGGCAACGACAACCTTCATNNKATGTACTTAAAGAACCTGTTCTCT |
| P721 | | |
| ERCAS12A- | 932 | /5Phos/ACGGGCAACGACAACCTTCATACANNKTACTTAAAGAACCTGTTCTCTGAA |
| P722 | | |
| ERCAS12A- | 933 | /5Phos/GGCAACGACAACCTTCATACAATGNNKTTAAAGAACCTGTTCTCTGAAGAA |
| P723 | | |
| ERCAS12A- | 934 | /5Phos/AACGACAACCTTCATACAATGTACNNKAAGAACCTGTTCTCTGAAGAAAAC |
| P724 | | |
| ERCAS12A- | 935 | /5Phos/GACAACCTTCATACAATGTACTTANNKAACCTGTTCTCTGAAGAAAACTTG |
| P725 | | |
| ERCAS12A- | 936 | /5Phos/AACCTTCATACAATGTACTTAAAGNNKCTGTTCTCTGAAGAAAACTTGAAA |
| P726 | | |
| ERCAS12A- | 937 | /5Phos/CTTCATACAATGTACTTAAAGAACNNKTTCTCTGAAGAAAACTTGAAAGAT |
| P727 | | |
| ERCAS12A- | 938 | /5Phos/CATACAATGTACTTAAAGAACCTGNNKTCTGAAGAAAACTTGAAAGATATC |
| P728 | | |
| ERCAS12A- | 939 | /5Phos/ACAATGTACTTAAAGAACCTGTTCNNKGAAGAAAACTTGAAAGATATCGTG |
| P729 | | |
| ERCAS12A- | 940 | /5Phos/ATGTACTTAAAGAACCTGTTCTCTNNKGAAAACTTGAAAGATATCGTGCTT |
| P730 | | |
| ERCAS12A- | 941 | /5Phos/TACTTAAAGAACCTGTTCTCTGAANNKAACTTGAAAGATATCGTGCTTAAA |
| P731 | | |
| ERCAS12A- | 942 | /5Phos/TTAAAGAACCTGTTCTCTGAAGAANNKTTGAAAGATATCGTGCTTAAATTG |
| P732 | | |
| ERCAS12A- | 943 | /5Phos/AAGAACCTGTTCTCTGAAGAAAACNNKAAAGATATCGTGCTTAAATTGAAT |
| P733 | | |
| ERCAS12A- | 944 | /5Phos/AACCTGTTCTCTGAAGAAAACTTGNNKGATATCGTGCTTAAATTGAATGGC |
| P734 | | |
| ERCAS12A- | 945 | /5Phos/CTGTTCTCTGAAGAAAACTTGAAANNKATCGTGCTTAAATTGAATGGCGAG |
| P735 | | |
| ERCAS12A- | 946 | /5Phos/TTCTCTGAAGAAAACTTGAAAGATNNKGTGCTTAAATTGAATGGCGAGGCC |
| P736 | | |
| ERCAS12A- | 947 | /5Phos/TCTGAAGAAAACTTGAAAGATATCNNKCTTAAATTGAATGGCGAGGCCGAG |
| P737 | | |
| ERCAS12A- | 948 | /5Phos/GAAGAAAACTTGAAAGATATCGTGNNKAAATTGAATGGCGAGGCCGAGATT |
| P738 | | |
| ERCAS12A- | 949 | /5Phos/GAAAACTTGAAAGATATCGTGCTTNNKTTGAATGGCGAGGCCGAGATTTTT |
| P739 | | |
| ERCAS12A- | 950 | /5Phos/AACTTGAAAGATATCGTGCTTAAANNKAATGGCGAGGCCGAGATTTTTTTC |
| P740 | | |
| ERCAS12A- | 951 | /5Phos/TTGAAAGATATCGTGCTTAAATTGNNKGGCGAGGCCGAGATTTTTTTCCGT |
| P741 | | |
| ERCAS12A- | 952 | /5Phos/AAAGATATCGTGCTTAAATTGAATNNKGAGGCCGAGATTTTTTTCCGTAAA |
| P742 | | |
| ERCAS12A- | 953 | /5Phos/GATATCGTGCTTAAATTGAATGGCNNKGCCGAGATTTTTTTCCGTAAAAGC |
| P743 | | |
| ERCAS12A- | 954 | /5Phos/ATCGTGCTTAAATTGAATGGCGAGNNKGAGATTTTTTTCCGTAAAAGCTCG |
| P744 | | |
| ERCAS12A | 955 | /5Phos/GTGCTTAAATTGAATGGCGAGGCCNNKATTTTTTTCCGTAAAAGCTCGATT |
| P745 | | |
| ERCAS12A | 956 | /5Phos/CTTAAATTGAATGGCGAGGCCGAGNNKTTTTTCCGTAAAAGCTCGATTAAG |
| P746 | | |
| ERCAS12A- | 957 | /5Phos/AAATTGAATGGCGAGGCCGAGATTNNKTTCCGTAAAAGCTCGATTAAGAAT |
| P747 | | |
| ERCAS12A- | 958 | /5Phos/TTGAATGGCGAGGCCGAGATTTTTNNKCGTAAAAGCTCGATTAAGAATCCC |
| P748 | | |
| ERCAS12A- | 959 | /5Phos/AATGGCGAGGCCGAGATTTTTTTCNNKAAAAGCTCGATTAAGAATCCCATC |
| P749 | | |
| ERCAS12A- | 960 | /5Phos/GGCGAGGCCGAGATTTTTTTCCGTNNKAGCTCGATTAAGAATCCCATCATC |
| P750 | | |
| ERCAS12A- | 961 | /5Phos/GAGGCCGAGATTTTTTTCCGTAAANNKTCGATTAAGAATCCCATCATCCAC |
| P751 | | |
| ERCAS12A- | 962 | /5Phos/GCCGAGATTTTTTTCCGTAAAAGCNNKATTAAGAATCCCATCATCCACAAG |
| P752 | | |
| ERCAS12A- | 963 | /5Phos/GAGATTTTTTTCCGTAAAAGCTCGNNKAAGAATCCCATCATCCACAAGAAA |
| P753 | | |
| ERCAS12A- | 964 | /5Phos/ATTTTTTTCCGTAAAAGCTCGATTNNKAATCCCATCATCCACAAGAAAGGC |
| P754 | | |
| ERCAS12A- | 965 | /5Phos/TTTTTCCGTAAAAGCTCGATTAAGNNKCCCATCATCCACAAGAAAGGCAGC |
| P755 | | |
| ERCAS12A- | 966 | /5Phos/TTCCGTAAAAGCTCGATTAAGAATNNKATCATCCACAAGAAAGGCAGCATT |
| P756 | | |
| ERCAS12A- | 967 | /5Phos/CGTAAAAGCTCGATTAAGAATCCCNNKATCCACAAGAAAGGCAGCATTCTG |
| P757 | | |
| ERCAS12A- | 968 | /5Phos/AAAAGCTCGATTAAGAATCCCATCNNKCACAAGAAAGGCAGCATTCTGGTA |
| P758 | | |
| ERCAS12A- | 969 | /5Phos/AGCTCGATTAAGAATCCCATCATCNNKAAGAAAGGCAGCATTCTGGTAAAT |
| P759 | | |
| ERCAS12A- | 970 | /5Phos/TCGATTAAGAATCCCATCATCCACNNKAAAGGCAGCATTCTGGTAAATCGT |
| P760 | | |
| ERCAS12A- | 971 | /5Phos/ATTAAGAATCCCATCATCCACAAGNNKGGCAGCATTCTGGTAAATCGTACC |
| P761 | | |
| ERCAS12A- | 972 | /5Phos/AAGAATCCCATCATCCACAAGAAANNKAGCATTCTGGTAAATCGTACCTAT |
| P762 | | |
| ERCAS12A- | 973 | /5Phos/AATCCCATCATCCACAAGAAAGGCNNKATTCTGGTAAATCGTACCTATGAA |
| P763 | | |
| ERCAS12A- | 974 | /5Phos/CCCATCATCCACAAGAAAGGCAGCNNKCTGGTAAATCGTACCTATGAAGCG |
| P764 | | |
| ERCAS12A- | 975 | /5Phos/ATCATCCACAAGAAAGGCAGCATTNNKGTAAATCGTACCTATGAAGCGGAG |
| P765 | | |
| ERCAS12A- | 976 | /5Phos/ATCCACAAGAAAGGCAGCATTCTGNNKAATCGTACCTATGAAGCGGAGGAA |
| P766 | | |
| ERCAS12A- | 977 | /5Phos/CACAAGAAAGGCAGCATTCTGGTANNKCGTACCTATGAAGCGGAGGAAAAG |
| P767 | | |
| ERCAS12A- | 978 | /5Phos/AAGAAAGGCAGCATTCTGGTAAATNNKACCTATGAAGCGGAGGAAAAGGAC |
| P768 | | |
| ERCAS12A- | 979 | /5Phos/AAAGGCAGCATTCTGGTAAATCGTNNKTATGAAGCGGAGGAAAAGGACCAA |
| P769 | | |
| ERCAS12A- | 980 | /5Phos/GGCAGCATTCTGGTAAATCGTACCNNKGAAGCGGAGGAAAAGGACCAATTT |
| P770 | | |
| ERCAS12A- | 981 | /5Phos/AGCATTCTGGTAAATCGTACCTATNNKGCGGAGGAAAAGGACCAATTTGGT |
| P771 | | |
| ERCAS12A- | 982 | /5Phos/ATTCTGGTAAATCGTACCTATGAANNKGAGGAAAAGGACCAATTTGGTAAT |
| P772 | | |
| ERCAS12A- | 983 | /5Phos/CTGGTAAATCGTACCTATGAAGCGNNKGAAAAGGACCAATTTGGTAATATT |
| P773 | | |
| ERCAS12A- | 984 | /5Phos/GTAAATCGTACCTATGAAGCGGAGNNKAAGGACCAATTTGGTAATATTCAG |
| P774 | | |
| ERCAS12A- | 985 | /5Phos/AATCGTACCTATGAAGCGGAGGAANNKGACCAATTTGGTAATATTCAGATT |
| P775 | | |
| ERCAS12A- | 986 | /5Phos/CGTACCTATGAAGCGGAGGAAAAGNNKCAATTTGGTAATATTCAGATTGTC |
| P776 | | |
| ERCAS12A- | 987 | /5Phos/ACCTATGAAGCGGAGGAAAAGGACNNKTTTGGTAATATTCAGATTGTCCGT |
| P777 | | |
| ERCAS12A- | 988 | /5Phos/TATGAAGCGGAGGAAAAGGACCAANNKGGTAATATTCAGATTGTCCGTAAG |
| P778 | | |
| ERCAS12A- | 989 | /5Phos/GAAGCGGAGGAAAAGGACCAATTTNNKAATATTCAGATTGTCCGTAAGAAC |
| P779 | | |
| ERCAS12A- | 990 | /5Phos/GCGGAGGAAAAGGACCAATTTGGTNNKATTCAGATTGTCCGTAAGAACATC |
| P780 | | |
| ERCAS12A- | 991 | /5Phos/GAGGAAAAGGACCAATTTGGTAATNNKCAGATTGTCCGTAAGAACATCCCT |
| P781 | | |
| ERCAS12A- | 992 | /5Phos/GAAAAGGACCAATTTGGTAATATTNNKATTGTCCGTAAGAACATCCCTGAA |
| P782 | | |
| ERCAS12A- | 993 | /5Phos/AAGGACCAATTTGGTAATATTCAGNNKGTCCGTAAGAACATCCCTGAAAAT |
| P783 | | |
| ERCAS12A- | 994 | /5Phos/GACCAATTTGGTAATATTCAGATTNNKCGTAAGAACATCCCTGAAAATATC |
| P784 | | |
| ERCAS12A- | 995 | /5Phos/CAATTTGGTAATATTCAGATTGTCNNKAAGAACATCCCTGAAAATATCTAT |
| P785 | | |
| ERCAS12A- | 996 | /5Phos/TTTGGTAATATTCAGATTGTCCGTNNKAACATCCCTGAAAATATCTATCAA |
| P786 | | |
| ERCAS12A- | 997 | /5Phos/GGTAATATTCAGATTGTCCGTAAGNNKATCCCTGAAAATATCTATCAAGAA |
| P787 | | |
| ERCAS12A- | 998 | /5Phos/AATATTCAGATTGTCCGTAAGAACNNKCCTGAAAATATCTATCAAGAATTA |
| P788 | | |
| ERCAS12A- | 999 | /5Phos/ATTCAGATTGTCCGTAAGAACATCNNKGAAAATATCTATCAAGAATTATAT |
| P789 | | |
| ERCAS12A | 1000 | /5Phos/CAGATTGTCCGTAAGAACATCCCTNNKAATATCTATCAAGAATTATATAAA |
| P790 | | |
| ERCAS12A | 1001 | /5Phos/ATTGTCCGTAAGAACATCCCTGAANNKATCTATCAAGAATTATATAAATAT |
| P791 | | |
| ERCAS12A- | 1002 | /5Phos/GTCCGTAAGAACATCCCTGAAAATNNKTATCAAGAATTATATAAATATTTC |
| P792 | | |
| ERCAS12A- | 1003 | /5Phos/CGTAAGAACATCCCTGAAAATATCNNKCAAGAATTATATAAATATTTCAAC |
| P793 | | |
| ERCAS12A- | 1004 | /5Phos/AAGAACATCCCTGAAAATATCTATNNKGAATTATATAAATATTTCAACGAT |
| P794 | | |
| ERCAS12A- | 1005 | /5Phos/AACATCCCTGAAAATATCTATCAANNKTTATATAAATATTTCAACGATAAG |
| P795 | | |
| ERCAS12A- | 1006 | /5Phos/ATCCCTGAAAATATCTATCAAGAANNKTATAAATATTTCAACGATAAGTCG |
| P796 | | |
| ERCAS12A- | 1007 | /5Phos/CCTGAAAATATCTATCAAGAATTANNKAAATATTTCAACGATAAGTCGGAC |
| P797 | | |
| ERCAS12A- | 1008 | /5Phos/GAAAATATCTATCAAGAATTATATNNKTATTTCAACGATAAGTCGGACAAG |
| P798 | | |
| ERCAS12A- | 1009 | /5Phos/AATATCTATCAAGAATTATATAAANNKTTCAACGATAAGTCGGACAAGGAA |
| P799 | | |
| ERCAS12A- | 1010 | /5Phos/ATCTATCAAGAATTATATAAATATNNKAACGATAAGTCGGACAAGGAACTT |
| P800 | | |
| ERCAS12A- | 1011 | /5Phos/TATCAAGAATTATATAAATATTTCNNKGATAAGTCGGACAAGGAACTTAGC |
| P801 | | |
| ERCAS12A- | 1012 | /5Phos/CAAGAATTATATAAATATTTCAACNNKAAGTCGGACAAGGAACTTAGCGAT |
| P802 | | |
| ERCAS12A- | 1013 | /5Phos/GAATTATATAAATATTTCAACGATNNKTCGGACAAGGAACTTAGCGATGAA |
| P803 | | |
| ERCAS12A- | 1014 | /5Phos/TTATATAAATATTTCAACGATAAGNNKGACAAGGAACTTAGCGATGAAGCC |
| P804 | | |
| ERCAS12A- | 1015 | /5Phos/TATAAATATTTCAACGATAAGTCGNNKAAGGAACTTAGCGATGAAGCCGCG |
| P805 | | |
| ERCAS12A- | 1016 | /5Phos/AAATATTTCAACGATAAGTCGGACNNKGAACTTAGCGATGAAGCCGCGAAG |
| P806 | | |
| ERCAS12A- | 1017 | /5Phos/TATTTCAACGATAAGTCGGACAAGNNKCTTAGCGATGAAGCCGCGAAGTTG |
| P807 | | |
| ERCAS12A- | 1018 | /5Phos/TTCAACGATAAGTCGGACAAGGAANNKAGCGATGAAGCCGCGAAGTTGAAA |
| P808 | | |
| ERCAS12A- | 1019 | /5Phos/AACGATAAGTCGGACAAGGAACTTNNKGATGAAGCCGCGAAGTTGAAAAAT |
| P809 | | |
| ERCAS12A | 1020 | /5Phos/GATAAGTCGGACAAGGAACTTAGCNNKGAAGCCGCGAAGTTGAAAAATGTT |
| P810 | | |
| ERCAS12A- | 1021 | /5Phos/AAGTCGGACAAGGAACTTAGCGATNNKGCCGCGAAGTTGAAAAATGTTGTT |
| P811 | | |
| ERCAS12A- | 1022 | /5Phos/TCGGACAAGGAACTTAGCGATGAANNKGCGAAGTTGAAAAATGTTGTTGGT |
| P812 | | |
| ERCAS12A- | 1023 | /5Phos/GACAAGGAACTTAGCGATGAAGCCNNKAAGTTGAAAAATGTTGTTGGTCAC |
| P813 | | |
| ERCAS12A- | 1024 | /5Phos/AAGGAACTTAGCGATGAAGCCGCGNNKTTGAAAAATGTTGTTGGTCACCAC |
| P814 | | |
| ERCAS12A- | 1025 | /5Phos/GAACTTAGCGATGAAGCCGCGAAGNNKAAAAATGTTGTTGGTCACCACGAA |
| P815 | | |
| ERCAS12A- | 1026 | /5Phos/CTTAGCGATGAAGCCGCGAAGTTGNNKAATGTTGTTGGTCACCACGAAGCA |
| P816 | | |
| ERCAS12A- | 1027 | /5Phos/AGCGATGAAGCCGCGAAGTTGAAANNKGTTGTTGGTCACCACGAAGCAGCA |
| P817 | | |
| ERCAS12A- | 1028 | /5Phos/GATGAAGCCGCGAAGTTGAAAAATNNKGTTGGTCACCACGAAGCAGCAACA |
| P818 | | |
| ERCAS12A- | 1029 | /5Phos/GAAGCCGCGAAGTTGAAAAATGTTNNKGGTCACCACGAAGCAGCAACAAAC |
| P819 | | |
| ERCAS12A- | 1030 | /5Phos/GCCGCGAAGTTGAAAAATGTTGTTNNKCACCACGAAGCAGCAACAAACATT |
| P820 | | |
| ERCAS12A- | 1031 | /5Phos/GCGAAGTTGAAAAATGTTGTTGGTNNKCACGAAGCAGCAACAAACATTGTG |
| P821 | | |
| ERCAS12A- | 1032 | /5Phos/AAGTTGAAAAATGTTGTTGGTCACNNKGAAGCAGCAACAAACATTGTGAAA |
| P822 | | |
| ERCAS12A- | 1033 | /5Phos/TTGAAAAATGTTGTTGGTCACCACNNKGCAGCAACAAACATTGTGAAAGAC |
| P823 | | |
| ERCAS12A- | 1034 | /5Phos/AAAAATGTTGTTGGTCACCACGAANNKGCAACAAACATTGTGAAAGACTAT |
| P824 | | |
| ERCAS12A- | 1035 | /5Phos/AATGTTGTTGGTCACCACGAAGCANNKACAAACATTGTGAAAGACTATCGC |
| P825 | | |
| ERCAS12A- | 1036 | /5Phos/GTTGTTGGTCACCACGAAGCAGCANNKAACATTGTGAAAGACTATCGCTAC |
| P826 | | |
| ERCAS12A- | 1037 | /5Phos/GTTGGTCACCACGAAGCAGCAACANNKATTGTGAAAGACTATCGCTACACT |
| P827 | | |
| ERCAS12A- | 1038 | /5Phos/GGTCACCACGAAGCAGCAACAAACNNKGTGAAAGACTATCGCTACACTTAC |
| P828 | | |
| ERCAS12A- | 1039 | /5Phos/CACCACGAAGCAGCAACAAACATTNNKAAAGACTATCGCTACACTTACGAT |
| P829 | | |
| ERCAS12A- | 1040 | /5Phos/CACGAAGCAGCAACAAACATTGTGNNKGACTATCGCTACACTTACGATAAG |
| P830 | | |
| ERCAS12A- | 1041 | /5Phos/GAAGCAGCAACAAACATTGTGAAANNKTATCGCTACACTTACGATAAGTAC |
| P831 | | |
| ERCAS12A- | 1042 | /5Phos/GCAGCAACAAACATTGTGAAAGACNNKCGCTACACTTACGATAAGTACTTT |
| P832 | | |
| ERCAS12A- | 1043 | /5Phos/GCAACAAACATTGTGAAAGACTATNNKTACACTTACGATAAGTACTTTTTA |
| P833 | | |
| ERCAS12A- | 1044 | /5Phos/ACAAACATTGTGAAAGACTATCGCNNKACTTACGATAAGTACTTTTTACAT |
| P834 | | |
| ERCAS12A- | 1045 | /5Phos/AACATTGTGAAAGACTATCGCTACNNKTACGATAAGTACTTTTTACATATG |
| P835 | | |
| ERCAS12A- | 1046 | /5Phos/ATTGTGAAAGACTATCGCTACACTNNKGATAAGTACTTTTTACATATGCCT |
| P836 | | |
| ERCAS12A- | 1047 | /5Phos/GTGAAAGACTATCGCTACACTTACNNKAAGTACTTTTTACATATGCCTATT |
| P837 | | |
| ERCAS12A- | 1048 | /5Phos/AAAGACTATCGCTACACTTACGATNNKTACTTTTTACATATGCCTATTACG |
| P838 | | |
| ERCAS12A- | 1049 | /5Phos/GACTATCGCTACACTTACGATAAGNNKTTTTTACATATGCCTATTACGATT |
| P839 | | |
| ERCAS12A- | 1050 | /5Phos/TATCGCTACACTTACGATAAGTACNNKTTACATATGCCTATTACGATTAAC |
| P840 | | |
| ERCAS12A- | 1051 | /5Phos/CGCTACACTTACGATAAGTACTTTNNKCATATGCCTATTACGATTAACTTT |
| P841 | | |
| ERCAS12A- | 1052 | /5Phos/TACACTTACGATAAGTACTTTTTANNKATGCCTATTACGATTAACTTTAAG |
| P842 | | |
| ERCAS12A- | 1053 | /5Phos/ACTTACGATAAGTACTTTTTACATNNKCCTATTACGATTAACTTTAAGGCA |
| P843 | | |
| ERCAS12A- | 1054 | /5Phos/TACGATAAGTACTTTTTACATATGNNKATTACGATTAACTTTAAGGCAAAT |
| P844 | | |
| ERCAS12A- | 1055 | /5Phos/GATAAGTACTTTTTACATATGCCTNNKACGATTAACTTTAAGGCAAATAAG |
| P845 | | |
| ERCAS12A- | 1056 | /5Phos/AAGTACTTTTTACATATGCCTATTNNKATTAACTTTAAGGCAAATAAGACA |
| P846 | | |
| ERCAS12A- | 1057 | /5Phos/TACTTTTTACATATGCCTATTACGNNKAACTTTAAGGCAAATAAGACAGGT |
| P847 | | |
| ERCAS12A- | 1058 | /5Phos/TTTTTACATATGCCTATTACGATTNNKTTTAAGGCAAATAAGACAGGTTTT |
| P848 | | |
| ERCAS12A- | 1059 | /5Phos/TTACATATGCCTATTACGATTAACNNKAAGGCAAATAAGACAGGTTTTATC |
| P849 | | |
| ERCAS12A- | 1060 | /5Phos/CATATGCCTATTACGATTAACTTTNNKGCAAATAAGACAGGTTTTATCAAC |
| P850 | | |
| ERCAS12A- | 1061 | /5Phos/ATGCCTATTACGATTAACTTTAAGNNKAATAAGACAGGTTTTATCAACGAC |
| P851 | | |
| ERCAS12A- | 1062 | /5Phos/CCTATTACGATTAACTTTAAGGCANNKAAGACAGGTTTTATCAACGACCGT |
| P852 | | |
| ERCAS12A- | 1063 | /5Phos/ATTACGATTAACTTTAAGGCAAATNNKACAGGTTTTATCAACGACCGTATC |
| P853 | | |
| ERCAS12A- | 1064 | /5Phos/ACGATTAACTTTAAGGCAAATAAGNNKGGTTTTATCAACGACCGTATCCTG |
| P854 | | |
| ERCAS12A- | 1065 | /5Phos/ATTAACTTTAAGGCAAATAAGACANNKTTTATCAACGACCGTATCCTGCAA |
| P855 | | |
| ERCAS12A- | 1066 | /5Phos/AACTTTAAGGCAAATAAGACAGGTNNKATCAACGACCGTATCCTGCAATAT |
| P856 | | |
| ERCAS12A- | 1067 | /5Phos/TTTAAGGCAAATAAGACAGGTTTTNNKAACGACCGTATCCTGCAATATATT |
| P857 | | |
| ERCAS12A- | 1068 | /5Phos/AAGGCAAATAAGACAGGTTTTATCNNKGACCGTATCCTGCAATATATTGCA |
| P858 | | |
| ERCAS12A- | 1069 | /5Phos/GCAAATAAGACAGGTTTTATCAACNNKCGTATCCTGCAATATATTGCAAAA |
| P859 | | |
| ERCAS12A- | 1070 | /5Phos/AATAAGACAGGTTTTATCAACGACNNKATCCTGCAATATATTGCAAAAGAG |
| P860 | | |
| ERCAS12A- | 1071 | /5Phos/AAGACAGGTTTTATCAACGACCGTNNKCTGCAATATATTGCAAAAGAGAAG |
| P861 | | |
| ERCAS12A- | 1072 | /5Phos/ACAGGTTTTATCAACGACCGTATCNNKCAATATATTGCAAAAGAGAAGGAC |
| P862 | | |
| ERCAS12A- | 1073 | /5Phos/GGTTTTATCAACGACCGTATCCTGNNKTATATTGCAAAAGAGAAGGACCTG |
| P863 | | |
| ERCAS12A- | 1074 | /5Phos/TTTATCAACGACCGTATCCTGCAANNKATTGCAAAAGAGAAGGACCTGCAT |
| P864 | | |
| ERCAS12A- | 1075 | /5Phos/ATCAACGACCGTATCCTGCAATATNNKGCAAAAGAGAAGGACCTGCATGTC |
| P865 | | |
| ERCAS12A- | 1076 | /5Phos/AACGACCGTATCCTGCAATATATTNNKAAAGAGAAGGACCTGCATGTCATT |
| P866 | | |
| ERCAS12A- | 1077 | /5Phos/GACCGTATCCTGCAATATATTGCANNKGAGAAGGACCTGCATGTCATTGGG |
| P867 | | |
| ERCAS12A- | 1078 | /5Phos/CGTATCCTGCAATATATTGCAAAANNKAAGGACCTGCATGTCATTGGGATT |
| P868 | | |
| ERCAS12A- | 1079 | /5Phos/ATCCTGCAATATATTGCAAAAGAGNNKGACCTGCATGTCATTGGGATTGAT |
| P869 | | |
| ERCAS12A- | 1080 | /5Phos/CTGCAATATATTGCAAAAGAGAAGNNKCTGCATGTCATTGGGATTGATCGC |
| P870 | | |
| ERCAS12A- | 1081 | /5Phos/CAATATATTGCAAAAGAGAAGGACNNKCATGTCATTGGGATTGATCGCGGT |
| P871 | | |
| ERCAS12A- | 1082 | /5Phos/TATATTGCAAAAGAGAAGGACCTGNNKGTCATTGGGATTGATCGCGGTGAA |
| P872 | | |
| ERCAS12A- | 1083 | /5Phos/ATTGCAAAAGAGAAGGACCTGCATNNKATTGGGATTGATCGCGGTGAACGC |
| P873 | | |
| ERCAS12A- | 1084 | /5Phos/GCAAAAGAGAAGGACCTGCATGTCNNKGGGATTGATCGCGGTGAACGCAAT |
| P874 | | |
| ERCAS12A- | 1085 | /5Phos/AAAGAGAAGGACCTGCATGTCATTNNKATTGATCGCGGTGAACGCAATCTG |
| P875 | | |
| ERCAS12A- | 1086 | /5Phos/GAGAAGGACCTGCATGTCATTGGGNNKGATCGCGGTGAACGCAATCTGATT |
| P876 | | |
| ERCAS12A- | 1087 | /5Phos/AAGGACCTGCATGTCATTGGGATTNNKCGCGGTGAACGCAATCTGATTTAC |
| P877 | | |
| ERCAS12A- | 1088 | /5Phos/GACCTGCATGTCATTGGGATTGATNNKGGTGAACGCAATCTGATTTACGTG |
| P878 | | |
| ERCAS12A- | 1089 | /5Phos/CTGCATGTCATTGGGATTGATCGCNNKGAACGCAATCTGATTTACGTGAGT |
| P879 | | |
| ERCAS12A- | 1090 | /5Phos/CATGTCATTGGGATTGATCGCGGTNNKCGCAATCTGATTTACGTGAGTGTT |
| P880 | | |
| ERCAS12A- | 1091 | /5Phos/GTCATTGGGATTGATCGCGGTGAANNKAATCTGATTTACGTGAGTGTTATT |
| P881 | | |
| ERCAS12A- | 1092 | /5Phos/ATTGGGATTGATCGCGGTGAACGCNNKCTGATTTACGTGAGTGTTATTGAT |
| P882 | | |
| ERCAS12A- | 1093 | /5Phos/GGGATTGATCGCGGTGAACGCAATNNKATTTACGTGAGTGTTATTGATACC |
| P883 | | |
| ERCAS12A- | 1094 | /5Phos/ATTGATCGCGGTGAACGCAATCTGNNKTACGTGAGTGTTATTGATACCTGC |
| P884 | | |
| ERCAS12A- | 1095 | /5Phos/GATCGCGGTGAACGCAATCTGATTNNKGTGAGTGTTATTGATACCTGCGGT |
| P885 | | |
| ERCAS12A- | 1096 | /5Phos/CGCGGTGAACGCAATCTGATTTACNNKAGTGTTATTGATACCTGCGGTAAT |
| P886 | | |
| ERCAS12A- | 1097 | /5Phos/GGTGAACGCAATCTGATTTACGTGNNKGTTATTGATACCTGCGGTAATATT |
| P887 | | |
| ERCAS12A- | 1098 | /5Phos/GAACGCAATCTGATTTACGTGAGTNNKATTGATACCTGCGGTAATATTGTT |
| P888 | | |
| ERCAS12A- | 1099 | /5Phos/CGCAATCTGATTTACGTGAGTGTTNNKGATACCTGCGGTAATATTGTTGAG |
| P889 | | |
| ERCAS12A- | 1100 | /5Phos/AATCTGATTTACGTGAGTGTTATTNNKACCTGCGGTAATATTGTTGAGCAA |
| P890 | | |
| ERCAS12A- | 1101 | /5Phos/CTGATTTACGTGAGTGTTATTGATNNKTGCGGTAATATTGTTGAGCAAAAG |
| P891 | | |
| ERCAS12A- | 1102 | /5Phos/ATTTACGTGAGTGTTATTGATACCNNKGGTAATATTGTTGAGCAAAAGTCT |
| P892 | | |
| ERCAS12A- | 1103 | /5Phos/TACGTGAGTGTTATTGATACCTGCNNKAATATTGTTGAGCAAAAGTCTTTT |
| P893 | | |
| ERCAS12A- | 1104 | /5Phos/GTGAGTGTTATTGATACCTGCGGTNNKATTGTTGAGCAAAAGTCTTTTAAT |
| P894 | | |
| ERCAS12A- | 1105 | /5Phos/AGTGTTATTGATACCTGCGGTAATNNKGTTGAGCAAAAGTCTTTTAATATC |
| P895 | | |
| ERCAS12A- | 1106 | /5Phos/GTTATTGATACCTGCGGTAATATTNNKGAGCAAAAGTCTTTTAATATCGTT |
| P896 | | |
| ERCAS12A- | 1107 | /5Phos/ATTGATACCTGCGGTAATATTGTTNNKCAAAAGTCTTTTAATATCGTTAAT |
| P897 | | |
| ERCAS12A- | 1108 | /5Phos/GATACCTGCGGTAATATTGTTGAGNNKAAGTCTTTTAATATCGTTAATGGT |
| P898 | | |
| ERCAS12A- | 1109 | /5Phos/ACCTGCGGTAATATTGTTGAGCAANNKTCTTTTAATATCGTTAATGGTTAC |
| P899 | | |
| ERCAS12A- | 1110 | /5Phos/TGCGGTAATATTGTTGAGCAAAAGNNKTTTAATATCGTTAATGGTTACGAC |
| P900 | | |
| ERCAS12A- | 1111 | /5Phos/GGTAATATTGTTGAGCAAAAGTCTNNKAATATCGTTAATGGTTACGACTAC |
| P901 | | |
| ERCAS12A- | 1112 | /5Phos/AATATTGTTGAGCAAAAGTCTTTTNNKATCGTTAATGGTTACGACTACCAA |
| P902 | | |
| ERCAS12A- | 1113 | /5Phos/ATTGTTGAGCAAAAGTCTTTTAATNNKGTTAATGGTTACGACTACCAAATT |
| P903 | | |
| ERCAS12A- | 1114 | /5Phos/GTTGAGCAAAAGTCTTTTAATATCNNKAATGGTTACGACTACCAAATTAAG |
| P904 | | |
| ERCAS12A- | 1115 | /5Phos/GAGCAAAAGTCTTTTAATATCGTTNNKGGTTACGACTACCAAATTAAGCTT |
| P905 | | |
| ERCAS12A- | 1116 | /5Phos/CAAAAGTCTTTTAATATCGTTAATNNKTACGACTACCAAATTAAGCTTAAA |
| P906 | | |
| ERCAS12A- | 1117 | /5Phos/AAGTCTTTTAATATCGTTAATGGTNNKGACTACCAAATTAAGCTTAAACAA |
| P907 | | |
| ERCAS12A- | 1118 | /5Phos/TCTTTTAATATCGTTAATGGTTACNNKTACCAAATTAAGCTTAAACAACAG |
| P908 | | |
| ERCAS12A- | 1119 | /5Phos/TTTAATATCGTTAATGGTTACGACNNKCAAATTAAGCTTAAACAACAGGAA |
| P909 | | |
| ERCAS12A- | 1120 | /5Phos/AATATCGTTAATGGTTACGACTACNNKATTAAGCTTAAACAACAGGAAGGT |
| P910 | | |
| ERCAS12A- | 1121 | /5Phos/ATCGTTAATGGTTACGACTACCAANNKAAGCTTAAACAACAGGAAGGTGCC |
| P911 | | |
| ERCAS12A- | 1122 | /5Phos/GTTAATGGTTACGACTACCAAATTNNKCTTAAACAACAGGAAGGTGCCCGC |
| P912 | | |
| ERCAS12A- | 1123 | /5Phos/AATGGTTACGACTACCAAATTAAGNNKAAACAACAGGAAGGTGCCCGCCAG |
| P913 | | |
| ERCAS12A- | 1124 | /5Phos/GGTTACGACTACCAAATTAAGCTTNNKCAACAGGAAGGTGCCCGCCAGATC |
| P914 | | |
| ERCAS12A- | 1125 | /5Phos/TACGACTACCAAATTAAGCTTAAANNKCAGGAAGGTGCCCGCCAGATCGCC |
| P915 | | |
| ERCAS12A- | 1126 | /5Phos/GACTACCAAATTAAGCTTAAACAANNKGAAGGTGCCCGCCAGATCGCCCGT |
| P916 | | |
| ERCAS12A- | 1127 | /5Phos/TACCAAATTAAGCTTAAACAACAGNNKGGTGCCCGCCAGATCGCCCGTAAG |
| P917 | | |
| ERCAS12A | 1128 | /5Phos/CAAATTAAGCTTAAACAACAGGAANNKGCCCGCCAGATCGCCCGTAAGGAG |
| P918 | | |
| ERCAS12A- | 1129 | /5Phos/ATTAAGCTTAAACAACAGGAAGGTNNKCGCCAGATCGCCCGTAAGGAGTGG |
| P919 | | |
| ERCAS12A- | 1130 | /5Phos/AAGCTTAAACAACAGGAAGGTGCCNNKCAGATCGCCCGTAAGGAGTGGAAG |
| P920 | | |
| ERCAS12A- | 1131 | /5Phos/CTTAAACAACAGGAAGGTGCCCGCNNKATCGCCCGTAAGGAGTGGAAGGAA |
| P921 | | |
| ERCAS12A- | 1132 | /5Phos/AAACAACAGGAAGGTGCCCGCCAGNNKGCCCGTAAGGAGTGGAAGGAAATC |
| P922 | | |
| ERCAS12A- | 1133 | /5Phos/CAACAGGAAGGTGCCCGCCAGATCNNKCGTAAGGAGTGGAAGGAAATCGGA |
| P923 | | |
| ERCAS12A- | 1134 | /5Phos/CAGGAAGGTGCCCGCCAGATCGCCNNKAAGGAGTGGAAGGAAATCGGAAAA |
| P924 | | |
| ERCAS12A- | 1135 | /5Phos/GAAGGTGCCCGCCAGATCGCCCGTNNKGAGTGGAAGGAAATCGGAAAAATT |
| P925 | | |
| ERCAS12A- | 1136 | /5Phos/GGTGCCCGCCAGATCGCCCGTAAGNNKTGGAAGGAAATCGGAAAAATTAAG |
| P926 | | |
| ERCAS12A- | 1137 | /5Phos/GCCCGCCAGATCGCCCGTAAGGAGNNKAAGGAAATCGGAAAAATTAAGGAA |
| P927 | | |
| ERCAS12A- | 1138 | /5Phos/CGCCAGATCGCCCGTAAGGAGTGGNNKGAAATCGGAAAAATTAAGGAAATC |
| P928 | | |
| ERCAS12A- | 1139 | /5Phos/CAGATCGCCCGTAAGGAGTGGAAGNNKATCGGAAAAATTAAGGAAATCAAA |
| P929 | | |
| ERCAS12A- | 1140 | /5Phos/ATCGCCCGTAAGGAGTGGAAGGAANNKGGAAAAATTAAGGAAATCAAAGAG |
| P930 | | |
| ERCAS12A- | 1141 | /5Phos/GCCCGTAAGGAGTGGAAGGAAATCNNKAAAATTAAGGAAATCAAAGAGGGA |
| P931 | | |
| ERCAS12A- | 1142 | /5Phos/CGTAAGGAGTGGAAGGAAATCGGANNKATTAAGGAAATCAAAGAGGGATAT |
| P932 | | |
| ERCAS12A- | 1143 | /5Phos/AAGGAGTGGAAGGAAATCGGAAAANNKAAGGAAATCAAAGAGGGATATCTG |
| P933 | | |
| ERCAS12A- | 1144 | /5Phos/GAGTGGAAGGAAATCGGAAAAATTNNKGAAATCAAAGAGGGATATCTGAGC |
| P934 | | |
| ERCAS12A- | 1145 | /5Phos/TGGAAGGAAATCGGAAAAATTAAGNNKATCAAAGAGGGATATCTGAGCTTG |
| P935 | | |
| ERCAS12A- | 1146 | /5Phos/AAGGAAATCGGAAAAATTAAGGAANNKAAAGAGGGATATCTGAGCTTGGTT |
| P936 | | |
| ERCAS12A- | 1147 | /5Phos/GAAATCGGAAAAATTAAGGAAATCNNKGAGGGATATCTGAGCTTGGTTATT |
| P937 | | |
| ERCAS12A- | 1148 | /5Phos/ATCGGAAAAATTAAGGAAATCAAANNKGGATATCTGAGCTTGGTTATTCAT |
| P938 | | |
| ERCAS12A- | 1149 | /5Phos/GGAAAAATTAAGGAAATCAAAGAGNNKTATCTGAGCTTGGTTATTCATGAA |
| P939 | | |
| ERCAS12A- | 1150 | /5Phos/AAAATTAAGGAAATCAAAGAGGGANNKCTGAGCTTGGTTATTCATGAAATC |
| P940 | | |
| ERCAS12A- | 1151 | /5Phos/ATTAAGGAAATCAAAGAGGGATATNNKAGCTTGGTTATTCATGAAATCTCA |
| P941 | | |
| ERCAS12A- | 1152 | /5Phos/AAGGAAATCAAAGAGGGATATCTGNNKTTGGTTATTCATGAAATCTCAAAG |
| P942 | | |
| ERCAS12A- | 1153 | /5Phos/GAAATCAAAGAGGGATATCTGAGCNNKGTTATTCATGAAATCTCAAAGATG |
| P943 | | |
| ERCAS12A- | 1154 | /5Phos/ATCAAAGAGGGATATCTGAGCTTGNNKATTCATGAAATCTCAAAGATGGTT |
| P944 | | |
| ERCAS12A- | 1155 | /5Phos/AAAGAGGGATATCTGAGCTTGGTTNNKCATGAAATCTCAAAGATGGTTATT |
| P945 | | |
| ERCAS12A- | 1156 | /5Phos/GAGGGATATCTGAGCTTGGTTATTNNKGAAATCTCAAAGATGGTTATTAAG |
| P946 | | |
| ERCAS12A- | 1157 | /5Phos/GGATATCTGAGCTTGGTTATTCATNNKATCTCAAAGATGGTTATTAAGTAC |
| P947 | | |
| ERCAS12A- | 1158 | /5Phos/TATCTGAGCTTGGTTATTCATGAANNKTCAAAGATGGTTATTAAGTACAAC |
| P948 | | |
| ERCAS12A- | 1159 | /5Phos/CTGAGCTTGGTTATTCATGAAATCNNKAAGATGGTTATTAAGTACAACGCC |
| P949 | | |
| ERCAS12A- | 1160 | /5Phos/AGCTTGGTTATTCATGAAATCTCANNKATGGTTATTAAGTACAACGCCATC |
| P950 | | |
| ERCAS12A- | 1161 | /5Phos/TTGGTTATTCATGAAATCTCAAAGNNKGTTATTAAGTACAACGCCATCATC |
| P951 | | |
| ERCAS12A- | 1162 | /5Phos/GTTATTCATGAAATCTCAAAGATGNNKATTAAGTACAACGCCATCATCGCT |
| P952 | | |
| ERCAS12A- | 1163 | /5Phos/ATTCATGAAATCTCAAAGATGGTTNNKAAGTACAACGCCATCATCGCTATG |
| P953 | | |
| ERCAS12A- | 1164 | /5Phos/CATGAAATCTCAAAGATGGTTATTNNKTACAACGCCATCATCGCTATGGAA |
| P954 | | |
| ERCAS12A- | 1165 | /5Phos/GAAATCTCAAAGATGGTTATTAAGNNKAACGCCATCATCGCTATGGAAGAC |
| P955 | | |
| ERCAS12A- | 1166 | /5Phos/ATCTCAAAGATGGTTATTAAGTACNNKGCCATCATCGCTATGGAAGACTTA |
| P956 | | |
| ERCAS12A- | 1167 | /5Phos/TCAAAGATGGTTATTAAGTACAACNNKATCATCGCTATGGAAGACTTATCG |
| P957 | | |
| ERCAS12A | 1168 | /5Phos/AAGATGGTTATTAAGTACAACGCCNNKATCGCTATGGAAGACTTATCGTAT |
| P958 | | |
| ERCAS12A- | 1169 | /5Phos/ATGGTTATTAAGTACAACGCCATCNNKGCTATGGAAGACTTATCGTATGGG |
| P959 | | |
| ERCAS12A- | 1170 | /5Phos/GTTATTAAGTACAACGCCATCATCNNKATGGAAGACTTATCGTATGGGTTT |
| P960 | | |
| ERCAS12A- | 1171 | /5Phos/ATTAAGTACAACGCCATCATCGCTNNKGAAGACTTATCGTATGGGTTTAAA |
| P961 | | |
| ERCAS12A | 1172 | /5Phos/AAGTACAACGCCATCATCGCTATGNNKGACTTATCGTATGGGTTTAAAAAG |
| P962 | | |
| ERCAS12A- | 1173 | /5Phos/TACAACGCCATCATCGCTATGGAANNKTTATCGTATGGGTTTAAAAAGGGT |
| P963 | | |
| ERCAS12A- | 1174 | /5Phos/AACGCCATCATCGCTATGGAAGACNNKTCGTATGGGTTTAAAAAGGGTCGT |
| P964 | | |
| ERCAS12A- | 1175 | /5Phos/GCCATCATCGCTATGGAAGACTTANNKTATGGGTTTAAAAAGGGTCGTTTC |
| P965 | | |
| ERCAS12A- | 1176 | /5Phos/ATCATCGCTATGGAAGACTTATCGNNKGGGTTTAAAAAGGGTCGTTTCAAA |
| P966 | | |
| ERCAS12A- | 1177 | /5Phos/ATCGCTATGGAAGACTTATCGTATNNKTTTAAAAAGGGTCGTTTCAAAGTT |
| P967 | | |
| ERCAS12A- | 1178 | /5Phos/GCTATGGAAGACTTATCGTATGGGNNKAAAAAGGGTCGTTTCAAAGTTGAG |
| P968 | | |
| ERCAS12A- | 1179 | /5Phos/ATGGAAGACTTATCGTATGGGTTTNNKAAGGGTCGTTTCAAAGTTGAGCGC |
| P969 | | |
| ERCAS12A- | 1180 | /5Phos/GAAGACTTATCGTATGGGTTTAAANNKGGTCGTTTCAAAGTTGAGCGCCAG |
| P970 | | |
| ERCAS12A- | 1181 | /5Phos/GACTTATCGTATGGGTTTAAAAAGNNKCGTTTCAAAGTTGAGCGCCAGGTC |
| P971 | | |
| ERCAS12A- | 1182 | /5Phos/TTATCGTATGGGTTTAAAAAGGGTNNKTTCAAAGTTGAGCGCCAGGTCTAC |
| P972 | | |
| ERCAS12A- | 1183 | /5Phos/TCGTATGGGTTTAAAAAGGGTCGTNNKAAAGTTGAGCGCCAGGTCTACCAA |
| P973 | | |
| ERCAS12A- | 1184 | /5Phos/TATGGGTTTAAAAAGGGTCGTTTCNNKGTTGAGCGCCAGGTCTACCAAAAG |
| P974 | | |
| ERCAS12A- | 1185 | /5Phos/GGGTTTAAAAAGGGTCGTTTCAAANNKGAGCGCCAGGTCTACCAAAAGTTT |
| P975 | | |
| ERCAS12A- | 1186 | /5Phos/TTTAAAAAGGGTCGTTTCAAAGTTNNKCGCCAGGTCTACCAAAAGTTTGAA |
| P976 | | |
| ERCAS12A- | 1187 | /5Phos/AAAAAGGGTCGTTTCAAAGTTGAGNNKCAGGTCTACCAAAAGTTTGAAACG |
| P977 | | |
| ERCAS12A- | 1188 | /5Phos/AAGGGTCGTTTCAAAGTTGAGCGCNNKGTCTACCAAAAGTTTGAAACGATG |
| P978 | | |
| ERCAS12A- | 1189 | /5Phos/GGTCGTTTCAAAGTTGAGCGCCAGNNKTACCAAAAGTTTGAAACGATGCTT |
| P979 | | |
| ERCAS12A- | 1190 | /5Phos/CGTTTCAAAGTTGAGCGCCAGGTCNNKCAAAAGTTTGAAACGATGCTTATT |
| P980 | | |
| ERCAS12A- | 1191 | /5Phos/TTCAAAGTTGAGCGCCAGGTCTACNNKAAGTTTGAAACGATGCTTATTAAT |
| P981 | | |
| ERCAS12A- | 1192 | /5Phos/AAAGTTGAGCGCCAGGTCTACCAANNKTTTGAAACGATGCTTATTAATAAA |
| P982 | | |
| ERCAS12A- | 1193 | /5Phos/GTTGAGCGCCAGGTCTACCAAAAGNNKGAAACGATGCTTATTAATAAATTA |
| P983 | | |
| ERCAS12A- | 1194 | /5Phos/GAGCGCCAGGTCTACCAAAAGTTTNNKACGATGCTTATTAATAAATTAAAC |
| P984 | | |
| ERCAS12A- | 1195 | /5Phos/CGCCAGGTCTACCAAAAGTTTGAANNKATGCTTATTAATAAATTAAACTAC |
| P985 | | |
| ERCAS12A- | 1196 | /5Phos/CAGGTCTACCAAAAGTTTGAAACGNNKCTTATTAATAAATTAAACTACTTG |
| P986 | | |
| ERCAS12A- | 1197 | /5Phos/GTCTACCAAAAGTTTGAAACGATGNNKATTAATAAATTAAACTACTTGGTT |
| P987 | | |
| ERCAS12A- | 1198 | /5Phos/TACCAAAAGTTTGAAACGATGCTTNNKAATAAATTAAACTACTTGGTTTTC |
| P988 | | |
| ERCAS12A- | 1199 | /5Phos/CAAAAGTTTGAAACGATGCTTATTNNKAAATTAAACTACTTGGTTTTCAAA |
| P989 | | |
| ERCAS12A- | 1200 | /5Phos/AAGTTTGAAACGATGCTTATTAATNNKTTAAACTACTTGGTTTTCAAAGAT |
| P990 | | |
| ERCAS12A- | 1201 | /5Phos/TTTGAAACGATGCTTATTAATAAANNKAACTACTTGGTTTTCAAAGATATC |
| P991 | | |
| ERCAS12A- | 1202 | /5Phos/GAAACGATGCTTATTAATAAATTANNKTACTTGGTTTTCAAAGATATCTCT |
| P992 | | |
| ERCAS12A- | 1203 | /5Phos/ACGATGCTTATTAATAAATTAAACNNKTTGGTTTTCAAAGATATCTCTATT |
| P993 | | |
| ERCAS12A- | 1204 | /5Phos/ATGCTTATTAATAAATTAAACTACNNKGTTTTCAAAGATATCTCTATTACC |
| P994 | | |
| ERCAS12A- | 1205 | /5Phos/CTTATTAATAAATTAAACTACTTGNNKTTCAAAGATATCTCTATTACCGAG |
| P995 | | |
| ERCAS12A- | 1206 | /5Phos/ATTAATAAATTAAACTACTTGGTTNNKAAAGATATCTCTATTACCGAGAAT |
| P996 | | |
| ERCAS12A- | 1207 | /5Phos/AATAAATTAAACTACTTGGTTTTCNNKGATATCTCTATTACCGAGAATGGA |
| P997 | | |
| ERCAS12A- | 1208 | /5Phos/AAATTAAACTACTTGGTTTTCAAANNKATCTCTATTACCGAGAATGGAGGT |
| P998 | | |
| ERCAS12A- | 1209 | /5Phos/TTAAACTACTTGGTTTTCAAAGATNNKTCTATTACCGAGAATGGAGGTTTA |
| P999 | | |
| ERCAS12A- | 1210 | /5Phos/AACTACTTGGTTTTCAAAGATATCNNKATTACCGAGAATGGAGGTTTACTG |
| P1000 | | |
| ERCAS12A- | 1211 | /5Phos/TACTTGGTTTTCAAAGATATCTCTNNKACCGAGAATGGAGGTTTACTGAAA |
| P1001 | | |
| ERCAS12A- | 1212 | /5Phos/TTGGTTTTCAAAGATATCTCTATTNNKGAGAATGGAGGTTTACTGAAAGGA |
| P1002 | | |
| ERCAS12A- | 1213 | /5Phos/GTTTTCAAAGATATCTCTATTACCNNKAATGGAGGTTTACTGAAAGGATAC |
| P1003 | | |
| ERCAS12A- | 1214 | /5Phos/TTCAAAGATATCTCTATTACCGAGNNKGGAGGTTTACTGAAAGGATACCAG |
| P1004 | | |
| ERCAS12A- | 1215 | /5Phos/AAAGATATCTCTATTACCGAGAATNNKGGTTTACTGAAAGGATACCAGTTA |
| P1005 | | |
| ERCAS12A- | 1216 | /5Phos/GATATCTCTATTACCGAGAATGGANNKTTACTGAAAGGATACCAGTTAACG |
| P1006 | | |
| ERCAS12A- | 1217 | /5Phos/ATCTCTATTACCGAGAATGGAGGTNNKCTGAAAGGATACCAGTTAACGTAT |
| P1007 | | |
| ERCAS12A- | 1218 | /5Phos/TCTATTACCGAGAATGGAGGTTTANNKAAAGGATACCAGTTAACGTATATC |
| P1008 | | |
| ERCAS12A- | 1219 | /5Phos/ATTACCGAGAATGGAGGTTTACTGNNKGGATACCAGTTAACGTATATCCCC |
| P1009 | | |
| ERCAS12A- | 1220 | /5Phos/ACCGAGAATGGAGGTTTACTGAAANNKTACCAGTTAACGTATATCCCCGAT |
| P1010 | | |
| ERCAS12A- | 1221 | /5Phos/GAGAATGGAGGTTTACTGAAAGGANNKCAGTTAACGTATATCCCCGATAAG |
| P1011 | | |
| ERCAS12A- | 1222 | /5Phos/AATGGAGGTTTACTGAAAGGATACNNKTTAACGTATATCCCCGATAAGCTT |
| P1012 | | |
| ERCAS12A- | 1223 | /5Phos/GGAGGTTTACTGAAAGGATACCAGNNKACGTATATCCCCGATAAGCTTAAA |
| P1013 | | |
| ERCAS12A- | 1224 | /5Phos/GGTTTACTGAAAGGATACCAGTTANNKTATATCCCCGATAAGCTTAAAAAC |
| P1014 | | |
| ERCAS12A- | 1225 | /5Phos/TTACTGAAAGGATACCAGTTAACGNNKATCCCCGATAAGCTTAAAAACGTG |
| P1015 | | |
| ERCAS12A- | 1226 | /5Phos/CTGAAAGGATACCAGTTAACGTATNNKCCCGATAAGCTTAAAAACGTGGGC |
| P1016 | | |
| ERCAS12A- | 1227 | /5Phos/AAAGGATACCAGTTAACGTATATCNNKGATAAGCTTAAAAACGTGGGCCAT |
| P1017 | | |
| ERCAS12A- | 1228 | /5Phos/GGATACCAGTTAACGTATATCCCCNNKAAGCTTAAAAACGTGGGCCATCAG |
| P1018 | | |
| ERCAS12A- | 1229 | /5Phos/TACCAGTTAACGTATATCCCCGATNNKCTTAAAAACGTGGGCCATCAGTGT |
| P1019 | | |
| ERCAS12A- | 1230 | /5Phos/CAGTTAACGTATATCCCCGATAAGNNKAAAAACGTGGGCCATCAGTGTGGC |
| P1020 | | |
| ERCAS12A- | 1231 | /5Phos/TTAACGTATATCCCCGATAAGCTTNNKAACGTGGGCCATCAGTGTGGCTGT |
| P1021 | | |
| ERCAS12A- | 1232 | /5Phos/ACGTATATCCCCGATAAGCTTAAANNKGTGGGCCATCAGTGTGGCTGTATT |
| P1022 | | |
| ERCAS12A- | 1233 | /5Phos/TATATCCCCGATAAGCTTAAAAACNNKGGCCATCAGTGTGGCTGTATTTTC |
| P1023 | | |
| ERCAS12A- | 1234 | /5Phos/ATCCCCGATAAGCTTAAAAACGTGNNKCATCAGTGTGGCTGTATTTTCTAT |
| P1024 | | |
| ERCAS12A- | 1235 | /5Phos/CCCGATAAGCTTAAAAACGTGGGCNNKCAGTGTGGCTGTATTTTCTATGTG |
| P1025 | | |
| ERCAS12A- | 1236 | /5Phos/GATAAGCTTAAAAACGTGGGCCATNNKTGTGGCTGTATTTTCTATGTGCCC |
| P1026 | | |
| ERCAS12A- | 1237 | /5Phos/AAGCTTAAAAACGTGGGCCATCAGNNKGGCTGTATTTTCTATGTGCCCGCG |
| P1027 | | |
| ERCAS12A- | 1238 | /5Phos/CTTAAAAACGTGGGCCATCAGTGTNNKTGTATTTTCTATGTGCCCGCGGCA |
| P1028 | | |
| ERCAS12A- | 1239 | /5Phos/AAAAACGTGGGCCATCAGTGTGGCNNKATTTTCTATGTGCCCGCGGCATAT |
| P1029 | | |
| ERCAS12A- | 1240 | /5Phos/AACGTGGGCCATCAGTGTGGCTGTNNKTTCTATGTGCCCGCGGCATATACA |
| P1030 | | |
| ERCAS12A- | 1241 | /5Phos/GTGGGCCATCAGTGTGGCTGTATTNNKTATGTGCCCGCGGCATATACATCA |
| P1031 | | |
| ERCAS12A- | 1242 | /5Phos/GGCCATCAGTGTGGCTGTATTTTCNNKGTGCCCGCGGCATATACATCAAAA |
| P1032 | | |
| ERCAS12A- | 1243 | /5Phos/CATCAGTGTGGCTGTATTTTCTATNNKCCCGCGGCATATACATCAAAAATT |
| P1033 | | |
| ERCAS12A- | 1244 | /5Phos/CAGTGTGGCTGTATTTTCTATGTGNNKGCGGCATATACATCAAAAATTGAC |
| P1034 | | |
| ERCAS12A- | 1245 | /5Phos/TGTGGCTGTATTTTCTATGTGCCCNNKGCATATACATCAAAAATTGACCCG |
| P1035 | | |
| ERCAS12A- | 1246 | /5Phos/GGCTGTATTTTCTATGTGCCCGCGNNKTATACATCAAAAATTGACCCGACG |
| P1036 | | |
| ERCAS12A- | 1247 | /5Phos/TGTATTTTCTATGTGCCCGCGGCANNKACATCAAAAATTGACCCGACGACG |
| P1037 | | |
| ERCAS12A- | 1248 | /5Phos/ATTTTCTATGTGCCCGCGGCATATNNKTCAAAAATTGACCCGACGACGGGT |
| P1038 | | |
| ERCAS12A- | 1249 | /5Phos/TTCTATGTGCCCGCGGCATATACANNKAAAATTGACCCGACGACGGGTTTC |
| P1039 | | |
| ERCAS12A- | 1250 | /5Phos/TATGTGCCCGCGGCATATACATCANNKATTGACCCGACGACGGGTTTCGTT |
| P1040 | | |
| ERCAS12A- | 1251 | /5Phos/GTGCCCGCGGCATATACATCAAAANNKGACCCGACGACGGGTTTCGTTAAT |
| P1041 | | |
| ERCAS12A- | 1252 | /5Phos/CCCGCGGCATATACATCAAAAATTNNKCCGACGACGGGTTTCGTTAATATC |
| P1042 | | |
| ERCAS12A- | 1253 | /5Phos/GCGGCATATACATCAAAAATTGACNNKACGACGGGTTTCGTTAATATCTTC |
| P1043 | | |
| ERCAS12A- | 1254 | /5Phos/GCATATACATCAAAAATTGACCCGNNKACGGGTTTCGTTAATATCTTCAAG |
| P1044 | | |
| ERCAS12A- | 1255 | /5Phos/TATACATCAAAAATTGACCCGACGNNKGGTTTCGTTAATATCTTCAAGTTC |
| P1045 | | |
| ERCAS12A- | 1256 | /5Phos/ACATCAAAAATTGACCCGACGACGNNKTTCGTTAATATCTTCAAGTTCAAA |
| P1046 | | |
| ERCAS12A- | 1257 | /5Phos/TCAAAAATTGACCCGACGACGGGTNNKGTTAATATCTTCAAGTTCAAAGAT |
| P1047 | | |
| ERCAS12A- | 1258 | /5Phos/AAAATTGACCCGACGACGGGTTTCNNKAATATCTTCAAGTTCAAAGATCTG |
| P1048 | | |
| ERCAS12A- | 1259 | /5Phos/ATTGACCCGACGACGGGTTTCGTTNNKATCTTCAAGTTCAAAGATCTGACA |
| P1049 | | |
| ERCAS12A- | 1260 | /5Phos/GACCCGACGACGGGTTTCGTTAATNNKTTCAAGTTCAAAGATCTGACAGTT |
| P1050 | | |
| ERCAS12A- | 1261 | /5Phos/CCGACGACGGGTTTCGTTAATATCNNKAAGTTCAAAGATCTGACAGTTGAT |
| P1051 | | |
| ERCAS12A- | 1262 | /5Phos/ACGACGGGTTTCGTTAATATCTTCNNKTTCAAAGATCTGACAGTTGATGCA |
| P1052 | | |
| ERCAS12A- | 1263 | /5Phos/ACGGGTTTCGTTAATATCTTCAAGNNKAAAGATCTGACAGTTGATGCAAAA |
| P1053 | | |
| ERCAS12A- | 1264 | /5Phos/GGTTTCGTTAATATCTTCAAGTTCNNKGATCTGACAGTTGATGCAAAACGC |
| P1054 | | |
| ERCAS12A- | 1265 | /5Phos/TTCGTTAATATCTTCAAGTTCAAANNKCTGACAGTTGATGCAAAACGCGAA |
| P1055 | | |
| ERCAS12A- | 1266 | /5Phos/GTTAATATCTTCAAGTTCAAAGATNNKACAGTTGATGCAAAACGCGAATTC |
| P1056 | | |
| ERCAS12A- | 1267 | /5Phos/AATATCTTCAAGTTCAAAGATCTGNNKGTTGATGCAAAACGCGAATTCATT |
| P1057 | | |
| ERCAS12A- | 1268 | /5Phos/ATCTTCAAGTTCAAAGATCTGACANNKGATGCAAAACGCGAATTCATTAAG |
| P1058 | | |
| ERCAS12A- | 1269 | /5Phos/TTCAAGTTCAAAGATCTGACAGTTNNKGCAAAACGCGAATTCATTAAGAAG |
| P1059 | | |
| ERCAS12A- | 1270 | /5Phos/AAGTTCAAAGATCTGACAGTTGATNNKAAACGCGAATTCATTAAGAAGTTC |
| P1060 | | |
| ERCAS12A- | 1271 | /5Phos/TTCAAAGATCTGACAGTTGATGCANNKCGCGAATTCATTAAGAAGTTCGAT |
| P1061 | | |
| ERCAS12A- | 1272 | /5Phos/AAAGATCTGACAGTTGATGCAAAANNKGAATTCATTAAGAAGTTCGATAGT |
| P1062 | | |
| ERCAS12A- | 1273 | /5Phos/GATCTGACAGTTGATGCAAAACGCNNKTTCATTAAGAAGTTCGATAGTATT |
| P1063 | | |
| ERCAS12A- | 1274 | /5Phos/CTGACAGTTGATGCAAAACGCGAANNKATTAAGAAGTTCGATAGTATTCGC |
| P1064 | | |
| ERCAS12A- | 1275 | /5Phos/ACAGTTGATGCAAAACGCGAATTCNNKAAGAAGTTCGATAGTATTCGCTAT |
| P1065 | | |
| ERCAS12A- | 1276 | /5Phos/GTTGATGCAAAACGCGAATTCATTNNKAAGTTCGATAGTATTCGCTATGAC |
| P1066 | | |
| ERCAS12A- | 1277 | /5Phos/GATGCAAAACGCGAATTCATTAAGNNKTTCGATAGTATTCGCTATGACTCC |
| P1067 | | |
| ERCAS12A- | 1278 | /5Phos/GCAAAACGCGAATTCATTAAGAAGNNKGATAGTATTCGCTATGACTCCGAG |
| P1068 | | |
| ERCAS12A- | 1279 | /5Phos/AAACGCGAATTCATTAAGAAGTTCNNKAGTATTCGCTATGACTCCGAGAAA |
| P1069 | | |
| ERCAS12A- | 1280 | /5Phos/CGCGAATTCATTAAGAAGTTCGATNNKATTCGCTATGACTCCGAGAAAAAT |
| P1070 | | |
| ERCAS12A- | 1281 | /5Phos/GAATTCATTAAGAAGTTCGATAGTNNKCGCTATGACTCCGAGAAAAATTTG |
| P1071 | | |
| ERCAS12A- | 1282 | /5Phos/TTCATTAAGAAGTTCGATAGTATTNNKTATGACTCCGAGAAAAATTTGTTT |
| P1072 | | |
| ERCAS12A- | 1283 | /5Phos/ATTAAGAAGTTCGATAGTATTCGCNNKGACTCCGAGAAAAATTTGTTTTGC |
| P1073 | | |
| ERCAS12A- | 1284 | /5Phos/AAGAAGTTCGATAGTATTCGCTATNNKTCCGAGAAAAATTTGTTTTGCTTC |
| P1074 | | |
| ERCAS12A- | 1285 | /5Phos/AAGTTCGATAGTATTCGCTATGACNNKGAGAAAAATTTGTTTTGCTTCACT |
| P1075 | | |
| ERCAS12A- | 1286 | /5Phos/TTCGATAGTATTCGCTATGACTCCNNKAAAAATTTGTTTTGCTTCACTTTT |
| P1076 | | |
| ERCAS12A- | 1287 | /5Phos/GATAGTATTCGCTATGACTCCGAGNNKAATTTGTTTTGCTTCACTTTTGAC |
| P1077 | | |
| ERCAS12A- | 1288 | /5Phos/AGTATTCGCTATGACTCCGAGAAANNKTTGTTTTGCTTCACTTTTGACTAC |
| P1078 | | |
| ERCAS12A- | 1289 | /5Phos/ATTCGCTATGACTCCGAGAAAAATNNKTTTTGCTTCACTTTTGACTACAAT |
| P1079 | | |
| ERCAS12A- | 1290 | /5Phos/CGCTATGACTCCGAGAAAAATTTGNNKTGCTTCACTTTTGACTACAATAAC |
| P1080 | | |
| ERCAS12A- | 1291 | /5Phos/TATGACTCCGAGAAAAATTTGTTTNNKTTCACTTTTGACTACAATAACTTT |
| P1081 | | |
| ERCAS12A- | 1292 | /5Phos/GACTCCGAGAAAAATTTGTTTTGCNNKACTTTTGACTACAATAACTTTATT |
| P1082 | | |
| ERCAS12A- | 1293 | /5Phos/TCCGAGAAAAATTTGTTTTGCTTCNNKTTTGACTACAATAACTTTATTACT |
| P1083 | | |
| ERCAS12A- | 1294 | /5Phos/GAGAAAAATTTGTTTTGCTTCACTNNKGACTACAATAACTTTATTACTCAG |
| P1084 | | |
| ERCAS12A- | 1295 | /5Phos/AAAAATTTGTTTTGCTTCACTTTTNNKTACAATAACTTTATTACTCAGAAT |
| P1085 | | |
| ERCAS12A- | 1296 | /5Phos/AATTTGTTTTGCTTCACTTTTGACNNKAATAACTTTATTACTCAGAATACA |
| P1086 | | |
| ERCAS12A- | 1297 | /5Phos/TTGTTTTGCTTCACTTTTGACTACNNKAACTTTATTACTCAGAATACAGTC |
| P1087 | | |
| ERCAS12A- | 1298 | /5Phos/TTTTGCTTCACTTTTGACTACAATNNKTTTATTACTCAGAATACAGTCATG |
| P1088 | | |
| ERCAS12A- | 1299 | /5Phos/TGCTTCACTTTTGACTACAATAACNNKATTACTCAGAATACAGTCATGAGT |
| P1089 | | |
| ERCAS12A- | 1300 | /5Phos/TTCACTTTTGACTACAATAACTTTNNKACTCAGAATACAGTCATGAGTAAA |
| P1090 | | |
| ERCAS12A- | 1301 | /5Phos/ACTTTTGACTACAATAACTTTATTNNKCAGAATACAGTCATGAGTAAAAGC |
| P1091 | | |
| ERCAS12A- | 1302 | /5Phos/TTTGACTACAATAACTTTATTACTNNKAATACAGTCATGAGTAAAAGCTCC |
| P1092 | | |
| ERCAS12A- | 1303 | /5Phos/GACTACAATAACTTTATTACTCAGNNKACAGTCATGAGTAAAAGCTCCTGG |
| P1093 | | |
| ERCAS12A- | 1304 | /5Phos/TACAATAACTTTATTACTCAGAATNNKGTCATGAGTAAAAGCTCCTGGTCG |
| P1094 | | |
| ERCAS12A- | 1305 | /5Phos/AATAACTTTATTACTCAGAATACANNKATGAGTAAAAGCTCCTGGTCGGTT |
| P1095 | | |
| ERCAS12A- | 1306 | /5Phos/AACTTTATTACTCAGAATACAGTCNNKAGTAAAAGCTCCTGGTCGGTTTAC |
| P1096 | | |
| ERCAS12A- | 1307 | /5Phos/TTTATTACTCAGAATACAGTCATGNNKAAAAGCTCCTGGTCGGTTTACACG |
| P1097 | | |
| ERCAS12A- | 1308 | /5Phos/ATTACTCAGAATACAGTCATGAGTNNKAGCTCCTGGTCGGTTTACACGTAC |
| P1098 | | |
| ERCAS12A- | 1309 | /5Phos/ACTCAGAATACAGTCATGAGTAAANNKTCCTGGTCGGTTTACACGTACGGG |
| P1099 | | |
| ERCAS12A- | 1310 | /5Phos/CAGAATACAGTCATGAGTAAAAGCNNKTGGTCGGTTTACACGTACGGGGTA |
| P1100 | | |
| ERCAS12A- | 1311 | /5Phos/AATACAGTCATGAGTAAAAGCTCCNNKTCGGTTTACACGTACGGGGTACGC |
| P1101 | | |
| ERCAS12A- | 1312 | /5Phos/ACAGTCATGAGTAAAAGCTCCTGGNNKGTTTACACGTACGGGGTACGCATC |
| P1102 | | |
| ERCAS12A- | 1313 | /5Phos/GTCATGAGTAAAAGCTCCTGGTCGNNKTACACGTACGGGGTACGCATCAAA |
| P1103 | | |
| ERCAS12A- | 1314 | /5Phos/ATGAGTAAAAGCTCCTGGTCGGTTNNKACGTACGGGGTACGCATCAAACGT |
| P1104 | | |
| ERCAS12A- | 1315 | /5Phos/AGTAAAAGCTCCTGGTCGGTTTACNNKTACGGGGTACGCATCAAACGTCGT |
| P1105 | | |
| ERCAS12A- | 1316 | /5Phos/AAAAGCTCCTGGTCGGTTTACACGNNKGGGGTACGCATCAAACGTCGTTTT |
| P1106 | | |
| ERCAS12A- | 1317 | /5Phos/AGCTCCTGGTCGGTTTACACGTACNNKGTACGCATCAAACGTCGTTTTGTG |
| P1107 | | |
| ERCAS12A- | 1318 | /5Phos/TCCTGGTCGGTTTACACGTACGGGNNKCGCATCAAACGTCGTTTTGTGAAT |
| P1108 | | |
| ERCAS12A- | 1319 | /5Phos/TGGTCGGTTTACACGTACGGGGTANNKATCAAACGTCGTTTTGTGAATGGT |
| P1109 | | |
| ERCAS12A- | 1320 | /5Phos/TCGGTTTACACGTACGGGGTACGCNNKAAACGTCGTTTTGTGAATGGTCGT |
| P1110 | | |
| ERCAS12A- | 1321 | /5Phos/GTTTACACGTACGGGGTACGCATCNNKCGTCGTTTTGTGAATGGTCGTTTT |
| P1111 | | |
| ERCAS12A- | 1322 | /5Phos/TACACGTACGGGGTACGCATCAAANNKCGTTTTGTGAATGGTCGTTTTAGT |
| P1112 | | |
| ERCAS12A- | 1323 | /5Phos/ACGTACGGGGTACGCATCAAACGTNNKTTTGTGAATGGTCGTTTTAGTAAT |
| P1113 | | |
| ERCAS12A- | 1324 | /5Phos/TACGGGGTACGCATCAAACGTCGTNNKGTGAATGGTCGTTTTAGTAATGAG |
| P1114 | | |
| ERCAS12A- | 1325 | /5Phos/GGGGTACGCATCAAACGTCGTTTTNNKAATGGTCGTTTTAGTAATGAGTCG |
| P1115 | | |
| ERCAS12A- | 1326 | /5Phos/GTACGCATCAAACGTCGTTTTGTGNNKGGTCGTTTTAGTAATGAGTCGGAT |
| P1116 | | |
| ERCAS12A- | 1327 | /5Phos/CGCATCAAACGTCGTTTTGTGAATNNKCGTTTTAGTAATGAGTCGGATACA |
| P1117 | | |
| ERCAS12A- | 1328 | /5Phos/ATCAAACGTCGTTTTGTGAATGGTNNKTTTAGTAATGAGTCGGATACAATT |
| P1118 | | |
| ERCAS12A- | 1329 | /5Phos/AAACGTCGTTTTGTGAATGGTCGTNNKAGTAATGAGTCGGATACAATTGAT |
| P1119 | | |
| ERCAS12A- | 1330 | /5Phos/CGTCGTTTTGTGAATGGTCGTTTTNNKAATGAGTCGGATACAATTGATATT |
| P1120 | | |
| ERCAS12A- | 1331 | /5Phos/CGTTTTGTGAATGGTCGTTTTAGTNNKGAGTCGGATACAATTGATATTACC |
| P1121 | | |
| ERCAS12A- | 1332 | /5Phos/TTTGTGAATGGTCGTTTTAGTAATNNKTCGGATACAATTGATATTACCAAG |
| P1122 | | |
| ERCAS12A- | 1333 | /5Phos/GTGAATGGTCGTTTTAGTAATGAGNNKGATACAATTGATATTACCAAGGAT |
| P1123 | | |
| ERCAS12A- | 1334 | /5Phos/AATGGTCGTTTTAGTAATGAGTCGNNKACAATTGATATTACCAAGGATATG |
| P1124 | | |
| ERCAS12A- | 1335 | /5Phos/GGTCGTTTTAGTAATGAGTCGGATNNKATTGATATTACCAAGGATATGGAG |
| P1125 | | |
| ERCAS12A- | 1336 | /5Phos/CGTTTTAGTAATGAGTCGGATACANNKGATATTACCAAGGATATGGAGAAG |
| P1126 | | |
| ERCAS12A- | 1337 | /5Phos/TTTAGTAATGAGTCGGATACAATTNNKATTACCAAGGATATGGAGAAGACT |
| P1127 | | |
| ERCAS12A- | 1338 | /5Phos/AGTAATGAGTCGGATACAATTGATNNKACCAAGGATATGGAGAAGACTCTG |
| P1128 | | |
| ERCAS12A- | 1339 | /5Phos/AATGAGTCGGATACAATTGATATTNNKAAGGATATGGAGAAGACTCTGGAA |
| P1129 | | |
| ERCAS12A- | 1340 | /5Phos/GAGTCGGATACAATTGATATTACCNNKGATATGGAGAAGACTCTGGAAATG |
| P1130 | | |
| ERCAS12A- | 1341 | /5Phos/TCGGATACAATTGATATTACCAAGNNKATGGAGAAGACTCTGGAAATGACA |
| P1131 | | |
| ERCAS12A- | 1342 | /5Phos/GATACAATTGATATTACCAAGGATNNKGAGAAGACTCTGGAAATGACAGAC |
| P1132 | | |
| ERCAS12A- | 1343 | /5Phos/ACAATTGATATTACCAAGGATATGNNKAAGACTCTGGAAATGACAGACATC |
| P1133 | | |
| ERCAS12A- | 1344 | /5Phos/ATTGATATTACCAAGGATATGGAGNNKACTCTGGAAATGACAGACATCAAT |
| P1134 | | |
| ERCAS12A- | 1345 | /5Phos/GATATTACCAAGGATATGGAGAAGNNKCTGGAAATGACAGACATCAATTGG |
| P1135 | | |
| ERCAS12A- | 1346 | /5Phos/ATTACCAAGGATATGGAGAAGACTNNKGAAATGACAGACATCAATTGGCGT |
| P1136 | | |
| ERCAS12A- | 1347 | /5Phos/ACCAAGGATATGGAGAAGACTCTGNNKATGACAGACATCAATTGGCGTGAT |
| P1137 | | |
| ERCAS12A- | 1348 | /5Phos/AAGGATATGGAGAAGACTCTGGAANNKACAGACATCAATTGGCGTGATGGA |
| P1138 | | |
| ERCAS12A- | 1349 | /5Phos/GATATGGAGAAGACTCTGGAAATGNNKGACATCAATTGGCGTGATGGACAT |
| P1139 | | |
| ERCAS12A- | 1350 | /5Phos/ATGGAGAAGACTCTGGAAATGACANNKATCAATTGGCGTGATGGACATGAT |
| P1140 | | |
| ERCAS12A- | 1351 | /5Phos/GAGAAGACTCTGGAAATGACAGACNNKAATTGGCGTGATGGACATGATTTG |
| P1141 | | |
| ERCAS12A- | 1352 | /5Phos/AAGACTCTGGAAATGACAGACATCNNKTGGCGTGATGGACATGATTTGCGT |
| P1142 | | |
| ERCAS12A- | 1353 | /5Phos/ACTCTGGAAATGACAGACATCAATNNKCGTGATGGACATGATTTGCGTCAG |
| P1143 | | |
| ERCAS12A- | 1354 | /5Phos/CTGGAAATGACAGACATCAATTGGNNKGATGGACATGATTTGCGTCAGGAC |
| P1144 | | |
| ERCAS12A- | 1355 | /5Phos/GAAATGACAGACATCAATTGGCGTNNKGGACATGATTTGCGTCAGGACATT |
| P1145 | | |
| ERCAS12A- | 1356 | /5Phos/ATGACAGACATCAATTGGCGTGATNNKCATGATTTGCGTCAGGACATTATC |
| P1146 | | |
| ERCAS12A- | 1357 | /5Phos/ACAGACATCAATTGGCGTGATGGANNKGATTTGCGTCAGGACATTATCGAC |
| P1147 | | |
| ERCAS12A- | 1358 | /5Phos/GACATCAATTGGCGTGATGGACATNNKTTGCGTCAGGACATTATCGACTAT |
| P1148 | | |
| ERCAS12A- | 1359 | /5Phos/ATCAATTGGCGTGATGGACATGATNNKCGTCAGGACATTATCGACTATGAG |
| P1149 | | |
| ERCAS12A- | 1360 | /5Phos/AATTGGCGTGATGGACATGATTTGNNKCAGGACATTATCGACTATGAGATT |
| P1150 | | |
| ERCAS12A- | 1361 | /5Phos/TGGCGTGATGGACATGATTTGCGTNNKGACATTATCGACTATGAGATTGTG |
| P1151 | | |
| ERCAS12A- | 1362 | /5Phos/CGTGATGGACATGATTTGCGTCAGNNKATTATCGACTATGAGATTGTGCAG |
| P1152 | | |
| ERCAS12A- | 1363 | /5Phos/GATGGACATGATTTGCGTCAGGACNNKATCGACTATGAGATTGTGCAGCAT |
| P1153 | | |
| ERCAS12A- | 1364 | /5Phos/GGACATGATTTGCGTCAGGACATTNNKGACTATGAGATTGTGCAGCATATT |
| P1154 | | |
| ERCAS12A- | 1365 | /5Phos/CATGATTTGCGTCAGGACATTATCNNKTATGAGATTGTGCAGCATATTTTC |
| P1155 | | |
| ERCAS12A- | 1366 | /5Phos/GATTTGCGTCAGGACATTATCGACNNKGAGATTGTGCAGCATATTTTCGAA |
| P1156 | | |
| ERCAS12A- | 1367 | /5Phos/TTGCGTCAGGACATTATCGACTATNNKATTGTGCAGCATATTTTCGAAATT |
| P1157 | | |
| ERCAS12A- | 1368 | /5Phos/CGTCAGGACATTATCGACTATGAGNNKGTGCAGCATATTTTCGAAATTTTT |
| P1158 | | |
| ERCAS12A- | 1369 | /5Phos/CAGGACATTATCGACTATGAGATTNNKCAGCATATTTTCGAAATTTTTCGC |
| P1159 | | |
| ERCAS12A- | 1370 | /5Phos/GACATTATCGACTATGAGATTGTGNNKCATATTTTCGAAATTTTTCGCCTG |
| P1160 | | |
| ERCAS12A- | 1371 | /5Phos/ATTATCGACTATGAGATTGTGCAGNNKATTTTCGAAATTTTTCGCCTGACG |
| P1161 | | |
| ERCAS12A- | 1372 | /5Phos/ATCGACTATGAGATTGTGCAGCATNNKTTCGAAATTTTTCGCCTGACGGTG |
| P1162 | | |
| ERCAS12A- | 1373 | /5Phos/GACTATGAGATTGTGCAGCATATTNNKGAAATTTTTCGCCTGACGGTGCAA |
| P1163 | | |
| ERCAS12A- | 1374 | /5Phos/TATGAGATTGTGCAGCATATTTTCNNKATTTTTCGCCTGACGGTGCAAATG |
| P1164 | | |
| ERCAS12A- | 1375 | /5Phos/GAGATTGTGCAGCATATTTTCGAANNKTTTCGCCTGACGGTGCAAATGCGT |
| P1165 | | |
| ERCAS12A- | 1376 | /5Phos/ATTGTGCAGCATATTTTCGAAATTNNKCGCCTGACGGTGCAAATGCGTAAT |
| P1166 | | |
| ERCAS12A- | 1377 | /5Phos/GTGCAGCATATTTTCGAAATTTTTNNKCTGACGGTGCAAATGCGTAATTCC |
| P1167 | | |
| ERCAS12A- | 1378 | /5Phos/CAGCATATTTTCGAAATTTTTCGCNNKACGGTGCAAATGCGTAATTCCTTA |
| P1168 | | |
| ERCAS12A- | 1379 | /5Phos/CATATTTTCGAAATTTTTCGCCTGNNKGTGCAAATGCGTAATTCCTTAAGC |
| P1169 | | |
| ERCAS12A- | 1380 | /5Phos/ATTTTCGAAATTTTTCGCCTGACGNNKCAAATGCGTAATTCCTTAAGCGAA |
| P1170 | | |
| ERCAS12A- | 1381 | /5Phos/TTCGAAATTTTTCGCCTGACGGTGNNKATGCGTAATTCCTTAAGCGAACTG |
| P1171 | | |
| ERCAS12A- | 1382 | /5Phos/GAAATTTTTCGCCTGACGGTGCAANNKCGTAATTCCTTAAGCGAACTGGAA |
| P1172 | | |
| ERCAS12A- | 1383 | /5Phos/ATTTTTCGCCTGACGGTGCAAATGNNKAATTCCTTAAGCGAACTGGAAGAT |
| P1173 | | |
| ERCAS12A- | 1384 | /5Phos/TTTCGCCTGACGGTGCAAATGCGTNNKTCCTTAAGCGAACTGGAAGATCGC |
| P1174 | | |
| ERCAS12A- | 1385 | /5Phos/CGCCTGACGGTGCAAATGCGTAATNNKTTAAGCGAACTGGAAGATCGCGAC |
| P1175 | | |
| ERCAS12A- | 1386 | /5Phos/CTGACGGTGCAAATGCGTAATTCCNNKAGCGAACTGGAAGATCGCGACTAT |
| P1176 | | |
| ERCAS12A- | 1387 | /5Phos/ACGGTGCAAATGCGTAATTCCTTANNKGAACTGGAAGATCGCGACTATGAC |
| P1177 | | |
| ERCAS12A- | 1388 | /5Phos/GTGCAAATGCGTAATTCCTTAAGCNNKCTGGAAGATCGCGACTATGACCGC |
| P1178 | | |
| ERCAS12A- | 1389 | /5Phos/CAAATGCGTAATTCCTTAAGCGAANNKGAAGATCGCGACTATGACCGCTTA |
| P1179 | | |
| ERCAS12A- | 1390 | /5Phos/ATGCGTAATTCCTTAAGCGAACTGNNKGATCGCGACTATGACCGCTTAATT |
| P1180 | | |
| ERCAS12A- | 1391 | /5Phos/CGTAATTCCTTAAGCGAACTGGAANNKCGCGACTATGACCGCTTAATTTCT |
| P1181 | | |
| ERCAS12A- | 1392 | /5Phos/AATTCCTTAAGCGAACTGGAAGATNNKGACTATGACCGCTTAATTTCTCCT |
| P1182 | | |
| ERCAS12A- | 1393 | /5Phos/TCCTTAAGCGAACTGGAAGATCGCNNKTATGACCGCTTAATTTCTCCTGTG |
| P1183 | | |
| ERCAS12A- | 1394 | /5Phos/TTAAGCGAACTGGAAGATCGCGACNNKGACCGCTTAATTTCTCCTGTGCTG |
| P1184 | | |
| ERCAS12A- | 1395 | /5Phos/AGCGAACTGGAAGATCGCGACTATNNKCGCTTAATTTCTCCTGTGCTGAAT |
| P1185 | | |
| ERCAS12A- | 1396 | /5Phos/GAACTGGAAGATCGCGACTATGACNNKTTAATTTCTCCTGTGCTGAATGAG |
| P1186 | | |
| ERCAS12A- | 1397 | /5Phos/CTGGAAGATCGCGACTATGACCGCNNKATTTCTCCTGTGCTGAATGAGAAC |
| P1187 | | |
| ERCAS12A- | 1398 | /5Phos/GAAGATCGCGACTATGACCGCTTANNKTCTCCTGTGCTGAATGAGAACAAC |
| P1188 | | |
| ERCAS12A- | 1399 | /5Phos/GATCGCGACTATGACCGCTTAATTNNKCCTGTGCTGAATGAGAACAACATC |
| P1189 | | |
| ERCAS12A- | 1400 | /5Phos/CGCGACTATGACCGCTTAATTTCTNNKGTGCTGAATGAGAACAACATCTTC |
| P1190 | | |
| ERCAS12A- | 1401 | /5Phos/GACTATGACCGCTTAATTTCTCCTNNKCTGAATGAGAACAACATCTTCTAT |
| P1191 | | |
| ERCAS12A- | 1402 | /5Phos/TATGACCGCTTAATTTCTCCTGTGNNKAATGAGAACAACATCTTCTATGAC |
| P1192 | | |
| ERCAS12A- | 1403 | /5Phos/GACCGCTTAATTTCTCCTGTGCTGNNKGAGAACAACATCTTCTATGACTCA |
| P1193 | | |
| ERCAS12A- | 1404 | /5Phos/CGCTTAATTTCTCCTGTGCTGAATNNKAACAACATCTTCTATGACTCAGCG |
| P1194 | | |
| ERCAS12A- | 1405 | /5Phos/TTAATTTCTCCTGTGCTGAATGAGNNKAACATCTTCTATGACTCAGCGAAA |
| P1195 | | |
| ERCAS12A- | 1406 | /5Phos/ATTTCTCCTGTGCTGAATGAGAACNNKATCTTCTATGACTCAGCGAAAGCC |
| P1196 | | |
| ERCAS12A- | 1407 | /5Phos/TCTCCTGTGCTGAATGAGAACAACNNKTTCTATGACTCAGCGAAAGCCGGG |
| P1197 | | |
| ERCAS12A- | 1408 | /5Phos/CCTGTGCTGAATGAGAACAACATCNNKTATGACTCAGCGAAAGCCGGGGAC |
| P1198 | | |
| ERCAS12A- | 1409 | /5Phos/GTGCTGAATGAGAACAACATCTTCNNKGACTCAGCGAAAGCCGGGGACGCT |
| P1199 | | |
| ERCAS12A- | 1410 | /5Phos/CTGAATGAGAACAACATCTTCTATNNKTCAGCGAAAGCCGGGGACGCTTTG |
| P1200 | | |
| ERCAS12A- | 1411 | /5Phos/AATGAGAACAACATCTTCTATGACNNKGCGAAAGCCGGGGACGCTTTGCCA |
| P1201 | | |
| ERCAS12A- | 1412 | /5Phos/GAGAACAACATCTTCTATGACTCANNKAAAGCCGGGGACGCTTTGCCAAAG |
| P1202 | | |
| ERCAS12A- | 1413 | /5Phos/AACAACATCTTCTATGACTCAGCGNNKGCCGGGGACGCTTTGCCAAAGGAT |
| P1203 | | |
| ERCAS12A- | 1414 | /5Phos/AACATCTTCTATGACTCAGCGAAANNKGGGGACGCTTTGCCAAAGGATGCA |
| P1204 | | |
| ERCAS12A- | 1415 | /5Phos/ATCTTCTATGACTCAGCGAAAGCCNNKGACGCTTTGCCAAAGGATGCAGAT |
| P1205 | | |
| ERCAS12A- | 1416 | /5Phos/TTCTATGACTCAGCGAAAGCCGGGNNKGCTTTGCCAAAGGATGCAGATGCT |
| P1206 | | |
| ERCAS12A- | 1417 | /5Phos/TATGACTCAGCGAAAGCCGGGGACNNKTTGCCAAAGGATGCAGATGCTAAC |
| P1207 | | |
| ERCAS12A- | 1418 | /5Phos/GACTCAGCGAAAGCCGGGGACGCTNNKCCAAAGGATGCAGATGCTAACGGT |
| P1208 | | |
| ERCAS12A- | 1419 | /5Phos/TCAGCGAAAGCCGGGGACGCTTTGNNKAAGGATGCAGATGCTAACGGTGCA |
| P1209 | | |
| ERCAS12A- | 1420 | /5Phos/GCGAAAGCCGGGGACGCTTTGCCANNKGATGCAGATGCTAACGGTGCATAT |
| P1210 | | |
| ERCAS12A- | 1421 | /5Phos/AAAGCCGGGGACGCTTTGCCAAAGNNKGCAGATGCTAACGGTGCATATTGT |
| P1211 | | |
| ERCAS12A- | 1422 | /5Phos/GCCGGGGACGCTTTGCCAAAGGATNNKGATGCTAACGGTGCATATTGTATT |
| P1212 | | |
| ERCAS12A- | 1423 | /5Phos/GGGGACGCTTTGCCAAAGGATGCANNKGCTAACGGTGCATATTGTATTGCT |
| P1213 | | |
| ERCAS12A- | 1424 | /5Phos/GACGCTTTGCCAAAGGATGCAGATNNKAACGGTGCATATTGTATTGCTCTG |
| P1214 | | |
| ERCAS12A- | 1425 | /5Phos/GCTTTGCCAAAGGATGCAGATGCTNNKGGTGCATATTGTATTGCTCTGAAA |
| P1215 | | |
| ERCAS12A- | 1426 | /5Phos/TTGCCAAAGGATGCAGATGCTAACNNKGCATATTGTATTGCTCTGAAAGGC |
| P1216 | | |
| ERCAS12A- | 1427 | /5Phos/CCAAAGGATGCAGATGCTAACGGTNNKTATTGTATTGCTCTGAAAGGCCTT |
| P1217 | | |
| ERCAS12A- | 1428 | /5Phos/AAGGATGCAGATGCTAACGGTGCANNKTGTATTGCTCTGAAAGGCCTTTAC |
| P1218 | | |
| ERCAS12A- | 1429 | /5Phos/GATGCAGATGCTAACGGTGCATATNNKATTGCTCTGAAAGGCCTTTACGAG |
| P1219 | | |
| ERCAS12A- | 1430 | /5Phos/GCAGATGCTAACGGTGCATATTGTNNKGCTCTGAAAGGCCTTTACGAGATC |
| P1220 | | |
| ERCAS12A- | 1431 | /5Phos/GATGCTAACGGTGCATATTGTATTNNKCTGAAAGGCCTTTACGAGATCAAA |
| P1221 | | |
| ERCAS12A- | 1432 | /5Phos/GCTAACGGTGCATATTGTATTGCTNNKAAAGGCCTTTACGAGATCAAACAA |
| P1222 | | |
| ERCAS12A- | 1433 | /5Phos/AACGGTGCATATTGTATTGCTCTGNNKGGCCTTTACGAGATCAAACAAATT |
| P1223 | | |
| ERCAS12A- | 1434 | /5Phos/GGTGCATATTGTATTGCTCTGAAANNKCTTTACGAGATCAAACAAATTACT |
| P1224 | | |
| ERCAS12A- | 1435 | /5Phos/GCATATTGTATTGCTCTGAAAGGCNNKTACGAGATCAAACAAATTACTGAA |
| P1225 | | |
| ERCAS12A- | 1436 | /5Phos/TATTGTATTGCTCTGAAAGGCCTTNNKGAGATCAAACAAATTACTGAAAAC |
| P1226 | | |
| ERCAS12A- | 1437 | /5Phos/TGTATTGCTCTGAAAGGCCTTTACNNKATCAAACAAATTACTGAAAACTGG |
| P1227 | | |
| ERCAS12A- | 1438 | /5Phos/ATTGCTCTGAAAGGCCTTTACGAGNNKAAACAAATTACTGAAAACTGGAAG |
| P1228 | | |
| ERCAS12A- | 1439 | /5Phos/GCTCTGAAAGGCCTTTACGAGATCNNKCAAATTACTGAAAACTGGAAGGAA |
| P1229 | | |
| ERCAS12A- | 1440 | /5Phos/CTGAAAGGCCTTTACGAGATCAAANNKATTACTGAAAACTGGAAGGAAGAT |
| P1230 | | |
| ERCAS12A- | 1441 | /5Phos/AAAGGCCTTTACGAGATCAAACAANNKACTGAAAACTGGAAGGAAGATGGA |
| P1231 | | |
| ERCAS12A- | 1442 | /5Phos/GGCCTTTACGAGATCAAACAAATTNNKGAAAACTGGAAGGAAGATGGAAAG |
| P1232 | | |
| ERCAS12A- | 1443 | /5Phos/CTTTACGAGATCAAACAAATTACTNNKAACTGGAAGGAAGATGGAAAGTTT |
| P1233 | | |
| ERCAS12A- | 1444 | /5Phos/TACGAGATCAAACAAATTACTGAANNKTGGAAGGAAGATGGAAAGTTTTCC |
| P1234 | | |
| ERCAS12A- | 1445 | /5Phos/GAGATCAAACAAATTACTGAAAACNNKAAGGAAGATGGAAAGTTTTCCCGT |
| P1235 | | |
| ERCAS12A- | 1446 | /5Phos/ATCAAACAAATTACTGAAAACTGGNNKGAAGATGGAAAGTTTTCCCGTGAT |
| P1236 | | |
| ERCAS12A- | 1447 | /5Phos/AAACAAATTACTGAAAACTGGAAGNNKGATGGAAAGTTTTCCCGTGATAAA |
| P1237 | | |
| ERCAS12A- | 1448 | /5Phos/CAAATTACTGAAAACTGGAAGGAANNKGGAAAGTTTTCCCGTGATAAATTG |
| P1238 | | |
| ERCAS12A- | 1449 | /5Phos/ATTACTGAAAACTGGAAGGAAGATNNKAAGTTTTCCCGTGATAAATTGAAG |
| P1239 | | |
| ERCAS12A- | 1450 | /5Phos/ACTGAAAACTGGAAGGAAGATGGANNKTTTTCCCGTGATAAATTGAAGATC |
| P1240 | | |
| ERCAS12A- | 1451 | /5Phos/GAAAACTGGAAGGAAGATGGAAAGNNKTCCCGTGATAAATTGAAGATCTCT |
| P1241 | | |
| ERCAS12A- | 1452 | /5Phos/AACTGGAAGGAAGATGGAAAGTTTNNKCGTGATAAATTGAAGATCTCTAAT |
| P1242 | | |
| ERCAS12A- | 1453 | /5Phos/TGGAAGGAAGATGGAAAGTTTTCCNNKGATAAATTGAAGATCTCTAATAAG |
| P1243 | | |
| ERCAS12A- | 1454 | /5Phos/AAGGAAGATGGAAAGTTTTCCCGTNNKAAATTGAAGATCTCTAATAAGGAC |
| P1244 | | |
| ERCAS12A- | 1455 | /5Phos/GAAGATGGAAAGTTTTCCCGTGATNNKTTGAAGATCTCTAATAAGGACTGG |
| P1245 | | |
| ERCAS12A- | 1456 | /5Phos/GATGGAAAGTTTTCCCGTGATAAANNKAAGATCTCTAATAAGGACTGGTTT |
| P1246 | | |
| ERCAS12A- | 1457 | /5Phos/GGAAAGTTTTCCCGTGATAAATTGNNKATCTCTAATAAGGACTGGTTTGAC |
| P1247 | | |
| ERCAS12A- | 1458 | /5Phos/AAGTTTTCCCGTGATAAATTGAAGNNKTCTAATAAGGACTGGTTTGACTTT |
| P1248 | | |
| ERCAS12A- | 1459 | /5Phos/TTTTCCCGTGATAAATTGAAGATCNNKAATAAGGACTGGTTTGACTTTATC |
| P1249 | | |
| ERCAS12A- | 1460 | /5Phos/TCCCGTGATAAATTGAAGATCTCTNNKAAGGACTGGTTTGACTTTATCCAA |
| P1250 | | |
| ERCAS12A- | 1461 | /5Phos/CGTGATAAATTGAAGATCTCTAATNNKGACTGGTTTGACTTTATCCAAAAT |
| P1251 | | |
| ERCAS12A- | 1462 | /5Phos/GATAAATTGAAGATCTCTAATAAGNNKTGGTTTGACTTTATCCAAAATAAA |
| P1252 | | |
| ERCAS12A- | 1463 | /5Phos/AAATTGAAGATCTCTAATAAGGACNNKTTTGACTTTATCCAAAATAAACGT |
| P1253 | | |
| ERCAS12A- | 1464 | /5Phos/TTGAAGATCTCTAATAAGGACTGGNNKGACTTTATCCAAAATAAACGTTAT |
| P1254 | | |
| ERCAS12A- | 1465 | /5Phos/AAGATCTCTAATAAGGACTGGTTTNNKTTTATCCAAAATAAACGTTATTTA |
| P1255 | | |
| ERCAS12A- | 1466 | /5Phos/ATCTCTAATAAGGACTGGTTTGACNNKATCCAAAATAAACGTTATTTAGGT |
| P1256 | | |
| ERCAS12A- | 1467 | /5Phos/TCTAATAAGGACTGGTTTGACTTTNNKCAAAATAAACGTTATTTAGGTCGT |
| P1257 | | |
| ERCAS12A- | 1468 | /5Phos/AATAAGGACTGGTTTGACTTTATCNNKAATAAACGTTATTTAGGTCGTAGC |
| P1258 | | |
| ERCAS12A- | 1469 | /5Phos/AAGGACTGGTTTGACTTTATCCAANNKAAACGTTATTTAGGTCGTAGCAGT |
| P1259 | | |
| ERCAS12A- | 1470 | /5Phos/GACTGGTTTGACTTTATCCAAAATNNKCGTTATTTAGGTCGTAGCAGTGAT |
| P1260 | | |
| ERCAS12A- | 1471 | /5Phos/TGGTTTGACTTTATCCAAAATAAANNKTATTTAGGTCGTAGCAGTGATGAT |
| P1261 | | |
| ERCAS12A- | 1472 | /5Phos/TTTGACTTTATCCAAAATAAACGTNNKTTAGGTCGTAGCAGTGATGATGAA |
| P1262 | | |
| ERCAS12A- | 1473 | /5Phos/GACTTTATCCAAAATAAACGTTATNNKGGTCGTAGCAGTGATGATGAAGCA |
| P1263 | | |

### Example 3

### Editing Efficiency of ErCas12a Variants in Human Cells

This example evaluates the DNA cleavage activity of 20 ErCas12a point mutations in the context of E. coli (FIG. 7). The cleavage activity of each mutant was quantified by calculating the survival rate of E. coli with or without the induction of toxic reporter gene. Compared to WT-ERCAS12A, 31 out of 40 point mutations showed improved DNA cleavage, thus further validating the result of sequencing based, high-throughput phenotype measurement in Example 1.

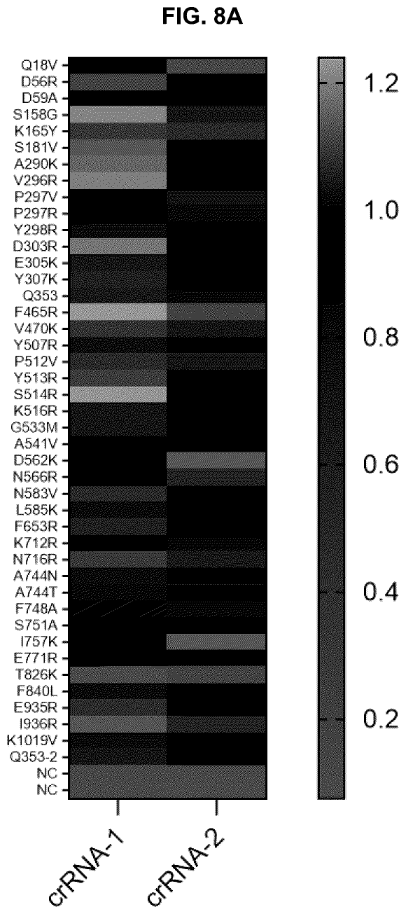

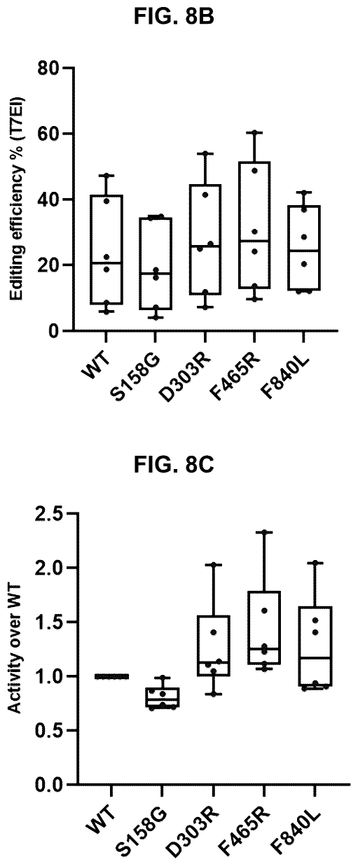

Eight representative ERCAS12A point mutations were selected for further characterization in human cells. These point mutations included 135L, G39Q, 140Q, S469K, Y507S, K516P, T826F and Y907V. Site directed mutagenesis was performed to introduce specific point mutations on the expression vector using primers listed in Table 4. Each protein was expressed in E. coli BL21DE3 cells, sequentially purified using nickel-affinity and heparin chromatography. To evaluate the genome editing efficiency of each variant, ERCAS12A-RNPs were assembled using 6 crRNAs targeting human HPRT gene (Table 5). The assembled RNPs (50 nM or 200 nM) were delivered into HEK293 cells by Lonza nucleofection (SF buffer, DS-150 program), and the editing efficiency was measured by T7 endonuclease I assay (T7EI) 48-hours post delivery (FIG. 8A-B). To facilitate the comparison, the efficiency of variant at each target was normalized to WT-ERCAS12A and presented as fold-improvement over WT (FIG. 8C-D). The single mutant variants showed improved activity over WT at one or multiple sites, especially at low RNP concentration (50 nM, FIG. 8B, D).

TABLE 4
Primers for Site Directed Mutagenesis of ErCas12a

| | SEQ ID | |
|---|---|---|
| Mutation | NO | DNA Primers (5′→3′) |
| I35L Fwd | 1474 | ACTGAGACAACACAGCAGTTCCTGGTTAAAAACGGCATCATTAAAGAAGATG |
| I35L Rev | 1475 | CATCTTCTTTAATGATGCCGTTTTTAACCAGGAACTGCTGTGTTGTCTCAGT |
| G39Q Fwd | 1476 | CACAGCAGTTCATCGTTAAAAACCAGATCATTAAAGAAGATGAGCTGCG |
| G39Q Rev | 1477 | CGCAGCTCATCTTCTTTAATGATCTGGTTTTTAACGATGAACTGCTGTG |
| I40Q Fwd | 1478 | CAGTTCATCGTTAAAAACGGCCAGATTAAAGAAGATGAGCTGCGC |
| I40Q Rev | 1479 | GCGCAGCTCATCTTCTTTAATCTGGCCGTTTTTAACGATGAACTG |
| S469K Fwd | 1480 | ACGCCTTCCATTGGTGCAAAGTGTTTATGACAGAGGAGTTAGTAGA |
| S469K Rev | 1481 | TCTACTAACTCCTCTGTCATAAACACTTTGCACCAATGGAAGGCGT |
| Y507S Fwd | 1482 | GATTTCACTGTATAACTTAGTCCGTAATAGCGTCACTCAGAAGCCGTACTC |
| Y507S Rev | 1483 | GAGTACGGCTTCTGAGTGACGCTATTACGGACTAAGTTATACAGTGAAATC |
| K516P Fwd | 1484 | TCAGAAGCCGTACTCAACTCCGAAGATCAAGTTAAATTTTGGAATTCCGA |
| K516P Rev | 1485 | TCGGAATTCCAAAATTTAACTTGATCTTCGGAGTTGAGTACGGCTTCTGA |
| T826F Fwd | 1486 | GTCACCACGAAGCAGCATTTAACATTGTGAAAGACTATCGCTAC |
| T826F Rev | 1487 | GTAGCGATAGTCTTTCACAATGTTAAATGCTGCTTCGTGGTGAC |
| Y907V Fwd | 1488 | AGCAAAAGTCTTTTAATATCGTTAATGGTGTGGACTACCAAATTAAGCTTAAACAA |
| | | CAGG |
| Y907V Rev | 1489 | CCTGTTGTTTAAGCTTAATTTGGTAGTCCACACCATTAACGATATTAAAAGACTTT |
| | | TGCT |

TABLE 5
crRNA and Primer Sequences for T7E1 Assay

| | SEQ ID | |
|---|---|---|
| crRNA | NO | crRNA RNA Sequences (5′→3′) |
| crRNA-1 | 1490 | UUAAUUUCUACUCUUGUAGAUCUUGGGUGUGUUAAAAGUGAC |
| crRNA-2 | 1491 | UUAAUUUCUACUCUUGUAGAUAUCCGUGCUGAGUGUACCAUG |
| crRNA-3 | 1492 | UUAAUUUCUACUCUUGUAGAUUAAUGCCCUGUAGUCUCUCUG |
| crRNA-4 | 1493 | UUAAUUUCUACUCUUGUAGAUUAAUUAACAGCUUGCUGGUGA |
| crRNA-5 | 1494 | UUAAUUUCUACUCUUGUAGAUUGUGAAAUGGCUUAUAAUUGC |
| crRNA-6 | 1495 | UUAAUUUCUACUCUUGUAGAUUUGUAGGAUAUGCCCUUGACU |
| | | DNA Primer Sequences (5′→3′) |
| HPRT7 Fwd | 1496 | AAGAATGTTGTGATAAAAGGTGATGCT |
| HPRT7 Rev | 1497 | ACACATCCATGGGACTTCTGCCTC |

### Example 4

### Editing Efficiency of ErCas12 Variants in Human Cells

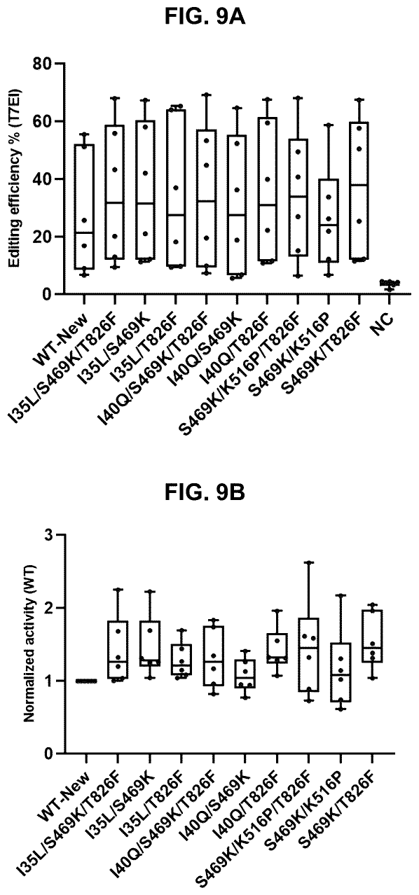

This example evaluates the editing efficiency of 42 additional ErCas12a point mutations selected based on previous E. coli screen data. Site directed mutagenesis was performed to introduce specific point mutations on the expression vector using primers listed in Table 6. Purified plasmids were lipofected into human HEK293 cells with 2 crRNAs targeting human HPRT gene (Table 7). The editing efficiency of HPRT was measured by T7EI assay 2 days post-delivery. To facilitate the comparison, the efficiency of variant at each target was normalized to WT ERCAS12A and presented as fold-improvement over WT (FIG. 9). 21 of 42 mutants improved the on-targeting efficiency of ERCAS12A when delivered as plasmid (FIG. 8, Table 7). Four point mutations, including S158G, D303R, F465R, and F840L, were selected for further characterization as RNP. 3 out of 4 mutations (D303R, F465R, F840L) improved the on-target editing efficiency in nearly all target sites (FIG. 8B-C).

TABLE 6
Primers for Site Directed Mutagenesis of ERCAS12A

| | SEQ ID | |
|---|---|---|
| Mutation | NO | Fwd Primers (5′→3′) |
| Q18V Fwd | 1498 | ATCGGCATCAGCAGCCTGGTGAAAACCCTGCGGAACGCT |
| Q18V Rev | 1499 | AGCGTTCCGCAGGGTTTTCACCAGGCTGCTGATGCCGAT |
| D56R Fwd | 1500 | CGAGAACCGGCAGATCCTGAAGCGGATCATGGACGACTACTACCGGG |
| D56R Rev | 1501 | CCCGGTAGTAGTCGTCCATGATCCGCTTCAGGATCTGCCGGTTCTCG |
| D59A Fwd | 1502 | GCAGATCCTGAAGGACATCATGGCCGACTACTACCGGGGCTTCATCA |
| D59A Rev | 1503 | TGATGAAGCCCCGGTAGTAGTCGGCCATGATGTCCTTCAGGATCTGC |
| S158G Fwd | 1504 | AGAAAACCCAAGTCATCAAGCTGTTCGGCAGATTCGCCACCAGCTTCAA |
| S158G Rev | 1505 | TTGAAGCTGGTGGCGAATCTGCCGAACAGCTTGATGACTTGGGTTTTCT |
| K165Y Fwd | 1506 | GCAGATTCGCCACCAGCTTCTACGACTACTTCAAGAACCGGGCCAAC |
| K165Y Rev | 1507 | GTTGGCCCGGTTCTTGAAGTAGTCGTAGAAGCTGGTGGCGAATCTGC |
| D178R Fwd | 1508 | GCCAACTGCTTCAGCGCCCGGGATATCAGCAGCAGCTCCTGC |
| D178R Rev | 1509 | GCAGGAGCTGCTGCTGATATCCCGGGCGCTGAAGCAGTTGGC |
| S181V Fwd | 1510 | GCTTCAGCGCCGACGATATCGTGAGCAGCTCCTGCCACAGA |
| S181V Rev | 1511 | TCTGTGGCAGGAGCTGCTCACGATATCGTCGGCGCTGAAGC |
| A290K Fwd | 1512 | GCTGCACAAGCAGATTCTGTGTATCAAGGACACCTCCTACGAGGTGCC |
| A290K Rev | 1513 | GGCACCTCGTAGGAGGTGTCCTTGATACACAGAATCTGCTTGTGCAGC |
| V296R Fwd | 1514 | TCGCCGACACCTCCTACGAGCGGCCCTACAAGTTCGAGAGCGAC |
| V296R Rev | 1515 | GTCGCTCTCGAACTTGTAGGGCCGCTCGTAGGAGGTGTCGGCGA |
| P297V Fwd | 1516 | CCGACACCTCCTACGAGGTGGTGTACAAGTTCGAGAGCGACGAAGAG |
| P297V Rev | 1517 | CTCTTCGTCGCTCTCGAACTTGTACACCACCTCGTAGGAGGTGTCGG |
| P297R Fwd | 1518 | CCGACACCTCCTACGAGGTGCGGTACAAGTTCGAGAGCGACGAAGAG |
| P297R Rev | 1519 | CTCTTCGTCGCTCTCGAACTTGTACCGCACCTCGTAGGAGGTGTCGG |
| Y298R Fwd | 1520 | CACCTCCTACGAGGTGCCCCGGAAGTTCGAGAGCGACGAAGAGG |
| Y298R Rev | 1521 | CCTCTTCGTCGCTCTCGAACTTCCGGGGCACCTCGTAGGAGGTG |
| D303R Fwd | 1522 | GTGCCCTACAAGTTCGAGAGCCGGGAAGAGGTGTACCAGAGCGT |
| D303R Rev | 1523 | ACGCTCTGGTACACCTCTTCCCGGCTCTCGAACTTGTAGGGCAC |
| E305K Fwd | 1524 | CCTACAAGTTCGAGAGCGACGAAAAGGTGTACCAGAGCGTGAAC |
| E305K Rev | 1525 | GTTCACGCTCTGGTACACCTTTTCGTCGCTCTCGAACTTGTAGG |
| Y307K Fwd | 1526 | TTCGAGAGCGACGAAGAGGTGAAGCAGAGCGTGAACGGCTTC |
| Y307K Rev | 1527 | GAAGCCGTTCACGCTCTGCTTCACCTCTTCGTCGCTCTCGAA |
| Q353R Fwd | 1528 | AAGTTCTACGAGAGCGTGTCCCGGAAAACCTACAGAGACTGGGAGAC |
| Q353R Rev | 1529 | GTCTCCCAGTCTCTGTAGGTTTTCCGGGACACGCTCTCGTAGAACTT |
| F465R Fwd | 1530 | TGGACGTGATCATGAACGCCCGGCACTGGTGCAGCGTGTTC |
| F465R Rev | 1531 | GAACACGCTGCACCAGTGCCGGGCGTTCATGATCACGTCCA |
| V470K Fwd | 1532 | GCCTTCCACTGGTGCAGCAAGTTCATGACCGAGGAACTGGTG |
| V470K Rev | 1533 | CACCAGTTCCTCGGTCATGAACTTGCTGCACCAGTGGAAGGC |
| Y507R Fwd | 1534 | GTACAACCTCGTGCGGAACCGGGTGACCCAGAAGCCTTACAG |
| Y507R Rev | 1535 | CTGTAAGGCTTCTGGGTCACCCGGTTCCGCACGAGGTTGTAC |
| P512V Fwd | 1536 | CGGAACTACGTGACCCAGAAGGTGTACAGCACCAAGAAGATCAAGCT |
| P512V Rev | 1537 | AGCTTGATCTTCTTGGTGCTGTACACCTTCTGGGTCACGTAGTTCCG |
| Y513R Fwd | 1538 | CTACGTGACCCAGAAGCCTCGGAGCACCAAGAAGATCAAGCTG |
| Y513R Rev | 1539 | CAGCTTGATCTTCTTGGTGCTCCGAGGCTTCTGGGTCACGTAG |
| S514R Fwd | 1540 | CTACGTGACCCAGAAGCCTTACCGGACCAAGAAGATCAAGCTGAACTTC |
| S514R Rev | 1541 | GAAGTTCAGCTTGATCTTCTTGGTCCGGTAAGGCTTCTGGGTCACGTAG |
| K516R Fwd | 1542 | CCCAGAAGCCTTACAGCACCCGGAAGATCAAGCTGAACTTCGGC |
| K516R Rev | 1543 | GCCGAAGTTCAGCTTGATCTTCCGGGTGCTGTAAGGCTTCTGGG |
| G553M Fwd | 1544 | GCGGGACAATCTGTACTACCTGATGATCTTCAACGCCAAGAACAAGC |
| G553M Rev | 1545 | GCTTGTTCTTGGCGTTGAAGATCATCAGGTAGTACAGATTGTCCCGC |
| A541K Fwd | 1546 | AAGAGCAAAGAGTACAGCAACAACGTGATCATCCTGATGCGGGACAA |
| A541K Rev | 1547 | TTGTCCCGCATCAGGATGATCACGTTGTTGCTGTACTCTTTGCTCTT |
| D562K Fwd | 1548 | TCAACGCCAAGAACAAGCCCAAGAAGAAGATTATCGAGGGCAACAC |
| D562K Rev | 1549 | GTGTTGCCCTCGATAATCTTCTTCTTGGGCTTGTTCTTGGCGTTGA |
| N583V Fwd | 1550 | GGCGACTACAAGAAGATGATCTATGTGCTGCTGCCTGGGCCG |
| N583V Rev | 1551 | CGGCCCAGGCAGCAGCACATAGATCATCTTCTTGTAGTCGCC |
| L585K Fwd | 1552 | GCGACTACAAGAAGATGATCTATAACCTGAAGCCTGGGCCGAACAAGATG |
| L585K Rev | 1553 | CATCTTGTTCGGCCCAGGCTTCAGGTTATAGATCATCTTCTTGTAGTCGC |
| F653R Fwd | 1554 | CCATCCATCCAGAGTGGAAGAATCGGGGCTTCGACTTCAGCGAC |
| F653R Rev | 1555 | GTCGCTGAAGTCGAAGCCCCGATTCTTCCACTCTGGATGGATGG |
| K712R Fwd | 1556 | CAGATCTACAACAAAGACTTCAGCAAGCGGTCCACCGGCAACGACAA |
| K712R Rev | 1557 | TTGTCGTTGCCGGTGGACCGCTTGCTGAAGTCTTTGTTGTAGATCTG |
| N716R Fwd | 1558 | AGCAAGAAGTCCACCGGCCGGGACAACCTGCACACCATGTAC |
| N716R Rev | 1559 | GTACATGGTGTGCAGGTTGTCCCGGCCGGTGGACTTCTTGCT |
| A744N Fwd | 1560 | CTGAAGCTGAACGGCGAGAACGAGATTTTTTTCAGAAAGTCCAGCATC |
| A744N Rev | 1561 | GATGCTGGACTTTCTGAAAAAAATCTCGTTCTCGCCGTTCAGCTTCAG |
| A744T Fwd | 1562 | CTGAAGCTGAACGGCGAGACCGAGATTTTTTTCAGAAAGTCCAGCATC |
| A744T Rev | 1563 | GATGCTGGACTTTCTGAAAAAAATCTCGGTCTCGCCGTTCAGCTTCAG |
| F748A Fwd | 1564 | CGGCGAGGCCGAGATTTTTGCCAGAAAGTCCAGCATCAAGAACC |
| F748A Rev | 1565 | GGTTCTTGATGCTGGACTTTCTGGCAAAAATCTCGGCCTCGCCG |
| S751A Fwd | 1566 | CGAGGCCGAGATTTTTTTCAGAAAGGCCAGCATCAAGAACCCCATCAT |
| S751A Rev | 1567 | ATGATGGGGTTCTTGATGCTGGCCTTTCTGAAAAAAATCTCGGCCTCG |
| I757K Fwd | 1568 | AGTCCAGCATCAAGAACCCCAAGATCCACAAAAAGGGCAGCAT |
| I757K Rev | 1569 | ATGCTGCCCTTTTTGTGGATCTTGGGGTTCTTGATGCTGGACT |
| E771R Fwd | 1570 | CATCCTGGTCAACCGGACATACCGGGCCGAAGAGAAGGATCAGTTCG |
| E771R Rev | 1571 | CGAACTGATCCTTCTCTTCGGCCCGGTATGTCCGGTTGACCAGGATG |
| T826K Fwd | 1572 | CACCACGAAGCCGCCAAGAATATCGTGAAGGACTACCGGT |
| T826K Rev | 1573 | ACCGGTAGTCCTTCACGATATTCTTGGCGGCTTCGTGGTG |
| F840L Fwd | 1574 | ACTATCGCTACACTTACGATAAGTACCTGTTACATATGCCTATTACGATTAACTT |
| | | TAAGG |
| F840L Rev | 1575 | CCTTAAAGTTAATCGTAATAGGCATATGTAACAGGTACTTATCGTAAGTGTAGCG |
| | | ATAGT |
| E935R Fwd | 1576 | GGAAAGAGATCGGCAAGATCAAACGGATCAAAGAGGGCTACCTGAGC |
| E935R Rev | 1577 | GCTCAGGTAGCCCTCTTTGATCCGTTTGATCTTGCCGATCTCTTTCC |
| I936R Fwd | 1578 | GAAAGAGATCGGCAAGATCAAAGAACGGAAAGAGGGCTACCTGAGCC |
| I936R Rev | 1579 | GGCTCAGGTAGCCCTCTTTCCGTTCTTTGATCTTGCCGATCTCTTTC |
| K1019V Fwd | 1580 | CTGACCTACATTCCCGACGTGCTGAAAAACGTGGGCCACCA |
| K1019V Rev | 1581 | TGGTGGCCCACGTTTTTCAGCACGTCGGGAATGTAGGTCAG |

TABLE 7
crRNA and Primer Sequences for T7E1 Assay

| | SEQ ID | |
|---|---|---|
| crRNA | NO | crRNA RNA Sequences (5′→3′) |
| crRNA-1 | 1490 | UUAAUUUCUACUCUUGUAGAUCUUGGGUGUGUUAAAAGUGAC |
| crRNA-2 | 1491 | UUAAUUUCUACUCUUGUAGAUAUCCGUGCUGAGUGUACCAUG |
| crRNA-3 | 1492 | UUAAUUUCUACUCUUGUAGAUUAAUGCCCUGUAGUCUCUCUG |
| crRNA-4 | 1493 | UUAAUUUCUACUCUUGUAGAUUAAUUAACAGCUUGCUGGUGA |
| crRNA-5 | 1494 | UUAAUUUCUACUCUUGUAGAUUGUGAAAUGGCUUAUAAUUGC |
| crRNA-6 | 1495 | UUAAUUUCUACUCUUGUAGAUUUGUAGGAUAUGCCCUUGACU |
| | | DNA Primer Sequences (5'->3') |
| HPRT7 Fwd | 1496 | AAGAATGTTGTGATAAAAGGTGATGCT |
| HPRT7 Rev | 1497 | ACACATCCATGGGACTTCTGCCTC |

With a large collection of single point mutations with enhanced activity available, mutations were combined or “stacked” to further improve the activity of ERCAS12A. The point mutations described in previous examples were stacked in 9 combinations and the editing efficiency of these ERCAS12A variants were evaluated in human cells as RNPs (FIG. 9A-B). Notably, all stacked variants enhanced the on-target editing efficiency of ERCAS12A over one or multiple target sites. Among these, a variant with double mutations (S469K/T826F) had superior performance.

### Example 5

### Editing Efficiency of ErCas12 Variants in Human Cell Lines

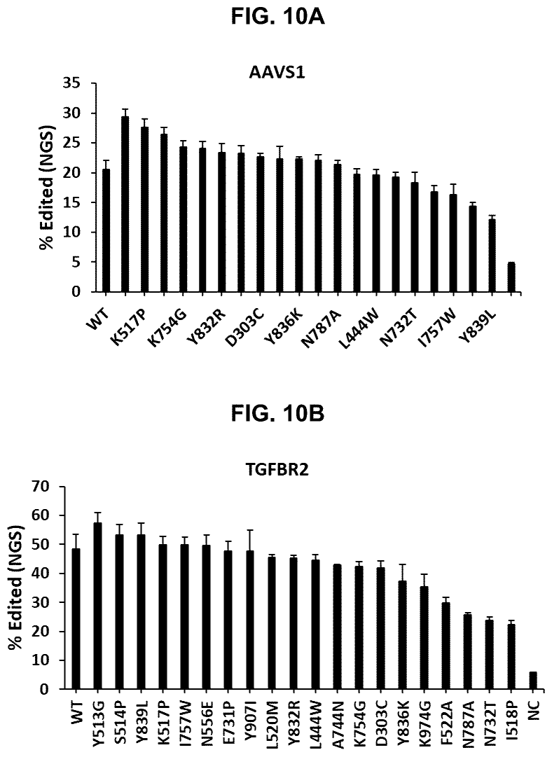

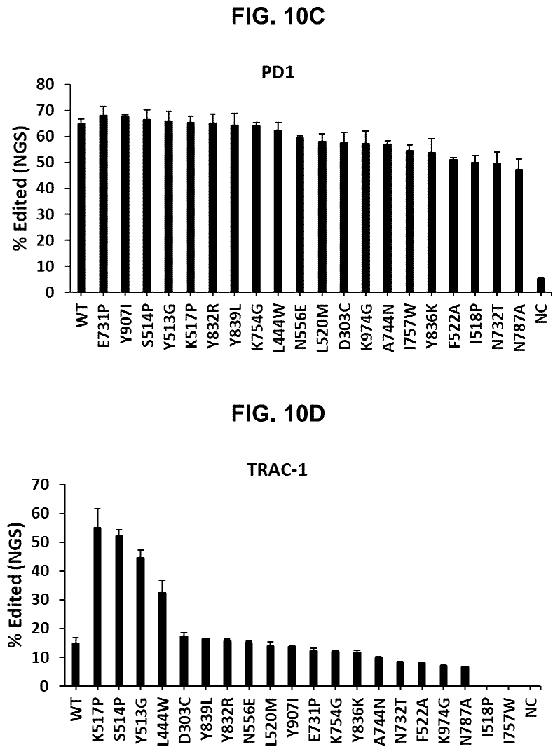

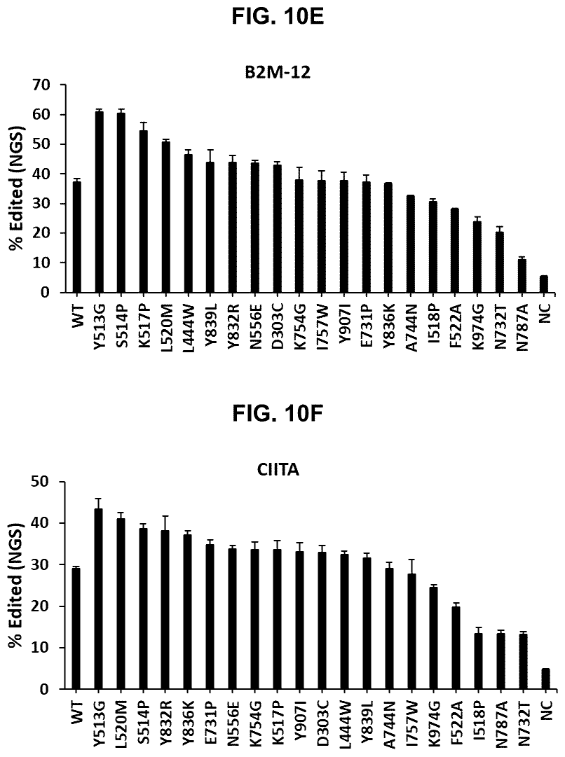

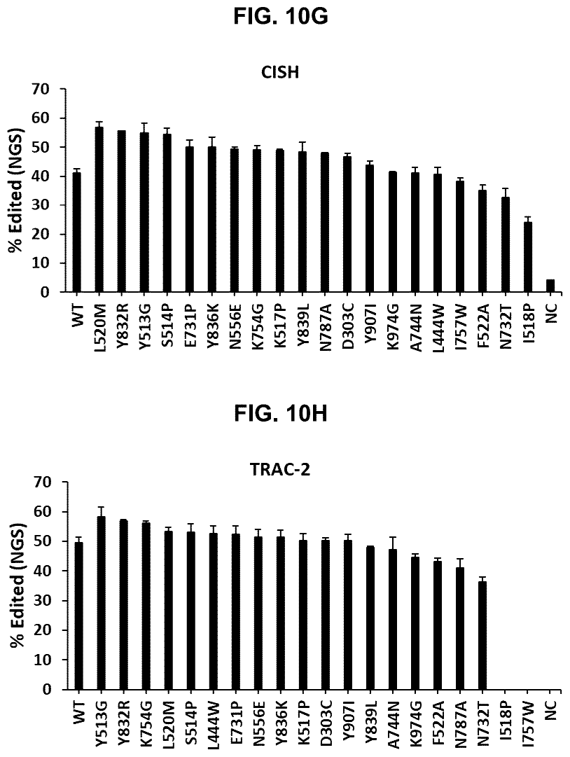

The editing efficiency of another 20 ERCAS12A point mutations was evaluated. The additional 20 ERCAS12A point mutations were selected based on the E. coli screening data. Site directed mutagenesis was performed to introduce specific point mutations on the expression vector using primers listed in Table 8. Purified plasmids were lipofected into human HEK293 cells with 8 crRNAs targeting human therapeutically relevant genes (Table 9). The editing efficiency of each gene was measured by NGS 3 days post-delivery (FIG. 10A-H).

TABLE 8
Primers for Site Directed Mutagenesis of ERCAS12A

| | SEQ ID | |
|---|---|---|
| Mutation | NO | Fwd Primers (5′→3′) |
| D303C Fwd | 1582 | GCCCTACAAGTTCGAGAGCTGCGAAGAGGTGTACCAGAGCG |
| D303C Rev | 1583 | CGCTCTGGTACACCTCTTCGCAGCTCTCGAACTTGTAGGGC |
| L444W Fwd | 1584 | AAGTACAACCCAGAGATCCACTGGGTGGAAAGCGAGCTGAAG |
| L444W Rev | 1585 | CTTCAGCTCGCTTTCCACCCAGTGGATCTCTGGGTTGTACTT |
| Y513G Fwd | 1586 | CGTGACCCAGAAGCCTGGCAGCACCAAGAAGATCAAGCT |
| Y513G Rev | 1587 | AGCTTGATCTTCTTGGTGCTGCCAGGCTTCTGGGTCACG |
| S514P Fwd | 1588 | CGTGACCCAGAAGCCTTACCCCACCAAGAAGATCAAGCTGAACT |
| S514P Rev | 1589 | AGTTCAGCTTGATCTTCTTGGTGGGGTAAGGCTTCTGGGTCACG |
| K517P Fwd | 1590 | GAAGCCTTACAGCACCAAGCCCATCAAGCTGAACTTCGGC |
| K517P Rev | 1591 | GCCGAAGTTCAGCTTGATGGGCTTGGTGCTGTAAGGCTTC |
| I518P Fwd | 1592 | GCCTTACAGCACCAAGAAGCCCAAGCTGAACTTCGGCATCC |
| I518P Rev | 1593 | GGATGCCGAAGTTCAGCTTGGGCTTCTTGGTGCTGTAAGGC |
| L520M Fwd | 1594 | CTTACAGCACCAAGAAGATCAAGATGAACTTCGGCATCCCCAC |
| L520M Rev | 1595 | GTGGGGATGCCGAAGTTCATCTTGATCTTCTTGGTGCTGTAAG |
| F522A Fwd | 1596 | CACCAAGAAGATCAAGCTGAACGCCGGCATCCCCACACTGG |
| F522A Rev | 1597 | CCAGTGTGGGGATGCCGGCGTTCAGCTTGATCTTCTTGGTG |
| N556E Fwd | 1598 | CTGTACTACCTGGGCATCTTCGAGGCCAAGAACAAGCCCG |
| N556E Rev | 1599 | CGGGCTTGTTCTTGGCCTCGAAGATGCCCAGGTAGTACAG |
| E731P Fwd | 1600 | GAAGAACCTGTTCAGCGAGCCCAACCTCAAGGACATCGTGC |
| E731P Rev | 1601 | GCACGATGTCCTTGAGGTTGGGCTCGCTGAACAGGTTCTTC |
| N732T Fwd | 1602 | AACCTGTTCAGCGAGGAAACCCTCAAGGACATCGTGCTGA |
| N732T Rev | 1603 | TCAGCACGATGTCCTTGAGGGTTTCCTCGCTGAACAGGTT |
| A744N Fwd | 1604 | GAAGCTGAACGGCGAGAACGAGATTTTTTTCAGAAAGTCCAGCA |
| A744N Rev | 1605 | TGCTGGACTTTCTGAAAAAAATCTCGTTCTCGCCGTTCAGCTTC |
| K754G Fwd | 1606 | GATTTTTTTCAGAAAGTCCAGCATCGGCAACCCCATCATCCACAAAAAG |
| K754G Rev | 1607 | CTTTTTGTGGATGATGGGGTTGCCGATGCTGGACTTTCTGAAAAAAATC |
| I757W Fwd | 1608 | TCCAGCATCAAGAACCCCTGGATCCACAAAAAGGGCAGCA |
| I757W Rev | 1609 | TGCTGCCCTTTTTGTGGATCCAGGGGTTCTTGATGCTGGA |
| N787A Fwd | 1610 | CATCCAGATTGTGCGGAAGGCCATCCCCGAGAACATCTATCAAG |
| N787A Rev | 1611 | CTTGATAGATGTTCTCGGGGATGGCCTTCCGCACAATCTGGATG |
| Y832R Fwd | 1612 | CCACCAATATCGTGAAGGACCGGCGGTATACCTACGATAAGTACTTCC |
| Y832R Rev | 1613 | GGAAGTACTTATCGTAGGTATACCGCCGGTCCTTCACGATATTGGTGG |
| Y836K Fwd | 1614 | GTGAAGGACTACCGGTATACCAAGGATAAGTACTTCCTGCACATGCC |
| Y836K Rev | 1615 | GGCATGTGCAGGAAGTACTTATCCTTGGTATACCGGTAGTCCTTCAC |
| Y839L Fwd | 1616 | GACTACCGGTATACCTACGATAAGCTGTTCCTGCACATGCCCA |
| Y839L Rev | 1617 | TGGGCATGTGCAGGAACAGCTTATCGTAGGTATACCGGTAGTC |
| Y907I Fwd | 1618 | GCTTCAACATCGTGAATGGCATCGACTACCAGATCAAACTGAAGCA |
| Y907I Rev | 1619 | TGCTTCAGTTTGATCTGGTAGTCGATGCCATTCACGATGTTGAAGC |
| K974G Fwd | 1620 | CTTCAAGAAAGGCCGCTTCGGCGTGGAAAGACAGGTCTACCAG |
| K974G Rev | 1621 | CTGGTAGACCTGTCTTTCCACGCCGAAGCGGCCTTTCTTGAAG |
| D303C Fwd | 1582 | GCCCTACAAGTTCGAGAGCTGCGAAGAGGTGTACCAGAGCG |
| D303C Rev | 1583 | CGCTCTGGTACACCTCTTCGCAGCTCTCGAACTTGTAGGGC |
| L444W Fwd | 1584 | AAGTACAACCCAGAGATCCACTGGGTGGAAAGCGAGCTGAAG |
| L444W Rev | 1585 | CTTCAGCTCGCTTTCCACCCAGTGGATCTCTGGGTTGTACTT |
| Y513G Fwd | 1586 | CGTGACCCAGAAGCCTGGCAGCACCAAGAAGATCAAGCT |
| Y513G Rev | 1587 | AGCTTGATCTTCTTGGTGCTGCCAGGCTTCTGGGTCACG |
| S514P Fwd | 1588 | CGTGACCCAGAAGCCTTACCCCACCAAGAAGATCAAGCTGAACT |
| S514P Rev | 1589 | AGTTCAGCTTGATCTTCTTGGTGGGGTAAGGCTTCTGGGTCACG |
| K517P Fwd | 1590 | GAAGCCTTACAGCACCAAGCCCATCAAGCTGAACTTCGGC |
| K517P Rev | 1591 | GCCGAAGTTCAGCTTGATGGGCTTGGTGCTGTAAGGCTTC |
| I518P Fwd | 1592 | GCCTTACAGCACCAAGAAGCCCAAGCTGAACTTCGGCATCC |
| I518P Rev | 1593 | GGATGCCGAAGTTCAGCTTGGGCTTCTTGGTGCTGTAAGGC |
| L520M Fwd | 1594 | CTTACAGCACCAAGAAGATCAAGATGAACTTCGGCATCCCCAC |
| L520M Rev | 1595 | GTGGGGATGCCGAAGTTCATCTTGATCTTCTTGGTGCTGTAAG |
| F522A Fwd | 1596 | CACCAAGAAGATCAAGCTGAACGCCGGCATCCCCACACTGG |
| F522A Rev | 1597 | CCAGTGTGGGGATGCCGGCGTTCAGCTTGATCTTCTTGGTG |
| N556E Fwd | 1598 | CTGTACTACCTGGGCATCTTCGAGGCCAAGAACAAGCCCG |
| N556E Rev | 1599 | CGGGCTTGTTCTTGGCCTCGAAGATGCCCAGGTAGTACAG |
| E731P Fwd | 1600 | GAAGAACCTGTTCAGCGAGCCCAACCTCAAGGACATCGTGC |
| E731P Rev | 1601 | GCACGATGTCCTTGAGGTTGGGCTCGCTGAACAGGTTCTTC |
| N732T Fwd | 1602 | AACCTGTTCAGCGAGGAAACCCTCAAGGACATCGTGCTGA |
| N732T Rev | 1603 | TCAGCACGATGTCCTTGAGGGTTTCCTCGCTGAACAGGTT |
| A744N Fwd | 1604 | GAAGCTGAACGGCGAGAACGAGATTTTTTTCAGAAAGTCCAGCA |
| A744N Rev | 1605 | TGCTGGACTTTCTGAAAAAAATCTCGTTCTCGCCGTTCAGCTTC |
| K754G Fwd | 1606 | GATTTTTTTCAGAAAGTCCAGCATCGGCAACCCCATCATCCACAAAAAG |
| K754G Rev | 1607 | CTTTTTGTGGATGATGGGGTTGCCGATGCTGGACTTTCTGAAAAAAATC |
| I757W Fwd | 1608 | TCCAGCATCAAGAACCCCTGGATCCACAAAAAGGGCAGCA |
| I757W Rev | 1609 | TGCTGCCCTTTTTGTGGATCCAGGGGTTCTTGATGCTGGA |
| N787A Fwd | 1610 | CATCCAGATTGTGCGGAAGGCCATCCCCGAGAACATCTATCAAG |
| N787A Rev | 1611 | CTTGATAGATGTTCTCGGGGATGGCCTTCCGCACAATCTGGATG |
| Y832R Fwd | 1612 | CCACCAATATCGTGAAGGACCGGCGGTATACCTACGATAAGTACTTCC |
| Y832R Rev | 1613 | GGAAGTACTTATCGTAGGTATACCGCCGGTCCTTCACGATATTGGTGG |
| Y836K Fwd | 1614 | GTGAAGGACTACCGGTATACCAAGGATAAGTACTTCCTGCACATGCC |
| Y836K Rev | 1615 | GGCATGTGCAGGAAGTACTTATCCTTGGTATACCGGTAGTCCTTCAC |
| Y839L Fwd | 1616 | GACTACCGGTATACCTACGATAAGCTGTTCCTGCACATGCCCA |
| Y839L Rev | 1617 | TGGGCATGTGCAGGAACAGCTTATCGTAGGTATACCGGTAGTC |
| Y907I Fwd | 1618 | GCTTCAACATCGTGAATGGCATCGACTACCAGATCAAACTGAAGCA |
| Y907I Rev | 1619 | TGCTTCAGTTTGATCTGGTAGTCGATGCCATTCACGATGTTGAAGC |
| K974G Fwd | 1620 | CTTCAAGAAAGGCCGCTTCGGCGTGGAAAGACAGGTCTACCAG |
| K974G Rev | 1621 | CTGGTAGACCTGTCTTTCCACGCCGAAGCGGCCTTTCTTGAAG |

TABLE 9
crRNA for Editing Experiments and NGS Primer Sequences

| | SEQ ID | |
|---|---|---|
| Target | NO | crRNA RNA Sequences (5′→3′) |
| AAVS1 | 1622 | UUUUUAAUUUCUACUCUUGUAGAUGACCCCUAAGGGAAGAAUGAG |
| PD1 | 1623 | UUUUUAAUUUCUACUCUUGUAGAUGCACGAAGCUCUCCGAUGUGU |
| TGFBR2 | 1624 | UUUUUAAUUUCUACUCUUGUAGAUUGAUGUGAGAUUUUCCACCUG |
| TRAC1 | 1625 | UUUUUAAUUUCUACUCUUGUAGAUCAGAUACGAACCUAAACUUUC |
| B2M-12 | 1626 | UUUUUAAUUUCUACUCUUGUAGAUUCACAGCCCAAGAUAGUUAAG |
| CIITA | 1627 | UUUUUAAUUUCUACUCUUGUAGAUCCGGCCUUUUUACCUUGGGGC |
| CISH | 1628 | UUUUUAAUUUCUACUCUUGUAGAUACUGACAGCGUGAACAGGUAG |
| TRAC2 | 1629 | UUUUUAAUUUCUACUCUUGUAGAUGAGUCUCUCAGCUGGUACACG |
| | SEQ ID | |
| Primer | NO | NGS DNA Primers (5′→3′) |
| AAVS1 Fwd | 1630 | acactctttccctacacgacgctcttccgatctGCTGTCACACTCCAGTT |
| AAVS1 Rev | 1631 | gtgactggagttcagacgtgtgctcttccgatctACCCAGTGTCCACAAG |
| PD1 Fwd | 1632 | acactctttccctacacgacgctcttccgatctAGCTTGTCCGTCTGGTT |
| PD1 Rev | 1633 | gtgactggagttcagacgtgtgctcttccgatctAGACTCCCCAGACAGG |
| TGFBR2 Fwd | 1634 | acactctttccctacacgacgctcttccgatctCATGATAGTCACTGACAACAAC |
| | | G |
| TGFBR2 Rev | 1635 | gtgactggagttcagacgtgtgctcttccgatctCTTACCATACAGCCACACAGA |
| TRAC1 Fwd | 1636 | acactctttccctacacgacgctcttccgatctACATGCAAGCCCATAACC |
| TRAC1 Rev | 1637 | gtgactggagttcagacgtgtgctcttccgatctGGTTTGCTCTCAGAGCTTA |
| B2M-12 Fwd | 1638 | acactctttccctacacgacgctcttccgatctCATCCGACATTGAAGTTGACTT |
| | | A |
| B2M-12 Rev | 1639 | gtgactggagttcagacgtgtgctcttccgatctCTACTCATACACAACTTTCAG |
| | | CA |
| CIITA Fwd | 1640 | acactctttccctacacgacgctcttccgatctTGGGATTCCTACACAATGC |
| CIITA Rev | 1641 | gtgactggagttcagacgtgtgctcttccgatctCCCATCCCCAATTCAGAATG |
| CISH Fwd | 1642 | acactctttccctacacgacgctcttccgatctCACAAGGCTGACCACATC |
| CISH Rev | 1643 | gtgactggagttcagacgtgtgctcttccgatctCAGAAGGCACGTTCTTAGT |
| TRAC2 Fwd | 1644 | acactctttccctacacgacgctcttccgatctATCCTCTTGTCCCACAGATATC |
| TRAC2 Rev | 1645 | gtgactggagttcagacgtgtgctcttccgatctCCATAGACCTCATGTCTAGCA |

### Example 6

### Editing Efficiency of ERCAS12A Variants in Human Cells

Evaluation of the editing efficiency of the ERCAS12A Y513G point mutation that was selected from plasmid screening was performed. Site directed mutagenesis was performed to introduce the point mutation on the protein expression vector using primers listed in Table 10. Purified protein was tested as RNP in human HEK293 cells with 7 crRNAs targeting human therapeutically relevant genes (Table 11). The editing efficiency of each gene was measured by NGS 2 days post-delivery (FIG. 11).

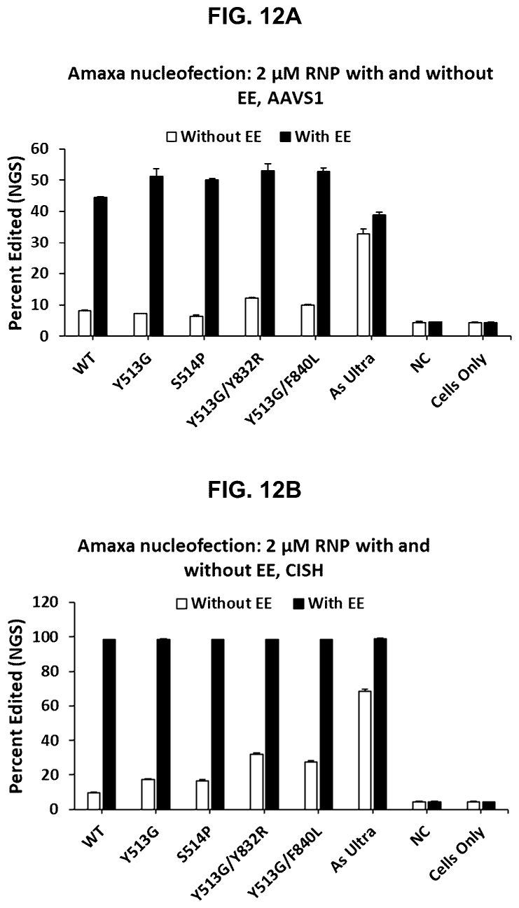

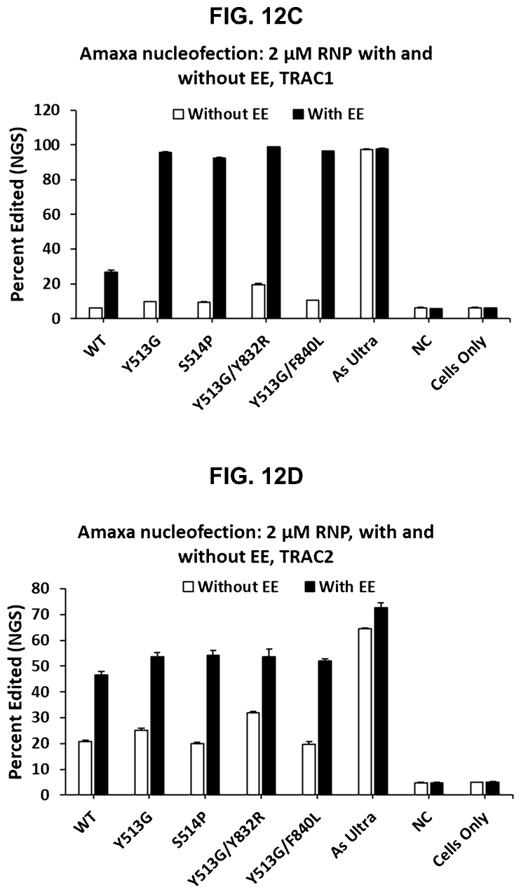

Site directed mutagenesis was then used to add additional mutations to ERCAS12A Y513G, Y832R, and F840L, and to make S514P, on the protein expression vector using primers listed in Table 10. Purified protein was tested as RNP in human HEK293 cells with 4 crRNAs targeting human therapeutically relevant genes (Table 11). The editing efficiency of each gene was measured by NGS 2 days post-delivery (FIG. 12A-D). This experiment also included Acidaminococcus sp. Cas12a M537R/F870L (AsCas12a-Ultra) as a positive control, and with and without electroporation enhancer.

TABLE 10
Primers for Site Directed Mutagenesis of ERCAS12A

| | SEQ ID | |
|---|---|---|
| Primer | NO | DNA Primers (5′→3′) |
| Y513G Fwd | 1646 | ACGTCACTCAGAAGCCGGGCTCAACTAAAAAGATCAAGTTAAATTTTGGAATT |
| Y513G Rev | 1647 | AATTCCAAAATTTAACTTGATCTTTTTAGTTGAGCCCGGCTTCTGAGTGACGT |
| S514P Fwd | 1648 | CGTCACTCAGAAGCCGTACCCGACTAAAAAGATCAAGTTAAATTTTGGAATTCC |
| S514P Rev | 1649 | GGAATTCCAAAATTTAACTTGATCTTTTTAGTCGGGTACGGCTTCTGAGTGACG |
| Y832R Fwd | 1650 | CAGCAACAAACATTGTGAAAGACCGTCGCTACACTTACGATAAGTACTTTTTAC |
| Y832R Rev | 1651 | GTAAAAAGTACTTATCGTAAGTGTAGCGACGGTCTTTCACAATGTTTGTTGCTG |
| F840L Fwd | 1652 | ACTATCGCTACACTTACGATAAGTACCTGTTACATATGCCTATTACGATTAACT |
| | | TTAAGG |
| F840L Rev | 1653 | CCTTAAAGTTAATCGTAATAGGCATATGTAACAGGTACTTATCGTAAGTGTAGC |
| | | GATAGT |

TABLE 11
crRNA for Editing Experiments and NGS Primer Sequences

| | SEQ ID | |
|---|---|---|
| Target | NO | crRNA RNA Sequences (5′→3′) |
| AAVS1 | 1622 | UUUUUAAUUUCUACUCUUGUAGAUGACCCCUAAGGGAAGAAUGAG |
| PD1 | 1623 | UUUUUAAUUUCUACUCUUGUAGAUGCACGAAGCUCUCCGAUGUGU |
| TGFBR2 | 1624 | UUUUUAAUUUCUACUCUUGUAGAUUGAUGUGAGAUUUUCCACCUG |
| TRAC1 | 1625 | UUUUUAAUUUCUACUCUUGUAGAUCAGAUACGAACCUAAACUUUC |
| B2M-12 | 1626 | UUUUUAAUUUCUACUCUUGUAGAUUCACAGCCCAAGAUAGUUAAG |
| CIITA | 1627 | UUUUUAAUUUCUACUCUUGUAGAUCCGGCCUUUUUACCUUGGGGC |
| CISH | 1628 | UUUUUAAUUUCUACUCUUGUAGAUACUGACAGCGUGAACAGGUAG |
| TRAC2 | 1629 | UUUUUAAUUUCUACUCUUGUAGAUGAGUCUCUCAGCUGGUACACG |
| | SEQ ID | |
| Primer | NO | NGS DNA Primers (5′→3′) |
| AAVS1 Fwd | 1630 | acactctttccctacacgacgctcttccgatctGCTGTCACACTCCAGTT |
| AAVS1 Rev | 1631 | gtgactggagttcagacgtgtgctcttccgatctACCCAGTGTCCACAAG |
| PD1 Fwd | 1632 | acactctttccctacacgacgctcttccgatctAGCTTGTCCGTCTGGTT |
| PD1 Rev | 1633 | gtgactggagttcagacgtgtgctcttccgatctAGACTCCCCAGACAGG |
| TGFBR2 Fwd | 1634 | acactctttccctacacgacgctcttccgatctCATGATAGTCACTGACAACAAC |
| | | G |
| TGFBR2 Rev | 1635 | gtgactggagttcagacgtgtgctcttccgatctCTTACCATACAGCCACACAGA |
| TRAC1 Fwd | 1636 | acactctttccctacacgacgctcttccgatctACATGCAAGCCCATAACC |
| TRAC1 Rev | 1637 | gtgactggagttcagacgtgtgctcttccgatctGGTTTGCTCTCAGAGCTTA |
| B2M-12 Fwd | 1638 | acactctttccctacacgacgctcttccgatctCATCCGACATTGAAGTTGACTT |
| | | A |
| B2M-12 Rev | 1639 | gtgactggagttcagacgtgtgctcttccgatctCTACTCATACACAACTTTCAG |
| | | CA |
| CIITA Fwd | 1640 | acactctttccctacacgacgctcttccgatctTGGGATTCCTACACAATGC |
| CIITA Rev | 1641 | gtgactggagttcagacgtgtgctcttccgatctCCCATCCCCAATTCAGAATG |
| CISH Fwd | 1642 | acactctttccctacacgacgctcttccgatctCACAAGGCTGACCACATC |
| CISH Rev | 1643 | gtgactggagttcagacgtgtgctcttccgatctCAGAAGGCACGTTCTTAGT |
| TRAC2 Fwd | 1644 | acactctttccctacacgacgctcttccgatctATCCTCTTGTCCCACAGATATC |
| TRAC2 Rev | 1645 | gtgactggagttcagacgtgtgctcttccgatctCCATAGACCTCATGTCTAGCA |

### Example 7

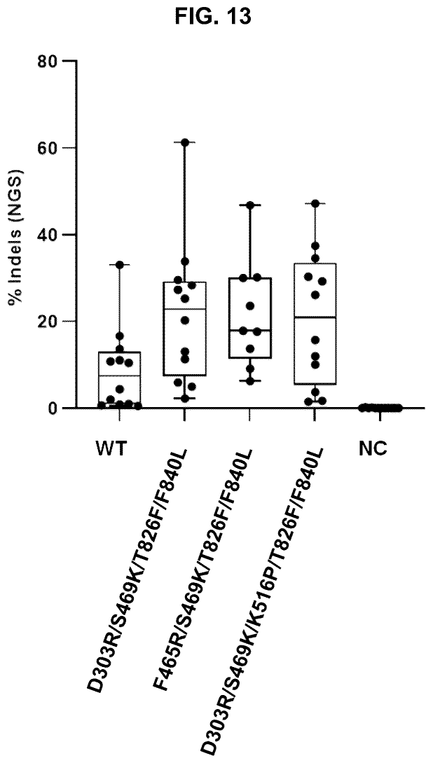

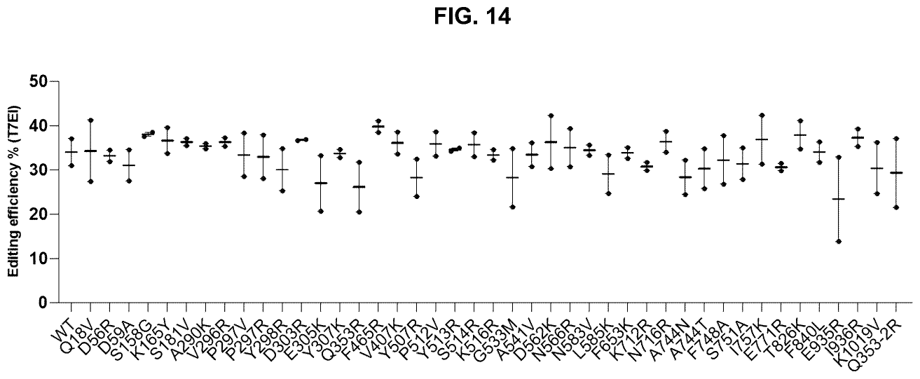

Another mutation, K516P, was stacked with the best ERCAS12A stacked mutants identified in previous examples (e.g., D303R/S469K/K516P/T826F/F840L). Site directed mutagenesis was used to add this point mutation and the primers are listed in Table 12. The protein was expressed in E. coli BL21 DE3 cells, sequentially purified using nickel-affinity, heparin chromatography and hydroxy-apatite chromatography. To evaluate the genome editing efficiency of each variant, ERCAS12A mutant-RNPs were assembled using crRNAs found in Table 13. The assembled RNPs (2 μM) were delivered into HEK293 cells by nucleofection, and the percent indels was measured by NGS 48-hours post-delivery (FIG. 13). Each of the stacked mutants (D303R/S469K/T826F/F840L; F465R/S469K/T826F/F840L; D303R/S469K/K516P/T826F/F840L) improved activity over ERCAS12A WT at these sites. Stacking these beneficial mutations led to more potent variants of ERCAS12A than any individual point mutations.

TABLE 12
Primers for Site Directed Mutagenesis of ERCAS12A

| | SEQ ID | |
|---|---|---|
| Primer | NO | DNA Primers (5′→3′) |
| K516P Fwd | 1654 | CTCAGAAGCCGTACTCAACTCCGAAGATCAAGTTAAATTTTGGAATTCCGA |
| K516P Rev | 1655 | TCGGAATTCCAAAATTTAACTTGATCTTCGGAGTTGAGTACGGCTTCTGAG |

TABLE 13
crRNA for Editing Experiments and NGS Primer Sequences

| | SEQ ID | |
|---|---|---|
| Name | NO | crRNA RNA Sequences (5′→3′) |
| crRNA-1 | 1656 | UUAAUUUCUACUCUUGUAGAUGACCCCUAAGGGAAGAAUGA |
| crRNA-2 | 1657 | UUAAUUUCUACUCUUGUAGAUCCGAUAUUCCUCAGGUACUC |
| crRNA-3 | 1658 | UUAAUUUCUACUCUUGUAGAUACUUUCCAUUCUCUGCUGGA |
| crRNA-4 | 1659 | UUAAUUUCUACUCUUGUAGAUCUGAAUUGCUAUGUGUCUGG |
| crRNA-5 | 1660 | UUAAUUUCUACUCUUGUAGAUAUCCAUCCGACAUUGAAGUU |
| crRNA-6 | 1661 | UUAAUUUCUACUCUUGUAGAUUCACAGCCCAAGAUAGUUAA |
| crRNA-7 | 1662 | UUAAUUUCUACUCUUGUAGAUCCGGCCUUUUUACCUUGGGG |
| crRNA-8 | 1663 | UUAAUUUCUACUCUUGUAGAUACUGACAGCGUGAACAGGUA |
| crRNA-9 | 1664 | UUAAUUUCUACUCUUGUAGAUGCACGAAGCUCUCCGAUGUG |
| crRNA-10 | 1665 | UUAAUUUCUACUCUUGUAGAUUGAUGUGAGAUUUUCCACCU |
| crRNA-11 | 1666 | UUAAUUUCUACUCUUGUAGAUCAGAUACGAACCUAAACUUU |
| crRNA-12 | 1667 | UUAAUUUCUACUCUUGUAGAUGAGUCUCUCAGCUGGUACAC |

The nucleotide and polypeptide sequences of selected ERCAS12A variants are provided in Table 14.

TABLE 14
Polynucleotide and Polypeptide Sequences of ERCAS12A Variants

ErCas12a-Q18V-NT (SEQ ID NO: 3)
atgAATAACGGTACCAATAACTTTCAAAATTTTATCGGGATCTCCTCGTTGGTGAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Q18V-AA (SEQ ID NO: 4)
MNNGTNNFQNFIGISSLVKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-I35L-NT (SEQ ID NO: 5)
atgAATAACGGTACCAATAACTTTCAAAATTTTATCGGGATCTCCTCGTTGCAAAAAACTCTTCGTAATGCTTTAATCCCGACT
GAGACAACACAGCAGTTCCTGGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I35L-AA (SEQ ID NO: 6)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-G39Q-NT (SEQ ID NO: 7)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACCAGATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-G39Q-AA (SEQ ID NO: 8)
MNNGTNNFQNFIGISSLQKTLRNALIPTETTQQFIVKNQIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-G39K-NT (SEQ ID NO: 9)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACAAAATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-G39K-AA (SEQ ID NO: 10)
MNNGTNNFQNFIGISSLQKTLRNALIPTETTQQFIVKNKIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-I40Q-NT (SEQ ID NO: 11)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCCAGATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I40Q-AA (SEQ ID NO: 12)
MNNGTNNFQNFIGISSLQKTLRNALIPTETTQQFIVKNGQIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-D56R-NT (SEQ ID NO: 13)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGCGT
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D56R-AA (SEQ ID NO: 14)
MNNGTNNFQNFIGISSLQKTLRNALIPTETTQQFIVKNGIIKEDELRGENRQILKRIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-D56P-NT (SEQ ID NO: 15)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGCCG
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D56P-AA (SEQ ID NO: 16)
MNNGTNNFQNFIGISSLQKTLRNALIPTETTQQFIVKNGIIKEDELRGENRQILKPIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-D59R-NT (SEQ ID NO: 17)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGCGTGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D59R-AA (SEQ ID NO: 18)
MNNGTNNFQNFIGISSLQKTLRNALIPTETTQQFIVKNGIIKEDELRGENRQILKDIMRDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-D59A-NT (SEQ ID NO: 19)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGCGGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D59A-AA (SEQ ID NO: 20)
MNNGTNNFQNFIGISSLQKTLRNALIPTETTQQFIVKNGIIKEDELRGENRQILKDIMADYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-S158G-NT (SEQ ID NO: 21)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTGGCCGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-S158G-AA (SEQ ID NO: 22)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFGRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDESDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-K165Y-NT (SEQ ID NO: 23)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTTATGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-K165Y-AA (SEQ ID NO: 24)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFYDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-S181V-NT (SEQ ID NO: 25)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTGTGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-S181V-AA (SEQ ID NO: 26)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDIVSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-A290K-NT (SEQ ID NO: 27)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCAAAGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-A290K-AA (SEQ ID NO: 28)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIKDTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-D291N-NT (SEQ ID NO: 29)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCACCACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D291N-AA (SEQ ID NO: 30)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIANTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-V296R-NT (SEQ ID NO: 31)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAACGTCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-V296R-AA (SEQ ID NO: 32)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYERPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-P297V-NT (SEQ ID NO: 33)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCGTGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-P297V-AA (SEQ ID NO: 34)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVVYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-P297R-NT (SEQ ID NO: 35)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCGTTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-P297R-AA (SEQ ID NO: 36)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVRYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-P297F-NT (SEQ ID NO: 37)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCTTTTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-P297F-AA (SEQ ID NO: 38)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMESAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVFYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDESDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-D303R-NT (SEQ ID NO: 39)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCCGTGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D303R-AA (SEQ ID NO: 40)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMESAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESREEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-D303C-NT (SEQ ID NO: 41)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCTGCGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D303C-AA (SEQ ID NO: 42)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESCEEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-E305K-NT (SEQ ID NO: 43)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAAAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-E305K-AA (SEQ ID NO: 44)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEKVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y307K-NT (SEQ ID NO: 45)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTAAAACAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y307K-AA (SEQ ID NO: 46)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVKQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Q353R-NT (SEQ ID NO: 47)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCGTAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Q353R-AA (SEQ ID NO: 48)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMESAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSRKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-L444W-NT (SEQ ID NO: 49)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATTGGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-L444W-AA (SEQ ID NO: 50)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHWVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-F465R-NT (SEQ ID NO: 51)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCCGTCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-F465R-AA (SEQ ID NO: 52)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNARHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-S469K-NT (SEQ ID NO: 53)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-S469K-AA (SEQ ID NO: 54)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCKVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-V470K-NT (SEQ ID NO: 55)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCAAATTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-V470K-AA (SEQ ID NO: 56)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSKFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-1498K-NT (SEQ ID NO: 57)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGAAATCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-1498K-AA (SEQ ID NO: 58)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVKSLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYENDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y507W-NT (SEQ ID NO: 59)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTGGGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y507W-AA (SEQ ID NO: 60)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNWVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDESDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-Y507S-NT (SEQ ID NO: 61)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATAGCGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y507S-AA (SEQ ID NO: 62)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNSVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-Y507R-NT (SEQ ID NO: 63)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATCGTGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y507R-AA (SEQ ID NO: 64)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNRVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Q510Y-NT (SEQ ID NO: 65)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTTATAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Q510Y-AA (SEQ ID NO: 66)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLKPYSTKKIKLNFGIPTLADGWSKSKEY
SNNAIILMRDNLYYLGIFNAKNKPDKKIIEGNTSENKGDYKKMIYNLLPGPNKMIPKVFLSSKTGVETYKPSAYILEGYKQNKH
IKSSKDFDITFCHDLIDYFKNCIAIHPEWKNFGFDFSDTSTYEDISGFYREVELQGYKIDWTYISEKDIDLLQEKGQLYLFQIY
NKDFSKKSTGNDNLHTMYLKNLFSEENLKDIVLKLNGEAEIFFRKSSIKNPIIHKKGSILVNRTYEAEEKDQFGNIQIVRKNIP
ENIYQELYKYFNDKSDKELSDEAAKLKNVVGHHEAATNIVKDYRYTYDKYFLHMPITINFKANKTGFINDRILQYIAKEKDLHV
IGIDRGERNLIYVSVIDTCGNIVEQKSFNIVNGYDYQIKLKQQEGARQIARKEWKEIGKIKEIKEGYLSLVIHEISKMVIKYNA
IIAMEDLSYGFKKGRFKVERQVYQKFETMLINKLNYLVFKDISITENGGLLKGYQLTYIPDKLKNVGHQCGCIFYVPAAYTSKI
DPTTGFVNIFKFKDLTVDAKREFIKKFDSIRYDSEKNLFCFTFDYNNFITQNTVMSKSSWSVYTYGVRIKRRFVNGRESNESDT
IDITKDMEKTLEMTDINWRDGHDLRQDIIDYEIVQHIFEIFRLTVQMRNSLSELEDRDYDRLISPVLNENNIFYDSAKAGDALP
KDADANGAYCIALKGLYEIKQITENWKEDGKFSRDKLKISNKDWFDFIQNKRYLQKSITEINELVSNYKLCSDDNIKAETYIHE
ISHILNNFEAQELKYNPEIHLVESELKASELKNVLDVIMNAFHWCSVFMTEELVDKDNNFYAELEEIYDEIYPVISLYNLVRNY
VTY
ErCas12a-Q510V-NT (SEQ ID NO: 67)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTGTGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Q510V-AA (SEQ ID NO: 68)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTVKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Q510K-NT (SEQ ID NO: 69)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTAAAAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Q510K-AA (SEQ ID NO: 70)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTKKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-P512V-NT (SEQ ID NO: 71)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGGTGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-P512V-AA (SEQ ID NO: 72)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKVYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-P512G-NT (SEQ ID NO: 73)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGGGCTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-P512G-AA (SEQ ID NO: 74)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKGYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y513S-NT (SEQ ID NO: 75)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGAGCTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y513S-AA (SEQ ID NO: 76)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPSSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-Y513R-NT (SEQ ID NO: 77)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGCGTTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y513R-AA (SEQ ID NO: 78)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPRSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y513L-NT (SEQ ID NO: 79)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGCTGTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y513L-AA (SEQ ID NO: 80)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPLSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y513G-NT (SEQ ID NO: 81)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGGGCTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y513G-AA (SEQ ID NO: 82)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPGSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRESNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-S514R-NT (SEQ ID NO: 83)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACCGTACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-S514R-AA (SEQ ID NO: 84)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYRTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-S514P-NT (SEQ ID NO: 85)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACCCGACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-S514P-AA (SEQ ID NO: 86)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYPTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-T515P-NT (SEQ ID NO: 87)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCACCGAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-T515P-AA (SEQ ID NO: 88)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSPKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-K516R-NT (SEQ ID NO: 89)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTCGTAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-K516R-AA (SEQ ID NO: 90)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTRKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-K516P-NT (SEQ ID NO: 91)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTCCGAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-K516P-AA (SEQ ID NO: 92)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTPKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-K517P-NT (SEQ ID NO: 93)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAACCGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-K517P-AA (SEQ ID NO: 94)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKPIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDESDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-I518P-NT (SEQ ID NO: 95)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGCCGAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I518P-AA (SEQ ID NO: 96)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKPKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-L520M-NT (SEQ ID NO: 97)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGATGAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-L520M-AA (SEQ ID NO: 98)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKMNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-F522A-NT (SEQ ID NO: 99)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATGCGGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-F522A-AA (SEQ ID NO: 100)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMESAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNAGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-A541V-NT (SEQ ID NO: 101)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGTGATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-A541V-AA (SEQ ID NO: 102)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNVIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-G553M-NT (SEQ ID NO: 103)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGATGATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-G553M-AA (SEQ ID NO: 104)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLMIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-N556R-NT (SEQ ID NO: 105)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCCGTGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-N556R-AA (SEQ ID NO: 106)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFRAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-N556E-NT (SEQ ID NO: 107)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCGAAGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-N556E-AA (SEQ ID NO: 108)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFEAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-D562V-NT (SEQ ID NO: 109)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGTGAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D562V-AA (SEQ ID NO: 110)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPVKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-D562K-NT (SEQ ID NO: 111)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCAAAAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D562K-AA (SEQ ID NO: 112)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPKKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-D562I-NT (SEQ ID NO: 113)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCATTAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D562I-AA (SEQ ID NO: 114)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPIKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-N583V-NT (SEQ ID NO: 115)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATGTGCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-N583V-AA (SEQ ID NO: 116)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYVLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-L585K-NT (SEQ ID NO: 117)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTAAACCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-L585K-AA (SEQ ID NO: 118)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLKPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-F653R-NT (SEQ ID NO: 119)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATCGTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-F653R-AA (SEQ ID NO: 120)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNRGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-K712R-NT (SEQ ID NO: 121)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAACGTTCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-K712R-AA (SEQ ID NO: 122)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKRSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-N716R-NT (SEQ ID NO: 123)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCCGTGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-N716R-AA (SEQ ID NO: 124)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGRDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-E731P-NT (SEQ ID NO: 125)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAACCGAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-E731P-AA (SEQ ID NO: 126)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMESAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEPNLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKEDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-N732T-NT (SEQ ID NO: 127)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAACCTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-N732T-AA (SEQ ID NO: 128)
MNNGTNNFQNFIGISSLQKTLRNALIPTETTQQFIVKNGIIKEDELRGENRQILKDIMDDYYRGFISETLSSIDDIDWTS
LFEKMEIQLKNGDNKDTLIKEQTEYRKAIHKKFANDDRFKNMFSAKLISDILPEFVIHNNNYSASEKEEKTQVIKLFSR
FATSFKDYFKNRANCFSADDISSSSCHRIVNDNAEIFFSNALVYRRIVKSLSNDDINKISGDMKDSLKEMSLEEIYSYE
KYGEFITQEGISFYNDICGKVNSFMNLYCQKNKENKNLYKLQKLHKQILCIADTSYEVPYKFESDEEVYQSVNGFLD
NISSKHIVERLRKIGDNYNGYNLDKIYIVSKFYESVSQKTYRDWETINTALEIHYNNILPGNGKSKADKVKKAVKNDLQ
KSITEINELVSNYKLCSDDNIKAETYIHEISHILNNFEAQELKYNPEIHLVESELKASELKNVLDVIMNAFHWCSVFMTE
ELVDKDNNFYAELEEIYDEIYPVISLYNLVRNYVTQKPYSTKKIKLNFGIPTLADGWSKSKEYSNNAIILMRDNLYYLGI
FNAKNKPDKKIIEGNTSENKGDYKKMIYNLLPGPNKMIPKVFLSSKTGVETYKPSAYILEGYKQNKHIKSSKDFDITFC
HDLIDYFKNCIAIHPEWKNFGFDFSDTSTYEDISGFYREVELQGYKIDWTYISEKDIDLLQEKGQLYLFQIYNKDFSKK
STGNDNLHTMYLKNLFSEETLKDIVLKLNGEAEIFFRKSSIKNPIIHKKGSILVNRTYEAEEKDQFGNIQIVRKNIPENIY
QELYKYFNDKSDKELSDEAAKLKNVVGHHEAATNIVKDYRYTYDKYFLHMPITINFKANKTGFINDRILQYIAKEKDL
HVIGIDRGERNLIYVSVIDTCGNIVEQKSFNIVNGYDYQIKLKQQEGARQIARKEWKEIGKIKEIKEGYLSLVIHEISKM
VIKYNAIIAMEDLSYGFKKGRFKVERQVYQKFETMLINKLNYLVFKDISITENGGLLKGYQLTYIPDKLKNVGHQCGCI
FYVPAAYTSKIDPTTGFVNIFKFKDLTVDAKREFIKKFDSIRYDSEKNLFCFTFDYNNFITQNTVMSKSSWSVYTYGV
RIKRRFVNGRFSNESDTIDITKDMEKTLEMTDINWRDGHDLRQDIIDYEIVQHIFEIFRLTVQMRNSLSELEDRDYDR
LISPVLNENNIFYDSAKAGDALPKDADANGAYCIALKGLYEIKQITENWKEDGKFSRDKLKISNKDWFDFIQNKRYL
ErCas12a-A744T-NT (SEQ ID NO: 129)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGACCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-A744T-AA (SEQ ID NO: 130)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGETEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-A744P-NT (SEQ ID NO: 131)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGCCGGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-A744P-AA (SEQ ID NO: 132)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEPEIFFRKSSIKNP
IIHKKGSILVNRTYEAEEKDQFGNIQIVRKNIPENIYQELYKYENDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-A744N-NT (SEQ ID NO: 133)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGAACGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-A744N-AA (SEQ ID NO: 134)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGENEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-F748A-NT (SEQ ID NO: 135)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTGCGCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-F748A-AA (SEQ ID NO: 136)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFARKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-S751A-NT (SEQ ID NO: 137)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAGCGTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-S751A-AA (SEQ ID NO: 138)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKASIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSENIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-K754G-NT (SEQ ID NO: 139)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTGGCAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-K754G-AA (SEQ ID NO: 140)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIGNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-I757W-NT (SEQ ID NO: 141)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
TGGATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I757W-AA (SEQ ID NO: 142)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
WIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-I757K-NT (SEQ ID NO: 143)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
AAAATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I757K-AA (SEQ ID NO: 144)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
KIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-E771R-NT (SEQ ID NO: 145)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATCGTGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-E771R-AA (SEQ ID NO: 146)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYRAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-N787A-NT (SEQ ID NO: 147)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGGCGATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-N787A-AA (SEQ ID NO: 148)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKAIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-A825R-NT (SEQ ID NO: 149)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCACGTACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-A825R-AA (SEQ ID NO: 150)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEARTNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-T826K-NT (SEQ ID NO: 151)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAAAAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-T826K-AA (SEQ ID NO: 152)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAKNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-T826F-NT (SEQ ID NO: 153)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCATTTAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-T826F-AA (SEQ ID NO: 154)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAFNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y832R-NT (SEQ ID NO: 155)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACCGTCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y832R-AA (SEQ ID NO: 156)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDRRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-Y836Q-NT (SEQ ID NO: 157)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTCAGGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y836Q-AA (SEQ ID NO: 158)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTQDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y836K-NT (SEQ ID NO: 159)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTAAAGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y836K-AA (SEQ ID NO: 160)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTKDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y839L-NT (SEQ ID NO: 161)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGCTGTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y839L-AA (SEQ ID NO: 162)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKLF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-F840L-NT (SEQ ID NO: 163)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACCTG
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-F840L-AA (SEQ ID NO: 164)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYL
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y907V-NT (SEQ ID NO: 165)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTGTGGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y907V-AA (SEQ ID NO: 166)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGVDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y907I-NT (SEQ ID NO: 167)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTATTGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y907I-AA (SEQ ID NO: 168)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGIDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-Q916Y-NT (SEQ ID NO: 169)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAATATGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Q916Y-AA (SEQ ID NO: 170)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQYEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-E935R-NT (SEQ ID NO: 171)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGCGTATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-E935R-AA (SEQ ID NO: 172)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKRIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-I936R-NT (SEQ ID NO: 173)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAACGTAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I936R-AA (SEQ ID NO: 174)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKERKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-K974G-NT (SEQ ID NO: 175)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCGGCGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-K974G-AA (SEQ ID NO: 176)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFGVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-K1019V-NT (SEQ ID NO: 177)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATGTGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-K1019V-AA (SEQ ID NO: 178)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDVLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-I35L/S469K-NT (SEQ ID NO: 179)
atgAATAACGGTACCAATAACTTTCAAAATTTTATCGGGATCTCCTCGTTGCAAAAAACTCTTCGTAATGCTTTAATCCCGACT
GAGACAACACAGCAGTTCCTGGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I35L/S469K-AA (SEQ ID NO: 180)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCKVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-I35L/T826F-NT (SEQ ID NO: 181)
atgAATAACGGTACCAATAACTTTCAAAATTTTATCGGGATCTCCTCGTTGCAAAAAACTCTTCGTAATGCTTTAATCCCGACT
GAGACAACACAGCAGTTCCTGGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCATTTAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I35L/T826F-AA (SEQ ID NO: 182)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVEMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYENDKSDKELSDEAAKLKNVVGHHEAAFNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-140Q/S469K-NT (SEQ ID NO: 183)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCCAGATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I40Q/S469K-AA (SEQ ID NO: 184)
MNNGTNNFQNFIGISSLQKTLRNALIPTETTQQFIVKNGQIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCKVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-I40Q/T826F-NT (SEQ ID NO: 185)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCCAGATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCATTTAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I40Q/T826F-AA (SEQ ID NO: 186)
MNNGTNNFQNFIGISSLQKTLRNALIPTETTQQFIVKNGQIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAFNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKEDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-S469K/K516P-NT (SEQ ID NO: 187)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTCCGAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-S469K/K516P-AA (SEQ ID NO: 188)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCKVEMTEELVDKDNNFYAELEEIYDEIYPVISLYNLV
RNYVTQKPYSTPKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-S469K/T826F-NT (SEQ ID NO: 189)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCATTTAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-S469K/T826F-AA (SEQ ID NO: 190)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCKVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAFNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y513G/Y832R-NT (SEQ ID NO: 191)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGGGCTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACCGTCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y513G/Y832R-AA (SEQ ID NO: 192)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPGSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDRRYTYDKYF
LHMPITINFKANKTGFINDRILQYIAKEKDLHVIGIDRGERNLIYVSVIDTCGNIVEQKSENIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-Y513G/F840L-NT (SEQ ID NO: 193)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAGCGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGGGCTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCAACAAACATTGTGAAAGACTATCGCTACACTTACGATAAGTACCTG
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-Y513G/F840L-AA (SEQ ID NO: 194)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCSVFMTEELVDKDNNFYAELEEIYDEIYPVISLYNLV
RNYVTQKPGSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDESDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAATNIVKDYRYTYDKYL
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-I35L/S469K/T826F-NT (SEQ ID NO: 195)
atgAATAACGGTACCAATAACTTTCAAAATTTTATCGGGATCTCCTCGTTGCAAAAAACTCTTCGTAATGCTTTAATCCCGACT
GAGACAACACAGCAGTTCCTGGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCATTTAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I35L/S469K/T826F-AA (SEQ ID NO: 196)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCKVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAFNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-I40Q/S469K/T826F-NT (SEQ ID NO: 197)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCCAGATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCATTTAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-I40Q/S469K/T826F-AA (SEQ ID NO: 198)
MNNGTNNFQNFIGISSLQKTLRNALIPTETTQQFIVKNGQIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCKVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAFNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL
ErCas12a-S469K/K516P/T826F-NT (SEQ ID NO: 199)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTCCGAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCATTTAACATTGTGAAAGACTATCGCTACACTTACGATAAGTACTTT
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-S469K/K516P/T826F-AA (SEQ ID NO: 200)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLESRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCKVFMTEELVDKDNNFYAELEEIYDEIYPVISLYNLV
RNYVTQKPYSTPKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAFNIVKDYRYTYDKYF
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-S469K/T826F/F840L-NT (SEQ ID NO: 201)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCATTTAACATTGTGAAAGACTATCGCTACACTTACGATAAGTACCTG
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-S469K/T826F/F840L-AA (SEQ ID NO: 202)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCKVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAFNIVKDYRYTYDKYL
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-D303R/S469K/T826F/F840L-NT (SEQ ID NO: 203)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCCGTGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCTTCCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCATTTAACATTGTGAAAGACTATCGCTACACTTACGATAAGTACCTG
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D303R/S469K/T826F/F840L-AA (SEQ ID NO: 204)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCESADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESREEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCKVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAFNIVKDYRYTYDKYL
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-F465R/S469K/T826F/F840L-NT (SEQ ID NO: 205)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCGACGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCCGTCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCATTTAACATTGTGAAAGACTATCGCTACACTTACGATAAGTACCTG
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-F465R/S469K/T826F/F840L-AA (SEQ ID NO: 206)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESDEEVYQSVNGFLDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNARHWCKVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAFNIVKDYRYTYDKYL
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-D303R/F465R/S469K/T826F/F840L-NT (SEQ ID NO: 207)
atgAATAACGGTACCAATAACTTTCAAAATTTTATCGGGATCTCCTCGTTGCAAAAAACTCTTCGTAATGCTTTAATCCCGACT
GAGACAACACAGCAGTTCATCGTTAAAAACGGCATCATTAAAGAAGATGAGCTGCGCGGCGAGAACCGTCAGATTTTAAAGGAC
ATCATGGACGACTATTACCGTGGGTTCATCTCAGAAACATTAAGCAGCATCGATGATATCGACTGGACCTCACTGTTTGAGAAG
ATGGAGATTCAACTTAAGAATGGAGACAACAAAGACACTTTAATTAAGGAGCAGACGGAATATCGTAAAGCTATCCATAAAAAA
TTTGCAAACGATGACCGTTTCAAAAATATGTTTTCCGCAAAATTGATTAGCGACATTTTACCTGAATTCGTAATCCATAACAAC
AACTATTCCGCATCTGAAAAAGAGGAGAAGACCCAAGTTATCAAACTTTTTTCACGTTTCGCGACATCTTTTAAAGACTACTTT
AAGAATCGTGCGAATTGTTTCAGTGCAGATGATATTTCGTCTAGTTCATGTCACCGTATCGTGAATGACAACGCTGAGATTTTC
TTTTCTAATGCGTTGGTGTATCGCCGCATCGTAAAGTCCTTGTCGAATGATGATATCAACAAAATTAGTGGTGACATGAAAGAC
TCCCTGAAAGAAATGTCATTAGAGGAGATTTACAGCTACGAAAAATACGGAGAGTTCATCACTCAGGAAGGTATCTCCTTTTAC
AACGATATTTGCGGCAAAGTTAATAGTTTCATGAATCTGTATTGTCAGAAGAATAAGGAAAACAAGAATCTGTACAAGTTACAA
AAGCTTCATAAACAGATCTTGTGCATCGCCGACACGTCTTATGAAGTCCCGTATAAATTTGAAAGCCGTGAAGAAGTATATCAG
AGTGTGAATGGATTCTTGGACAATATCTCATCAAAACACATTGTTGAACGTTTGCGCAAAATCGGCGACAACTATAACGGATAC
AACTTGGATAAAATTTATATCGTATCAAAATTTTATGAATCAGTATCCCAAAAGACTTACCGTGATTGGGAGACTATTAATACG
GCGTTAGAAATCCATTACAACAACATTCTGCCGGGTAACGGTAAATCGAAAGCGGACAAAGTGAAGAAGGCGGTTAAGAACGAT
CTTCAGAAGTCTATCACGGAGATTAATGAGCTGGTTTCCAATTACAAGTTGTGCTCGGATGATAACATCAAAGCTGAAACCTAT
ATCCATGAAATTTCTCATATTCTTAATAACTTCGAGGCACAAGAATTAAAATATAACCCAGAAATTCATCTGGTTGAGAGCGAA
CTTAAAGCTTCAGAATTAAAAAATGTTTTGGACGTAATTATGAACGCCCGTCATTGGTGCAAAGTGTTTATGACAGAGGAGTTA
GTAGATAAGGATAACAACTTTTATGCAGAATTGGAAGAGATCTATGATGAGATCTATCCAGTGATTTCACTGTATAACTTAGTC
CGTAATTACGTCACTCAGAAGCCGTACTCAACTAAAAAGATCAAGTTAAATTTTGGAATTCCGACATTGGCGGATGGTTGGAGT
AAGTCTAAGGAATACTCAAATAACGCTATTATCTTAATGCGTGACAACCTGTATTACTTGGGTATTTTCAATGCCAAAAATAAG
CCCGATAAAAAAATCATTGAAGGGAATACTTCAGAAAATAAGGGAGATTACAAAAAAATGATTTATAACCTTTTGCCAGGCCCA
AACAAAATGATCCCCAAGGTGTTTTTGAGTAGTAAGACGGGAGTAGAAACATATAAGCCATCAGCGTACATTCTTGAAGGGTAC
AAACAAAATAAACACATCAAGAGCAGCAAGGACTTTGACATTACATTTTGTCATGACTTAATCGACTACTTTAAAAATTGCATT
GCCATTCACCCTGAGTGGAAAAATTTTGGTTTCGACTTCAGTGATACCTCTACCTATGAAGATATCTCTGGTTTTTATCGTGAG
GTAGAACTTCAGGGTTACAAGATCGATTGGACGTACATTTCCGAGAAAGACATTGATCTTTTACAAGAGAAGGGTCAGCTTTAC
TTATTTCAGATCTACAATAAAGACTTTTCGAAAAAATCCACGGGCAACGACAACCTTCATACAATGTACTTAAAGAACCTGTTC
TCTGAAGAAAACTTGAAAGATATCGTGCTTAAATTGAATGGCGAGGCCGAGATTTTTTTCCGTAAAAGCTCGATTAAGAATCCC
ATCATCCACAAGAAAGGCAGCATTCTGGTAAATCGTACCTATGAAGCGGAGGAAAAGGACCAATTTGGTAATATTCAGATTGTC
CGTAAGAACATCCCTGAAAATATCTATCAAGAATTATATAAATATTTCAACGATAAGTCGGACAAGGAACTTAGCGATGAAGCC
GCGAAGTTGAAAAATGTTGTTGGTCACCACGAAGCAGCATTTAACATTGTGAAAGACTATCGCTACACTTACGATAAGTACCTG
TTACATATGCCTATTACGATTAACTTTAAGGCAAATAAGACAGGTTTTATCAACGACCGTATCCTGCAATATATTGCAAAAGAG
AAGGACCTGCATGTCATTGGGATTGATCGCGGTGAACGCAATCTGATTTACGTGAGTGTTATTGATACCTGCGGTAATATTGTT
GAGCAAAAGTCTTTTAATATCGTTAATGGTTACGACTACCAAATTAAGCTTAAACAACAGGAAGGTGCCCGCCAGATCGCCCGT
AAGGAGTGGAAGGAAATCGGAAAAATTAAGGAAATCAAAGAGGGATATCTGAGCTTGGTTATTCATGAAATCTCAAAGATGGTT
ATTAAGTACAACGCCATCATCGCTATGGAAGACTTATCGTATGGGTTTAAAAAGGGTCGTTTCAAAGTTGAGCGCCAGGTCTAC
CAAAAGTTTGAAACGATGCTTATTAATAAATTAAACTACTTGGTTTTCAAAGATATCTCTATTACCGAGAATGGAGGTTTACTG
AAAGGATACCAGTTAACGTATATCCCCGATAAGCTTAAAAACGTGGGCCATCAGTGTGGCTGTATTTTCTATGTGCCCGCGGCA
TATACATCAAAAATTGACCCGACGACGGGTTTCGTTAATATCTTCAAGTTCAAAGATCTGACAGTTGATGCAAAACGCGAATTC
ATTAAGAAGTTCGATAGTATTCGCTATGACTCCGAGAAAAATTTGTTTTGCTTCACTTTTGACTACAATAACTTTATTACTCAG
AATACAGTCATGAGTAAAAGCTCCTGGTCGGTTTACACGTACGGGGTACGCATCAAACGTCGTTTTGTGAATGGTCGTTTTAGT
AATGAGTCGGATACAATTGATATTACCAAGGATATGGAGAAGACTCTGGAAATGACAGACATCAATTGGCGTGATGGACATGAT
TTGCGTCAGGACATTATCGACTATGAGATTGTGCAGCATATTTTCGAAATTTTTCGCCTGACGGTGCAAATGCGTAATTCCTTA
AGCGAACTGGAAGATCGCGACTATGACCGCTTAATTTCTCCTGTGCTGAATGAGAACAACATCTTCTATGACTCAGCGAAAGCC
GGGGACGCTTTGCCAAAGGATGCAGATGCTAACGGTGCATATTGTATTGCTCTGAAAGGCCTTTACGAGATCAAACAAATTACT
GAAAACTGGAAGGAAGATGGAAAGTTTTCCCGTGATAAATTGAAGATCTCTAATAAGGACTGGTTTGACTTTATCCAAAATAAA
CGTTATTTA
ErCas12a-D303R/F465R/S469K/T826F/F840L-AA (SEQ ID NO: 208)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESREEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNARHWCKVFMTEELVDKDNNFYAELEEIYDEIYPVISLYNLV
RNYVTQKPYSTKKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAFNIVKDYRYTYDKYL
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTFDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWFDFIQNK
RYL
ErCas12a-D303R/S469K/K516P/T826F/F840L-NT (SEQ ID NO: 209)
atgAATAACGGCACCAACAACTTCCAGAACTTTATTGGTATTAGCAGCCTGCAGAAAACCCTGCGTAATGCACTGATTCCGACC
GAAACCACACAGCAGTTTATTGTGAAAAACGGCATCATCAAAGAAGATGAACTTCGCGGAGAAAATCGCCAGATTCTGAAAGAT
ATCATGGATGATTATTATCGCGGTTTTATCAGCGAAACCCTGAGCAGCATTGATGATATTGATTGGACCAGCCTGTTCGAGAAA
ATGGAAATCCAGCTGAAAAATGGCGATAACAAAGACACCCTGATTAAAGAACAGACGGAATATCGTAAAGCCATCCACAAAAAG
TTCGCCAATGATGACCGCTTTAAAAACATGTTCAGCGCAAAACTGATTAGCGATATTCTGCCGGAATTTGTGATCCACAATAAT
AACTATAGCGCCAGCGAGAAAGAAGAGAAAACCCAGGTTATTAAACTGTTTAGCCGTTTTGCGACCAGCTTCAAAGATTACTTC
AAAAATCGTGCCAACTGCTTCAGCGCAGATGATATTAGCAGTAGCAGCTGTCATCGTATTGTGAATGATAACGCCGAAATCTTT
TTCAGCAATGCCCTGGTTTATCGTCGCATTGTTAAAAGCCTGAGCAACGACGACATCAACAAAATCAGCGGTGATATGAAAGAT
AGCCTGAAAGAAATGAGCCTGGAAGAAATCTATAGCTACGAAAAATATGGCGAGTTCATTACCCAAGAAGGCATCAGCTTTTAT
AACGATATTTGCGGCAAAGTGAACAGCTTTATGAATCTGTATTGCCAGAAAAACAAAGAGAACAAAAACCTGTACAAACTGCAG
AAACTGCATAAGCAGATTCTGTGTATTGCAGATACCAGCTATGAAGTGCCGTATAAATTCGAAAGTCGTGAAGAAGTGTACCAG
AGCGTTAATGGTTTTCTGGATAACATCAGCAGCAAACACATTGTTGAACGTCTGCGTAAAATTGGCGACAACTATAATGGCTAT
AACCTGGACAAAATCTACATCGTGAGCAAATTCTATGAAAGCGTGAGCCAGAAAACATATCGTGATTGGGAAACAATTAATACC
GCCTTGGAGATCCACTACAATAACATTCTGCCTGGTAATGGTAAAAGCAAAGCCGACAAAGTTAAAAAGGCCGTGAAAAATGAT
CTGCAGAAATCCATTACCGAAATCAACGAACTGGTCAGCAACTATAAACTGTGTTCCGATGATAATATCAAGGCCGAAACCTAT
ATTCACGAAATCTCCCATATCCTGAACAACTTTGAAGCACAAGAGCTGAAATATAACCCGGAAATTCATCTGGTTGAAAGCGAA
CTGAAAGCCAGTGAACTGAAAAACGTTCTGGATGTGATTATGAACGCCTTTCATTGGTGTAAAGTTTTTATGACCGAAGAACTG
GTGGATAAGGATAACAACTTTTATGCCGAACTGGAAGAAATCTACGATGAAATTTATCCGGTGATCAGCCTGTATAATCTGGTG
CGTAATTATGTGACCCAGAAACCGTATAGCACCCCGAAGATCAAACTGAACTTTGGTATTCCGACACTGGCAGATGGTTGGAGC
AAAAGTAAAGAATATAGCAACAACGCCATTATCCTGATGCGCGATAATCTGTACTATCTGGGTATCTTTAACGCCAAAAACAAA
CCGGACAAGAAAATCATTGAAGGCAATACCAGCGAAAACAAAGGCGACTATAAGAAAATGATTTATAATCTGCTGCCTGGTCCG
AACAAAATGATTCCGAAAGTTTTTCTGAGCAGTAAGACCGGTGTGGAAACATATAAACCGAGCGCATATATTCTGGAAGGCTAC
AAACAGAACAAGCATATCAAAAGCAGCAAAGACTTTGATATCACCTTTTGCCATGATCTGATCGACTATTTCAAAAACTGCATT
GCCATTCATCCGGAATGGAAAAATTTCGGCTTTGATTTCAGCGATACCAGCACCTATGAAGATATTAGCGGTTTTTATCGTGAA
GTGGAACTGCAGGGCTATAAAATCGATTGGACCTATATTAGCGAGAAGGATATTGATCTGCTGCAAGAAAAAGGTCAGCTGTAC
CTGTTTCAGATCTACAACAAAGACTTCAGCAAGAAAAGCACCGGCAATGATAATCTGCATACCATGTATCTGAAGAACCTGTTT
AGCGAAGAGAACCTGAAAGACATTGTGCTGAAACTGAATGGTGAAGCCGAGATTTTCTTTCGCAAATCCAGCATTAAAAACCCG
ATCATCCATAAAAAGGGTAGCATTCTGGTTAATCGTACGTATGAAGCCGAAGAGAAAGATCAGTTTGGCAACATTCAGATTGTG
CGTAAAAACATCCCGGAAAACATCTATCAAGAGCTGTACAAATACTTTAACGATAAAAGCGATAAAGAACTGTCCGATGAAGCA
GCGAAACTGAAGAATGTTGTTGGTCATCATGAAGCCGCATTTAACATTGTGAAAGATTATCGTTACACCTATGATAAATACCTG
CTGCACATGCCGATCACCATCAACTTTAAAGCAAATAAGACGGGTTTCATCAACGATCGCATTCTGCAGTATATCGCCAAAGAA
AAAGACCTGCATGTGATTGGTATTGATCGTGGTGAACGCAATCTGATTTATGTGAGCGTTATTGATACCTGCGGCAATATTGTG
GAACAGAAAAGCTTTAATATCGTGAACGGCTATGACTACCAGATCAAGCTGAAACAGCAAGAAGGTGCACGTCAGATTGCACGT
AAAGAATGGAAAGAAATCGGCAAAATCAAAGAAATTAAAGAAGGCTATCTGAGCCTGGTGATTCATGAGATTAGCAAAATGGTG
ATCAAGTATAACGCCATCATTGCCATGGAAGATCTGAGTTATGGTTTTAAGAAAGGTCGCTTTAAGGTGGAACGTCAGGTGTAT
CAGAAATTTGAAACCATGCTGATCAACAAACTGAATTATCTGGTGTTCAAGGACATCAGCATTACGGAAAATGGTGGTCTGCTG
AAAGGTTATCAGCTGACGTATATTCCGGATAAGCTTAAAAATGTGGGTCATCAGTGTGGCTGCATCTTTTATGTTCCGGCAGCC
TATACCAGCAAAATTGATCCGACCACCGGTTTTGTGAACATCTTCAAATTCAAAGATCTGACCGTTGATGCCAAACGCGAATTC
ATCAAAAAGTTTGATAGCATCCGTTACGACTCCGAGAAAAACCTTTTCTGCTTTACCTTCGACTACAACAACTTTATTACCCAG
AATACGGTGATGAGCAAAAGCAGCTGGTCAGTTTATACCTATGGTGTGCGTATTAAACGTCGCTTTGTGAATGGTCGTTTTAGC
AATGAAAGCGACACCATCGATATCACCAAAGATATGGAAAAGACCCTGGAAATGACCGATATTAACTGGCGTGATGGTCATGAT
CTGCGTCAGGATATTATCGATTATGAAATCGTGCAGCACATCTTTGAAATCTTTCGTCTGACCGTGCAGATGCGTAATAGCCTG
AGCGAGCTGGAAGATCGCGATTATGATCGTCTGATTAGTCCGGTTCTGAACGAAAACAACATCTTCTATGATAGCGCAAAAGCC
GGTGATGCACTGCCGAAAGATGCCGATGCAAATGGTGCATATTGTATTGCACTGAAAGGCCTGTATGAAATCAAGCAGATTACC
GAGAATTGGAAAGAGGATGGTAAATTCAGCCGTGATAAACTGAAAATTAGCAACAAGGATTGGTTCGATTTCATTCAGAACAAA
CGCTACCTG
ErCas12a-D303R/S469K/K516P/T826F/F840L-AA (SEQ ID NO: 210)
MNNGTNNFQNFIGISSLQKTLRNALIPTETTQQFIVKNGIIKEDELRGENRQILKDIMDDYYRGFISETLSSIDDIDWTSLFEK
MEIQLKNGDNKDTLIKEQTEYRKAIHKKFANDDRFKNMFSAKLISDILPEFVIHNNNYSASEKEEKTQVIKLFSRFATSFKDYF
KNRANCFSADDISSSSCHRIVNDNAEIFFSNALVYRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFESREEVYQSVNGELDNISSKHIVERLRKIGDNYNGY
NLDKIYIVSKFYESVSQKTYRDWETINTALEIHYNNILPGNGKSKADKVKKAVKNDLQKSITEINELVSNYKLCSDDNIKAETY
IHEISHILNNFEAQELKYNPEIHLVESELKASELKNVLDVIMNAFHWCKVFMTEELVDKDNNFYAELEEIYDEIYPVISLYNLV
RNYVTQKPYSTPKIKLNFGIPTLADGWSKSKEYSNNAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLIDYFKNCIAIHPEWKNFGFDFSDTSTYEDISGFYRE
VELQGYKIDWTYISEKDIDLLQEKGQLYLFQIYNKDFSKKSTGNDNLHTMYLKNLFSEENLKDIVLKLNGEAEIFFRKSSIKNP
IIHKKGSILVNRTYEAEEKDQFGNIQIVRKNIPENIYQELYKYFNDKSDKELSDEAAKLKNVVGHHEAAFNIVKDYRYTYDKYL
LHMPITINFKANKTGFINDRILQYIAKEKDLHVIGIDRGERNLIYVSVIDTCGNIVEQKSFNIVNGYDYQIKLKQQEGARQIAR
KEWKEIGKIKEIKEGYLSLVIHEISKMVIKYNAIIAMEDLSYGFKKGRFKVERQVYQKFETMLINKLNYLVFKDISITENGGLL
KGYQLTYIPDKLKNVGHQCGCIFYVPAAYTSKIDPTTGFVNIFKFKDLTVDAKREFIKKFDSIRYDSEKNLFCFTEDYNNFITQ
NTVMSKSSWSVYTYGVRIKRRFVNGRFSNESDTIDITKDMEKTLEMTDINWRDGHDLRQDIIDYEIVQHIFEIFRLTVQMRNSL
SELEDRDYDRLISPVLNENNIFYDSAKAGDALPKDADANGAYCIALKGLYEIKQITENWKEDGKFSRDKLKISNKDWEDFIQNK
RYL

1. An isolated polypeptide comprising an Eubacterium rectale Cas12a (ERCAS12A) mutant having one or more amino acid substitutions introduced into a wild-type ERCAS12A polypeptide sequence of SEQ ID NO: 2, where the ERCAS12A mutant polypeptide provides an improvement in nuclease activity as compared to the wild-type ERCAS12A polypeptide.

2. The isolated polypeptide of claim 1, wherein the ERCAS12A mutant has 98% to 99% identity to a polypeptide sequence selected from the even numbered sequences of SEQ ID NO: 4-210.

3. The isolated polypeptide of claim 1, wherein the ERCAS12A mutant has a polypeptide sequence selected from the even numbered sequences of SEQ ID NO: 4-210.

4. The isolated polypeptide of claim 1, wherein the ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353,444, 465, 469, 470,498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2.

5. The isolated polypeptide of claim 1, wherein the ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q51OK, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2.

6. The isolated polypeptide of claim 1, wherein the ERCAS12A mutant is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).

7. The isolated polypeptide of claim 1, wherein the ERCAS12A mutant is selected from D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).

8. An isolated polynucleotide sequence encoding an Eubacterium rectale Cas12a (ERCAS12A) mutant having one or more amino acid substitutions introduced into a wild-type ERCAS12A polypeptide sequence of SEQ ID NO: 2, where the ERCAS12A variant polypeptide provides an improvement in nuclease activity as compared to the wild-type ERCAS12A polypeptide.

9. The isolated polynucleotide sequence of claim 8, wherein the encoded ERCAS12A mutant has 98% to 99% identity to a polypeptide sequence selected from the even numbered sequences of SEQ ID NO: 4-210.

10. The isolated polynucleotide sequence of claim 8, wherein the encoded ERCAS12A mutant has a polypeptide sequence selected from the even numbered sequences of SEQ ID NO: 4-210.

11. The isolated polynucleotide sequence of claim 8, wherein the ERCAS12A mutant polynucleotide sequence has 95% to 99% identity to a nucleotide sequence selected from the odd numbered sequences of SEQ ID NO: 3-209.

12. The isolated polynucleotide sequence of claim 8, wherein the ERCAS12A mutant polynucleotide has a nucleotide sequence selected from the odd numbered sequences of SEQ ID NO: 3-209.

13. The isolated polynucleotide sequence of claim 8, wherein the encoded ERCAS12A mutant comprises one or more amino acid substitution at positions 18, 35, 39, 39, 40, 56, 56, 59, 59, 158, 165, 181, 290, 291, 296, 297, 297, 297, 303, 303, 305, 307, 353, 444, 465, 469, 470, 498, 507, 507, 507, 510, 510, 510, 512, 512, 513, 513, 513, 513, 514, 514, 515, 516, 516, 517, 518, 520, 522, 541, 553, 556, 556, 562, 562, 562, 583, 585, 653, 712, 716, 731, 732, 744, 744, 744, 748, 751, 754, 757, 757, 771, 787, 825, 826, 826, 832, 836, 836, 839, 840, 907, 907, 916, 935, 936, 974, or 1019 of the wild-type ERCAS12A polypeptide sequence of SEQ ID NO: 2.

14. The isolated polynucleotide sequence of claim 8, wherein the encoded ERCAS12A mutant comprises one or more amino acid substitutions selected from Q18V, 135L, G39K, G39Q, 140Q, D56P, D56R, D59A, D59R, S158G, K165Y, S181V, A290K, D291N, V296R, P297F, P297R, P297V, D303C, D303R, E305K, Y307K, Q353R, L444W, F465R, S469K, V470K, 1498K, Y507R, Y507S, Y507W, Q51OK, Q510V, Q510Y, P512G, P512V, Y513G, Y513L, Y513R, Y513S, S514P, S514R, T515P, K516P, K516R, K517P, 1518P, L520M, F522A, A541V, G553M, N556E, N556R, D5621, D562K, D562V, N583V, L585K, F653R, K712R, N716R, E731P, N732T, A744N, A744P, A744T, F748A, S751A, K754G, 1757K, 1757W, E771R, N787A, A825R, T826F, T826K, Y832R, Y836K, Y836Q, Y839L, F840L, Y9071, Y907V, Q916Y, E935R, 1936R, K974G, K1019V, or combinations thereof of the wild-type ERCAS12A polypeptide sequence of SEQ ID NO: 2.

15. The isolated polynucleotide sequence of claim 8, wherein encoded ERCAS12A mutant polynucleotide is selected from is selected from: Q18V (SEQ ID NO: 4); 135L (SEQ ID NO: 6); G39Q (SEQ ID NO: 8); G39K (SEQ ID NO: 10); 140Q (SEQ ID NO: 12); D56R (SEQ ID NO: 14); D56P (SEQ ID NO: 16); D59R (SEQ ID NO: 18); D59A (SEQ ID NO: 20); S158G (SEQ ID NO: 22); K165Y (SEQ ID NO: 24); S181V (SEQ ID NO: 26); A290K (SEQ ID NO: 28); D291N (SEQ ID NO: 30); V296R (SEQ ID NO: 32); P297V (SEQ ID NO: 34); P297R (SEQ ID NO: 36); P297F (SEQ ID NO: 38); D303R (SEQ ID NO: 40); D303C (SEQ ID NO: 42); E305K (SEQ ID NO: 44); Y307K (SEQ ID NO: 46); Q353R (SEQ ID NO: 48); L444W (SEQ ID NO: 50); F465R (SEQ ID NO: 52); S469K (SEQ ID NO: 54); V470K (SEQ ID NO: 56); 1498K (SEQ ID NO: 58); Y507W (SEQ ID NO: 60); Y507S (SEQ ID NO: 62); Y507R (SEQ ID NO: 64); Q510Y (SEQ ID NO: 66); Q510V (SEQ ID NO: 68); Q510K (SEQ ID NO: 70); P512V (SEQ ID NO: 72); P512G (SEQ ID NO: 74); Y513S (SEQ ID NO: 76); Y513R (SEQ ID NO: 78); Y513L (SEQ ID NO: 80); Y513G (SEQ ID NO: 82); S514R (SEQ ID NO: 84); S514P (SEQ ID NO: 86); T515P (SEQ ID NO: 88); K516R (SEQ ID NO: 90); K516P (SEQ ID NO: 92); K517P (SEQ ID NO: 94); 1518P (SEQ ID NO: 96); L520M (SEQ ID NO: 98); F522A (SEQ ID NO: 100); A541V (SEQ ID NO: 102); G553M (SEQ ID NO: 104); N556R (SEQ ID NO: 106); N556E (SEQ ID NO: 108); D562V (SEQ ID NO: 110); D562K (SEQ ID NO: 112); D5621 (SEQ ID NO: 114); N583V (SEQ ID NO: 116); L585K (SEQ ID NO: 118); F653R (SEQ ID NO: 120); K712R (SEQ ID NO: 122); N716R (SEQ ID NO: 124); E731P (SEQ ID NO: 126); N732T (SEQ ID NO: 128); A744T (SEQ ID NO: 130); A744P (SEQ ID NO: 132); A744N (SEQ ID NO: 134); F748A (SEQ ID NO: 136); S751A (SEQ ID NO: 138); K754G (SEQ ID NO: 140); 1757W (SEQ ID NO: 142); 1757K (SEQ ID NO: 144); E771R (SEQ ID NO: 146); N787A (SEQ ID NO: 148); A825R (SEQ ID NO: 150); T826K (SEQ ID NO: 152); T826F (SEQ ID NO: 154); Y832R (SEQ ID NO: 156); Y836Q (SEQ ID NO: 158); Y836K (SEQ ID NO: 160); Y839L (SEQ ID NO: 162); F840L (SEQ ID NO: 164); Y907V (SEQ ID NO: 166); Y9071 (SEQ ID NO: 168); Q916Y (SEQ ID NO: 170); E935R (SEQ ID NO: 172); 1936R (SEQ ID NO: 174); K974G (SEQ ID NO: 176); K1019V (SEQ ID NO: 178); 135L/S469K (SEQ ID NO: 180); 135L/T826F (SEQ ID NO: 182); 140Q/S469K (SEQ ID NO: 184); 140Q/T826F (SEQ ID NO: 186); S469K/K516P (SEQ ID NO: 188); S469K/T826F (SEQ ID NO: 190); Y513G/Y832R (SEQ ID NO: 192); Y513G/F840L (SEQ ID NO: 194); 135L/S469K/T826F (SEQ ID NO: 196); 140Q/S469K/T826F (SEQ ID NO: 198); S469K/K516P/T826F (SEQ ID NO: 200); S469K/T826F/F840L (SEQ ID NO: 202); D303R/S469K/T826F/F840L (SEQ ID NO: 204); F465R/S469K/T826F/F840L (SEQ ID NO: 206); D303R/F465R/S469K/T826F/F840L (SEQ ID NO: 208); or D303R/S469K/K516P/T826F/F840L (SEQ ID NO: 210).

16. The isolated polynucleotide sequence of claim 8, wherein the ERCAS12A mutant poly nucleotide is selected from Q18V (SEQ ID NO: 3); 135L (SEQ ID NO: 5); G39Q (SEQ ID NO: 7); G39K (SEQ ID NO: 9); 140Q (SEQ ID NO: 11); D56R (SEQ ID NO: 13); D56P (SEQ ID NO: 15); D59R (SEQ ID NO: 17); D59A (SEQ ID NO: 19); S158G (SEQ ID NO: 21); K165Y (SEQ ID NO: 23); S181V (SEQ ID NO: 25); A290K (SEQ ID NO: 27); D291N (SEQ ID NO: 29); V296R (SEQ ID NO: 31); P297V (SEQ ID NO: 33); P297R (SEQ ID NO: 35); P297F (SEQ ID NO: 37); D303R (SEQ ID NO: 39); D303C (SEQ ID NO: 41); E305K (SEQ ID NO: 43); Y307K (SEQ ID NO: 45); Q353R (SEQ ID NO: 47); L444W (SEQ ID NO: 49); F465R (SEQ ID NO: 51); S469K (SEQ ID NO: 53); V470K (SEQ ID NO: 55); 1498K (SEQ ID NO: 57); Y507W (SEQ ID NO: 59); Y507S (SEQ ID NO: 61); Y507R (SEQ ID NO: 63); Q510Y (SEQ ID NO: 65); Q510V (SEQ ID NO: 67); Q510K (SEQ ID NO: 69); P512V (SEQ ID NO: 71); P512G (SEQ ID NO: 73); Y513S (SEQ ID NO: 75); Y513R (SEQ ID NO: 77); Y513L (SEQ ID NO: 79); Y513G (SEQ ID NO: 81); S514R (SEQ ID NO: 83); S514P (SEQ ID NO: 85); T515P (SEQ ID NO: 87); K516R (SEQ ID NO: 89); K516P (SEQ ID NO: 91); K517P (SEQ ID NO: 93); 1518P (SEQ ID NO: 95); L520M (SEQ ID NO: 97); F522A (SEQ ID NO: 99); A541V (SEQ ID NO: 101); G553M (SEQ ID NO: 103); N556R (SEQ ID NO: 105); N556E (SEQ ID NO: 107); D562V (SEQ ID NO: 109); D562K (SEQ ID NO: 111); D5621 (SEQ ID NO: 113); N583V (SEQ ID NO: 115); L585K (SEQ ID NO: 117); F653R (SEQ ID NO: 119); K712R (SEQ ID NO: 121); N716R (SEQ ID NO: 123); E731P (SEQ ID NO: 125); N732T (SEQ ID NO: 127); A744T (SEQ ID NO: 129); A744P (SEQ ID NO: 131); A744N (SEQ ID NO: 132); F748A (SEQ ID NO: 135); S751A (SEQ ID NO: 137); K754G (SEQ ID NO: 139); 1757W (SEQ ID NO: 141); 1757K (SEQ ID NO: 143); E771R (SEQ ID NO: 145); N787A (SEQ ID NO: 147); A825R (SEQ ID NO: 149); T826K (SEQ ID NO: 151); T826F (SEQ ID NO: 153); Y832R (SEQ ID NO: 155); Y836Q (SEQ ID NO: 157); Y836K (SEQ ID NO: 159); Y839L (SEQ ID NO: 161); F840L (SEQ ID NO: 163); Y907V (SEQ ID NO: 165); Y9071 (SEQ ID NO: 167); Q916Y (SEQ ID NO: 169); E935R (SEQ ID NO: 171); 1936R (SEQ ID NO: 173); K974G (SEQ ID NO: 175); K1019V (SEQ ID NO: 177); 135L/S469K (SEQ ID NO: 179); 135L/T826F (SEQ ID NO: 181); 140Q/S469K (SEQ ID NO: 183); 140Q/T826F (SEQ ID NO: 185); S469K/K516P (SEQ ID NO: 187); S469K/T826F (SEQ ID NO: 189); Y513G/Y832R (SEQ ID NO: 191); Y513G/F840L (SEQ ID NO: 193); 135L/S469K/T826F (SEQ ID NO: 195); 140Q/S469K/T826F (SEQ ID NO: 197); S469K/K516P/T826F (SEQ ID NO: 199); S469K/T826F/F840L (SEQ ID NO: 201); D303R/S469K/T826F/F840L (SEQ ID NO: 203); F465R/S469K/T826F/F840L (SEQ ID NO: 205); D303R/F465R/S469K/T826F/F840L (SEQ ID NO: 207); or D303R/S469K/K516P/T826F/F840L (SEQ ID NO: 209).

17. The isolated polynucleotide sequence of claim 8 wherein the ERCAS12A mutant polynucleotide is selected from is selected from: D303R/S469K/T826F/F840L (SEQ ID NO: 203); F465R/S469K/T826F/F840L (SEQ ID NO: 205); D303R/F465R/S469K/T826F/F840L (SEQ ID NO: 207); or D303R/S469K/K516P/T826F/F840L (SEQ ID NO: 209).

18. A vector or plasmid comprising the polynucleotide sequence of claim 8.

19-31. (canceled)

32. A method for performing gene editing comprising:
contacting a candidate editing target site locus with a CRISPR ribonucleoprotein complex comprising a crRNA and an ERCAS12A mutant polypeptide comprising having one or more amino acid substitutions introduced into a wild-type ERCAS12A polypeptide sequence of SEQ ID NO: 2, where the ERCAS12A mutant provides improved gene editing as compared to the wild-type ERCAS12A polypeptide.

33. The method of claim 32, wherein the ERCAS12A mutant has a polypeptide sequence selected from the even numbered sequences of SEQ ID NO: 4-210.

34-45. (canceled)
